(19) United States
(12) Patent Application Publication
(10) Pub. No.: US 2026/0357232 A1
(43) Pub. Date: Nov. 12, 2026

(54) REVERSE TRANSCRIPTASE-MEDIATED GENETIC EDITING OF TRANSTHYRETIN (TTR) AND USES THEREOF

(71) Applicant: ARBOR BIOTECHNOLOGIES, INC., Cambridge, MA (US)

(72) Inventors: Jeffrey Raymond HASWELL, Cambridge, MA (US); Fu-Kai HSIEH, Cambridge, MA (US); Noah Michael JAKIMO, Cambridge, MA (US)

(21) Appl. No.: 19/134,086

(22) PCT Filed: Nov. 29, 2023
(86) PCT No.: PCT/US2023/081560

§ 371 Date: May 29, 2025

Related U.S. Application Data
(60) Provisional application No. 63/385,604, filed on Nov. 30, 2022.

(51) Int. Cl. C12N 9/22; A61K 9/00; A61K 48/00; A61P 25/28; C12N 9/12; C12N 15/10; C12N 15/113; C12N 15/52; C12N 15/62

(52) U.S. Cl. CPC C12N 9/226; A61K 9/0019; A61K 48/0025; A61K 48/005; A61K 48/0075; A61K 48/0083; A61P 25/28; C12N 9/1276; C12N 15/102; C12N 15/113; C12N 15/52; C12N 15/62; C12Y 207/07049; C12N 2310/20; C12N 2310/315; C12N 2310/317; C12N 2310/321

(57) ABSTRACT

A system for genetic editing of a transthyretin (TTR) gene, comprising (a) a Cas12i2 polypeptide or a first nucleic acid encoding the Cas12i2 polypeptide; (b) a reverse transcriptase (RT) polypeptide or a second nucleic acid encoding the RT polypeptide; (c) an RNA guide or a third nucleic acid encoding the RNA guide, and (d) a reverse transcription donor RNA (RT donor RNA) or a fourth nucleic acid encoding the RT donor RNA. Also provided herein are methods for editing a TTR gene using the gene editing system disclosed herein and/or for treating diseases associated with the TTR gene, for example, amyloidogenic transthyretin.

### CROSS REFERENCE TO RELATED APPLICATIONS

This application claims the benefit under 35 U.S.C. § 119(e) of U.S. Provisional Application No. 63/385,604, filed Nov. 30, 2023, the contents of which are incorporated by reference herein in its entirety.

### SEQUENCE LISTING

The instant application contains a Sequence Listing which has been filed electronically in XML format and is hereby incorporated by reference in its entirety. Said XML copy, created on Nov. 20, 2023, is named 063586-509001WO_SL.xml and is 787,348 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY

The present disclosure is based, at least in part, on the development of a gene editing system for introducing desired mutations into the transthyretin (TTR) gene, which is mediated by a Type V CRISPR nuclease polypeptide such as a Cas12i polypeptide (e.g., a Cas12i2 or a Cas12i4 polypeptide) and a reverse transcriptase (RT). In addition to the enzymes, the TTR gene editing system disclosed herein also comprises a guide RNA (gRNA) mediating cleavage at a genetic site of the TTR gene by the Cas12i polypeptide, and a reverse transcription donor RNA mediating synthesis of desired sequences to be incorporated into the genomic site of the TTR gene. As reported herein, the gene editing system disclosed herein has achieved successful gene editing of the TTR gene with high editing efficiency and accuracy, for example, by incorporating the T119M mutation in the encoded TTR protein.

Accordingly, provided herein are gene editing systems for editing a TTR gene, pharmaceutical compositions or kits comprising such, methods of using the gene editing systems to produce genetically modified cells, and the resultant cells thus produced. Also provided herein are uses of the gene editing systems disclosed herein, the pharmaceutical compositions and kits comprising such, and/or the genetically modified cells thus produced for treating amyloidogenic transthyretin (ATTR) in a subject.

In some aspects, the present disclosure provides a gene editing system for genetic editing of a transthyretin (TTR) gene, comprising: (a) a Cas12i polypeptide or a first nucleic acid encoding the Cas12i polypeptide: (b) a reverse transcriptase (RT) polypeptide or a second nucleic acid encoding the RT polypeptide: (c) an RNA guide or a third nucleic acid encoding the RNA guide, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence; and (d) a reverse transcription donor RNA (RT donor RNA) or a fourth nucleic acid encoding the RT donor RNA, wherein the RT donor RNA comprises a primer binding site (PBS) and a template sequence.

In some embodiments, the Cas12i polypeptide is a Cas12i2 polypeptide, which may comprise an amino acid sequence at least 95% identical to SEQ ID NO: 9 and comprises one or more mutations relative to SEQ ID NO: 9. In some examples, the one or more mutations in the Cas12i2 polypeptide are at positions H485, D581, G624, F626, P868, I926, V1030, E1035, and/or S1046 of SEQ ID NO: 9. For example, the one or more mutations are amino acid substitutions, which optionally are H485A, D581R, G624R, F626R, P868T, I926R, V1030G, E1035R, S1046G, or a combination thereof.

In some examples, the variant Cas12i2 polypeptide comprises a mutation at position H485, e.g., H485A substitution. In some examples, the variant Cas12i2 polypeptide comprises mutations at positions D581, D911, I926, and V1030, which optionally are amino acid substitutions of D581R. D911R, I926R, and V1030G. In some examples, the variant Cas12i2 polypeptide comprises mutations at positions D581, I926, and V1030, which optionally are amino acid substitutions of D581R, I926R, and V1030G. In some examples, the variant Cas12i2 polypeptide comprises D581, I926, V1030, and S1046, which optionally are amino acid substitutions of D581R, I926R, V1030G, and S1046G. In some examples, the variant Cas12i2 polypeptide comprises mutations at positions D581, G624. F626, I926, V1030, E1035, and S1046, which optionally are amino acid substitutions of D581R, G624R, F626R, I926R, V1030G, E1035R, and S1046G. In some examples, the variant Cas12i2 polypeptide comprises mutations at positions D581. G624, F626, P868, I926, V1030, E1035, and S1046, which optionally are amino acid substitutions of D581R, G624R. F626R, P868T, I926R, V1030G, E1035R, and S1046G. Any of the Cas12i2 polypeptides, including variant Cas12i2 polypeptides listed in Table 2 is within the scope of the present disclosure. In one specific example, the variant Cas12i2 polypeptide comprises the amino acid sequence of SEQ ID NO: 11. In another specific example, the variant Cas12i2 polypeptide comprises the amino acid sequence of SEQ ID NO: 14. In some instances, such a variant may further comprise a mutation at position H485, e.g., the H485A substitution. For example, the H485A-containing variant may comprise the amino acid sequence of SEQ ID NO: 352 or SEQ ID NO: 355.

In other embodiments, the Cas12i polypeptide is a Cas12i4 polypeptide, which may comprise an amino acid sequence at least 95% identical to SEQ ID NO: 15 and comprises one or more mutations relative to SEQ ID NO: 15. In some instances, the one or more mutations in the Cas12i4 polypeptide are at positions E480. G564, V592, and E1042 of SEQ ID NO: 15. For example, the one or more mutations are amino acid substitutions of E480R, G564R, V592R, and E1042R. In one specific example, the Cas12i4 polypeptide comprises the amino acid sequence of SEQ ID NO: 16.

In some embodiments the gene editing system disclosed herein comprises the first nucleic acid encoding the Cas12i2 or Cas12i4 polypeptide. In some examples, the first nucleic acid is a messenger RNA (mRNA). In some examples, the first nucleic acid is included in a viral vector.

In some embodiments, the RT polypeptide in any of the gene editing systems disclosed herein is Moloney Murine Leukemia Virus (MMLV)-RT, mouse mammary tumor virus (MMTV)-RT, Marathon-RT, or RTx-RT. In some examples, the system comprises the RT polypeptide. In other examples, the system comprises the second nucleic acid encoding the RT polypeptide.

In some instances, the gene editing system disclosed herein comprises a fusion polypeptide or a nucleic acid encoding the fusion polypeptide, which comprises the Cas12i polypeptide and the RT polypeptide. In some examples, the Cas12i polypeptide in the fusion polypeptide is a Cas12i2 polypeptide, e.g., a variant Cas12i2 polypeptide as disclosed herein. Such a fusion polypeptide may comprise the amino acid sequence set forth in SEQ ID NO: 59 or 353. Alternatively, the Cas12i polypeptide in any of the fusion polypeptides disclosed herein can be a Cas12i4 polypeptide such as a variant Cas12i4 disclosed herein. Such a fusion polypeptide may comprise the amino acid sequence set forth in SEQ ID NO: 238.

In some examples, the gene editing system disclosed herein may comprise a nucleic acid encoding the fusion polypeptide as disclosed herein. In some instances, the nucleic acid is a mRNA molecule.

In some embodiments, the spacer in the RNA guide of the gene editing system is specific to a target sequence within exon 4 of the TTR gene. Exemplary target sequences within exon 4 include, but are not limited to:

(i)
(SEQ ID NO: 60)
CACCACGGCTGTCGTCACCA
(ii)
(SEQ ID NO: 61)
GGATTGGTGACGACAGCCGT,
(iii)
(SEQ ID NO: 62)
CTTGGGATTGGTGACGACAG,
(iv)
(SEQ ID NO: 240)
CTCCTATTCCACCACGGCTG,
(v)
(SEQ ID NO: 241)
CTATTCCACCACGGCTGTCG,
(vi)
(SEQ ID NO: 242)
TTCCACCACGGCTGTCGTCA,
(vii)
(SEQ ID NO: 243)
CCACCACGGCTGTCGTCACC,
(viii)
(SEQ ID NO: 244)
GGGATTGGTGACGACAGCCG,
(ix)
(SEQ ID NO: 245)
GGTGACGACAGCCGTGGTGG,
(x)
(SEQ ID NO: 246)
GTGACGACAGCCGTGGTGGA,
or
(xi)
(SEQ ID NO: 247)
ACGACAGCCGTGGTGGAATA

In one specific example, the target sequence is SEQ ID NO: 60.

Exemplar spacer sequences specific to the above noted target sequences include:

(i)
(SEQ ID NO: 63)
CACCACGGCUGUCGUCACCA
(ii)
(SEQ ID NO: 64)
GGAUUGGUGACGACAGCCGU,
(iii)
(SEQ ID NO: 65)
CUUGGGAUUGGUGACGACAG,
(iv)
(SEQ ID NO: 248)
CUCCUAUUCCACCACGGCUG,
(v)
(SEQ ID NO: 249)
CUAUUCCACCACGGCUGUCG,
(vi)
(SEQ ID NO: 250)
UUCCACCACGGCUGUCGUCA,
(vii)
(SEQ ID NO: 251)
CCACCACGGCUGUCGUCACC,
(viii)
(SEQ ID NO: 252)
GGGAUUGGUGACGACAGCCG,
(ix)
(SEQ ID NO: 253)
GGUGACGACAGCCGUGGUGG,
(x)
(SEQ ID NO: 254)
GUGACGACAGCCGUGGUGGA,
or
(xi)
(SEQ ID NO: 255)
ACGACAGCCGUGGUGGAAUA

In one example, the spacer sequence comprises SEQ ID NO: 63.

In some embodiments, the RNA guide of the gene editing system disclosed herein comprises the spacer sequence and a direct repeat (DR) sequence. Exemplary DR sequences include AGAAAUCCGUCUUUCAUUGACGG (SEQ ID NO: 39) (DNA counterpart AGAAATCCGTCTTTCATTGACGG (SEQ ID NO: 356) or AGACAUGUGUCCUCAGUGACAC (SEQ ID NO: 58) (DNA counterpart AGACATGTGTCCTCAGTGACAC (SEQ ID NO: 357). In some instances, the gene editing system comprises the RNA guide. Alternatively, the system comprises the third nucleic acid encoding the RNA guide.

In some embodiments, the RT donor RNA in the gene editing system disclosed herein comprises a PBS, which may be 15-75-nucleotide in length. In some examples, the PBS is 20-60-nucleotide in length. In other examples, the PBS is 20-50-nucleotide in length. In specific examples, the PBS is 20-50-nucleotide in length, for example, 20-, 35- or 50-nucleotide in length. Exemplary PBS sequences are provided in Table 10 and Table 16, any of which is within the scope of the present disclosure. In some instances, the PBS binds a PBS-targeting site that is adjacent to the complementary region of the target sequence. The PBS-targeting site may be upstream to the complementary region of the target sequence.

In some instances, the RT donor RNA comprises a template sequence, which may be 25-75-nucleotide in length, for example, 30-60-nucleotide in length. In some examples, the template sequence is 34-, 44- or 54-nucleotide in length.

In some embodiments, the template sequence is homologous to the genomic site of interest and comprises at least two nucleotide variations relative to the genomic site of interest. For example, the template sequence comprises a nucleotide variation encoding the T119M substitution and one or more silent mutations, e.g., those described herein. In some examples, at least one nucleotide variation is located within the region of the template sequence that is complementary to the target sequence. In some examples, the codon including the at least one nucleotide variation encodes the T119M substitution. In some examples, at least one nucleotide variation is located in the PAM. Exemplary template sequences are provided in Table 10 and Table 16, any of which is within the scope of the present disclosure.

In some examples, one or more mutations in the template sequence occur at position C₆ of SEQ ID NO: 351. In some examples, the one or more mutations further occur at positions C₄, C₁₀, C₁₃, C₁₆, and/or C₁₉ of SEQ ID NO: 351.

In some embodiments, the gene editing system disclosed herein comprises the RT donor RNA. Alternatively, the gene editing system comprises the fourth nucleic acid encoding the RT donor RNA. In some examples, the gene editing system comprises a polyribonucleotide comprising the RNA guide and the RT donor RNA. The RNA guide comprises the spacer sequence and the DR sequence and the RT donor RNA comprises the PBS and the template sequence. In specific examples, the polyribonucleotide may comprise, from 5′ to 3′, the template sequence, the PBS, the direct repeat sequence, and the spacer sequence. In some instances, the polyribonucleotide further comprises a 5′ end protection means, a 3′ end protection means, or both. Exemplary polyribonucleotide (e.g., editing template RNAs) may comprise the nucleotide sequence of any one of those listed in Table 11 and Table 17 (e.g., editing template RNA 60, editing template RNA 64, or editing template RNA 98). In some instances, the polyribonucleotide comprises one or more modifications, which optionally comprise 2′-O-methylation, PS bond, or a combination thereof.

In some embodiments, the gene editing system disclosed herein may comprise one or more lipid nanoparticles (LNPs), which are associated with element (a), (b), (c), (d), or any combination thereof. For example, the gene editing system may comprise (i) mRNA molecules encoding any of the fusion polypeptides disclosed herein and (ii) any of the polyribonucleotides also disclosed herein. The LNPs or a portion thereof may be associated with the mRNAs and the polyribonucleotide(s). In some instances, at least a portion of the mRNA molecules and/or at least a portion of the polyribonucleotide is encapsulated by the LNPs.

In other embodiments, the gene editing system may comprise (i) a DNA molecule encoding the fusion polypeptide as disclosed herein, and (ii) the polyribonucleotide as also disclosed herein.

In addition, the present disclosure features a pharmaceutical composition or a combination of pharmaceutical compositions, which collectively comprise any of the gene editing systems disclosed herein. The present disclosure also features a kit comprising the elements of (a)-(d) of the gene editing system disclosed herein.

In other aspects, the present disclosure features a method for editing a transthyretin (TTR) gene in a cell, the method comprising contacting a host cell with the gene editing system for editing the TTR gene as disclosed herein to genetically edit the TTR gene in the host cell. In some instances, the host cell is cultured in vitro. In other examples, the contacting step is performed by administering the system for editing the TTR gene to a subject comprising the host cell.

Also provided herein is a cell comprising a mutated transthyretin (TTR) gene, for example, a cell produced by contacting a host cell with the gene editing system disclosed herein to genetically edit the TTR gene in the host cell, thereby mutating the TTR gene. Such a genetically engineered cell may comprise a disrupted TTR gene. Alternatively, the genetically engineered cell may comprise a modified TTR gene, which expresses a mutated TTR relative to a wild-type counterpart cell.

In yet other aspects, the present disclosure provides a method for treating amyloidogenic transthyretin (ATTR) in a subject in need thereof, comprising administering to the subject any of the gene editing systems disclosed herein for editing a transthyretin (TTR) gene or any of the genetically engineered cells as also disclosed herein. In some embodiments, the subject may be a human patient having hereditary ATTR (hATTR) or wild-type ATTR amyloidosis.

Also within the scope of the present disclosure are any of the gene editing systems disclosed herein or genetically engineered cells produced by such a gene editing system for use in treating amyloidogenic transthyretin (ATTR) in a subject, as well as uses of the gene editing systems or the genetically engineered cells for manufacturing a medicament for the intended therapeutic purposes.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram illustrating configuration of an exemplary RT editing template RNA for use in the gene editing system disclosed herein. DR: direct repeat. PBS: primer binding site. RTT: reverse transcription template.

FIG. 2 is a diagram showing positions within the region (SEQ ID NO: 351 below) where the codon encoding T119 is located for introducing single nucleotide polymorphism (SNP) variation that leads to the T119M mutation and SNP positions for introducing one or more silent mutations for enhancing the editing efficiency and prevent re-targeting of the editing template RNA to the edited genomic site. Reference sequence encompassing the codon for T119:
A₇C₆T₅C₄C₃T₂A₁T₁T2C₃C₄A₅C₆C₇A₈C₉G₁₀G₁₁C₁₂T₁₃G₁₄T₁₅C₁₆G₁₇T₁₈C₁₉A₂₀C₂₁C₂₂A₂₃A₂₄T₂₅C₂₆C₂₇ (SEQ ID NO: 351). The codon encoding T117M mutation is at positions A₅-C₇ (underlined with the C₆ for SNP variation in boldface). The PAM motif is located at positions A₁ to C₃ (ATTC).

FIG. 3 is a diagram showing that an exemplary Cas12i2-RT fusion polypeptide in the presence of various editing template RNAs was capable of introducing mutations leading to the designed amino acid substitutions at the TTR locus in HEK293T cells. Percentage of NGS reads is shown on the y-axis, total edits are shown as in grey bars, and precise edits are shown as black bars. The data shown is an average of two biological replicates, each of which had three technical replicates.

FIG. 4 is a diagram showing that guides that contained PS-2′OMe modifications on the first three and last three nucleotides increased RT editing by up to 13% in HEK293T cells, with similar patterns observed for RTT and PBS lengths.

FIG. 5 is a diagram showing T119M editing efficacy using TTR gene editing systems comprising an mRNA encoding the Cas12i2-MMLV fusion polypeptide in the presence of an editing template RNA in HEK293T cells. Percentage of NGS reads is shown on the y-axis, total edits are shown as in grey bars, and precise edits are shown as black bars. The data shown is an average of three technical replicates across one bioreplicate.

FIG. 6 is a diagram showing T119M editing efficacy using TTR gene editing systems comprising an mRNA encoding the Cas12i2-MMLV fusion polypeptide in the presence of an editing template RNA of SEQ ID NO: 214 or SEQ ID NO: 218 in iPSC cells. Percentage of NGS reads is shown on the v-axis, total edits are shown as in grey bars, and precise edits are shown as black bars. The data shown is an average of three technical replicates across one bioreplicates.

FIG. 7 is a diagram showing T119M editing efficacy using TTR gene editing systems comprising an mRNA encoding the Cas12i2-MMLV fusion polypeptide in the presence of an editing template RNA of SEQ ID NO: 214 or SEQ ID NO: 218 in T cells. Percentage of NGS reads is shown on the y-axis, total edits are shown as in grey bars, and precise edits are shown as black bars. The data shown is an average of three technical replicates across one bioreplicate.

FIG. 8 is a diagram showing that an exemplary Cas12i4-RT fusion polypeptide in the presence of various editing template RNAs was capable of introducing the encoded substitutions at the TTR locus. Percentage of NGS reads is shown on the y-axis, total edits are shown as in grey bars, and encoded edits are shown as black bars.

### DETAILED DESCRIPTION

The present disclosure relates to gene editing systems for, e.g., introducing into one or more mutations into a transthyretin (TTR) gene, for example, leading to the amino acid mutation at position T119 (e.g., the T119M substitution). The TTR gene editing systems disclosed herein comprise a Type V nuclease (e.g., a Cas12i2 or a Cas12i4 polypeptide) or a nucleic acid encoding such, an RNA guide or a nucleic acid encoding such, a reverse transcriptase or a nucleic acid encoding such, and an RT donor RNA or a nucleic acid encoding such. Also provided herein are pharmaceutical compositions and kits comprising any of the gene editing systems disclosed herein, methods for genetically editing a cell using any of the gene editing systems disclosed herein, genetically engineered cells thus produced, and gene editing RNA molecules or a set of RNA molecules involved in the gene editing system, as well as DNA molecule(s) for producing such.

### DEFINITIONS

The present disclosure will be described with respect to particular embodiments and with reference to certain Figures, but the present disclosure is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of a Cas12i polypeptide. For example, activity can include nuclease activity.

As used herein the term “TTR” refers to “transthyretin.” TTR is a transport protein in the serum and cerebrospinal fluid that carries the thyroid hormone thyroxin (T4) and retinol-binding protein bound to retinol (vitamin A). TTR misfolding and aggregation is associated with amyloid disease, senile systemic amyloidosis, familial amyloid polyneuropathy, and familial amyloid cardiomyopathy. Sequence information of the human TTR gene (including various exons) and the TTR protein is provided in Table 1 below.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any of the Cas12i polypeptides provided in U.S. Pat. No. 10,808,245, which is incorporated by reference for the subject matter and purpose referenced herein. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 9-14, 352, or 353. In some embodiments, a Cas12i polypeptide of the disclosure is a Cas12i2 polypeptide as described in WO/2021/202800, the relevant disclosures of which are incorporated by reference for the subject matter and purpose referenced herein. In some embodiments, a Cas12i polypeptide of the disclosure is a Cas12i4 polypeptide as described in WO/2022/174099, the relevant disclosures of which are incorporated by reference for the subject matter and purpose referenced herein. In some embodiments, the Cas12i polypeptide cleaves a target nucleic acid (e.g., as a nick or a double strand break).

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the term “complex” refers to a grouping of two or more molecules. In some embodiments, the complex comprises a polypeptide and a nucleic acid molecule interacting with (e.g., binding to, coming into contact with, adhering to) one another. In some embodiments, the term “complex” is used to refer to association of a CRISPR nuclease (e.g. a Cas12i polypeptide such as a Cas12i2 or a Cas12i4 polypeptide as disclosed herein) and a reverse transcriptase polypeptide. For example, a complex of a CRISPR nuclease such as a Cas12i polypeptide (e.g., a Cas12i2 or Cas12i4 polypeptide as disclosed herein) and a reverse transcriptase polypeptide may be a heterodimer of the two polypeptides, e.g., via a dimerization domain (e.g., a leucine zipper), an antibody, a nanobody, or an aptamer. In some embodiments, the term “complex” is used to refer to association of an RNA guide and an RT donor RNA. In some embodiments, the term “complex” is used to refer to association of a CRISPR nuclease (e.g., Cas12i polypeptide such as a Cas12i2 or Cas12i4 polypeptide), a reverse transcriptase polypeptide, an RNA guide, and an RT donor RNA. In some embodiments, the term “complex” is used to refer to association of a reverse transcriptase polypeptide and an RT donor RNA.

As used herein, the term “binding site recognizable by a nuclease” or “nuclease binding sequence” refers to a sequence that is capable of binding to a CRISPR nuclease. In some embodiments, the nuclease binding sequence is an RNA sequence. In some embodiments, the nuclease binding sequence is a direct repeat sequence. In some embodiments, a nuclease binding sequence is capable of binding to a Cas12i2 polypeptide (e.g., binding site recognizable by a Cas12i2 or Cas12i4 polypeptide).

As used herein, the term “deletion” refers to a loss of a nucleotide or nucleotides in a nucleic acid sequence, relative to a reference sequence. No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to an insertion of up to several kilobases.

As used herein, the term “edit” refers to one or more modifications introduced into a nucleotide sequence in a TTR target nucleic acid such as in a genomic site of interest. The edit may occur within a TTR target sequence as defined herein. Alternatively, the edit may occur outside the TTR target sequence (e.g., adjacent to the TTR target sequence). The edit can be one or more substitutions, one or more insertions, one or more deletions, or a combination thereof.

As used herein, the terms “fusion” and “fused” refer to the joining of at least two nucleotide or protein molecules. For example, “fusion” and “fused” can refer to the joining of at least two polypeptide domains that are encoded by separate genes (e.g., a Cas12i polypeptide such as a Cas12i2 or Cas12i4 polypeptide, and a reverse transcriptase polypeptide) in nature. The fusion can be an N-terminal fusion, a C-terminal fusion, or an intramolecular fusion. In some aspects, the domains are transcribed and translated to produce a single polypeptide. Also as used herein, the terms “fusion” and “fused” are used to refer to the joining of two nucleic acid molecules, such as two RNA molecules (e.g., an RNA guide and an RT donor RNA). The fusion can be a 5′ fusion, a 3′ fusion, or an intramolecular fusion.

As used herein, the term “insertion” refers to a gain of a nucleotide or nucleotides in a nucleic acid sequence, relative to a reference sequence. No particular process is implied in how to make a sequence comprising an insertion. For instance, a sequence comprising an insertion can be synthesized directly from individual nucleotides. In other embodiments, an insertion is made by providing and then altering a reference sequence. The nucleic acid sequence can be in a genome of an organism. The nucleic acid sequence can be in a cell. The nucleic acid sequence can be a DNA sequence. The insertion can be a frameshift mutation or a non-frameshift mutation. An insertion described herein refers to an insertion of up to several kilobases.

As used herein, the term “protospacer adjacent motif” or “PAM” refers to a DNA sequence adjacent to a target sequence (e.g., a TTR target sequence) to which a complex comprising an RNA guide (e.g., a TTR-targeting RNA guide) and a Cas12i polypeptide binds. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein.

In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif.

Alternatively, the target sequence and the PAM may be separately by a small sequence segment (e.g., up to 5 nucleotides, for example, up to 4, 3, 2, or 1 nucleotide). A target sequence is located at the 3′ end of a PAM motif for a Cas12i polypeptide (e.g., a Cas12i2 or Cas12i4 polypeptide such as those disclosed herein). In some embodiments, the target sequence is a sequence within a TTR gene sequence, including, but not limited to, any of the target sequences provided in Table 1 and Table 2.

As used herein, the term “RNA guide” or “RNA guide sequence” refers to any RNA molecule or a modified RNA molecule that facilitates the targeting of a polypeptide (e.g., a Cas12i polypeptide) described herein to a target sequence (e.g., a sequence of a TTR gene). For example, an RNA guide can be a molecule that is designed to be complementary to a specific nucleic acid sequence (a target sequence such as a target sequence with a TTR gene). An RNA guide may comprise a spacer sequence and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA and mature crRNA are also used herein to refer to an RNA guide. The 5′ end or 3′ end of an RNA guide may be fused to an RT donor RNA as disclosed herein. In some instances, the RNA guide can be a modified RNA molecule comprising one or more deoxyribonucleotides, for example, in a DNA-binding sequence contained in the RNA guide, which binds the complementary sequence of the target sequence. In some examples, the DNA-binding sequence may contain a DNA sequence or a DNA/RNA hybrid sequence.

As used herein, the term “spacer” or “spacer sequence” is a portion in an RNA guide that is the RNA equivalent of the TTR target sequence (a DNA sequence). The spacer is capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide (e.g., a Cas12i2 polypeptide, a Cas12i2-reverse transcriptase fusion polypeptide, a Cas12i4 polypeptide, a Cas12i4-reverse transcriptase fusion polypeptide, or a variant thereof) that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

As used herein, the terms “reverse transcriptase” and “RT” refer to a multi-functional enzyme that typically has three enzymatic activities including RNA- and DNA-dependent DNA polymerization activity and an RNase H activity that catalyzes the cleavage of RNA in RNA-DNA hybrids. A reverse transcriptase can generate DNA from an RNA template.

As used herein, the terms “reverse transcription donor RNA” and “RT donor RNA” refer to an RNA molecule comprising a reverse transcription template sequence (template sequence) and a primer binding site (PBS). An RT donor RNA may be fused to an RNA guide at either the 5′ end or 3′ end of the RNA guide.

As used herein, the term “PBS-targeting site” refers to the region, to which a PBS binds. The PBS-targeting site may be adjacent to (e.g., upstream to) a region of the non-PAM strand that is complementary to the target sequence. For example, the PBS-targeting site can be 3-10 nucleotides (e.g., 3-nucleotide or 4-nucleotide) upstream to the region that is complementary to the target sequence. In some instances, the PBS-targeting site may be immediately adjacent to the region of the non-PAM stand that is complementary to the target sequence. In other examples, the PBS-targeting site may overlap with the region of the non-PAM strand that is complementary to the target sequence. Alternatively, the PBS-targeting site may be adjacent to, upstream to, or overlap with the target sequence on the PAM strand.

As used herein, the term “reverse transcription template sequence” or “template sequence” refers to an RNA molecule or a fragment of an RT donor RNA that serves as a template for DNA synthesis by a reverse transcriptase. In some embodiments, a portion of the template sequence is an RNA counterpart of a target sequence or a fragment thereof. In some embodiments, the reverse transcription template sequence comprises an edit to be incorporated into a genomic site where gene editing is needed. In some instances, an edit mediated by the reverse transcription template sequence in the RT donor RNA disrupts or removes the PAM sequence, the target sequence, or both.

As used herein, the term “editing template RNA” or “gene editing RNA” (used herein interchangeably) refers to an RNA molecule or a set of RNA molecules comprising an RNA guide (comprising a spacer and one or more binding site recognizable by a Cas12i polypeptide such as those disclosed herein) and a RT doner RNA (comprising a PBS and a reverse transcription template sequence). A gene editing RNA is capable of mediating cleavage at a TTR target sequence within a genomic site of interest by a Cas12i polypeptide (e.g., a Cas12i2 or Cas12i4 polypeptide) and synthesis of a DNA fragment from a free 3′ end of a free DNA strand generated by the Cas12i polypeptide cleavage based on the template sequence in the gene editing RNA. In some embodiments, an editing template RNA or gene editing RNA is a single RNA molecule comprising the RNA guide linked (e.g., fused) to the RT donor RNA. In some embodiments, an editing template RNA from 5′ to 3′ comprises one or more binding site recognizable by a Cas12i polypeptide, a spacer sequence, a PBS, and an RT donor RNA. In some embodiments, an editing template RNA or gene editing RNA from 5′ to 3′ comprises one or more binding site recognizable by a Cas12i polypeptide, a spacer, a template sequence, and a PBS. In some embodiments, an editing template RNA or gene editing RNA from 5′ to 3′ comprises a template sequence, a PBS, one or more binding site recognizable by a Cas12i polypeptide, and a spacer sequence. In some embodiments, an editing template RNA further comprises a linker. For example, in some embodiments, an editing template RNA comprises a linker between the one or more binding site recognizable by a Cas12i polypeptide and the PBS or between the spacer sequence and the RT donor RNA.

As used herein, the term “substitution” refers to a replacement of a nucleotide or nucleotides with a different nucleotide or nucleotides, relative to a reference sequence. No particular process is implied in how to make a sequence comprising a substitution. For instance, a sequence comprising a substitution can be synthesized directly from individual nucleotides. In other embodiments, a substitution is made by providing and then altering a reference sequence. The nucleic acid sequence can be in a genome of an organism. The nucleic acid sequence can be in a cell. The nucleic acid sequence can be a DNA sequence. The substitution described herein refers to a substitution of up to several kilobases.

As used herein, the terms “upstream” and “downstream” refer to relative positions within a single nucleic acid (e.g., DNA) sequence. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the terms “upstream” and downstream” are used in reference to a non-PAM strand. For example, in some embodiments, a PBS is complementary to a non-PAM strand sequence that is upstream of a target sequence. As such, in some embodiments, a PBS binds to a sequence upstream of a sequence to which a spacer sequence binds, and the spacer sequence binds downstream of a sequence to which the PBS binds.

### I. TTR Gene Editing Systems

In some aspects, the present disclosure provides Cas112i/reverse transcriptase-mediated gene editing systems comprising an RNA guide targeting a TTR gene and an RT template (RTT) for introducing variations into the TTR gene. Such a gene editing system can be used to edit the TTR target gene, e.g., to introduce mutations into the TTR gene.

Transthyretin “TTR” is a transport protein in the serum and cerebrospinal fluid that carries the thyroid hormone thyroxin (T4) and vitamin A. TTR misfolding and aggregation is associated with amyloid disease, senile systemic amyloidosis familial amyloid polyneuropathy, and familial amyloid cardiomyopathy. Accordingly, the gene editing systems disclosed here, targeting the TTR gene, could be used to treat amyloid diseases in a subject in need of the treatment. Table 1 below provides sequence information of human TTR gene and components thereof, and the TTR protein.

TABLE 1
Sequences of TTR Gene and Protein

| | | SEQ ID |
|---|---|---|
| Description | Sequences | NO |
| TTR Gene | ACAGAAGTCCACTCATTCTTGGCAGGATGGCTTCTCATCGTCTGCTCCTC | 1 |
| (genomic) | CTCTGCCTTGCTGGACTGGTATTTGTGTCTGAGGCTGGCCCTACGGTGAG | |
| | TGTTTCTGTGACATCCCATTCCTACATTTAAGATTCACGCTAAATGAAGT | |
| | AGAAGTGACTCCTTCCAGCTTTGCCAACCAGCTTTTATTACTAGGGCAAG | |
| | GGTACCCAGCATCTATTTTTAATATAATTAATTCAAACTTCAAAAAGAAT | |
| | GAAGTTCCACTGAGCTTACTGAGCTGGGACTTGAACTCTGAGCATTCTAC | |
| | CTCATTGCTTTGGTGCATTAGGTTTGTAATATCTGGTACCTCTGTTTCCT | |
| | CAGATAGATGATAGAAATAAAGATATGATATTAAGGAAGCTGTTAATACT | |
| | GAATTTTCAGAAAAGTATCCCTCCATAAAATGTATTTGGGGGACAAACTG | |
| | CAGGAGATTATATTCTGGCCCTATAGTTATTCAAAACGTATTTATTGATT | |
| | AATCTTTAAAAGGCTTAGTGAACAATATTCTAGTCAGATATCTAATTCTT | |
| | AAATCCTCTAGAAGAATTAACTAATACTATAAAATGGGTCTGGATGTAGT | |
| | TCTGACATTATTTTATAACAACTGGTAAGAGGGAGTGACTATAGCAACAA | |
| | CTAAAATGATCTCAGGAAAACCTGTTTGGCCCTATGTATGGTACATTACA | |
| | TCTTTTCAGTAATTCCACTCAAATGGAGACTTTTAACAAAGCAACTGTTC | |
| | TCAGGGGACCTATTTTCTCCCTTAAAATTCATTATACACATCCCTGGTTG | |
| | ATAGCAGTGTGTCTGGAGGCAGAAACCATTCTTGCTTTGGAAACAATTAC | |
| | GTCTGTGTTATACTGAGTAGGGAAGCTCATTAATTGTCGACACTTACGTT | |
| | CCTGATAATGGGATCAGTGTGTAATTCTTGTTTCGCTCCAGATTTCTAAT | |
| | ACCACAAAGAATAAATCCTTTCACTCTGATCAATTTTGTTAACTTCTCAC | |
| | GTGTCTTCTCTACACCCAGGGCACCGGTGAATCCAAGTGTCCTCTGATGG | |
| | TCAAAGTTCTAGATGCTGTCCGAGGCAGTCCTGCCATCAATGTGGCCGTG | |
| | CATGTGTTCAGAAAGGCTGCTGATGACACCTGGGAGCCATTTGCCTCTGG | |
| | GTAAGTTGCCAAAGAACCCTCCCACAGGACTTGGTTTTATCTTCCCGTTT | |
| | GCCCCTCACTTGGTAGAGAGAGGCTCACATCATCTGCTAAAGAATTTACA | |
| | AGTAGATTGAAAAACGTAGGCAGAGGTCAAGTATGCCCTCTGAAGGATGC | |
| | CCTCTTTTTGTTTTGCTTAGCTAGGAAGTGACCAGGAACCTGAGCATCAT | |
| | TTAGGGGCAGACAGTAGAGAAAAGAAGGAATCAGAACTCCTCTCCTCTAG | |
| | CTGTGGTTTGCAACCCTTTTGGGTCACAGAACACTTTATGTAGGTGATGA | |
| | AAAGTAAACATTCTATGCCCAGAAAAAATGCACAGATACACACACATACA | |
| | AAATCATATATGTGATTTTAGGAGTTTCACAGATTCCCTGGTGTCCCTGG | |
| | GTAACACCAAAGCTAAGTGTCCTTGTCTTAGAATTTTAGGAAAAGGTATA | |
| | ATGTGTATTAACCCATTAACAAAAGGAAAGGAATTCAGAAATATTATTAA | |
| | CCAGGCATCTGTCTGTAGTTAATATGGATCACCCAAAACCCAAGGCTTTT | |
| | GCCTAATGAACACTTTGGGGCACCTACTGTGTGCAAGGCTGGGGGCTGTC | |
| | AAGCTCAGTTAAAAAAAAAAAGATAGAAGAGATGGATCCATGAGGCAAAG | |
| | TACAGCCCCAGGCTAATCCCACGATCACCCGACTTCATGTCCAAGAGTGG | |
| | CTTCTCACCTTCATTAGCCAGTTCACAATTTTCATGGAGTTTTTCTACCT | |
| | GCACTAGCAAAAACTTCAAGGAAAATACATATTAATAAATCTAAGCAAAG | |
| | TGACCAGAAGACAGAGCAATCAGGAGACCCTTTGCATCCAGCAGAAGAGG | |
| | AACTGCTAAGTATTTACATCTCCACAGAGAAGAATTTCTGTTGGGTTTTA | |
| | ATTGAACCCCAAGAACCACATGATTCTTCAACCATTATTGGGAAGATCAT | |
| | TTTCTTAGGTCTGGTTTTAACTGGCTTTTTATTTGGGAATTCATTTATGT | |
| | TTATATAAAATGCCAAGCATAACATGAAAAGTGGTTACAGGACTATTCTA | |
| | AGGGAGAGACAGAATGGACACCAAAAATATTCCAATGTTCTTGTGAATCT | |
| | TTTCCTTGCACCAGGACAAAAAAAAAAAGAAGTGAAAAGAAGAAAGGAGG | |
| | AGGGGCATAATCAGAGTCAGTAAAGACAACTGCTATTTTTATCTATCGTA | |
| | GCTGTTGCAGTCAAATGGGAAGCAATTTCCAACATTCAACTATGGAGCTG | |
| | GTACTTACATGGAAATAGAAGTTGCCTAGTGTTTGTTGCTGGCAAAGAGT | |
| | TATCAGAGAGGTTAAATATATAAAAGGGAAAAGAGTCAGATACAGGTTCT | |
| | TCTTCCTACTTTAGGTTTTCCACTGTGTGTGCAAATGATACTCCCTGGTG | |
| | GTGTGCAGATGCCTCAAAGCTATCCTCACACCACAAGGGAGAGGAGCGAG | |
| | ATCCTGCTGTCCTGGAGAAGTGCAGAGTTAGAACAGCTGTGGCCACTTGC | |
| | ATCCAATCATCAATCTTGAATCACAGGGACTCTTTCTTAAGTAAACATTA | |
| | TACCTGGCCGGGCACGGTGGCTCACGCCTGTAATCCCAGCACTTTGGGAT | |
| | GCCAAAGTGGGCATATCATCTGAGGTCAGGAGTTCAAGACCAGCCTGGCC | |
| | AACATGGCAAAACTCCGTCTTTATGAAAAATACAAAAATTAGCCAGGCAT | |
| | GGTGGCAGGCGCCTGTAATCCCAGCTAATTGGGAGGCTGAGGCTGGAGAA | |
| | TCCCTTGAATCTAGGAGGCAGAGGTTGCAGTGAGCTGAGATCGTGCCATT | |
| | GCACTCCAGCCTGGGTGACAAGAGTAAAACTCTGTCTCAAAAAAAAAAAA | |
| | TTATACCTACATTCTCTTCTTATCAGAGAAAAAAATCTACAGTGAGCTTT | |
| | TCAAAAAGTTTTTACAAACTTTTTGCCATTTAATTTCAGTTAGGAGTTTT | |
| | CCCTACTTCTGACTTAGTTGAGGGGAAATGTTCATAACATGTTTATAACA | |
| | TGTTTATGTGTGTTAGTTGGTGGGGGTGTATTACTTTGCCATGCCATTTG | |
| | TTTCCTCCATGCGTAACTTAATCCAGACTTTCACACCTTATAGGAAAACC | |
| | AGTGAGTCTGGAGAGCTGCATGGGCTCACAACTGAGGAGGAATTTGTAGA | |
| | AGGGATATACAAAGTGGAAATAGACACCAAATCTTACTGGAAGGCACTTG | |
| | GCATCTCCCCATTCCATGAGCATGCAGAGGTGAGTATACAGACCTTCGAG | |
| | GGTTGTTTTGGTTTTGGTTTTTGCTTTTGGCATTCCAGGAAATGCACAGT | |
| | TTTACTCAGTGTACCACAGAAATGTCCTAAGGAAGGTGATGAATGACCAA | |
| | AGGTTCCCTTTCCTATTATACAAGAAAAAATTCACAACACTCTGAGAAGC | |
| | AAATTTCTTTTTGACTTTGATGAAAATCCACTTAGTAACATGACTTGAAC | |
| | TTACATGAAACTACTCATAGTCTATTCATTCCACTTTATATGAATATTGA | |
| | TGTATCTGCTGTTGAAATAATAGTTTATGAGGCAGCCCTCCAGACCCCAC | |
| | GTAGAGTGTATGTAACAAGAGATGCACCATTTTATTTCTCGAAAACCCGT | |
| | AACATTCTTCATTCCAAAACACATCTGGCTTCTCGGAGGTCTGGACAAGT | |
| | GATTCTTGGCAACACATACCTATAGAGACAATAAAATCAAAGTAATAATG | |
| | GCAACACAATAGATAACATTTACCAAGCATACACCATGTGGCAGACACAA | |
| | TTATAAGTGTTTTCCATATTTAACCTACTTAATCCTCAGGAATAAGCCAC | |
| | TGAGGTCAGTCCTATTATTATCCCCATCTTATAGATGAAGAAAATGAGGC | |
| | ACCAGGAAGTCAAATAACTTGTCAAAGGTCACAAGACTAGGAAATACACA | |
| | AGTAGAAATGTTTACAATTAAGGCCCAGGCTGGGTTTGCCCTCAGTTCTG | |
| | CTATGCCTCGCATTATGCCCCAGGAAACTTTTTCCCTTGTGAAAGCCAAG | |
| | CTTAAAAAAAGAAAAGCCACATTTGTAACGTGCTCTGTTCCCCTGCCTAT | |
| | GGTGAGGATCTTCAAACAGTTATACATGGACCCAGTCCCCCTGCCTTCTC | |
| | CTTAATTTCTTAAGTCATTTGAAACAGATGGCTGTCATGGAAATAGAATC | |
| | CAGACATGTTGGTCAGAGTTAAAGATCAACTAATTCCATCAAAAATAGCT | |
| | CGGCATGAAAGGGAACTATTCTCTGGCTTAGTCATGGATGAGACTTTCAA | |
| | TTGCTATAAAGTGGTTCCTTTATTAGACAATGTTACCAGGGAAACAACAG | |
| | GGGTTTGTTTGACTTCTGGGGCCCACAAGTCAACAAGAGAGCCCCATCTA | |
| | CCAAGGAGCATGTCCCTGACTACCCCTCAGCCAGCAGCAAGACATGGACC | |
| | CCAGTCAGGGCAGGAGCAGGGTTTCGGCGGCGCCCAGCACAAGACATTGC | |
| | CCCTAGAGTCTCAGCCCCTACCCTCGAGTAATAGATCTGCCTACCTGAGA | |
| | CTGTTGTTTGCCCAAGAGCTGGGTCTCAGCCTGATGGGAACCATATAAAA | |
| | AGGTTCACTGACATACTGCCCACATGTTGTTCTCTTTCATTAGATCTTAG | |
| | CTTCCTTGTCTGCTCTTCATTCTTGCAGTATTCATTCAACAAACATTAAA | |
| | AAAAAAAAAAAGCATTCTATGTGTGGAACACTCTGCTAGATGCTGTGGAT | |
| | TTAGAAATGAAAATACATCCCGACCCTTGGAATGGAAGGGAAAGGACTGA | |
| | AGTAAGACAGATTAAGCAGGACCGTCAGCCCAGCTTGAAGCCCAGATAAA | |
| | TACGGAGAACAAGAGAGAGCGAGTAGTGAGAGATGAGTCCCAATGCCTCA | |
| | CTTTGGTGACGGGTGCGTGGTGGGCTTCATGCAGCTTCTTCTGATAAATG | |
| | CCTCCTTCAGAACTGGTCAACTCTACCTTGGCCAGTGACCCAGGTGGTCA | |
| | TAGTAGATTTACCAAGGGAAAATGGAAACTTTTATTAGGAGCTCTTAGGC | |
| | CTCTTCACTTCATGGATTTTTTTTTCCTTTTTTTTTGAGATGGAGTTTTG | |
| | CCCTGTCACCCAGGCTGGAATGCAGTGGTGCAATCTCAGCTCACTGCAAC | |
| | CTCCGCCTCCCAGGTTCAAGCAATTCTCCTGCCTCAGCCTCCCGAGTAGC | |
| | TGGGACTACAGGTGTGCGCCACCACACCAGGCTAATTTTTGTATTTTTTG | |
| | TAAAGACAGGTTTTCACCACGTTGGCCAGGCTGGTCTGAACTCCAGACCT | |
| | CAGGTGATTCACCTGTCTCAGCCTCCCAAAGTGCTGGGATTACAGGTGTG | |
| | AGCCACCGTGCCCGGCTACTTCATGGATTTTTGATTACAGATTATGCCTC | |
| | TTACAATTTTTAAGAAGAATCAAGTGGGCTGAAGGTCAATGTCACCATAA | |
| | GACAAAAGACATTTTTATTAGTTGATTCTAGGGAATTGGCCTTAAGGGGA | |
| | GCCCTTTCTTCCTAAGAGATTCTTAGGTGATTCTCACTTCCTCTTGCCCC | |
| | AGTATTATTTTTGTTTTTGGTATGGCTCACTCAGATCCTTTTTTCCTCCT | |
| | ATCCCTAAGTAATCCGGGTTTCTTTTTCCCATATTTAGAACAAAATGTAT | |
| | TTATGCAGAGTGTGTCCAAACCTCAACCCAAGGCCTGTATACAAAATAAA | |
| | TCAAATTAAACACATCTTTACTGTCTTCTACCTCTTTCCTGACCTCAATA | |
| | TATCCCAACTTGCCTCACTCTGAGAACCAAGGCTGTCCCAGCACCTGAGT | |
| | CGCAGATATTCTACTGATTTGACAGAACTGTGTGACTATCTGGAACAGCA | |
| | TTTTGATCCACAATTTGCCCAGTTACAAAGCTTAAATGAGCTCTAGTGCA | |
| | TGCATATATATTTCAAAATTCCACCATGATCTTCCACACTCTGTATTGTA | |
| | AATAGAGCCCTGTAATGCTTTTACTTCGTATTTCATTGCTTGTTATACAT | |
| | AAAAATATACTTTTCTTCTTCATGTTAGAAAATGCAAAGAATAGGAGGGT | |
| | GGGGGAATCTCTGGGCTTGGAGACAGGAGACTTGCCTTCCTACTATGGTT | |
| | CCATCAGAATGTAGACTGGGACAATACAATAATTCAAGTCTGGTTTGCTC | |
| | ATCTGTAAATTGGGAAGAATGTTTCCAGCTCCAGAATGCTAAATCTCTAA | |
| | GTCTGTGGTTGGCAGCCACTATTGCAGCAGCTCTTCAATGACTCAATGCA | |
| | GTTTTGCATTCTCCCTACCTTTTTTTTCTAAAACCAATAAAATAGATACA | |
| | GCCTTTAGGCTTTCTGGGATTTCCCTTAGTCAAGCTAGGGTCATCCTGAC | |
| | TTTCGGCGTGAATTTGCAAAACAAGACCTGACTCTGTACTCCTGCTCTAA | |
| | GGACTGTGCATGGTTCCAAAGGCTTAGCTTGCCAGCATATTTGAGCTTTT | |
| | TCCTTCTGTTCAAACTGTTCCAAAATATAAAAGAATAAAATTAATTAAGT | |
| | TGGCACTGGACTTCCGGTGGTCAGTCATGTGTGTCATCTGTCACGTTTTT | |
| | CGGGCTCTGGTGGAAATGGATCTGTCTGTCTTCTCTCATAGGTGGTATTC | |
| | ACAGCCAACGACTCCGGCCCCCGCCGCTACACCATTGCCGCCCTGCTGAG | |
| | CCCCTACTCCTATTCCACCACGGCTGTCGTCACCAATCCCAAGGAATGAG | |
| | GGACTTCTCCTCCAGTGGACCTGAAGGACGAGGGATGGGATTTCATGTAA | |
| | CCAAGAGTATTCCATTTTTACTAAAGCAGTGTTTTCACCTCATATGCTAT | |
| | GTTAGAAGTCCAGGCAGAGACAATAAAACATTCCTGTGAAAGGCA | |
| TTR exon 1 | ACAGAAGTCCACTCATTCTTGGCAGGATGGCTTCTCATCGTCTGCTCCTC | 2 |
| | CTCTGCCTTGCTGGACTGGTATTTGTGTCTGAGGCTGGCCCTACGGTGAG | |
| | TGTTTCTGTGACATCCCATTCCTACATTTAAGATTCACGCTAAAT | |
| TTR exon 2 | TTCACTCTGATCAATTTTGTTAACTTCTCACGTGTCTTCTCTACACCCAG | 3 |
| | GGCACCGGTGAATCCAAGTGTCCTCTGATGGTCAAAGTTCTAGATGCTGT | |
| | CCGAGGCAGTCCTGCCATCAATGTGGCCGTGCATGTGTTCAGAAAGGCTG | |
| | CTGATGACACCTGGGAGCCATTTGCCTCTGGGTAAGTTGCCAAAGAACCC | |
| | TCCCACAGGACTTGGTTTTATCTTCCCGTTT | |
| TTR exon 3 | CCATTTGTTTCCTCCATGCGTAACTTAATCCAGACTTTCACACCTTATAG | 4 |
| | GAAAACCAGTGAGTCTGGAGAGCTGCATGGGCTCACAACTGAGGAGGAAT | |
| | TTGTAGAAGGGATATACAAAGTGGAAATAGACACCAAATCTTACTGGAAG | |
| | GCACTTGGCATCTCCCCATTCCATGAGCATGCAGAGGTGAGTATACAGAC | |
| | CTTCGAGGGTTGTTTTGGTTTTGGTTTTTGCTTTTG | |
| TTR exon 4 | CACGTTTTTCGGGCTCTGGTGGAAATGGATCTGTCTGTCTTCTCTCATAG | 5 |
| | GTGGTATTCACAGCCAACGACTCCGGCCCCCGCCGCTACACCATTGCCGC | |
| | CCTGCTGAGCCCCTACTCCTATTCCACCACGGCTGTCGTCACCAATCCCA | |
| | AGGAATGAGGGACTTCTCCTCCAGTGGACCTGAAGGACGAGGGATGGGAT | |
| | TTCATGTAACCAAGAGTATTCCATTTTTACTAAAGCAGTGTTTTCACCTC | |
| | ATATGCTATGTTAGAAGTCCAGGCAGAGACAATAAAACATTCCTGTGAAA | |
| | GGCA | |
| TTR (full | MASHRLLLLCLAGLVFVSEAGPTGTGESKCPLMVKVLDAVRGSPAINVAV | 6 |
| protein | HVFRKAADDTWEPFASGKTSESGELHGLTTEEEFVEGIYKVEIDTKSYWK | |
| sequence) | ALGISPFHEHAEVVFTANDSGPRRYTIAALLSPYSYSTTAVVTNPKE | |
| TTR (post- | GPTGTGESKCPLMVKVLDAVRGSPAINVAVHVFRKAADDTWEPFASGKTS | 7 |
| translationally | ESGELHGLTTEEEFVEGIYKVEIDTKSYWKALGISPFHEHAEVVFTANDS | |
| cleaved | GPRRYTIAALLSPYSYSTTAVVTNPKE | |
| sequence) | | |
| T119 shown in | | |
| bold) | | |
| TTR coding | ATGGCTTCTCATCGTCTGCTCCTCCTCTGCCTTGCTGGACTGGTATTTGT | 8 |
| sequence | GTCTGAGGCTGGCCCTACGGGCACCGGTGAATCCAAGTGTCCTCTGATGG | |
| | TCAAAGTTCTAGATGCTGTCCGAGGCAGTCCTGCCATCAATGTGGCCGTG | |
| | CATGTGTTCAGAAAGGCTGCTGATGACACCTGGGAGCCATTTGCCTCTGG | |
| | GAAAACCAGTGAGTCTGGAGAGCTGCATGGGCTCACAACTGAGGAGGAAT | |
| | TTGTAGAAGGGATATACAAAGTGGAAATAGACACCAAATCTTACTGGAAG | |
| | GCACTTGGCATCTCCCCATTCCATGAGCATGCAGAGGTGGTATTCACAGC | |
| | CAACGACTCCGGCCCCCGCCGCTACACCATTGCCGCCCTGCTGAGCCCCT | |
| | ACTCCTATTCCACCACGGCTGTCGTCACCAATCCCAAGGAATGA | |

The TTR gene editing system disclosed herein comprises protein components or nucleic acids (e.g., mRNAs) encoding such and RNA components or nucleic acids encoding such.

The protein components include a Type V nuclease such as a Cas12i polypeptide (e.g., a Cas12i2 polypeptide or a Cas12i4 polypeptide) as known in the art or disclosed herein, and a reverse transcriptase (RI) polypeptide. See, e.g., International Patent Application No. PCT/US22/32107 and International Patent Application No. PCT/US22/31821, the relevant disclosures of each of which are incorporated by reference for the subject matter and purpose referenced herein. In some instances, the Cas12i polypeptide and the RT polypeptide may form a fusion protein.

The RNA components (the editing template RNA in the TTR gene editing system disclosed herein comprises an RNA guide that targets a genomic site within the TTR gene (e.g., targeting a site within exon 4, such as the region nearby the codon encoding the T119 residue), and a RT donor RNA for introducing desired mutation(s) into the TTR gene. In some instances, the RNA guide and the RT donor may form a single polyribonucleotide as disclosed herein.

### A. Protein Components

### Cas12i CRISPR Nucleases

In some embodiments, the TTR gene editing system disclosed herein comprises a Cas12i CRISPR nuclease. In some embodiments, the Cas12i CRISPR nuclease is a wild-type Cas12i2 CRISPR nuclease, e.g., comprising an amino acid sequence such as SEQ ID NO: 9 provided in Table 2 below. In some embodiments, the CRISPR nuclease of the present invention is a variant of a wildtype CRISPR Cas12i2 nuclease, e.g., a variant of SEQ ID NO: 11. In some embodiments, the Cas12i CRISPR nuclease is a wild-type Cas12i4 CRISPR nuclease, e.g., comprising an amino acid sequence such as SEQ ID NO: 15 provided in Table 2 below. In some embodiments, the CRISPR nuclease of the present invention is a variant of a wildtype CRISPR Cas12i4 nuclease, e.g., a variant of SEQ ID NO: 15.

TABLE 2
Exemplary Cas12i Polypeptides

| | | SEQ ID |
|---|---|---|
| Descriptions | Sequence | NO |
| Cas12i2 | MSSAIKSYKSVLRPNERKNQLLKSTIQCLEDGSAFFFKMLQGLEGGI | 9 |
| | TPEIVRFSTEQEKQQQDIALWCAVNWFRPVSQDSLTHTIASDNLVEK | |
| | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFDVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVFNKIRQLAPMHNITLFGCGSLYTSHQDPLVHRNP | |
| | DKAMKCRWAAIPVKDIGDWVLRKLSQNLRAKNIGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTVKGIG | |
| | EQSSDEENPDGSRIKLQLTS | |
| D581R/D911R/I | MSSAIKSYKSVLRPNERKNQLLKSTIQCLEDGSAFFFKMLQGLEGGI | 10 |
| 926R/V1030G | TPEIVRFSTEQEKQQQDIALWCAVNWFRPVSQDSLTHTIASDNLVEK | |
| Cas12i2 Variant | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPREG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLAPMHNITLFGCGSLYTSHQDPLVHRNP | |
| | DKAMKCRWAAIPVKDIGRWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | EQSSDEENPDGSRIKLQLTS | |
| D581R/1926R/V | MSSAIKSYKSVLRPNERKNQLLKSTIQCLEDGSAFFFKMLQGLEGGI | 11 |
| 1030G Cas 1212 | TPEIVRESTEQEKQQQDIALWCAVNWFRPVSQDSLTHTIASDNLVEK | |
| Variant | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLAPMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | EQSSDEENPDGSRIKLQLTS | |
| D581R/1926/V1 | MSSAIKSYKSVLRPNERKNQLLKSTIQCLEDGSAFFFKMLQGLEGGI | 12 |
| 030G/ S1046G | TPEIVRFSTEQEKQQQDIALWCAVNWFRPVSQDSLTHTIASDNLVEK | |
| Cas12i2 Variant | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMFNKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLAPMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | EQSSDEENPDGGRIKLQLTS | |
| D581R/G624R/F | MSSAIKSYKSVLRPNERKNQLLKSTIQCLEDGSAFFFKMLQGLEGGI | 13 |
| 626R/1926R | TPEIVRESTEQEKQQQDIALWCAVNWFRPVSQDSLTHTIASDNLVEK | |
| /V1030G/ | FEEYYGGTASDAIKQYFSASIGESYYWNDCRQQYYDLCRELGVEVSD | |
| E1035R/S1046G | LTHDLEILCREKCLAVATESNQNNSIISVLFGTGEKEDRSVKLRITK | |
| Cas12i2 Variant | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELRCRVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVFNKIRQLAPMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | RQSSDEENPDGGRIKLQLTS | |
| D581R/G624R/ | MSSAIKSYKSVLRPNERKNQLLKSTIQCLEDGSAFFFKMLQGLEGGI | 14 |
| F626R/P868T/ | TPEIVRESTEQEKQQQDIALWCAVNWFRPVSQDSLTHTIASDNLVEK | |
| I926R/V1030G, | FEEYYGGTASDAIKQYFSASIGESYYWNDCRQQYYDLCRELGVEVSD | |
| E1035R/S1046G | LTHDLEILCREKCLAVATESNQNNSIISVLFGTGEKEDRSVKLRITK | |
| Cas12i2 Variant | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELRCRVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLATMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | RQSSDEENPDGGRIKLQLTS | |
| H485A/D581R/ | MSSAIKSYKSVLRPNERKNQLLKSTIQCLEDGSAFFFKMLQGLEGGI | 352 |
| I926R/V1030G | TPEIVRESTEQEKQQQDIALWCAVNWFRPVSQDSLTHTIASDNLVEK | |
| Cas12i2 Variant | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKAHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLAPMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | EQSSDEENPDGSRIKLQLTS | |
| H485A/D581R/ | MSSAIKSYKSVLRPNERKNQLLKSTIQCLEDGSAFFFKMLQGLEGGI | 355 |
| G624R/ | TPEIVRESTEQEKQQQDIALWCAVNWFRPVSQDSLTHTIASDNLVEK | |
| F626R/P868T/ | FEEYYGGTASDAIKQYFSASIGESYYWNDCRQQYYDLCRELGVEVSD | |
| I926R/V1030G, | LTHDLEILCREKCLAVATESNQNNSIISVLFGTGEKEDRSVKLRITK | |
| E1035R/S1046G | KILEAISNLKEIPKNVAPIQEIILNVAKATKETFRQVYAGNLGAPST | |
| Cas12i2 Variant | LEKFIAKDGQKEFDLKKLQTDLKKVIRGKSKERDWCCQEELRSYVEQ | |
| | NTIQYDLWAWGEMENKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKAHIPFYDTRFFQEIYAAGNSPVDTCQFRTPREG | |
| | YHLPKLTDQTAIRVNKKHVKAAKTEARIRLAIQQGTLPVSNLKITEI | |
| | SATINSKGQVRIPVKFRVGRQKGTLQIGDRFCGYDQNQTASHAYSLW | |
| | EVVKEGQYHKELRCRVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVENKIRQLATMHNITLFGCGSLYTSHQDPLVHRNP | |
| | DKAMKCRWAAIPVKDIGDWVLRKLSQNLRAKNRGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTGKGIG | |
| | RQSSDEENPDGGRIKLQLTS | |
| Cas12i4 | MASISRPYGTKLRPDARKKEMLDKFFNTLTKGQRVFADLALCIYGSL | 15 |
| | TLEMAKSLEPESDSELVCAIGWFRLVDKTIWSKDGIKQENLVKQYEA | |
| | YSGKEASEVVKTYLNSPSSDKYVWIDCRQKFLRFQRELGTRNLSEDE | |
| | ECMLFEQYIRLTKGEIEGYAAISNMFGNGEKEDRSKKRMYATRMKDW | |
| | LEANENITWEQYREALKNQLNAKNLEQVVANYKGNAGGADPFFKYSF | |
| | SKEGMVSKKEHAQQLDKFKTVLKNKARDLNFPNKEKLKQYLEAEIGI | |
| | PVDANVYSQMFSNGVSEVQPKTTRNMSFSNEKLDLLTELKDLNKGDG | |
| | FEYAREVLNGFFDSELHTTEDKFNITSRYLGGDKSNRLSKLYKIWKK | |
| | EGVDCEEGIQQFCEAVKDKMGQIPIRNVLKYLWQFRETVSAEDFEAA | |
| | AKANHLEEKISRVKAHPIVISNRYWAFGTSALVGNIMPADKRHQGEY | |
| | AGQNFKMWLEAELHYDGKKAKHHLPFYNARFFEEVYCYHPSVAEITP | |
| | FKTKQFGCEIGKDIPDYVSVALKDNPYKKATKRILRAIYNPVANTTG | |
| | VDKTTNCSFMIKRENDEYKLVINRKISVDRPKRIEVGRTIMGYDRNQ | |
| | TASDTYWIGRLVPPGTRGAYRIGEWSVQYIKSGPVLSSTQGVNNSTT | |
| | DQLVYNGMPSSSERFKAWKKARMAFIRKLIRQLNDEGLESKGQDYIP | |
| | ENPSSFDVRGETLYVFNSNYLKALVSKHRKAKKPVEGILDEIEAWTS | |
| | KDKDSCSLMRLSSLSDASMQGIASLKSLINSYFNKNGCKTIEDKEKF | |
| | NPVLYAKLVEVEQRRTNKRSEKVGRIAGSLEQLALLNGVEVVIGEAD | |
| | LGEVEKGKSKKQNSRNMDWCAKQVAQRLEYKLAFHGIGYFGVNPMYT | |
| | SHQDPFEHRRVADHIVMRARFEEVNVENIAEWHVRNFSNYLRADSGT | |
| | GLYYKQATMDFLKHYGLEEHAEGLENKKIKFYDFRKILEDKNLTSVI | |
| | IPKRGGRIYMATNPVTSDSTPITYAGKTYNRCNADEVAAANIVISVL | |
| | APRSKKNEEQDDIPLITKKAESKSPPKDRKRSKTSQLPQK | |
| E480R/G564R/ | MASISRPYGTKLRPDARKKEMLDKFFNTLTKGQRVFADLALCIYGSL | 16 |
| V592R/E1042R | TLEMAKSLEPESDSELVCAIGWFRLVDKTIWSKDGIKQENLVKQYEA | |
| Cas1214 Variant | YSGKEASEVVKTYLNSPSSDKYVWIDCRQKFLRFQRELGTRNLSEDF | |
| | ECMLFEQYIRLTKGEIEGYAAISNMFGNGEKEDRSKKRMYATRMKDW | |
| | LEANENITWEQYREALKNQLNAKNLEQVVANYKGNAGGADPFFKYSF | |
| | SKEGMVSKKEHAQQLDKFKTVLKNKARDLNFPNKEKLKQYLEAEIGI | |
| | PVDANVYSQMFSNGVSEVQPKTTRNMSFSNEKLDLLTELKDLNKGDG | |
| | FEYAREVLNGFFDSELHTTEDKFNITSRYLGGDKSNRLSKLYKIWKK | |
| | EGVDCEEGIQQFCEAVKDKMGQIPIRNVLKYLWQFRETVSAEDFEAA | |
| | AKANHLEEKISRVKAHPIVISNRYWAFGTSALVGNIMPADKRHQGEY | |
| | AGQNFKMWLRAELHYDGKKAKHHLPFYNARFFEEVYCYHPSVAEITP | |
| | FKTKQFGCEIGKDIPDYVSVALKDNPYKKATKRILRAIYNPVANTTR | |
| | VDKTTNCSFMIKRENDEYKLVINRKISRDRPKRIEVGRTIMGYDRNQ | |
| | TASDTYWIGRLVPPGTRGAYRIGEWSVQYIKSGPVLSSTQGVNNSTT | |
| | DQLVYNGMPSSSERFKAWKKARMAFIRKLIRQLNDEGLESKGQDYIP | |
| | ENPSSFDVRGETLYVFNSNYLKALVSKHRKAKKPVEGILDEIEAWTS | |
| | KDKDSCSLMRLSSLSDASMQGIASLKSLINSYFNKNGCKTIEDKEKF | |
| | NPVLYAKLVEVEQRRTNKRSEKVGRIAGSLEQLALLNGVEVVIGEAD | |
| | LGEVEKGKSKKQNSRNMDWCAKQVAQRLEYKLAFHGIGYFGVNPMYT | |
| | SHQDPFEHRRVADHIVMRARFEEVNVENIAEWHVRNFSNYLRADSGT | |
| | GLYYKQATMDFLKHYGLEEHAEGLENKKIKFYDERKILEDKNLTSVI | |
| | IPKRGGRIYMATNPVTSDSTPITYAGKTYNRCNADEVAAANIVISVL | |
| | APRSKKNREQDDIPLITKKAESKSPPKDRKRSKTSQLPQK | |

Any of the Cas12i polypeptides provided in Table 2 above can be used in the TTR gene editing system disclosed herein. Additional Cas12i polypeptides for use herein are described in WO2019178427, the relevant disclosures of which are incorporated by reference for the subject mailer and purpose referenced herein.

In some embodiments, the Cas12i polypeptide is a wild-type Cas12i2 polypeptide (e.g., comprising the amino acid sequence of SEQ ID NO: 9). In other embodiments, the Cas12i2 polypeptide may be a variant of the wild-type Cas12i2, which may exhibit similar enzymatic activity, e.g., nuclease or endonuclease activity, and comprising an amino acid sequence differing from the amino acid sequences of SEQ ID NO: 9 by 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods. For example, the Cas12i2 variant may contain one or more mutations at position H485, D581, G624, F626, P868, I926, V1030, E1035, S1046 of SEQ ID NO: 9., or any combination thereof. In some instances, the one or more mutations are amino acid substitutions, for example, H485A, D581R, G624R, F626R, P868T, I926R, V1030G, E1035R, S1046G, or a combination thereof.

In some examples, the Cas12i2 polypeptide contains mutations at positions D581, D911, I926, and V1030 of SEQ ID NO: 9. Such a Cas12i2 polypeptide may contain amino acid substitutions of D581R, D911R, I926R, and V1030G (e.g., SEQ ID NO: 10).

In some examples, the Cas12i2 polypeptide contains mutations at positions D581, I926, and V1030 of SEQ ID NO: 9. Such a Cas12i2 polypeptide may contain amino acid substitutions of D581R, I926R, and V1030G (e.g., SEQ ID NO: 11).

In some examples, the Cas12i2 polypeptide may contain mutations at positions D581, I926, V1030, and S1046 of SEQ ID NO: 9. Such a Cas12i2 polypeptide may contain amino acid substitutions of D581R. I926R. V1030G. and S1046G (e.g., SEQ ID NO: 12).

In some examples, the Cas12i2 polypeptide may contain mutations at positions D581, G624, F626, I926, V1030, E1035, and S1046 of SEQ ID NO: 9. Such a Cas12i2 polypeptide may contain amino acid substitutions of D581R, G624R, F626R, I926R, V1030G, E1035R, and S1046G (e.g., SEQ ID NO: 13).

In some examples, the Cas12i2 polypeptide may contain mutations at positions D581. G624, F626, P868, I926, V1030, E1035, and S1046 of SEQ ID NO: 9. Such a Cas12i2 polypeptide may contain amino acid substitutions of D581R, G624R, F626R, P868T, I926R, V1030G. E1035R, and S1046G (e.g., SEQ ID NO: 14).

Alternatively or in addition, the Cas12i2 polypeptide may contain a mutation at position H485, for example, the H485A substitution. In some examples, the variant Cas12i2 polypeptide may contain only the H485A substitution relative to SEQ ID NO:9. In other examples, the H485A substitution may be in combination with any of the other mutations disclosed herein. In one specific example, the H485A-containing variant Cas12i2 comprises the amino acid sequence of SEQ ID NO: 352. In another specific example, the H485A-containing variant Cas12i2 comprises the amino acid sequence of SEQ ID NO: 355.

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to any one of SEQ ID NOs: 9-14, 352, and 355. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to any one of SEQ ID NOs: 9-14, 352, and 355 maintains the amino acid changes (or at least 1, 2, 3 etc. of these changes) that differentiate the polypeptide from its respective parent/reference sequence.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of any one of SEQ ID NOs: 9-14, 352, and 355. Homology or identity can be determined by amino acid sequence alignment, e.g., using a program such as BLAST, ALIGN, or CLUSTAL, as described herein.

In some embodiments, the composition of the present disclosure includes a Cas12i4 polypeptide described herein (e.g., a polypeptide comprising SEQ ID NO: 15). In some embodiments, the Cas12i4 polypeptide comprises at least one RuvC domain.

In some embodiments, the Cas12i polypeptide is a wild-type Cas12i4 polypeptide (e.g., comprising the amino acid sequence of SEQ ID NO: 15). In other embodiments, the Cas12i4 polypeptide may be a variant of the wild-type Cas12i4, which may exhibit similar enzymatic activity, e.g., nuclease or endonuclease activity, and comprising an amino acid sequence differing from the amino acid sequences of SEQ ID NO: 15 by 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some examples, the Cas12i4 polypeptide may contain mutations at positions E480, G564, V592, and E1042 of SEQ ID NO: 15. Such a Cas12i2 polypeptide may contain amino acid substitutions of E480R, G564R, V592R, and E1042R (e.g., SEQ ID NO: 16).

In some embodiments, the Cas12i4 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 15 or 16. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 15 or 16, maintains the amino acid changes (or at least 1, 2, 3 etc. of these changes) that differentiate the polypeptide from its respective parent/reference sequence.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 910%, at least 92%, at least 93%, at least 94%, at least 95%, at least 96%, at least 97%, at least 98%, or even at least 99%, but not 100%, sequence identity to the amino acid sequence of SEQ ID NO: 15-16. Homology or identity can be determined by amino acid sequence alignment, e.g., using a program such as BLAST, ALIGN, or CLUSTAL, as described herein.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at the world wide web site of kazusa.orjp/codon/and these tables can be adapted in a number of ways. See Nakamura et al. Nucl. Acids Res. 28:292 (2000), which is incorporated herein by reference in its entirety.

Computer algorithms for codon optimizing a particular sequence for expression in a particular host cell are also available, such as Gene Forge (Aptagen; Jacobus, PA). In some examples, the nucleic acid encoding the Cas12i polypeptides such as Cas12i2 polypeptides as disclosed herein can be an mRNA molecule, which can be codon optimized.

In some embodiments, the gene editing system disclosed herein may comprise a Cas12i polypeptide as disclosed herein. In other embodiments, the gene editing system may comprise a nucleic acid encoding the Cas12i polypeptide. For example, the gene editing system may comprise a vector (e.g., a viral vector such as an AAV vector, such as AAV1, AAV2, AAV3, AAV4, AAV5, AAV6, AAV7, AAV8, AAV9, AAV10, AAV11 and AAV12) encoding the Cas12i polypeptide. Alternatively, the gene editing system may comprise a mRNA molecule encoding the Cas12i polypeptide. In some instances, the mRNA molecule may be codon-optimized.

### Reverse Transcriptase

The TTR gene editing system disclosed herein also comprise a reverse transcriptase (RT) polypeptide, which may be a wild-type RT or a variant thereof. In some instances, the RT polypeptide and the Type V CRISPR nuclease (e.g., a Cas12i polypeptide such as a Cas12i2 or Cas12i4 polypeptide) may form a fusion protein.

In some embodiments, the reverse transcriptase polypeptide is any wild-type reverse transcriptase obtained from any naturally-occurring organism or virus, or obtained from a commercial or non-commercial source. The reverse transcriptase polypeptide may also be a variant reverse transcriptase polypeptide.

The reverse transcriptase polypeptide can be obtained from a number of different sources. For instance, the gene may be obtained from eukaryotic cells which are infected with retrovirus or from a plasmid that comprises either a portion of or the entire retrovirus genome. In addition, RNA that comprises the reverse transcriptase gene can be obtained from retroviruses. In some embodiments, the reverse transcriptase is expressed or otherwise provided as an individual component, i.e., not as a fusion protein with a CRISPR nuclease (e.g., a Cas12i) polypeptide.

A person of ordinary skill in the art will recognize that reverse transcriptases are known in the art, including, but not limited to, Moloney Murine Leukemia Virus (MMLV) reverse transcriptase, Human Immunodeficiency Virus (HIV) reverse transcriptase, and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase. Avian Sarcoma Virus UR2 Helper Virus UR2AV reverse transcriptase, Avian Sarcoma Virus Y73 Helper Virus YAV reverse transcriptase. Rous Associated Virus (RAV) reverse transcriptase, and Myeloblastosis Associated Virus (MAV) reverse transcriptase may be suitably used in the composition described herein.

In some embodiments, the reverse transcriptase is MMLV-RT, MarathonRT from Eubacterium rectale, or RTX reverse transcriptase or a variant of MMLV-RT, MarathonRT, or RTX reverse transcriptase. In some embodiments, the reverse transcriptase is a sequence shown in Table 3, a variant thereof, or an ortholog thereof.

TABLE 3
Reverse Transcriptase Sequences.

| Reverse | | SEQ |
|---|---|---|
| Transcriptase | Sequence | ID NO |
| MMLV-RT | MTLNIEDEYRLHETSKEPDVSLGSTWLSDFPQAWAETGGMGLAVRQAPLI | 17 |
| | IPLKATSTPVSIKQYPMSQEARLGIKPHIQRLLDQGILVPCQSPWNTPLL | |
| | PVKKPGTNDYRPVQDLREVNKRVEDIHPTVPNPYNLLSGLPPSHQWYTVL | |
| | DLKDAFFCLRLHPTSQPLFAFEWRDPEMGISGQLTWTRLPQGFKNSPTLF | |
| | NEALHRDLADFRIQHPDLILLQYVDDLLLAATSELDCQQGTRALLQTLGN | |
| | LGYRASAKKAQICQKQVKYLGYLLKEGQRWLTEARKETVMGQPTPKTPRQ | |
| | LREFLGKAGFCRLFIPGFAEMAAPLYPLTKPGTLENWGPDQQKAYQEIKQ | |
| | ALLTAPALGLPDLTKPFELFVDEKQGYAKGVLTQKLGPWRRPVAYLSKKL | |
| | DPVAAGWPPCLRMVAAIAVLTKDAGKLTMGQPLVILAPHAVEALVKQPPD | |
| | RWLSNARMTHYQALLLDTDRVQFGPVVALNPATLLPLPEEGLQHNCLDIL | |
| | AEAHGTRPDLTDQPLPDADHTWYTDGSSLLQEGQRKAGAAVTTETEVIWA | |
| | KALPAGTSAQRAELIALTQALKMAEGKKLNVYTDSRYAFATAHIHGEIYR | |
| | RRGWLTSEGKEIKNKDEILALLKALFLPKRLSIIHCPGHQKGHSAEARGN | |
| | RMADQAARKAAITETPDTSTLLIENSSP | |
| MMLV-RT_2 | MTWLSDFPQAWAETGGMGLAVRQAPLIIPLKATSTPVSIKQYPMSQEARL | 18 |
| | GIKPHIQRLLDQGILVPCQSPWNTPLLPVKKPGTNDYRPVQDLREVNKRV | |
| | EDIHPTVPNPYNLLSGLPPSHQWYTVLDLKDAFFCLRLHPTSQPLFAFEW | |
| | RDPEMGISGQLTWTRLPQGFKNSPTLFDEALHRDLADFRIQHPDLILLQY | |
| | VDDLLLAATSELDCQQGTRALLQTLGNLGYRASAKKAQICQKQVKYLGYL | |
| | LKEGQRWLTEARKETVMGQPTPKTPRQLREFLGTAGFCRLWIPGFAEMAA | |
| | PLYPLTKTGTLFNWGPDQQKAYQEILQALLTAPALGLPDLTKPFELFVDE | |
| | KQGYAKGVLTQKLGPWRRPVAYLSKKLDPVAAGWPPCLRMVAAIAVLTKD | |
| | AGKLTMGQPLVILAPHAVEALVKQPPDRWLSNARMTHYQALLLDTDRVQF | |
| | GPVVALNPATLLPLPEEGLQHNCLDILAEAHGTRPDL | |
| MMTV-RT | MFTLWGRDIMKDIKVRLMTDSPDDSQDLMIGAIESNLFADQISWKSDQPV | 19 |
| | WLNQWPLKQEKLQALQQLVTEQLQLGHLEESNSPWNTPVFVIKKKSGKWR | |
| | LLQDLRAVNATMHDMGALQPGLPSPVAVPKGWEIIIIDLQDCFENIKLHP | |
| | EDCKRFAFSVPSPNFKRPYQRFQWKVLPQGMKNSPTLCQKFVDKAILTVR | |
| | DKYQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFLKL | |
| | TTGELKPLFEILNGDSNPISTRKLTPEACKALQLMNERLSTARVKRLDLS | |
| | QPWSLCILKTEYTPTACLWQDGVVEWIHLPHISPKVITPYDIFCTQLIIK | |
| | GRHRSKELFSKDPDYIVVPYTKVQFDLLLQEKEDWPISLLGELGEVHFHL | |
| | PKDPLLTFTLQTAIIFPHMTSTTPLEKGIVIFTDGSANGRSVTYIQGREP | |
| | IIKENTQNTAQQAEIVAVITAFEEVSQPENLYTDSKYVTGLFPEIETATL | |
| | SPRTKIYTELKHLQRLIHKRQEKFYIGHIRGHTGLPGPLAQGNAYADSLT | |
| | RIL | |
| MarathonRT | MDTSNLMEQILSSDNLNRAYLQVVRNKGAEGVDGMKYTELKEHLAKNGET | 20 |
| | IKGQLRTRKYKPQPARRVEIPKPDGGVRNLGVPTVTDRFIQQAIAQVLTP | |
| | IYEEQFHDHSYGFRPNRCAQQAILTALNIMNDGNDWIVDIDLEKFFDTVN | |
| | HDKLMTLIGRTIKDGDVISIVRKYLVSGIMIDDEYEDSIVGTPQGGNLSP | |
| | LLANIMLNELDKEMEKRGLNFVRYADDCIIMVGSEMSANRVMRNISRFIE | |
| | EKLGLKVNMTKSKVDRPSGLKYLGFGFYFDPRAHQFKAKPHAKSVAKFKK | |
| | RMKELTCRSWGVSNSYKVEKLNQLIRGWINYFKIGSMKTLCKELDSRIRY | |
| | RLRMCIWKQWKTPQNQEKNLVKLGIDRNTARRVAYTGKRIAYVCNKGAVN | |
| | VAISNKRLASFGLISMLDYYIEKCVTC | |
| RTX reverse | MILDTDYITEDGKPVIRIFKKENGEFKIEYDRTFEPYLYALLKDDSAIEE | 21 |
| transcriptase | VKKITAERHGTVVTVKRVEKVQKKFLGRPVEVWKLYFTHPQDVPAIMDKI | |
| | REHPAVIDIYEYDIPFAIRYLIDKGLVPMEGDEELKLLAFDIETLYHEGE | |
| | EFAEGPILMISYADEEGARVITWKNVDLPYVDVVSTEREMIKRFLRVVKE | |
| | KDPDVLITYNGDNFDFAYLKKRCEKLGINFALGRDGSEPKIQRMGDRFAV | |
| | EVKGRIHFDLYPVIRRTINLPTYTLEAVYEAVFGQPKEKVYAEEITTAWE | |
| | TGENLERVARYSMEDAKVTYELGKEFLPMEAQLSRLIGQSLWDVSRSSTG | |
| | NLVEWELLRKAYERNELAPNKPDEKELARRHQSHEGGYIKEPERGLWENI | |
| | VYLDERSLYPSIIITHNVSPDTLNREGCKEYDVAPQVGHRFCKDFPGFIP | |
| | SLLGDLLEERQKIKKRMKATIDPIERKLLDYRQRAIKILANSLYGYYGYA | |
| | RARWYCKECAESVIAWGREYLTMTIKEIEEKYGFKVIYSDTDGFFATIPG | |
| | ADAETVKKKAMEFLKYINAKLPGALELEYEGFYKRGLFVTKKKYAVIDEE | |
| | GKITTRGLEIVRRDWSEIAKETQARVLEALLKDGDVEKAVRIVKEVTEKL | |
| | SKYEVPPEKLVIHKQITRDLKDYKATGPHVAVAKRLAARGVKIRPGTVIS | |
| | YIVLKGSGRIVDRAIPFDEFDPTKHKYDAEYYIEKQVLPAVERILRAFGY | |
| | RKEDLRYQKTRQVGLSARLKPKGT | |
| RTX-exoMinus | MILDTDYITEDGKPVIRIFKKENGEFKIEYDRTFEPYLYALLKDDSAIEE | 22 |
| | VKKITAERHGTVVTVKRVEKVQKKFLGRPVEVWKLYFTHPQDVPAIMDKI | |
| | REHPAVIDIYEYDIPFAIRYLIDKGLVPMEGDEELKLLAFDIETLYHEGE | |
| | EFAEGPILMISYADEEGARVITWKNVDLPYVDVVSTEREMIKRFLRVVKE | |
| | KDPDVLITYDGDNFDFAYLKKRCEKLGINFALGRDGSEPKIQRMGDRFAV | |
| | EVKGRIHFDLYPVIRRTINLPTYTLEAVYEAVFGQPKEKVYAEEITTAWE | |
| | TGENLERVARYSMEDAKVTYELGKEFLPMEAQLSRLIGQSLWDVSRSSTG | |
| | NLVEWELLRKAYERNELAPNKPDEKELARRHQSHEGGYIKEPERGLWENI | |
| | VYLDERSLYPSIIITHNVSPDTLNREGCKEYDVAPQVGHRFCKDFPGFIP | |
| | SLLGDLLEERQKIKKRMKATIDPIERKLLDYRQRAIKILANSLYGYYGYA | |
| | RARWYCKECAESVIAWGREYLTMTIKEIEEKYGFKVIYSDTDGFFATIPG | |
| | ADAETVKKKAMEFLKYINAKLPGALELEYEGFYKRGLFVTKKKYAVIDEE | |
| | GKITTRGLEIVRRDWSEIAKETQARVLEALLKDGDVEKAVRIVKEVTEKL | |
| | SKYEVPPEKLVIHKQITRDLKDYKATGPHVAVAKRLAARGVKIRPGTVIS | |
| | YIVLKGSGRIVDRAIPFDEFDPTKHKYDAEYYIEKQVLPAVERILRAFGY | |
| | RKEDLRYQKTRQVGLSARLKPKGT | |
| B11 RT | MILDTDYITEDGKPVIRIFKKENGEFKIEYDRTFEPYLYALLKDDSAIEE | 23 |
| | VKKITAERHSTVVTVKRVEKVQKKFLGRSVEVWKLYFTHPQDVPAIMDKI | |
| | REHPAVIDIYEYDIPFAIRYLIDKGLVPMEGDEELKLLALDIGTPCHEGE | |
| | VFAEGPILMISYADEEGTRVITWRNVDLPYVDVLSTEREMIQRFLRVVKE | |
| | KDPDVLITYNGDNFDFAYLKKRCEKLGINFTLGREGSEPKIQRMGDRFAV | |
| | EVKGRIHFDLYPVIRRTVNLPIYTLEAVYEAVFGQPKEKVYAEEITTAWE | |
| | TGENLERVARYSMEDAKVTYELGKEFMPMEAQLSRLIGQSLWDVSRSSTG | |
| | NLVEWELLRKAYERNELAPNKPDEKELARRHQSHEGGYIKEPERGLWENI | |
| | VYLDERSLYPSIIITHNVSPDTLNREGCKEYDVAPQVGHRFCKDFPGFIP | |
| | SLLGDLLEERQKIKKRMKATIDPIERKLLDYRQRAIKILANSLYGYYGYA | |
| | RARWYCKECAESVIAWGREYITMTIKEIEEKYGFKLIYSDTDGFFATIPG | |
| | AEAETVKKKAMEFLKYINAKLPGALELEYEGFYKRGLFVTKKKYAVIDEE | |
| | GKITTRGLEIVRRDWSEIAKETQARVLEALLKDGDVEKAVRIVKEVTEKL | |
| | SKYEVPPEKLVIHKQITRDLKDYKATGPHVAVAKRLAARGVKIRPGTVIS | |
| | YIVLKGSGRIVDRAIPFDEFDPTKHKYDAEYYIENQVLPAVERILRAYGY | |
| | RKEDLWYQKTRQVGLSARLKPKGT | |
| RT CaMV | MDHLLLKTQTQTEQVMNVTNPNSIYIKGRLYFKGYKKIELHCFVDTGASL | 24 |
| | CIASKFVIPEEHWVNAERPIMVKIADGSSITISKVCKDIDLIIAGEIFRI | |
| | PTVYQQESGIDFIIGNNFCQLYEPFIQFTDRVIFTKNKSYPVHIAKLTRA | |
| | VRVGTEGFLESMKKRSKTQQPEPVNISTNKIENPLEEIAILSEGRRLSEE | |
| | KLFITQQRMQKIEELLEKVCSENPLDPNKTKQWMKASIKLSDPSKAIKVK | |
| | PMKYSPMDREEFDKQIKELLDLKVIKPSKSPHMAPAFLVNNEAEKRRGKK | |
| | RMVVNYKAMNKATVGDAYNLPNKDELLTLIRGKKIFSSFDCKSGFWQVLL | |
| | DQESRPLTAFTCPQGHYEWNVVPFGLKQAPSIFQRHMDEAFRVERKFCCV | |
| | YVDDILVFSNNEEDHLLHVAMILQKCNQHGIILSKKKAQLFKKKINFLGL | |
| | EIDEGTHKPQGHILEHINKFPDTLEDKKQLQRFLGILTYASDYIPKLAQI | |
| | RKPLQAKLKENVPWRWTKEDTLYMQKVKKNLQGFPPLHHPLPEEKLIIET | |
| | DASDDYWGGMLKAIKINEGTNTELICRYASGSFKAAEKNYHSNDKETLAV | |
| | INTIKKFSIYLTPVHFLIRTDNTHFKSFVNLNYKGDSKLGRNIRWQAWLS | |
| | HYSFDVEHIKGTDNHFADFLSREFNKVNSSGGS | |
| RT retron | MGIHGVPAAMKSAEYLNTFRLRNLGLPVMNNLHDMSKATRISVETLRLLI | 25 |
| | YTADFRYRIYTVEKKGPEKRMRTIYQPSRELKALQGWVLRNILDKLSSSP | |
| | FSIGFEKHQSILNNATPHIGANFILNIDLEDFFPSLTANKVFGVFHSLGY | |
| | NRLISSVLTKICCYKNLLPQGAPSSPKLANLICSKLDYRIQGYAGSRGLI | |
| | YTRYADDLTLSAQSMKKVVKARDFLFSIIPSEGLVINSKKTCISGPRSQR | |
| | KVTGLVISQEKVGIGREKYKEIRAKIHHIFCGKSSEIEHVRGWLSFILSV | |
| | DSKSHRRLITYISKLEKKYGKNPLNKAKT | |
| HIV2 | MEKEGQLEEAPPTNPYNTPTFAIKKKDKNKWRMLIDFRELNKVTQDFTEI | 26 |
| | QLGIPHPAGLAKKRRITVLDVGDAYFSIPLHEDFRPYTAFTLPSVNNAEP | |
| | GKRYIYKVLPQGWKGSPAIFQHTMRQVLEPFRKANKDVIIIQYMDDILIA | |
| | SDRTDLEHDRVVLQLKELLNGLGFSTPDEKFQKDPPIHWMGYELWPTKWK | |
| | LQKIQLPQKEIWTVNDIQKLVGVLNWAAQLYPGIK | |
| HIV1 | MEEEGKISKIGPENPYNTPVFAIKKKDSTKWRKLVDFRELNKRTQDFWEV | 27 |
| | QLGIPHPAGLKKKKSVTVLDVGDAYFSVPLDESFRKYTAFTIPSINNETP | |
| | GIRYQYNVLPQGWKGSPAIFQSSMTKILEPFRIKNPEIVIYQYMDDLYVG | |
| | SDLEIGQHRTKIEELRAHLLSWGFTTPDKKHQKEPPFLWMGYELHPDRWT | |
| | VQPIDLPEKDSWTVNDIQKLVGKLNWASQIYAGIK | |
| HIV-1 RT p51 | MPISPIETVPVKLKPGMDGPKVKQWPLTEEKIKALVEICTEMEKEGKISK | 28 |
| | IGPENPYNTPVFAIKKKDSTKWRKLVDFRELNKRTQDFWEVQLGIPHPAG | |
| | LKKKKSVTVLDVGDAYFSVPLDEDERKYTAFTIPSINNETPGIRYQYNVL | |
| | PQGWKGSPAIFQSSMTKILEPFRKQNPDIVIYQYMDDLYVGSDLEIGQHR | |
| | TKIEELRQHLLRWGLTTPDKKHQKEPPFLWMGYELHPDKWTVQPIVLPEK | |
| | DSWTVNDIQKLVGKLNWASQIYPGIKVRQLCKLLRGTKALTEVIPLTEEA | |
| | ELELAENREILKEPVHGVYYDPSKDLIAEIQKQGQGQWTYQIYQEPFKNL | |
| | KTGKYARMRGAHTNDVKQLTEAVQKITTESIVIWGKTPKFKLPIQKETWE | |
| | TWWTEYWQATWIPEWEFVNTPPLVKLWYQLEKEPIVGAETF | |
| MMLV RT | STLNIEDEYRLHETSKEPDVSLGSTWLSDFPQAWAETGGMGLAVRQAPLI | 29 |
| lacking the | IPLKATSTPVSIKQYPMSQEARLGIKPHIQRLLDQGILVPCQSPWNTPLL | |
| RNase H domain | PVKKPGTNDYRPVQDLREVNKRVEDIHPTVPNPYNLLSGLPPSHQWYTVL | |
| | DLKDAFFCLRLHPTSQPLFAFEWRDPEMGISGQLTWTRLPQGFKNSPTLF | |
| | NEALHRDLADFRIQHPDLILLQYVDDLLLAATSELDCQQGTRALLQTLGN | |
| | LGYRASAKKAQICQKQVKYLGYLLKEGQRWLTEARKETVMGQPTPKTPRQ | |
| | LREFLGKAGFCRLFIPGFAEMAAPLYPLTKPGTLENWGPDQQKAYQEIKQ | |
| | ALLTAPALGLPDLTKPFELFVDEKQGYAKGVLTQKLGPWRRPVAYLSKKL | |
| | DPVAAGWPPCLRMVAAIAVLTKDAGKLTMGQPLVILAPHAVEALVKQPPD | |
| | RWLSNARMTHYQALLLDTDRVQFGPVVALNPATLLP | |

In some embodiments, the reverse transcriptase polypeptide is fused to a Cas12i polypeptide (e.g., a Cas12i2 or Cas12i4 polypeptide such as those disclosed herein) as in any one of the embodiments described herein. In some embodiments, the reverse transcriptase polypeptide comprises an N-terminal Cas12i polypeptide. In some embodiments, the reverse transcriptase polypeptide comprises a C-terminal Cas12i polypeptide. In some embodiments, the reverse transcriptase polypeptide comprises a Cas12i polypeptide at an intramolecular position within the reverse transcriptase polypeptide (e.g., the Cas12i polypeptide) is within a loop of the reverse transcriptase polypeptide.

In some embodiments, the reverse transcriptase polypeptide comprises a dimerization domain. In some embodiments, a dimerization domain is a leucine zipper, nanobody, or antibody. In some embodiments, the dimerization domain recruits a Cas12i polypeptide.

In some embodiments, the reverse transcriptase polypeptide is an “error-prone” reverse transcriptase variant. Error-prone reverse transcriptases that are known and/or available in the art may be used. It will be appreciated that reverse transcriptases naturally do not have any proofreading function; thus, the error rate of reverse transcriptases is generally higher than DNA polymerases comprising a proofreading activity. In some embodiments, the reverse transcriptase is considered to be “error-prone” if it has an error rate that is less than one error in about 15,000 nucleotides synthesized.

In some embodiments, the reverse transcriptase polypeptide has a mutation or mutations in the RNase H domain. In some embodiments, the reverse transcriptase polypeptide does not comprise an RNase H domain (e.g., the RNase H domain has been removed from the reverse transcriptase polypeptide). In some embodiments, the RNase H domain is truncated in a reverse transcriptase polypeptide. In some embodiments, the reverse transcriptase polypeptide has a mutation or mutations in the RNA-dependent DNA polymerase domain. In some embodiments, the reverse transcriptase polypeptide is a variant that has altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields. Wild-type M-MLV reverse transcriptase typically has an optimal temperature in the range of 37-48 °C; however, mutations may be introduced that allow for the reverse transcription activity at higher temperatures of over 48 °C, including 49 °C, 50 °C, 51C, 52 °C, 53 °C, 54 °C, 55 °C, 56 °C, 57 °C, 58 °C, 59 °C, 60 °C, 61 °C, 62 °C, 63 °C, 64 °C, 65 °C, 66 °C, and higher.

Variant reverse transcriptase polypeptides used herein may be at least about 20% identical, at least about 25% identical, at least about 30% identical, at least about 35% identical, at least about 40% identical, at least about 45% identical, at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to any reference reverse transcriptase polypeptide, including any wild-type reverse transcriptase, mutant reverse transcriptase, or fragment of a reverse transcriptase, or other reverse transcriptase variant disclosed or contemplated herein or known in the art. In some embodiments, a reverse transcriptase variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference reverse transcriptase.

In some embodiments, the reverse transcriptase variant comprises a fragment of a reference reverse transcriptase, such that the fragment is at least about 20% identical, at least about 25% identical, at least about 30% identical, at least about 35% identical, at least about 40% identical, at least about 45% identical, at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference reverse transcriptase.

Variant reverse transcriptases, including error-prone reverse transcriptases, thermostable reverse transcriptases, and reverse transcriptases with increased processivity, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

In some embodiments, the reverse transcriptase polypeptides comprise or is fused to a domain to improve extension rates and/or efficiency of the reverse transcriptase. In some embodiments, the reverse transcriptase polypeptide is fused to an Sso7d polypeptide such as an Sso7d polypeptide from Sulfolobus solfataricus. See, e.g., Wang et al., Nucleic Acids Res. 32(3): 1197-207 (2004).

In some embodiments, a Cas12i-reverse transcriptase fusion polypeptide as described elsewhere herein is capable of binding and binds to at least one nuclease binding sequence in the editing template RNA. In some embodiments, the Cas12i-reverse transcriptase fusion polypeptide is capable of binding and binds to a target sequence through at least one DNA-binding sequence in the editing template RNA. In such embodiments, the Cas12i-reverse transcriptase fusion polypeptide is recruited to or brought in close proximity to the target sequence through binding of the Cas12i via the nuclease binding sequence and the DNA-binding sequence of the editing template RNA.

In some embodiments, the reverse transcriptase transcribes the reverse transcription template sequence into the non-PAM strand of a target nucleic acid starting at the 5′ end of a PBS. In some embodiments, the reverse transcriptase transcribes the reverse transcription template sequence into the non-PAM strand of a target nucleic acid starting at the 3′ end of a PBS. In some embodiments, the reverse transcriptase transcribes the reverse transcription template sequence into the PAM strand of a target nucleic acid starting at the 5′ end of a PBS. In some embodiments, the reverse transcriptase transcribes the reverse transcription template sequence into the PAM strand of a target nucleic acid starting at the 3′ end of a PBS. In some embodiments, following binding of a PBS to a non-PAM strand of a target nucleic acid, the reverse transcriptase transcribes the reverse transcription template sequence from a free 3′ end of the non-PAM strand. In some embodiments, following hybridization of a PBS to a PAM strand of a target nucleic acid, the reverse transcriptase transcribes the reverse transcription template sequence from a free 3′ end of the PAM strand.

In some embodiments, the reverse transcriptase as in any one of the embodiments described herein interacts with a ligase, an integrase, and/or a recombinase. In some embodiments, the reverse transcriptase as in any one of the embodiments described herein is fused to a ligase, an integrase, and/or a recombinase. In some embodiments, the ligase, integrase, and/or recombinase is fused to the N-terminus or C-terminus of the reverse transcriptase. In some embodiments, the ligase, integrase, and/or recombinase is fused internally to the reverse transcriptase. In some embodiments, the integrase is a serine integrase. In some embodiments, the integrase is a Bxb1, TP901, or PhiBT1 integrase. In some embodiments, the recombinase is a serine recombinase or a tyrosine recombinase. In some embodiments, the recombinase is a CRE recombinase. In some embodiments, a reverse transcriptase that interacts with or is fused to a ligase, integrase, and/or recombinase further interacts with or is fused to a Cas12i polypeptide such as a Cas12i2 or Cas12i4 polypeptide.

### B. RNA Components

### RNA Guide

In some embodiments, the gene editing system described herein comprises an RNA guide targeting a TTR gene, for example, targeting exon 2, exon 3, or exon 4 of the TTR gene. In some embodiments, the gene editing system described herein may comprise two or more (e.g., 2, 3, 4, 5, 6, 7, 8, 9, or more) RNA guides targeting TTR.

The RNA guide may direct the Cas12i polypeptide contained in the gene editing system as described herein to an TTR target sequence. Two or more RNA guides may direct two or more separate Cas12i polypeptides (e.g., Cas12i polypeptides having the same or different sequence) as described herein to two or more (e.g., 2, 3, 4, 5, 6, 7, 8, 9, or more) TTR target sequences. Those skilled in the art reading the below examples of particular kinds of RNA guides will understand that, in some embodiments, an RNA guide is TTR target-specific. That is, in some embodiments, an RNA guide binds specifically to one or more TTR target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

### (i). Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some instances, the direct repeat (DR) sequence is recognizable by a Cas12i2 polypeptide. Examples of such DR sequences are provided in Table 4 below.

TABLE 4
Cas12i2 Direct Repeat Sequences

| SEQ ID NO | Direct Repeat Sequence |
|---|---|
| 30 | GUUGCAAAACCCAAGAAAUCCGUCUUUCAUUGACGG |
| 31 | AAUAGCGGCCCUAAGAAAUCCGUCUUUCAUUGACGG |
| 32 | AUUGGAACUGGCGAGAAAUCCGUCUUUCAUUGACGG |
| 33 | CCAGCAACACCUAAGAAAUCCGUCUUUCAUUGACGG |
| 34 | CGGCGCUCGAAUAGGAAAUCCGUCUUUCAUUGACGG |
| 35 | GUGGCAACACCUAAGAAAUCCGUCUUUCAUUGACGG |
| 36 | GUUGCAACACCUAAGAAAUCCGUCUUUCAUUGACGG |
| 37 | GUUGCAAUGCCUAAGAAAUCCGUCUUUCAUUGACGG |
| 38 | GCAACACCUAAGAAAUCCGUCUUUCAUUGACGGG |
| 39 | AGAAAUCCGUCUUUCAUUGACGG |

In some instances, the direct repeat (DR) sequence is recognizable by a Cas12i4 polypeptide. Examples of such DR sequences are provided in Table 5 below.

TABLE 5
Cas12i4 Direct Repeat Sequences

| SEQ ID NO | Direct Repeat Sequence |
|---|---|
| 40 | UCUCAACGAUAGUCAGACAUGUGUCCUCAGUGACAC |
| 41 | UUUUAACAACACUCAGGCAUGUGUCCACAGUGACAC |
| 42 | UUGAACGGAUACUCAGACAUGUGUUUCCAGUGACAC |
| 43 | UGCCCUCAAUAGUCAGAUGUGUGUCCACAGUGACAC |
| 44 | UCUCAAUGAUACUUAGAUACGUGUCCUCAGUGACAC |
| 45 | UCUCAAUGAUACUCAGACAUGUGUCCCCAGUGACAC |
| 46 | UCUCAAUGAUACUAAGACAUGUGUCCUCAGUGACAC |
| 47 | UCUCAACUAUACUCAGACAUGUGUCCUCAGUGACAC |
| 48 | UCUCAACGAUACUCAGACAUGUGUCCUCAGUGACAC |
| 49 | UCUCAACGAUACUAAGAUAUGUGUCCUCAGCGACAC |
| 50 | UCUCAACGAUACUAAGAUAUGUGUCCCCAGUGACAC |
| 51 | UCUCAACGAUACUAAGAUAUGUGUCCACAGUGACAC |
| 52 | UCUCAACAAUACUCAGACAUGUGUCCCCAGUGACAC |
| 53 | UCUCAACAAUACUAAGGCAUGUGUCCCCAGUGACCC |
| 54 | UCUCAAAGAUACUCAGACACGUGUCCCCAGUGACAC |
| 55 | UCUCAAAAAUACUCAGACAUGUGUCCUCAGUGACAC |
| 56 | GCGAAACAACAGUCAGACAUGUGUCCCCAGUGACAC |
| 57 | CCUCAACGAUAUUAAGACAUGUGUCCGCAGUGACAC |
| 58 | AGACAUGUGUCCUCAGUGACAC |

In some embodiments, the direct repeat sequence is a sequence of Table 4 or Table 5 or a portion of a sequence of Table 4 or Table 5. In some embodiments, the direct repeat sequence has at least 95% identity (e.g., at least 95%, 96%, 97%, 98% or 99% identity) to a sequence of Table 4 or Table 5 or a portion of a sequence of Table 4 or Table 5. In some embodiments, the direct repeat sequence has at least 90% identity (e.g., at least 90%, 91%, 92%, 93%, 94%, 95%, 96%, 97%, 98% or 99% identity) to a sequence of Table 4 or Table 5 or a portion of a sequence of Table 4 or Table 5. In some embodiments, the direct repeat sequence is at least 90% identical to the reverse complement of any one of the sequences provided in Table 4 or Table 5. In some embodiments, the direct repeat sequence is at least 95% identical to the reverse complement of any one of the sequences provided in Table 4 or Table 5. In some embodiments, the direct repeat sequence is the reverse complement of any one of the sequences provided in Table 4 or Table 5.

In some embodiments, a direct repeat sequence described herein comprises a uracil (U). In some embodiments, a direct repeat sequence described herein comprises a thymine (T). In some embodiments, a direct repeat sequence according to Tables 4-5 comprises a sequence comprising a thymine in one or more places indicated as uracil in Tables 4-5.

### (ii). Spacer Sequences

In some embodiments, the RNA guide comprises a spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary to a non-PAM strand sequence. In some embodiments, the spacer sequence is designed to be complementary to a specific DNA strand, e.g., of a genomic locus within the TTR gene.

In some embodiments, the RNA guide spacer sequence is substantially identical to a complementary strand of a TTR target sequence. In some embodiments, the RNA guide comprises a sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., a TTR target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a region on the non-PAM strand that is complementary to the TTR target sequence. In some embodiments, the RNA guide comprises a sequence at least 80%, at least 90%, at least 95%, at least 96%, at least 97%, at least 98%, at least 99% complementary to a TTR target DNA sequence. In some embodiments, the RNA guide comprises a sequence at least 80%, at least 90%, at least 95%, at least 96%, at least 97%, at least 98%, at least 99% complementary to a TTR target genomic sequence.

In some instances, the spacer sequence is specific to a target sequence within exon 1, exon 2, exon 3, or exon 4 of the TTR gene. In some examples, the spacer sequence is specific to a target sequence within exon 4. Examples of such spacer sequences are provided in Tables 10 and 16 in Examples 1 and 5 below.

The present disclosure includes RNA guides that comprise any and all combinations of the direct repeats and spacers described herein (e.g., as set forth in Tables 10 and 16). In some embodiments, the RNA guide has at least 90% identity (e.g., at least 90%, 91%, 92%, 93%, 94%, 95%, 96%, 97%, 98% or 99% identity to those provided in Tables 10 and 16 below. In some embodiments, the RNA guide has at least 95% identity (e.g., at least 95%, 96%, 97%, 98% or 99% identity to any one of those provided in Tables 10 and 16 below. In some embodiments, the RNA guide has a sequence set forth in any one of those provided in Tables 10 and 16 below.

### RNA Reverse Transcriptase Donor or RT Donor RNA

The editing template RNA in any of the TTR gene editing systems disclosed herein may also comprise an RNA reverse transcriptase (RT) donor (RT donor RNA). The RT donor RNA may comprise: (i) a primer binding site (PBS), and (ii) a reverse transcription template sequence. In some instances, the RT donor RNA may further comprise: (iii) a nucleotide linker sequence, (iv) a 5′ end and/or 3′ end protection fragment (see disclosures herein), or a combination thereof. In some embodiments, the editing template RNA comprises one or more RT donor RNAs. In some embodiments, the editing template RNA comprises one or more PBS, one or more reverse transcription template sequences, and/or one or more nucleotide linker sequences. In some embodiments, a first editing template RNA comprises one or more PBS and a second editing template RNA comprises one or more reverse transcription template sequences.

In some embodiments, a RT donor RNA comprises an aptamer. In some embodiments, the aptamer recruits a reverse transcriptase polypeptide.

### (i) Primer Binding Site (PBS)

In some embodiments, the PBS in an RT donor RNA as disclosed herein is an RNA sequence capable of binding to a DNA strand via base-paring. The DNA strand has been or can be nicked or cleaved by a Cas12i polypeptide. In some embodiments, the PBS comprises an RNA sequence capable of binding to a DNA strand (a PBS-targeting site) via base-pairing. The DNA strand may have a free 3′ free end or a 3′ free end can be generated via cleavage by a Cas12i polypeptide contained in the same gene editing system. In some examples, the PBS-targeting site may be located on the same DNA strand as the PAM sequence (the PAM strand). In some examples, the PBS-targeting site may be located on the complementary strand of the PAM strand (the non-PAM strand).

In some embodiments, the PBS is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the PBS is about 3 nucleotides to about 200 nucleotides in length (e.g., about 3 nucleotides, 5 nucleotides, 8 nucleotides, 10 nucleotides, 13 nucleotides, 15 nucleotides, 20 nucleotides, 30 nucleotides, 40 nucleotides, 50 nucleotides, 60 nucleotides, 70 nucleotides, 80 nucleotides, 90 nucleotides, 100 nucleotides. 110 nucleotides, 120 nucleotides, 130 nucleotides, 140 nucleotides. 150 nucleotides, 160 nucleotides, 170 nucleotides, 180 nucleotides, 190 nucleotides, 200 nucleotides or any length in between). In some embodiments, the PBS is about 3 nucleotides to about 100 nucleotides in length (e.g., about 3 nucleotides, 5 nucleotides, 8 nucleotides, 10 nucleotides, 13 nucleotides, 15 nucleotides, 20 nucleotides, 30 nucleotides, 40 nucleotides, 50 nucleotides. 60 nucleotides. 70 nucleotides, 80 nucleotides, 90 nucleotides, or 100 nucleotides or any length in between).

In some embodiments, the PBS is about 10 nucleotides to about 50 nucleotides in length. In some embodiments, the PBS is about 10 nucleotides to about 40 nucleotides in length. In some embodiments, the PBS is about 10 nucleotides to about 30 nucleotides in length. In some embodiments, the PBS is about 10 nucleotides to about 20 nucleotides in length. In some embodiments, the PBS is about 10 nucleotides to about 15 nucleotides in length. In some embodiments, the PBS is about 11 nucleotides in length. In some embodiments, the PBS is about 12 nucleotides in length. In some embodiments, the PBS is about 13 nucleotides in length. In some embodiments, the PBS is about 14 nucleotides in length. In some embodiments, the PBS is about 30 nucleotides in length.

In some examples, the PBS is about 20 nucleotides, about 25 nucleotides, about 30 nucleotides, about 35 nucleotides, about 40 nucleotides, about 45 nucleotides, or about 50 nucleotides in length.

In a gene editing system comprising a Cas12i polypeptide (e.g., a Cas12i2 polypeptide as those disclosed herein), the PBS in the RT donor RNA may bind to a region (the PBS-targeting site) on the non-PAM strand. In some instances, the PBS-targeting site may be located upstream to the complementary region of a target sequence. For example, the PBS-targeting site may be up to 20 nucleotides upstream to the complementary region, for example, up to 15 nucleotides, up to 10 nucleotides, or up to 5 nucleotides. In specific examples, the PBS-targeting site may be about 3 nucleotides to about 10 nucleotides upstream of the complementary region. In specific examples, the PBS-targeting site may be 1 nucleotide, 1-2 nucleotides, 1-3 nucleotides, 1-4 nucleotides, 1-5 nucleotides, 1-6 nucleotides, 1-7 nucleotides, 1-8 nucleotides, 1-9 nucleotides, 1-10 nucleotides, 2-3 nucleotides, 2-4 nucleotides, 2-5 nucleotides, 2-6 nucleotides, 2-7 nucleotides, 2-8 nucleotides, 2-9 nucleotides, 2-10 nucleotides, 3-4 nucleotides, 3-5 nucleotides, 3-6 nucleotides, 3-7 nucleotides, 3-8 nucleotides. 3-9 nucleotides, 3-10 nucleotides, 4-5 nucleotides, 4-6 nucleotides, 4-7 nucleotides, 4-8 nucleotides, 4-9 nucleotides, 4-10 nucleotides, 5-6 nucleotides, 5-7 nucleotides, 5-8 nucleotides, 5-9 nucleotides, 5-10 nucleotides, 6-7 nucleotides, 6-8 nucleotides, 6-9 nucleotides, 6-10 nucleotides, 7-8 nucleotides, 7-9 nucleotides, 7-10 nucleotides, 8-9 nucleotides, 8-10 nucleotides, 9-10 nucleotides, or 10 nucleotides upstream of the complementary region. In other instances, the PBS-targeting site may overlap with the complementary region. When a free 3′ end is generated by the Cas12i polypeptide in the gene editing system within or nearby the target sequence and the complementary region, the PBS binding to the non-PAM strand at a site upstream to or overlapping with the complementary region could efficiently facilitate DNA synthesis by the RT polypeptide in the gene editing system, starting from the free 3′ end generated in the non-PAM strand.

Exemplary PBS sequences for use in the TTR gene editing system disclosed herein are provided in Table 10 and Table 16, any of which is within the scope of the present disclosure.

### (ii) Reverse Transcription Template Sequence

The reverse transcription template sequence (template sequence) serves as the template for the reverse transcription mediated by the RT polypeptide in the gene editing system disclosed herein. In some embodiments, the reverse transcription template sequence comprises a sequence with at least one encoded edit. In some embodiments, the reverse transcription template sequence comprises sequence homology to a target sequence or its complementary region with at least one encoded edit. In some embodiments, the reverse transcription template sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the reverse transcription template sequence is about 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, or 120 nucleotides in length or any length in between.

In some embodiments, the reverse transcription template sequence is about 25 nucleotides. In some embodiments, the reverse transcription template sequence is about 26 nucleotides. In some embodiments, the reverse transcription template sequence is about 27 nucleotides. In some embodiments, the reverse transcription template sequence is about 28 nucleotides. In some embodiments, the reverse transcription template sequence is about 29 nucleotides. In some embodiments, the reverse transcription template sequence is about 30 nucleotides. In some embodiments, the reverse transcription template sequence is about 31 nucleotides. In some embodiments, the reverse transcription template sequence is about 32 nucleotides. In some embodiments, the reverse transcription template sequence is about 33 nucleotides. In some embodiments, the reverse transcription template sequence is about 34 nucleotides. In some embodiments, the reverse transcription template sequence is about 35 nucleotides. In some embodiments, the reverse transcription template sequence is about 36 nucleotides. In some embodiments, the reverse transcription template sequence is about 37 nucleotides. In some embodiments, the reverse transcription template sequence is about 38 nucleotides. In some embodiments, the reverse transcription template sequence is about 39 nucleotides. In some embodiments, the reverse transcription template sequence is about 40 nucleotides. In some embodiments, the reverse transcription template sequence is about 41 nucleotides. In some embodiments, the reverse transcription template sequence is about 42 nucleotides. In some embodiments, the reverse transcription template sequence is about 43 nucleotides. In some embodiments, the reverse transcription template sequence is about 44 nucleotides. In some embodiments, the reverse transcription template sequence is about 45 nucleotides. In some embodiments, the reverse transcription template sequence is about 46 nucleotides. In some embodiments, the reverse transcription template sequence is about 47 nucleotides. In some embodiments, the reverse transcription template sequence is about 48 nucleotides. In some embodiments, the reverse transcription template sequence is about 49 nucleotides. In some embodiments, the reverse transcription template sequence is about 50 nucleotides. In some embodiments, the reverse transcription template sequence is about 51 nucleotides. In some embodiments, the reverse transcription template sequence is about 52 nucleotides. In some embodiments, the reverse transcription template sequence is about 53 nucleotides. In some embodiments, the reverse transcription template sequence is about 54 nucleotides. In some embodiments, the reverse transcription template sequence is about 55 nucleotides. In some embodiments, the reverse transcription template sequence is about 56 nucleotides. In some embodiments, the reverse transcription template sequence is about 57 nucleotides. In some embodiments, the reverse transcription template sequence is about 58 nucleotides. In some embodiments, the reverse transcription template sequence is about 59 nucleotides. In some embodiments, the reverse transcription template sequence is about 60 nucleotides.

In some embodiments, the reverse transcription template sequence comprises at least one encoded edit (e.g., at least two) relative to a target sequence. In other embodiments, the reverse transcription template sequence comprises at least one encoded edit (e.g., at least two) relative to the complementary region of a target sequence. In some embodiments, the at least one encoded edit comprises at least one substitution, insertion, and/or deletion. In some embodiments, the edit in the target sequence comprises a substitution, an insertion, and/or a deletion relative to the sequence of a target sequence. In some embodiments, the reverse transcription template sequence comprises at least one LoxP site.

In some embodiments, the edit can be a single or multi-nucleotide substitution, such as a G to T substitution, a G to A substitution, a G to C substitution, a T to G substitution, a T to A substitution, a T to C substitution, a C to G substitution, a C to T substitution, a C to A substitution, an A to T substitution, an A to G substitution, or an A to C substitution. In some embodiments, the change in sequence can convert a G:C base pair to a T:A base pair, a G:C base pair to an A:T base pair, a G:C base pair to C:G base pair, a T:A base pair to a G:C base pair, a T:A base pair to an A:T base pair, a T:A base pair to a C:G base pair, a C:G base pair to a G:C base pair, a C:G base pair to a T:A base pair, a C:G base pair to an A:T base pair, an A:T base pair to a T:A base pair, an A:T base pair to a G:C base pair, or an A:T base pair to a C:G base pair.

The reverse transcription template sequence can be transcribed into DNA by the reverse transcriptase of the gene editing system described herein. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is transcribed from 5′ to 3′ into DNA of the PAM strand. In some embodiments, the reverse transcription template sequence is transcribed from 5′ to 3′ into DNA of the non-PAM strand. In some embodiments, the reverse transcription template sequence is 5′ of the PBS. In some embodiments, the reverse transcription template sequence is 3′ of the PBS. In some embodiments, the reverse transcription template sequence is transcribed into DNA of the PAM strand through 3′ extension from the PBS. In some embodiments, the reverse transcription template sequence is transcribed into DNA of the non-PAM strand through 3′ extension from the PBS.

In some embodiments, the reverse transcription template sequence described herein is designed to correct a TTR mutation associated with a disease. In some embodiments, the mutation is V30M, V122I, T60A, L58H, or I84S, and the disease is hATTR, FAP, or SSA. In some embodiments, mutations V30M. V122I. T60A, L58H, or I84S are in reference to the post-translationally cleaved TTR sequence of SEQ ID NO: 7. In some embodiments, these mutations are referred to as V50M, V1421, T80A, L78H, and I104S with respect to the full-length TTR sequence of SEQ ID NO: 6.

In some embodiments, the target sequence in the TTR gene encodes a V30M mutation, and the template sequence (or its complementary sequence) comprises a mutation to revert M to V at position 30. In some embodiments, the target sequence comprises a 148G>A mutation in the TTR coding sequence of SEQ ID NO: 8 and the template sequence (or its complementary sequence) comprises a 148A>G mutation in the corresponding region of the TTR coding sequence of SEQ ID NO: 8.

In some embodiments, the target sequence encodes a V122I mutation, and the template sequence (or its complementary sequence) comprises a mutation to revert I to V at position 122. In some embodiments, the target sequence comprises a 424G>A mutation in the TTR coding sequence of SEQ ID NO: 8 and the template sequence (or its complementary sequence) comprises a 424A>G mutation in the corresponding region of the TTR coding sequence of SEQ ID NO: 8.

In some embodiments, the target sequence encodes a T60A mutation, and the template sequence (or its complementary sequence) comprises a mutation to revert A to T at position 60. In some embodiments, the target sequence comprises a 238A>G mutation in the TTR coding sequence of SEQ ID NO: 8 and the template sequence (or its complementary sequence) comprises a 238G>A mutation in the corresponding region of the TTR coding sequence of SEQ ID NO: 8.

In some embodiments, the target sequence encodes an L58H mutation, and the template sequence (or its complementary sequence) comprises a mutation to revert H to L at position 58. In some embodiments, the target sequence comprises a 233T>A mutation in the TTR coding sequence of SEQ ID NO: 8 and the template sequence (or its complementary sequence) comprises a 233A>T mutation in the corresponding region of the TTR coding sequence of SEQ ID NO: 8.

In some embodiments, the target sequence encodes an I84S mutation, and the template sequence (or its complementary sequence) comprises a mutation to revert S to I at position 84. In some embodiments, the target sequence comprises a 311T>G mutation in the TTR coding sequence of SEQ ID NO: 8 and template sequence (or its complementary sequence) comprises a 311G>T mutation in the corresponding region of the TTR coding sequence of SEQ ID NO: 8.

In some embodiments, a template sequence described herein is used to introduce a protective mutation associated with a disease. In some embodiments, the mutation is T119M, and the disease is hATTR. In some embodiments, the mutation is T119M, and the disease is wild-type ATTR amyloidosis. In some embodiments, the T119M mutation is in reference to the post-translationally cleaved TTR sequence of SEQ ID NO: 7. In some embodiments, the T119M mutation is referred to as T139M with respect to the full-length TTR sequence of SEQ ID NO: 6. In some embodiments, a target sequence encodes a T119 residue, and the template sequence (or its complementary sequence) comprises a mutation to introduce the T119M substitution in the encoded TTR protein. In some embodiments, the target sequence comprises a C in the 416 position of the TTR coding sequence of SEQ ID NO: 8 and the template sequence (or its complementary sequence) comprises a 416C>T mutation in the corresponding region of the TTR coding sequence of SEQ ID NO: 8.

In some embodiments, a template sequence described herein may further introduce one or more silent mutations. As used herein, a silent mutation refers to a mutation that does not change the amino acid residue encoded by the codon comprising the mutation. Without wishing to be bound by theory, introduction of one or more silent mutations into a TTR target reduces or eliminates re-targeting of the resultant edited genomic site by an editing template RNA (e.g., the same editing template RNA). In some examples, one or more silent mutations are introduced into the genomic site where the codon for T119 locates, together with the mutation to introduce the T119M substitution.

Exemplary targeting sequences for use in introducing the T119M mutation to the TTR gene are provided in Table 10 and Table 16, any of which is within the scope of the present disclosure.

### Additional Elements

In some embodiments, the editing template RNA may comprise one or more additional elements. For example, the editing template RNA, or the gRNA and/or the RT donor RNA thereof, may comprise one or more protection fragments at either or both ends of the RNA molecules. Alternatively or in addition, the editing template RNA, or the gRNA and/or the RT donor RNA thereof, may comprise additional elements internal to the RNA molecule (e.g., between one or more of the sequences in the editing template RNA, e.g., between a PBS and a reverse transcription template sequence, e.g., a linker). In some embodiments, the editing template RNA comprises additional elements between one or more sequence of the editing template RNA, e.g., such as an RNA guide (a nuclease binding sequence or a DNA-binding sequence) or an RT donor RNA (a PBS or a reverse transcription template sequence).

In some embodiments, the editing template RNA comprises additional elements, e.g., a direct repeat sequence, at one or more ends. In some embodiments, the direct repeat sequence may recruit a Cas12i polypeptide (e.g., a variant Cas12i2 polypeptide, a variant Cas12i2-reverse transcriptase fusion polypeptide, a variant Cas12i4 polypeptide, or a Cas12i4-reverse transcriptase fusion polypeptide).

In some embodiment, the additional elements may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In some examples, the editing template RNA may comprise an optional nucleotide linker. Such an optional nucleotide linker sequence may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length. In some embodiments, the optional nucleotide linker is between any of the nuclease binding sequence, the DNA-binding sequence, the PBS and/or reverse transcription template sequence.

In some examples, the 5′ end and/or the 3′ end of the editing template RNA, or the gRNA and/or the RT donor RNA thereof, may contain a protection fragment, which may enhance resistance of the RNA molecule to exonuclease activity. In some instances, the end protection fragment may comprise a nucleotide sequence capable of forming a secondary structure, such as hairpin, a pseudoknot, or a triplex structure. In other instances, the end protection fragment may comprise the sequence of an exoribonuclease-resistant RNA (xrRNA), a transfer RNA (tRNA), or a truncated tRNA. In some embodiments, the modification is a Zika-like pseudoknot, a murine leukemia virus pseudoknot (MLV-PK) sequence, a red clover necrotic mosaic virus (RCNMV) sequence, a sweet clover necrotic mosaic virus (SCNMV) sequence, a carnation ringspot virus (CRSV) sequence, preQ sequence, or an RNA bacteriophage MS2 sequence. In specific examples, the end protection fragment may comprise one or more CRISPR nuclease binding sites (e.g., bindings sites for a Cas12i polypeptide such as a Cas12i2 polypeptide), and optionally one or more segments (e.g., spacers) that share no homology with any human sequences. In some instances, the one or more segments bind to a sequence that is no more than 85% identical to any sequence of the human genome. Such an end protection fragment can recruit the CRISPR nuclease contained in the same gene editing system to inhibit exoribonuclease activity without inducing off-target gene edits.

### Nucleic Acid Modifications

The RNA guide or template DNA may include one or more covalent modifications with respect to a reference sequence, in particular the parent polyribonucleotide, which are included within the scope of this disclosure.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide or template DNA may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210. Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone. Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In some embodiments, the template DNA comprises one or more internucleoside modifications (e.g., phosphorothioate modifications). In some embodiments, the left homology arm comprises one or more internucleoside modifications (e.g., phosphorothioate modifications). In some embodiments, the right homology arm comprises one or more internucleoside modifications (e.g., phosphorothioate modifications). In some embodiments, the left homology arm comprises one or more internucleoside modifications (e.g., phosphorothioate modifications) and the right homology arm comprises one or more internucleoside modifications (e.g., phosphorothioate modifications). In some embodiments, the left homology arm comprises two internucleoside modifications (e.g., phosphorothioate modifications) and the right homology arm comprises two internucleoside modifications (e.g., phosphorothioate modifications). In some embodiments, the phosphorothioate modifications are at the 5′ end of the left homology arm and the 3′ end of the right homology arm. In some embodiments, the left homology arm is 5′ of the right homology arm and the left homology arm comprises two phosphorothioate modifications at the 5′ end of the left homology arm, the right homology arm comprises two phosphorothioate modifications at the 3′ end of the right homology arm. In some embodiments, the template DNA is a double stranded DNA that comprises a first strand and a second strand, wherein the first strand comprises at least one (e.g., 2) phosphorothioate modifications at the 5′ end of the first strand or at least one (e.g., 2) phosphorothioate modifications at the 3′ end of the first strand, or both. In some embodiments, the second strand comprises at least one (e.g., 2) phosphorothioate modifications at the 5′ end of the second strand or at least one (e.g., 2) phosphorothioate modifications at the 3′ end of the second strand, or both.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J. Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine. 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine. 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine. 5-methyl-zebularine. 5-aza-2-thio-zebularine. 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine. 7-deaza-adenine. 7-deaza-8-aza-adenine, 7-deaza-2-aminopurine. 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine. 7-methyl-8-oxo-guanosine, 1-methyl-6-thio-guanosine. N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

When a gene editing system disclosed herein comprises nucleic acids encoding the Cas12i polypeptide disclosed herein, e.g., mRNA molecules, such nucleic acid molecules may contain any of the modifications disclosed herein, where applicable.

### C. Exemplary Gene Editing Systems

The exemplary gene editing systems described herein are meant to be illustrative only. An exemplary TTR gene editing system disclosed herein comprises a Cas12i2 or Cas12i4 polypeptide (e.g., a Cas12i2 variant such as SEQ ID NO: 11, 14, 352, or 355 or a Cas12i4 variant such as SEQ ID NO: 16), an RT polypeptide (e.g., the mutant MMLV RT provided herein), or nucleic acid(s) encoding the enzymes, an RNA guide targeting a genomic site in the TTR gene (e.g., exon 4), and an RT donor RNA comprising a template sequence designed to introduce one or more mutations into the genomic site of the TTR gene (e.g., introducing the T119M substitution at the encoded TTR protein).

In some examples, the TTR gene editing system comprises a fusion polypeptide comprising the Cas12i2 or Cas12i4 polypeptide and the RT polypeptide (e.g., the fusion polypeptide comprising SEQ ID NO: 59, 353, or 238), or a nucleic acid (e.g., an mRNA) encoding the fusion polypeptide.

In Alternatively or in addition, the TTR gene editing system comprises an editing template RNA, which can be a single polyribonucleotide comprising the RNA guide and the RT donor RNA. In some instances, the editing template RNA has the configuration of, from 5′ end to 3′ end, a template sequence (e.g., those provided in Table 10 or Table 16), a PBS (e.g., those provided in Table 10 or Table 16), a direct repeat sequence (e.g., those provided in Table 10 or Table 16), and a spacer sequence (e.g., those provided in Table 10 or Table 16). In some examples, the template sequence is designed to introduce a mutation that would lead to the T119M substation in the encoded TTR protein and one or more additional mutations (e.g., silence mutations, see, e.g., FIG. 2) to reduce or eliminate re-targeting of the edited TTR gene. Exemplary editing template RNAs are provided in Table 11 and Table 17 (e.g., editing template RNA 60, editing template RNA 64, or editing template RNA 98).

In some embodiments, a gene editing system as disclosed herein may comprise the protein components of the Cas12i polypeptide, the RT polypeptide, or both. Alternatively, the gene editing system may comprise one or more nucleic acids (e.g., vectors such as viral vectors) encoding the protein components. In some examples, the gene editing system may comprise one vector encoding both the Cas12i polypeptide and the RT polypeptide.

Alternatively or in addition, a gene editing system as disclosed herein may comprise the RNA components of the gene editing RNA, the guide RNA, or both. Alternatively, the gene editing system may comprise one or more nucleic acids (vectors) encoding the RNA components. For example, the gene editing system may comprise one vector (e.g., a viral vector such as an AAV vector, e.g., AAV1, AAV2, AAV3, AAV4, AAV5, AAV6, AAV7, AAV8, AAV9, AAVrh10, AAV11 and AAV12) coding for both the gene editing RNA and the RNA guide. Alternatively, the gene editing system may comprise one or more mRNA molecules encoding the Cas12i polypeptide and the RT polypeptide as disclosed herein.

In some examples, a gene editing system as disclosed herein may comprise the protein components of the Cas12i polypeptide, the RT polypeptide, or both, and the RNA components of gene editing RNA and the RNA guide. In other examples, a gene editing system as disclosed herein may comprise the protein components of the Cas12i polypeptide, the RT polypeptide, or both, and one or more nucleic acids encoding the RNA components of gene editing RNA and the RNA guide. In yet other examples, a gene editing system as disclosed herein may comprise one or more nucleic acids encoding the protein components of the Cas12i polypeptide, the RT polypeptide, or both, and the RNA components of gene editing RNA and the RNA guide. Alternatively, a gene editing system as disclosed herein may comprise one or more nucleic acids encoding the protein components of the Cas12i polypeptide, the RT polypeptide, or both, and one of more nucleic acids encoding the RNA components of gene editing RNA and the RNA guide. In some instances, the gene editing system may comprise one vector encoding multiple components of the gene editing system. In some instances, the nucleic acid(s) encoding the Cas12i polypeptide, the RT polypeptide, and/or a fusion polypeptide thereof can be one or more mRNA molecules. In some examples, the mRNA molecule(s) may be codon optimized.

In some embodiments, the gene editing system disclosed herein comprises one or more lipid nanoparticles (LNPs) encompassing one or more of the protein and/or RNA components of the gene editing system, or their encoding nucleic acids. In other embodiments, the gene editing system may comprise one or more LNPs encompass a portion the components and one or more vectors encoding the remaining components.

### II. Preparation of Gene Editing System Components

The protein components, the RNA components, or their encoding nucleic acids (e.g., vectors or mRNAs) may be prepared by conventional methods of the methods disclosed herein.

In some embodiments, a Cas12i polypeptide, a reverse transcriptase, or a Cas12i-reverse transcriptase fusion can be prepared by (a) culturing host cells such as bacteria cells or mammalian cells, capable of producing the proteins, isolating the proteins thus produced, and optionally, purifying the proteins. The Cas12i polypeptide, the reverse transcriptase, or the fusion protein thus prepared may be complexed with the editing template RNA.

The Cas12i polypeptide and the reverse transcriptase can be also prepared by (b) a known genetic engineering technique, specifically, by isolating a gene encoding the Cas12i polypeptide and the reverse transcriptase of the present invention from bacteria, constructing a recombinant expression vector, and then transferring the vector into an appropriate host cell that expresses the editing template RNA for expression of a recombinant protein that complexes with the editing template RNA in the host cell. Alternatively, the Cas12i polypeptide and the reverse transcriptase can be prepared by (c) an in vitro coupled transcription-translation system and then complexes with editing template RNA. Bacteria that can be used for preparation of the Cas12i polypeptide and the reverse transcriptase of the present invention are not particularly limited as long as they can produce the Cas12i polypeptide and the reverse transcriptase of the present invention. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### A. Vectors

The present disclosure provides one or more vectors for expressing the Cas12i polypeptide, the reverse transcriptase, or their fusion polypeptide described herein or nucleic acids encoding the components described herein may be incorporated into a vector. In some embodiments, a vector disclosed herein includes a nucleotide sequence encoding Cas12i polypeptide, the reverse transcriptase, or the fusion polypeptide. The present disclosure also provides one or more vectors encoding the editing template RNA or any portion thereof, e.g., the RNA guide, or the RT donor RNA. In some embodiments, the vector comprises a Pol II promoter or a Pol III promoter.

Expression of natural or synthetic polynucleotides is typically achieved by operably linking a polynucleotide encoding the gene of interest, e.g., nucleotide sequence encoding the Cas12i polypeptide, the reverse transcriptase, or the fusion polypeptide, and/or the editing template RNA, to a promoter and incorporating the construct into an expression vector. The expression vector is not particularly limited as long as it includes a polynucleotide encoding the Cas12i polypeptide and the reverse transcriptase and/or the editing template RNA of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.), may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method for recombinant expression vectors is not particularly limited, and examples thereof include methods using a plasmid, a phage or a cosmid.

### B. Methods of Expression

The present disclosure includes a method for protein expression, comprising translating the Cas12i polypeptide and the reverse transcriptase, and expressing the editing template RNA described herein.

In some embodiments, a host cell described herein is used to express the Cas12i polypeptide and the reverse transcriptase and/or the editing template RNA. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells. i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide, the reverse transcriptase and/or the editing template RNA. After expression of the Cas12i polypeptide, the reverse transcriptase and/or the editing template RNA, the host cells can be collected and Cas12i polypeptide, the reverse transcriptase and/or the editing template RNA purified from the cultures etc. according to conventional methods (for example, filtration, centrifugation, cell disruption, gel filtration chromatography, ion exchange chromatography, etc.).

In some embodiments, the methods for Cas12i polypeptide and the reverse transcriptase expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the polypeptide(s). In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide and the reverse transcriptase.

A variety of methods can be used to determine the level of production of a mature Cas12i polypeptide, the reverse transcriptase and/or the editing template RNA in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i polypeptide and the reverse transcriptase and/or the editing template RNA in a cell, comprising providing a polyribonucleotide encoding the Cas12i polypeptide, the reverse transcriptase and/or the editing template RNA to a host cell wherein the polyribonucleotide encodes the Cas12i polypeptide, the reverse transcriptase and/or the editing template RNA, expressing the Cas12i polypeptide, the reverse transcriptase and/or the editing template RNA in the cell, and obtaining the Cas12i polypeptide, the reverse transcriptase and/or the editing template RNA from the cell.

### III. Genetic Editing Methods

Any of the gene editing systems can be used to genetically modify (edit) a target nucleic acid, which can be a genetic site within the TTR gene, e.g., a genetic site where genetic editing is needed, for example, to fix a genetic mutation, to introduce a protective mutation, to introduce modifications for modulating expression of a gene, etc.

### A. TTR Gene Editing

The gene editing systems and compositions disclosed herein are applicable for editing and introducing edits into a variety of target sequences.

In some embodiments, the methods comprise introducing a TTR-targeting RNA guide, a Cas12i polypeptide, a reverse transcriptase, and an RT donor RNA into a cell. The TTR-targeting RNA guide and Cas12i polypeptide can be introduced as a ribonucleoprotein complex into a cell. The TTR-targeting RNA guide, RT donor RNA, Cas12i polypeptide, and the RT polypeptide can be introduced on a nucleic acid vector. The Cas12i polypeptide and/or the RT polypeptide (e.g., a fusion polypeptide comprising both) can be introduced as an mRNA. The RNA guide and RT donor RNA can be introduced directly into the cell. In some instances, LNPs can be used to facilitate delivery of one or more of the TTR gene editing systems into target cells.

In some embodiments, the TTR gene editing system described herein is used to introduce a protective mutation associated with a disease. In some embodiments, the mutation is T119M, and the disease is hATTR. In some embodiments, the mutation is T119M. and the disease is wild-type ATTR amyloidosis. In some embodiment, the T119M mutation is in reference to the post-translationally cleaved TTR sequence of SEQ ID NO: 7. In some embodiments, the T119M mutation is referred to as T139M with respect to the full-length TTR sequence of SEQ ID NO: 6.

In some embodiments, the TTR gene editing system disclosed herein is used to correct a mutation associated with a disease. In some embodiments, the mutation is V30M, V122I, T60A, L58H, or I84S, and the disease is hATTR, FAP, or SSA. In some embodiments, mutations V30M, V122I, T60A, L58H, or I84S are in reference to the post-translationally cleaved TTR sequence of SEQ ID NO: 7. When the full length TTR sequence (SEQ ID NO: 6) is used as the reference sequence, these mutations are referred to as V50M, V1421, T80A, L78H, and I104S with respect to the full-length TTR sequence of SEQ ID NO: 6.

In some embodiments, the TTR gene editing system described herein is used to correct a mutation associated with a disease and to introduce a protective mutation. In some embodiments, the mutation associated with disease is V30M, V122I, T60A, L58H, or I84S and the disease is (hATTR). In some embodiments, the protective mutation is T119M. In some embodiments, the disease is hATTR or wild-type ATTR amyloidosis. For example, in some embodiments, a template DNA described herein is used to correct a V30M mutation and to introduce a T119M mutation. In some embodiments, a template DNA described herein is used to correct a V122I mutation and to introduce a T119M mutation. In some embodiments, a template DNA described herein is used to correct a T60A mutation and to introduce a T119M mutation. In some embodiments, a template DNA described herein is used to correct an L58H mutation and to introduce a T119M mutation. In some embodiments, a template DNA described herein is used to correct an I84S mutation and to introduce a T119M mutation.

In some embodiments, a composition described herein is introduced into a population of cells. In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%, 2%, 3%, 4%, 5%, 6%. 7%, 8%, 9%, 10%, 11%, 12%, 13%, 14%, 15%, 16%, 17%, 18%, 19%, 20%, 21%, 22%, 23%, 24%, or 25% of the cells comprise a wild-type TTR gene (e.g., one of more of the following mutations are corrected: V30M. V122I, T60A, L58H, and I84S and/or one or more of desired mutations are introduced, such as T119M).

In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%, 2%, 3%, 4%, 5%, 6%, 7%, 8%, 9%, 10%, 11%, 11%, 12%, 13%, 14%, 15%, 16%, 17%, 18%, 19%, 20%, 21%, 22%, 23%, 24%, or 25% of the cells comprise a T119M mutation.

In some embodiments, at least about 10%, 15%, 20%, 25%, 30%, 35%, 40%, 45%, 50%, 55%, 60%, 65%, 70%, 75%, 80%, 85%, 90%, 95%, or 100% of the cells comprise a deletion described herein. In some embodiments, at least about 1%. 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of the cells comprise a wild-type TTR gene (e.g., one of more of the following mutations were corrected: V30M, V122I, T60A, L58H, and I84S) as well as a T119M mutation.

In some embodiments, the RNA guide targeting TTR is encoded in a plasmid. In some embodiments, the RNA guide targeting TTR is synthetic or purified RNA. In some embodiments, the Cas12i polypeptide is encoded in a plasmid. In some embodiments, the Cas12i polypeptide is encoded by an RNA that is synthetic or purified.

### B. Delivery of TTR Gene Editing System

Components of any of the gene editing systems disclosed herein may be formulated, for example, including a carrier, such as a carrier and/or a polymeric carrier, e.g., a liposome, and delivered by known methods to a cell (e.g., a mammalian cell). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, adeno-associated virus (AAV)), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. In some examples, the delivery method involves the use of lipid nanoparticles to mediate delivery of one or more components of the TTR gene editing system disclosed herein.

In some embodiments, the method comprises delivering one or more nucleic acids (e.g., nucleic acids encoding the Cas12i polypeptide, RT polypeptide, RNA guide, RT donor RNA, etc.), one or more transcripts thereof, and/or a pre-formed RNA guide/Cas12i polypeptide/RT polypeptide complex to a cell, where a ternary complex is formed. In some embodiments, an RNA guide and/or RT donor RNA, or a fusion thereof, and an RNA encoding a Cas12i polypeptide or a RT polypeptide, or a fusion polypeptide comprising both, are delivered together in a single composition. In some embodiments, an RNA guide and an RNA encoding a Cas12i polypeptide are delivered in separate compositions. In some embodiments, an RNA guide/RT donor RNA and an RNA encoding a Cas12i polypeptide/RT polypeptide delivered in separate compositions are delivered using the same delivery technology. In some embodiments, an RNA guide/RT donor RNA and an RNA encoding a Cas12i polypeptide/RT polypeptide delivered in separate compositions are delivered using different delivery technologies.

In some embodiments, one or more of the protein components and one or more of the RNA components are delivered together. For example, the Cas12i and/or RT polypeptide and the RNA guide and/or RT donor RNA are packaged together in a single AAV particle. In another example, the Cas12i and/or RT polypeptide and the RNA guide and/or RT donor RNA are delivered together via lipid nanoparticles (LNPs). In some embodiments, the Cas12i and/or RT polypeptides and the RNA guide and/or RT donor RNA are delivered separately. For example, the Cas12i and/or RT polypeptides and the RNA guide and/or RT donor RNA are packaged into separate AAV particles. In another example, the Cas12i and/or RT polypeptides is delivered by a first delivery mechanism and the RNA guide and/or RT donor RNA is delivered by a second delivery mechanism.

Exemplary intracellular delivery methods, include, but are not limited to: viruses, such as AAV, or virus-like agents; chemical-based transfection methods, such as those using calcium phosphate, dendrimers, liposomes, or cationic polymers (e.g., DEAE-dextran or polyethylenimine): non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, a lipid nanoparticle comprises an mRNA encoding a Cas12i-RT fusion polypeptide, an editing template RNA, or an mRNA encoding such. In some embodiments, the present application further provides cells produced by such methods, and organisms (such as animals, plants, or fungi) comprising or produced from such cells.

### C. Genetically Modified Cells

Any of the TTR gene editing systems disclosed herein can be delivered to a variety of cells (e.g., to mammalian cells such as a mouse cell, a non-human primate cell, or a human cell). In some embodiments, the cell is in cell culture or a co-culture of two or more cell types. In some embodiments, the cell is ex vivo. In some embodiments, the cell is obtained from a living organism and maintained in a cell culture.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

In some examples, any of the TTR gene editing systems may be applied to a population of stem cells such as iPSC cells and the genetically modified stem cells (e.g., iPSC cells) thus obtained may be differentiated into a suitable type of cells, for example, liver cells, which may be used for therapeutic purposes.

Any of the genetically modified cells produced using any of the gene editing system disclosed herein is also within the scope of the present disclosure. Such modified cells may comprise a disrupted TTR gene.

Any of the gene editing systems, compositions comprising such, vectors, nucleic acids, RNA guides and cells disclosed herein may be used in therapy. Gene editing systems, compositions, vectors, nucleic acids, RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a TTR sequence as disclosed herein. In some embodiments, a cell engineered using an RNA guide disclosed herein is used for ex vivo gene therapy.

### IV. Therapeutic Applications

Any of the TTR gene editing systems or modified cells generated using such a gene editing system as disclosed herein may be used for treating a disease that is associated with the TTR gene, for example, amyloidogenic transthyretin (ATTR). In some instances, the ATTR is hereditary ATTR (hATTR) or wild-type ATTR amyloidosis.

hATTR amyloidosis (also referred to as transthyretin familial amyloid polyneuropathy (TTR-FAP) or familial amyloid cardiomyopathy (TTR-FAC)) is a systemic disorder characterized by the extracellular deposition of misfolded transthyretin (TTR) protein. Normally, TTR is a tetramer made up of 4 single-chain monomers. TTR gene mutations are thought to destabilize the protein and cause tetramer dissociation into monomers, which aggregate into amyloid fibrils. These amyloid fibrils then accumulate in multiple organs throughout the body.

hATTR amyloidosis is an autosomal dominant disease with variable penetrance. Amyloid deposition or symptomatic disease typically occurs in adults ranging from 30 to 70 years of age, depending on mutation. Over 120 amyloidogenic TTR mutations have been identified. Some hATTR disease causing/associated SNPs are shown below in Table 6. The positions of the DNA mutations are relative to the TTR coding sequence of SEQ ID NO: 8. The positions of the amino acid mutations are relative to the post-translationally cleaved TTR protein sequence of SEQ ID NO: 7 (top mutation) or the full-length TTR protein sequence of SEQ ID NO: 6 (bottom mutation in parentheses).

TABLE 6
hATTR Disease Causing/Associated SNPs

| DNA | Amino acid | Age of | Disease | | Cases |
|---|---|---|---|---|---|
| mutation | mutation | onset | course | Location | published |
| None | WT | 80 | Late-onset | Worldwide | ~25% of |
| | | | cardiac | | people over |
| | | | involvement | | age 80 |
| c. 148G > A | V30M | 30-60 | Presents with | Worldwide | >400 |
| | (V50M) | | polyneuropathy | | |
| | | | and autonomic | | |
| | | | dysfunction, | | |
| | | | then systemic | | |
| | | | involvement | | |
| c. 424G > A | V122I | 60s | Late-onset | African- | Most |
| | (V142I) | | cardiac | American | common |
| | | | amyloidosis | | variant |
| c. 238G > A | T60A | 50s | Late-onset | Appalachian, | >40 |
| | (T80A) | | cardiac | Ireland | |
| | | | amyloidosis | | |
| | | | with some | | |
| | | | neurologic | | |
| | | | involvement | | |
| c. 233T > A | L58H | 40s | Usually | Maryland, | >50 |
| | (L78H) | | presents with | German | |
| | | | CTS and is | | |
| | | | slowly | | |
| | | | progressive | | |
| | | | over two | | |
| | | | decades | | |
| c. 311T > G | I84S | 20-30 | CTS at 30, | Indiana, | >30 |
| | (I104S) | | vitreous | Switzerland | |
| | | | opacites 40- | | |
| | | | 50s, then heart | | |
| | | | involvement | | |

The T119M mutation is considered to be non-amyloidogenic and stabilize the TTR tetramer in patients with hATTR. See, e.g., Batista et al., Gene Therapy 21: 1041-50 (2014) and Yee et al., Nature Communications 10: 925 (2019). Therefore, T119M is considered a protective mutation. The T119M mutation can be introduced to treat hATTR in subjects having an amyloidogenic TTR mutation or to treat patients having wild-type ATTR amyloidosis.

In some embodiments, provided herein is a method for treating a target disease as disclosed herein (e.g., amyloidogenic transthyretin (ATTR) such as hATTR) comprising administering to a subject (e.g., a human patient) in need of the treatment any of the gene editing systems disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by a Cas12i polypeptide (e.g., a Cas12i2 or Cas12i4 polypeptide as disclosed herein), reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edit in the TTR gene. In some examples, the cells can be suspended in a suitable media.

In some examples, the modified cells disclosed herein, comprising one particular gene edit in the TTR gene such as those disclosed herein, may be liver cells. In some instances, such modified liver cells may be derived via differentiation of stem cells, for example, iPSC cells, which can be genetically modified by any of the TTR gene editing systems provided herein. In some instances, an effective amount of the modified liver cells may be administered to a human patient in need of the treatment via a suitable route for treating a disease associated with the TTR gene, for example, amyloidogenic transthyretin (ATTR) such as hATTR.

In some embodiments, provided herein is a composition comprising the gene editing system or components thereof. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition (e.g., the gene editing system or components thereof), which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits that can be used, for example, to carry out a method described herein for genetical modification of the TTR gene. In some embodiments, the kits include an editing template RNA (including an RNA guide and an RT donor RNA), a Cas12i polypeptide, and an RT polypeptide, or a fusion polypeptide thereof. In some embodiments, the kits include the editing template RNA and a Cas12i-RT fusion polypeptide. In some embodiments, the kits include a polynucleotide that encodes a Cas12i polypeptide, an RT polypeptide, or a fusion thereof, and optionally the polynucleotide is comprised within a vector, e.g., as described herein. In some embodiments, the kits include a polynucleotide that encodes an editing template RNA(s) disclosed herein. The Cas12i polypeptide, the RT polypeptide, or a fusion polypeptide thereof (or polynucleotide encoding such) and the editing template RNA (e.g., as a ribonucleoprotein) can be packaged within the same or other vessel within a kit or can be packaged in separate vials or other vessels, the contents of which can be mixed prior to use.

The Cas12i polypeptide, the RT polypeptide, and the editing template RNA can be packaged within the same or other vessel within a kit or can be packaged in separate vials or other vessels, the contents of which can be mixed prior to use. The kits can additionally include, optionally, a buffer and/or instructions for use of the editing template RNA, the Cas12i polypeptide, and the RT polypeptide.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts. 1998) Plenum Press: Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press. 1988-1989); Monoclonal antibodies: a practical approach (P. Shepherd and C. Dean, eds., Oxford University Press, 2000): Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the present disclosure in any way whatsoever. All publications cited herein are incorporated by reference for the purposes or subject matter referenced herein.

### EXAMPLES

The following examples are provided to further illustrate some embodiments of the present disclosure but are not intended to limit the scope of the present disclosure; it will be understood by their exemplary nature that other procedures, methodologies, or techniques known to those skilled in the art may alternatively be used.

### Example 1—RNA-Templated Editing of the TTR Gene in HEK293T Cells Using Chemically Modified Editing Template RNAs and DNA Delivery of a Cas12i2-RT Fusion Polypeptide

This Example describes editing of the human TTR gene with a plasmid-encoded Cas12i2-RT fusion protein and editing template RNAs containing terminal phosphorothioate backbone linkages and 2′O-methyl nucleotides.

A fusion of a variant Cas12i2 (SEQ ID NO. 352) with mutant MMLV reverse transcriptase (RT) was cloned into the pcda3.1 backbone (Invitrogen). The configuration of the C-terminal RT fusion to variant Cas12i2 and the amino acid sequence of the fusion polypeptide are shown in Table 7. A working solution of plasmid for expression of RT fusion with variant Cas12i2 was prepared in water (variant Cas12i2-RT fusion working solution).

TABLE 7
Cas12i2-RT Fusion Polypeptide Exemplary Design and Sequence

| | | | SEQ |
|---|---|---|---|
| Full | Shorthand | | ID |
| Description | Description | Sequence | NO |
| Cas12i2 variant | C_MMLV Cas | MSSAIKSYKSVLRPNERKNQLLKSTIQCLEDGSAFFFKMLQG | 59 |
| (H485A/D581R/ | 12i2 | LEGGITPEIVRESTEQEKQQQDIALWCAVNWFRPVSQDSLTH | |
| I926R/V1030G) | variant | TIASDNLVEKFEEYYGGTASDAIKQYFSASIGESYYWNDCRQ | |
| -NLS-2xSGGS | | QYYDLCRELGVEVSDLTHDLEILCREKCLAVATESNQNNSII | |
| -XTEN (16 aa)- | | SVLFGTGEKEDRSVKLRITKKILEAISNLKEIPKNVAPIQEI | |
| 2xSGGS- | | ILNVAKATKETFRQVYAGNLGAPSTLEKFIAKDGQKEFDLKK | |
| mutMMLVRT- | | LQTDLKKVIRGKSKERDWCCQEELRSYVEQNTIQYDLWAWGE | |
| NLS | | MFNKAHTALKIKSTRNYNFAKQRLEQFKEIQSLNNLLVVKKL | |
| | | NDFFDSEFFSGEETYTICVHHLGGKDLSKLYKAWEDDPADPE | |
| | | NAIVVLCDDLKNNFKKEPIRNILRYIFTIRQECSAQDILAAA | |
| | | KYNQQLDRYKSQKANPSVLGNQGFTWTNAVILPEKAQRNDRP | |
| | | NSLDLRIWLYLKLRHPDGRWKKAHIPFYDTRFFQEIYAAGNS | |
| | | PVDTCQFRTPRFGYHLPKLTDQTAIRVNKKHVKAAKTEARIR | |
| | | LAIQQGTLPVSNLKITEISATINSKGQVRIPVKFRVGRQKGT | |
| | | LQIGDRFCGYDQNQTASHAYSLWEVVKEGQYHKELGCFVRFI | |
| | | SSGDIVSITENRGNQFDQLSYEGLAYPQYADWRKKASKFVSL | |
| | | WQITKKNKKKEIVTVEAKEKFDAICKYQPRLYKFNKEYAYLL | |
| | | RDIVRGKSLVELQQIRQEIFRFIEQDCGVTRLGSLSLSTLET | |
| | | VKAVKGIIYSYFSTALNASKNNPISDEQRKEFDPELFALLEK | |
| | | LELIRTRKKKQKVERIANSLIQTCLENNIKFIRGEGDLSTTN | |
| | | NATKKKANSRSMDWLARGVENKIRQLAPMHNITLFGCGSLYT | |
| | | SHQDPLVHRNPDKAMKCRWAAIPVKDIGDWVLRKLSQNLRAK | |
| | | NRGTGEYYHQGVKEFLSHYELQDLEEELLKWRSDRKSNIPCW | |
| | | VLQNRLAEKLGNKEAVVYIPVRGGRIYFATHKVATGAVSIVE | |
| | | DQKQVWVCNADHVAAANIALTGKGIGEQSSDEENPDGSRIKL | |
| | | QLTSKRPAATKKAGQAKKKKSGGSSGGSSGSETPGTSESATP | |
| | | ESSGGSSGGSTLNIEDEYRLHETSKEPDVSLGSTWLSDFPQA | |
| | | WAETGGMGLAVRQAPLIIPLKATSTPVSIKQYPMSQEARLGI | |
| | | KPHIQRLLDQGILVPCQSPWNTPLLPVKKPGTNDYRPVQDLR | |
| | | EVNKRVEDIHPTVPNPYNLLSGLPPSHQWYTVLDLKDAFFCL | |
| | | RLHPTSQPLFAFEWRDPEMGISGQLTWTRLPQGFKNSPTLEN | |
| | | EALHRDLADFRIQHPDLILLQYVDDLLLAATSELDCQQGTRA | |
| | | LLQTLGNLGYRASAKKAQICQKQVKYLGYLLKEGQRWLTEAR | |
| | | KETVMGQPTPKTPRQLREFLGKAGFCRLFIPGFAEMAAPLYP | |
| | | LTKPGTLFNWGPDQQKAYQEIKQALLTAPALGLPDLTKPFEL | |
| | | FVDEKQGYAKGVLTQKLGPWRRPVAYLSKKLDPVAAGWPPCL | |
| | | RMVAAIAVLTKDAGKLTMGQPLVILAPHAVEALVKQPPDRWL | |
| | | SNARMTHYQALLLDTDRVQFGPVVALNPATLLPLPEEGLQHN | |
| | | CLDILAEAHGTRPDLTDQPLPDADHTWYTDGSSLLQEGQRKA | |
| | | GAAVTTETEVIWAKALPAGTSAQRAELIALTQALKMAEGKKL | |
| | | NVYTDSRYAFATAHIHGEIYRRRGWLTSEGKEIKNKDEILAL | |
| | | LKALFLPKRLSIIHCPGHQKGHSAEARGNRMADQAARKAAIT | |
| | | ETPDTSTLLIENSSPMKRTADGSEFESPKKKRKV | |

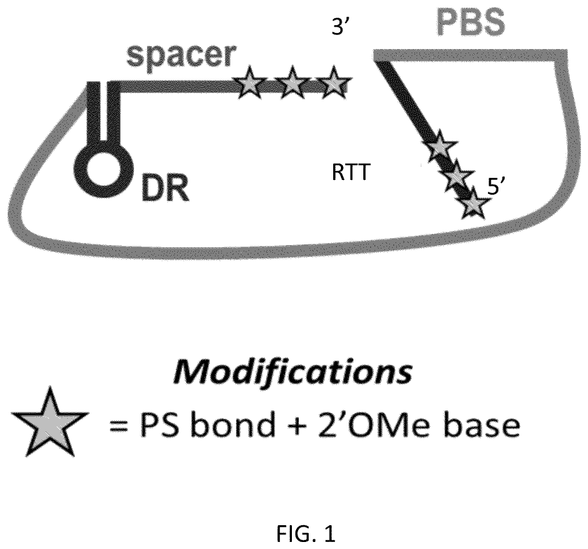

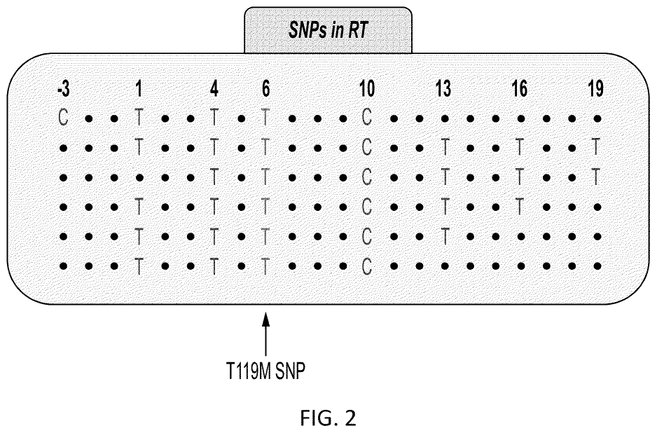

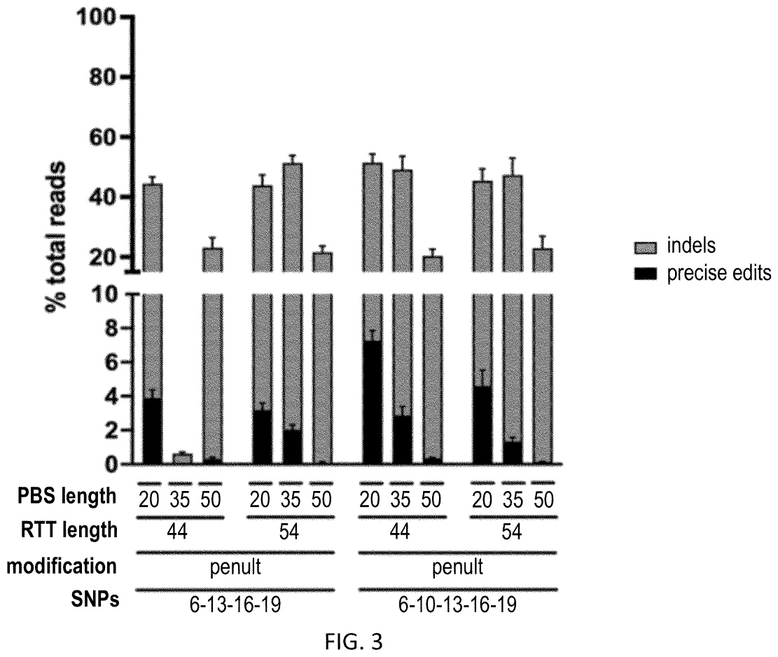

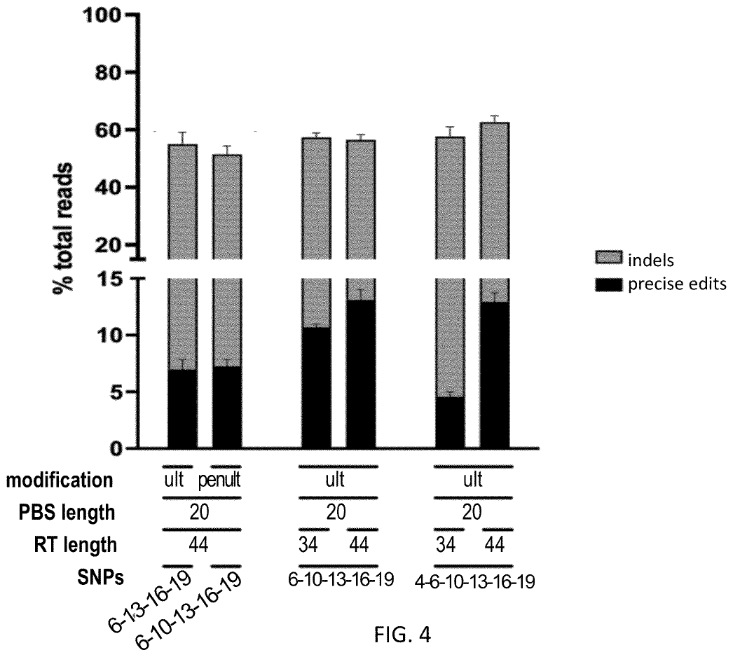

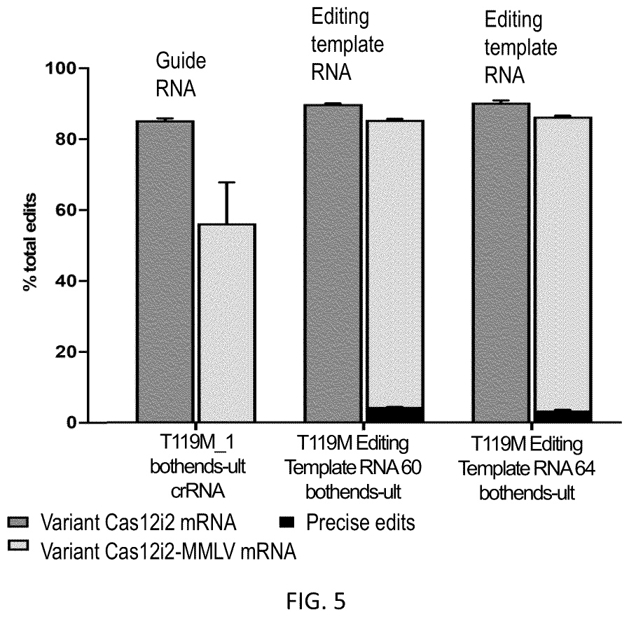

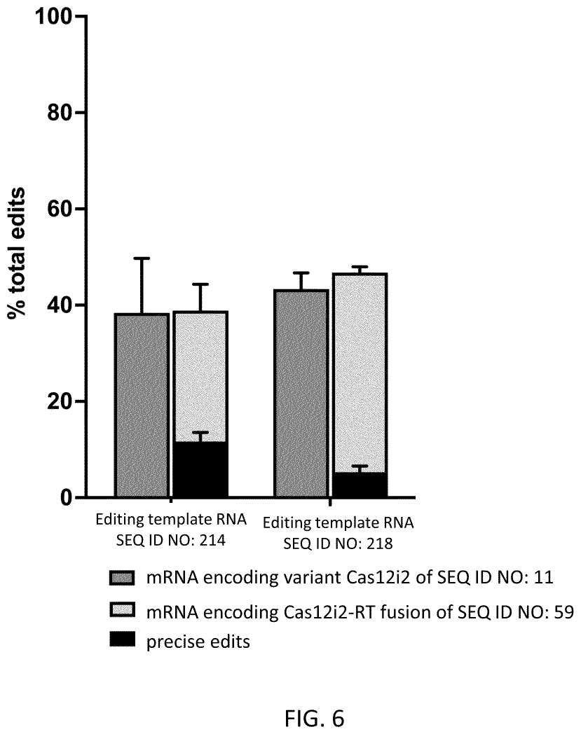

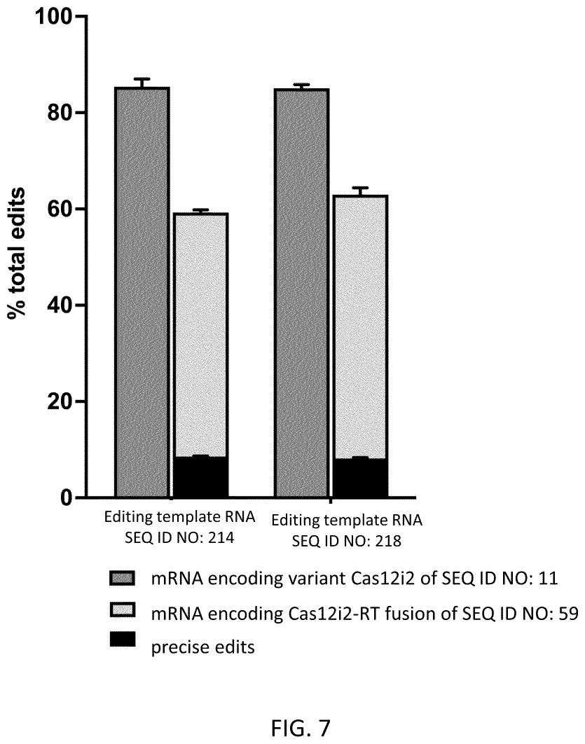

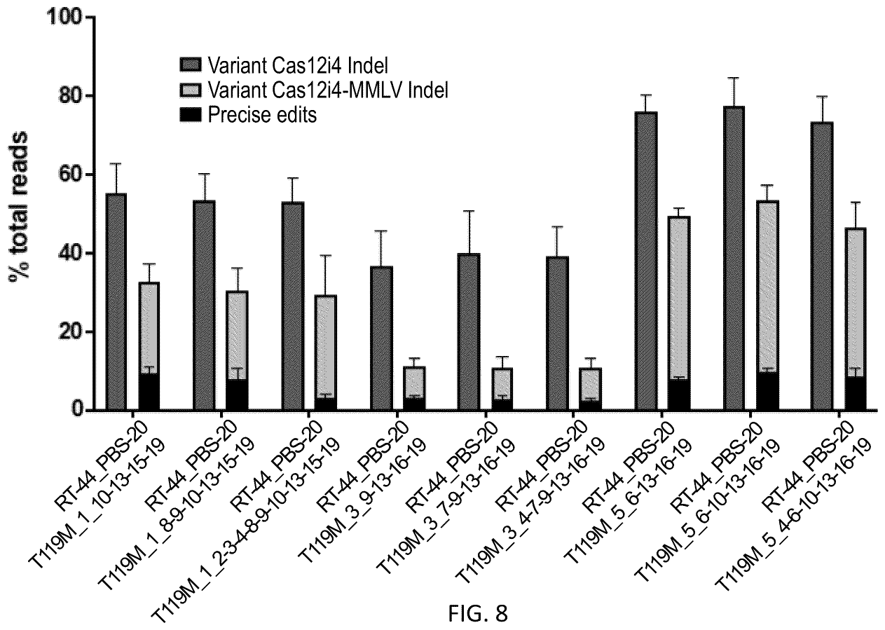

Configurations of tested editing template RNAs are shown in Table 8 and depicted in FIG. 1. The editing template RNAs were synthesized by IDT. A reverse transcription template (RTT) sequence and primer binding site (PBS) were fused to the 5′ end of an RNA guide in the editing template RNAs. The RNA guide may contain a 5′ direct repeat sequence and a spacer sequence. The RTT sequence was designed to introduce a specific substitution to change the amino acid at position 119 from threonine to methionine (T119M) in the human TTR gene. The RTT sequence was further designed to introduce various silent mutations nearby to enhance the editing efficiency and prevent re-targeting of the editing template RNA to the edited genomic site (FIG. 2). The sequences of the editing template RNAs are shown in Table 9, in the PBS length, the RTT length of the donor RNA, and the positions of substitutions relative to PAM sequence are indicated. A working solution of synthetic RNA composed of each editing template RNA was prepared in water (editing template RNA working solution).

TABLE 8
Editing Template RNA Exemplary Designs

| Configuration | Description |
|---|---|
| RTT sequence - PBS - | RTT sequence: 34, 44 or 54 nucleotides |
| DR - spacer | PBS: 20, 35 or 50 nucleotides |

Tables 9-11 provide information of exemplary editing template RNAs for use in editing TTR via the Cas12i2-RT fusion polypeptide disclosed herein.

TABLE 9
Targets of Exemplary Editing Template RNAs for Cas12i2/RT-
Mediated TTR Editing

| | | | position | | total | | |
|---|---|---|---|---|---|---|---|
| Editing | | | of SNP | position of | number of | | SEQ |
| Template | | Target | within | silent | mis- | | ID |
| RNA | PAM | strand* | target | mutations | matches | Target Sequence | NO |
| 1 | ATTC | S | 6 | 13, 16, 19 | 4 | CACCACGGCTGTCGTCACCA | 60 |
| 2 | ATTC | S | 6 | 13, 16, 19 | 4 | CACCACGGCTGTCGTCACCA | 60 |
| 3 | ATTC | S | 6 | 13, 16.19 | 4 | CACCACGGCTGTCGTCACCA | 60 |
| 4 | ATTC | S | 6 | 13, 16, 19 | 4 | CACCACGGCTGTCGTCACCA | 60 |
| 5 | ATTC | S | 6 | 13, 16, 19 | 4 | CACCACGGCTGTCGTCACCA | 60 |
| 6 | ATTC | S | 6 | 13, 16, 19 | 4 | CACCACGGCTGTCGTCACCA | 60 |
| 7 | ATTC | S | 6 | 13, 16, 19 | 4 | CACCACGGCTGTCGTCACCA | 60 |
| 8 | ATTC | S | 6 | 13, 16, 19 | 4 | CACCACGGCTGTCGTCACCA | 60 |
| 9 | ATTC | S | 6 | 13, 16, 19 | 4 | CACCACGGCTGTCGTCACCA | 60 |
| 10 | ATTC | S | 6 | 10, 13, 16, 19 | 5 | CACCACGGCTGTCGTCACCA | 60 |
| 11 | ATTC | S | 6 | 10, 13, 16, 19 | 5 | CACCACGGCTGTCGTCACCA | 60 |
| 12 | ATTC | S | 6 | 10, 13, 16, 19 | 5 | CACCACGGCTGTCGTCACCA | 60 |
| 13 | ATTC | S | 6 | 10, 13, 16, 19 | 5 | CACCACGGCTGTCGTCACCA | 60 |
| 14 | ATTC | S | 6 | 10, 13, 16, 19 | 5 | CACCACGGCTGTCGTCACCA | 60 |
| 15 | ATTC | S | 6 | 10, 13, 16, 19 | 5 | CACCACGGCTGTCGTCACCA | 60 |
| 16 | ATTC | S | 6 | −21, −20, −19, −18, | 12 | CACCACGGCTGTCGTCACCA | 60 |
| | | | | −17, −16, −15, | | | |
| | | | | 10, 13, 16, 19 | | | |
| 17 | ATTC | S | 6 | −21, −20, −19, −18, | 12 | CACCACGGCTGTCGTCACCA | 60 |
| | | | | −17, −16, −15, | | | |
| | | | | 10, 13, 16, 19 | | | |
| 18 | ATTC | S | 6 | −21, −20, −19, −18, | 12 | CACCACGGCTGTCGTCACCA | 60 |
| | | | | −17, −16, −15, | | | |
| | | | | 10, 13, 16, 19 | | | |
| 19 | ATTC | S | 6 | 13, 16, 19 | 4 | CACCACGGCTGTCGTCACCA | 60 |
| 20 | ATTC | S | 6 | 13, 16, 19 | 4 | CACCACGGCTGTCGTCACCA | 60 |
| 21 | ATTC | S | 6 | 13, 16, 19 | 4 | CACCACGGCTGTCGTCACCA | 60 |
| 22 | ATTC | S | 6 | 13, 16.19 | 4 | CACCACGGCTGTCGTCACCA | 60 |
| 23 | ATTC | S | 6 | 13, 16, 19 | 4 | CACCACGGCTGTCGTCACCA | 60 |
| 24 | ATTC | S | 6 | 13, 16, 19 | 4 | CACCACGGCTGTCGTCACCA | 60 |
| 25 | CTTG | AS | 19 | 12, 15 | 3 | GGATTGGTGACGACAGCCGT | 61 |
| 26 | CTTG | AS | 19 | 12, 15 | 3 | GGATTGGTGACGACAGCCGT | 61 |
| 27 | CTTG | AS | 19 | 12, 15 | 3 | GGATTGGTGACGACAGCCGT | 61 |
| 28 | CTTG | AS | 19 | 12, 15 | 3 | GGATTGGTGACGACAGCCGT | 61 |
| 29 | CTTG | AS | 19 | 12, 15 | 3 | GGATTGGTGACGACAGCCGT | 61 |
| 30 | CTTG | AS | 19 | 12, 15 | 3 | GGATTGGTGACGACAGCCGT | 61 |
| 31 | CTTG | AS | 19 | 12, 15 | 3 | GGATTGGTGACGACAGCCGT | 61 |
| 32 | CTTG | AS | 19 | 12.15 | 3 | GGATTGGTGACGACAGCCGT | 61 |
| 33 | CTTG | AS | 19 | 12, 15 | 3 | GGATTGGTGACGACAGCCGT | 61 |
| 34 | ATTC | AS | 23 | 13, 16.19 | 4 | CTTGGGATTGGTGACGACAG | 62 |
| 35 | ATTC | AS | 23 | 13, 16, 19 | 4 | CTTGGGATTGGTGACGACAG | 62 |
| 36 | ATTC | AS | 23 | 13, 16, 19 | 4 | CTTGGGATTGGTGACGACAG | 62 |
| 37 | ATTC | AS | 23 | 13, 16, 19 | 4 | CTTGGGATTGGTGACGACAG | 62 |
| 38 | ATTC | AS | 23 | 13, 16, 19 | 4 | CTTGGGATTGGTGACGACAG | 62 |
| 39 | ATTC | AS | 23 | 13, 16, 19 | 4 | CTTGGGATTGGTGACGACAG | 62 |
| 40 | ATTC | AS | 23 | 13, 16, 19 | 4 | CTTGGGATTGGTGACGACAG | 62 |
| 41 | ATTC | AS | 23 | 13, 16, 19 | 4 | CTTGGGATTGGTGACGACAG | 62 |
| 42 | ATTC | AS | 23 | 13, 16, 19 | 4 | CTTGGGATTGGTGACGACAG | 62 |
| 54 | ATTC | S | 6 | 13, 16, 19 | 4 | CACCACGGCTGTCGTCACCA | 60 |
| 55 | ATTC | S | 6 | 13, 16, 19 | 4 | CACCACGGCTGTCGTCACCA | 60 |
| 56 | ATTC | S | 6 | 13, 16, 19 | 4 | CACCACGGCTGTCGTCACCA | 60 |
| 57 | ATTC | S | 6 | 10, 13, 16, 19 | 5 | CACCACGGCTGTCGTCACCA | 60 |
| 58 | ATTC | S | 6 | 10, 13, 16, 19 | 5 | CACCACGGCTGTCGTCACCA | 60 |
| 59 | ATTC | S | 6 | 10, 13, 16, 19 | 5 | CACCACGGCTGTCGTCACCA | 60 |
| 60 | ATTC | S | 6 | 10, 13, 16, 19 | 5 | CACCACGGCTGTCGTCACCA | 60 |
| 61 | ATTC | S | 6 | 4, 10, 13, 16, 19 | 6 | CACCACGGCTGTCGTCACCA | 60 |
| 62 | ATTC | S | 6 | 4, 10, 13, 16, 19 | 6 | CACCACGGCTGTCGTCACCA | 60 |
| 63 | ATTC | S | 6 | 4, 10, 13, 16, 19 | 6 | CACCACGGCTGTCGTCACCA | 60 |
| 64 | ATTC | S | 6 | 4, 10, 13, 16, 19 | 6 | CACCACGGCTGTCGTCACCA | 60 |
| 65 | ATTC | S | 6 | 1, 4, 10, 13, 16, 19 | 7 | CACCACGGCTGTCGTCACCA | 60 |
| 66 | ATTC | S | 6 | 1, 4, 10, 13, 16, 19 | 7 | CACCACGGCTGTCGTCACCA | 60 |
| 67 | ATTC | S | 6 | 1, 4, 10, 13, 16, 19 | 7 | CACCACGGCTGTCGTCACCA | 60 |
| 68 | ATTC | S | 6 | 1, 4, 10, 13, 16, 19 | 7 | CACCACGGCTGTCGTCACCA | 60 |
| 69 | ATTC | S | 6 | 1.4.10, 13, 16 | 6 | CACCACGGCTGTCGTCACCA | 60 |
| 70 | ATTC | S | 6 | 1, 4, 10, 13, 16 | 6 | CACCACGGCTGTCGTCACCA | 60 |
| 71 | ATTC | S | 6 | 1, 4, 10, 13, 16 | 6 | CACCACGGCTGTCGTCACCA | 60 |
| 72 | ATTC | S | 6 | 1, 4, 10, 13, 16 | 6 | CACCACGGCTGTCGTCACCA | 60 |
| 73 | ATTC | S | 6 | 1, 4, 10, 13 | 5 | CACCACGGCTGTCGTCACCA | 60 |
| 74 | ATTC | S | 6 | 1, 4, 10, 13 | 5 | CACCACGGCTGTCGTCACCA | 60 |
| 75 | ATTC | S | 6 | 1, 4, 10, 13 | 5 | CACCACGGCTGTCGTCACCA | 60 |
| 76 | ATTC | S | 6 | 1, 4, 10, 13 | 5 | CACCACGGCTGTCGTCACCA | 60 |
| 77 | ATTC | S | 6 | 1, 4, 10 | 4 | CACCACGGCTGTCGTCACCA | 60 |
| 78 | ATTC | S | 6 | 1, 4, 10 | 4 | CACCACGGCTGTCGTCACCA | 60 |
| 79 | ATTC | S | 6 | 1.4.10 | 4 | CACCACGGCTGTCGTCACCA | 60 |
| 80 | ATTC | S | 6 | 1, 4, 10 | 4 | CACCACGGCTGTCGTCACCA | 60 |
| 81 | ATTC | S | 6 | −3, 1.4.10 | 5 | CACCACGGCTGTCGTCACCA | 60 |
| 82 | ATTC | S | 6 | −3, 1, 4, 10 | 5 | CACCACGGCTGTCGTCACCA | 60 |
| 83 | ATTC | S | 6 | −3, 1.4, 10 | 5 | CACCACGGCTGTCGTCACCA | 60 |
| 84 | ATTC | S | 6 | −3, 1, 4, 10 | 5 | CACCACGGCTGTCGTCACCA | 60 |

*Target stand refers to the strand where the target sequence locates.
S: sense strand.
AS: antisense strand

TABLE 10
Sequences of Functional Elements in Exemplary Editing Template RNAs

| Editing | | | | |
|---|---|---|---|---|
| Template | | | | |
| RNAS | Spacer | DR | RT Template | PBS |
| 1 | CACCACGGCUGUC | AGAAAUCCGUCUUU | ACUCCUAUUCCACCAUGGCUGU | CAAGGAAUGAGGGACUUC |
| | GUCACCA (SEQ | CAUUGACGG (SEQ | UGUUACUAAUCC (SEQ ID | UC (SEQ ID NO: 91) |
| | ID NO: 63) | ID NO: 39) | NO: 66) | |
| 2 | CACCACGGCUGUC | AGAAAUCCGUCUUU | ACUCCUAUUCCACCAUGGCUGU | CAAGGAAUGAGGGACUUC |
| | GUCACCA (SEQ | CAUUGACGG (SEQ | UGUUACUAAUCC (SEQ ID | UCCUCCAGUGGACCUGA |
| | ID NO: 63 | ID NO: 39) | NO: 66) | (SEQ ID NO: 92) |
| 3 | CACCACGGCUGUC | AGAAAUCCGUCUUU | ACUCCUAUUCCACCAUGGCUGU | CAAGGAAUGAGGGACUUC |
| | GUCACCA (SEQ | CAUUGACGG (SEQ | UGUUACUAAUCC (SEQ ID | UCCUCCAGUGGACCUGAA |
| | ID NO: 63 | ID NO: 39) | NO: 66) | GGACGAGGGAUGGG |
| | | | | (SEQ ID NO: 93) |
| 4 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEç | CCAUGGCUGUUGUUACUAAUCC | UC (SEQ ID NO: 91) |
| | ID NO: 63) | ID NO: 39) | (SEQ ID NO: 67) | |
| 5 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCUGUUGUUACUAAUCC | UCCUCCAGUGGACCUGA |
| | ID NO: 63) | ID NO: 39) | (SEQ ID NO: 67) | (SEQ ID NO: 92) |
| 6 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCUGUUGUUACUAAUCC | UCCUCCAGUGGACCUGAA |
| | ID NO: 63) | ID NO: 39) | (SEQ ID NO: 67) | GGACGAGGGAUGGG |
| | | | | (SEQ ID NO: 93) |
| 7 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UC (SEQ ID NO: 91) |
| | ID NO: 63) | ID NO: 39) | UUACUAAUCC (SEQ ID NO: | |
| | | | 68) | |
| 8 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UCCUCCAGUGGACCUGA |
| | ID NO: 63) | ID NO: 39) | UUACUAAUCC (SEQ ID NO: | (SEQ ID NO: 92) |
| | | | 68) | |
| 9 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UCCUCCAGUGGACCUGAA |
| | ID NO: 63) | ID NO: 39) | UUACUAAUCC (SEQ ID NO: | GGACGAGGGAUGGG |
| | | | 68) | (SEQ ID NO: 93) |
| 10 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCCGUUG | UC (SEQ ID NO: 91) |
| | ID NO: 63) | ID NO: 39) | UUACUAAUCC (SEQ ID NO: | |
| | | | 69) | |
| 11 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCCGUUG | UCCUCCAGUGGACCUGA |
| | ID NO: 63) | ID NO: 39) | UUACUAAUCC (SEQ ID NO: | (SEQ ID NO: 92) |
| | | | 69) | |
| 12 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCCGUUG | UCCUCCAGUGGACCUGAA |
| | ID NO: 63) | ID NO: 39) | UUACUAAUCC (SEQ ID NO: | GGACGAGGGAUGGG |
| | | | 69) | (SEQ ID NO: 93) |
| 13 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCCGUUGUUACUAAUCC | UC (SEQ ID NO: 91) |
| | ID NO: 63) | ID NO: 39) | (SEQ ID NO: 70) | |
| 14 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCCGUUGUUACUAAUCC | UCCUCCAGUGGACCUGA |
| | ID NO: 63) | ID NO: 39) | (SEQ ID NO: 70) | (SEQ ID NO: 92) |
| 15 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCCGUUGUUACUAAUCC | UCCUCCAGUGGACCUGAA |
| | ID NO: 63) | ID NO: 39) | (SEQ ID NO: 70) | GGACGAGGGAUGGG |
| | | | | (SEQ ID NO: 93) |
| 16 | CACCACGGCUGUC | AGAAAUCCGUCUUU | UGCCGCCCUAUUAUCACCCUAC | CAAGGAAUGAGGGACUUC |
| | GUCACCA (SEQ | CAUUGACGG (SEQ | UCCUAUUCCACCAUGGCCGUUG | UC (SEQ ID NO: 91) |
| | ID NO: 63) | ID NO: 39) | UUACUAAUCC (SEQ ID NO: | |
| | | | 71) | |
| 17 | CACCACGGCUGUC | AGAAAUCCGUCUUU | UGCCGCCCUAUUAUCACCCUAC | CAAGGAAUGAGGGACUUC |
| | GUCACCA (SEQ | CAUUGACGG (SEQ | UCCUAUUCCACCAUGGCCGUUG | UCCUCCAGUGGACCUGA |
| | ID NO: 63) | ID NO: 39) | UUACUAAUCC (SEQ ID NO: | (SEQ ID NO: 92) |
| | | | 71) | |
| 18 | CACCACGGCUGUC | AGAAAUCCGUCUUU | UGCCGCCCUAUUAUCACCCUAC | CAAGGAAUGAGGGACUUC |
| | GUCACCA (SEQ | CAUUGACGG (SEQ | UCCUAUUCCACCAUGGCCGUUG | UCCUCCAGUGGACCUGAA |
| | ID NO: 63) | ID NO: 39) | UUACUAAUCC (SEQ ID NO: | GGACGAGGGAUGGG |
| | | | 71) | (SEQ ID NO: 93) |
| 19 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCUGUUGUUACUAAUCC | UC (SEQ ID NO: 91) |
| | ID NO: 63) | ID NO: 39) | (SEQ ID NO: 67) | |
| 20 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCUGUUGUUACUAAUCC | UCCUCCAGUGGACCUGA |
| | ID NO: 63) | ID NO: 39) | (SEQ ID NO: 67) | (SEQ ID NO: 92) |
| 21 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCUGUUGUUACUAAUCC | UCCUCCAGUGGACCUGAA |
| | ID NO: 63) | ID NO: 39) | (SEQ ID NO: 67) | GGACGAGGGAUGGG |
| | | | | (SEQ ID NO: 93) |
| 22 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UC (SEQ ID NO: 91) |
| | ID NO: 63) | ID NO: 39) | UUACUAAUCC (SEQ ID NO: | |
| | | | 68) | |
| 23 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UCCUCCAGUGGACCUGA |
| | ID NO: 63) | ID NO: 39) | UUACUAAUCC (SEQ ID NO: | (SEQ ID NO: 92) |
| | | | 68) | |
| 24 | CACCACGGCUGUC | AGAAAUCCGUCUUU | UGCCGCCCUGCUGAGCCCCUAC | CAAGGAAUGAGGGACUUC |
| | GUCACCA (SEQ | CAUUGACGG (SEQ | UCCUAUUCCACCAUGGCUGUUG | UCCUCCAGUGGACCUGAA |
| | ID NO: 63) | ID NO: 39) | UUACUAAUCC (SEQ ID NO: | GGACGAGGGAUGGG |
| | | | 68) | (SEQ ID NO: 93) |
| 25 | GGAUUGGUGACGA | AGAAAUCCGUCUUU | UCAUUCCUUGGGAUUGGUGACA | GAAUAGGAGUAGGGGCUC |
| | CAGCCGU (SEQ | CAUUGACGG (SEQ | ACGGCCAUGGUG (SEQ ID | AG (SEQ ID NO: 94) |
| | ID NO: 64) | ID NO: 39) | NO: 72) | |
| 26 | GGAUUGGUGACGA | AGAAAUCCGUCUUU | UCAUUCCUUGGGAUUGGUGACA | GAAUAGGAGUAGGGGCUC |
| | CAGCCGU (SEQ | CAUUGACGG (SEQ | ACGGCCAUGGUG (SEQ ID | AGCAGGGCGGCAAUGGU |
| | ID NO: 64) | ID NO: 39) | NO: 72) | (SEQ ID NO: 95) |
| 27 | GGAUUGGUGACGA | AGAAAUCCGUCUUU | UCAUUCCUUGGGAUUGGUGACA | GAAUAGGAGUAGGGGCUC |
| | CAGCCGU (SEQ | CAUUGACGG (SEQ | ACGGCCAUGGUG (SEQ ID | AGCAGGGCGGCAAUGGUG |
| | ID NO: 64) | ID NO: 39) | NO: 72) | UAGCGGCGGGGGCC |
| | | | | (SEQ ID NO: 96) |
| 28 | GGAUUGGUGACGA | AGAAAUCCGUCUUU | GAGAAGUCCCUCAUUCCUUGGG | GAAUAGGAGUAGGGGCUC |
| | CAGCCGU (SEQ | CAUUGACGG (SEQ | AUUGGUGACAACGGCCAUGGUG | AG (SEQ ID NO: 94) |
| | ID NO: 64) | ID NO: 39) | (SEQ ID NO: 73) | |
| 29 | GGAUUGGUGACGA | AGAAAUCCGUCUUU | GAGAAGUCCCUCAUUCCUUGGG | GAAUAGGAGUAGGGGCUC |
| | CAGCCGU (SEQ | CAUUGACGG (SEQ | AUUGGUGACAACGGCCAUGGUG | AGCAGGGCGGCAAUGGU |
| | ID NO: 64) | ID NO: 39) | (SEQ ID NO: 73) | (SEQ ID NO: 95) |
| 30 | GGAUUGGUGACGA | AGAAAUCCGUCUUU | GAGAAGUCCCUCAUUCCUUGGG | GAAUAGGAGUAGGGGCUC |
| | CAGCCGU (SEQ | CAUUGACGG (SEQ | AUUGGUGACAACGGCCAUGGUG | AGCAGGGCGGCAAUGGUG |
| | ID NO: 64) | ID NO: 39) | (SEQ ID NO: 73) | UAGCGGCGGGGGCC |
| | | | | (SEQ ID NO: 96) |
| 31 | GGAUUGGUGACGA | AGAAAUCCGUCUUU | AUUCCUUGGGAUUGGUGACAAC | GAAUAGGAGUAGGGGCUC |
| | CAGCCGU (SEQ | CAUUGACGG (SEQ | UCCACUGGAGGAGAAGUCCCUC | AG (SEQ ID NO: 94) |
| | ID NO: 64) | ID NO: 39) | GGCCAUGGUG | |
| | | | (SEQ ID NO: 74) | |
| 32 | GGAUUGGUGACGA | AGAAAUCCGUCUUU | AUUCCUUGGGAUUGGUGACAAC | GAAUAGGAGUAGGGGCUC |
| | CAGCCGU (SEQ | CAUUGACGG (SEQ | UCCACUGGAGGAGAAGUCCCUC | AGCAGGGCGGCAAUGGU |
| | ID NO: 64) | ID NO: 39) | GGCCAUGGUG | (SEQ ID NO: 95) |
| | | | (SEQ ID NO: 74) | |
| 33 | GGAUUGGUGACGA | AGAAAUCCGUCUUU | UCCACUGGAGGAGAAGUCCCUC | GAAUAGGAGUAGGGGCUC |
| | CAGCCGU (SEQ | CAUUGACGG (SEQ | AUUCCUUGGGAUUGGUGACAAC | AGCAGGGCGGCAAUGGUG |
| | ID NO: 64) | ID NO: 39) | GGCCAUGGUG (SEQ ID NO: | UAGCGGCGGGGGCC |
| | | | 74) | (SEQ ID NO: 96) |
| 34 | CUUGGGAUUGGUG | AGAAAUCCGUCUUU | UCCCUCAUUCCUUGGGAUUGGU | GGUGGAAUAGGAGUAGGG |
| | ACGACAG (SEQ | CAUUGACGG (SEQ | AACAACGGCCAU (SEQ ID | GC (SEQ ID NO: 97) |
| | ID NO: 65) | ID NO: 39) | NO: 75) | |
| 35 | CUUGGGAUUGGUG | AGAAAUCCGUCUUU | UCCCUCAUUCCUUGGGAUUGGU | GGUGGAAUAGGAGUAGGG |
| | ACGACAG (SEQ | CAUUGACGG (SEQ | AACAACGGCCAU (SEQ ID | GCUCAGCAGGGCGGCAA |
| | ID NO: 65) | ID NO: 39) | NO: 75) | (SEQ ID NO: 98) |
| 36 | CUUGGGAUUGGUG | AGAAAUCCGUCUUU | UCCCUCAUUCCUUGGGAUUGGU | GGUGGAAUAGGAGUAGGG |
| | ACGACAG (SEQ | CAUUGACGG (SEQ | AACAACGGCCAU (SEQ ID | GCUCAGCAGGGCGGCAAU |
| | ID NO: 65) | ID NO: 39) | NO: 75) | GGUGUAGCGGCGGG |
| | | | | (SEQ ID NO: 99) |
| 37 | CUUGGGAUUGGUG | AGAAAUCCGUCUUU | GGAGGAGAAGUCCCUCAUUCCU | GGUGGAAUAGGAGUAGGG |
| | ACGACAG (SEQ | CAUUGACGG (SEQ | UGGGAUUGGUAACAACGGCCAU | GC (SEQ ID NO: 97) |
| | ID NO: 65) | ID NO: 39) | (SEQ ID NO: 76) | |
| 38 | CUUGGGAUUGGUG | AGAAAUCCGUCUUU | GGAGGAGAAGUCCCUCAUUCCU | GGUGGAAUAGGAGUAGGG |
| | ACGACAG (SEQ | CAUUGACGG (SEQ | UGGGAUUGGUAACAACGGCCAU | GCUCAGCAGGGCGGCAA |
| | ID NO: 65) | ID NO: 39) | (SEQ ID NO: 76) | (SEQ ID NO: 98) |
| 39 | CUUGGGAUUGGUG | AGAAAUCCGUCUUU | GGAGGAGAAGUCCCUCAUUCCU | GGUGGAAUAGGAGUAGGG |
| | ACGACAG (SEQ | CAUUGACGG (SEQ | UGGGAUUGGUAACAACGGCCAU | GCUCAGCAGGGCGGCAAU |
| | ID NO: 65) | ID NO: 39) | (SEQ ID NO: 76) | GGUGUAGCGGCGGG |
| | | | | (SEQ ID NO: 99) |
| 40 | CUUGGGAUUGGUG | AGAAAUCCGUCUUU | CAGGUCCACUGGAGGAGAAGUC | GGUGGAAUAGGAGUAGGG |
| | ACGACAG (SEQ | CAUUGACGG (SEQ | CCUCAUUCCUUGGGAUUGGUAA | GC (SEQ ID NO: 97) |
| | ID NO: 65) | ID NO: 39) | CAACGGCCAU (SEQ ID NO: | |
| | | | 77) | |
| 41 | CUUGGGAUUGGUG | AGAAAUCCGUCUUU | CAGGUCCACUGGAGGAGAAGUC | GGUGGAAUAGGAGUAGGG |
| | ACGACAG (SEQ | CAUUGACGG (SEQ | CCUCAUUCCUUGGGAUUGGUAA | GCUCAGCAGGGCGGCAA |
| | ID NO: 65) | ID NO: 39) | CAACGGCCAU (SEQ ID NO: | (SEQ ID NO: 98) |
| | | | 77) | |
| 42 | CUUGGGAUUGGUG | AGAAAUCCGUCUUU | CAGGUCCACUGGAGGAGAAGUC | GGUGGAAUAGGAGUAGGG |
| | ACGACAG (SEQ | CAUUGACGG (SEQ | CCUCAUUCCUUGGGAUUGGUAA | GCUCAGCAGGGCGGCAAU |
| | ID NO: 65) | ID NO: 39) | CAACGGCCAU (SEQ ID NO: | GGUGUAGCGGCGGG |
| | | | 77) | (SEQ ID NO: 99) |
| 54 | CACCACGGCUGUC | AGAAAUCCGUCUUU | ACUCCUAUUCCACCAUGGCUGU | CAAGGAAUGAGGG (SEQ |
| | GUCACCA (SEQ | CAUUGACGG (SEQ | UGUUACUAAUCC (SEQ ID | ID NO: 100) |
| | ID NO: 63) | ID NO: 39) | NO: 66) | |
| 55 | CACCACGGCUGUC | AGAAAUCCGUCUUU | ACUCCUAUUCCACCAUGGCUGU | CAAGGAAUGAGGGACUUC |
| | GUCACCA (SEQ | CAUUGACGG (SEQ | UGUUACUAAUCC (SEQ ID | UC (SEQ ID NO: 91) |
| | ID NO: 63) | ID NO: 39) | NO: 66) | |
| 56 | CACCACGGCUGUC | AGAAAUCCGUCUUU | CUGAGCCCCUACUCCUAUUCCA | CAAGGAAUGAGGG (SEQ |
| | GUCACCA (SEQ | CAUUGACGG (SEQ | CCAUGGCUGUUGUUACUAAUCC | ID NO: 100) |
| | ID NO: 63) | ID NO: 39) | (SEQ ID NO: 67) | |
| 57 | CACCACGGCUGUC | AGAAAUCCGUCUUU | ACUCCUAUUCCACCAUGGCCGU | CAAGGAAUGAGGG (SEQ |
| | GUCACCA (SEQ | CAUUGACGG (SEQ | UGUUACUAAUCC (SEQ ID | ID NO: 100) |
| | ID NO: 63) | ID NO: 39) | NO: 78) | |
| 58 | CACCACGGCUGUC | AGAAAUCCGUCUUU | ACUCCUAUUCCACCAUGGCCGU | CAAGGAAUGAGGGACUUC |
| | GUCACCA (SEQ | CAUUGACGG (SEQ | UGUUACUAAUCC (SEQ ID | UC (SEQ ID NO: 91) |
| | ID NO: 63) | ID NO: 39) | NO: 78) | |
| 59 | CACCACGGCUGUC | AGAAAUCCGUCUUU | CUGAGCCCCUACUCCUAUUCCA | CAAGGAAUGAGGG (SEQ |
| | GUCACCA (SEQ | CAUUGACGG (SEQ | CCAUGGCCGUUGUUACUAAUCC | ID NO: 100) |
| | ID NO: 63) | ID NO: 39) | (SEQ ID NO: 70) | |
| 60 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CCAUGGCCGUUGUUACUAAUCC | UC (SEQ ID NO: 91) |
| | ID NO: 63) | ID NO: 39) | (SEQ ID NO: 70) | |
| 61 | CACCACGGCUGUC | AGAAAUCCGUCUUU | ACUCCUAUUCCACUAUGGCCGU | CAAGGAAUGAGGG (SEQ |
| | GUCACCA (SEQ | CAUUGACGG (SEQ | UGUUACUAAUCC (SEQ ID | ID NO: 100) |
| | ID NO: 63) | ID NO: 39) | NO: 79) | |
| 62 | CACCACGGCUGUC | AGAAAUCCGUCUUU | ACUCCUAUUCCACUAUGGCCGU | CAAGGAAUGAGGGACUUC |
| | GUCACCA (SEQ | CAUUGACGG (SEQ | UGUUACUAAUCC (SEQ ID | UC (SEQ ID NO: 91) |
| | ID NO: 63) | ID NO: 39) | NO: 79) | |
| 63 | CACCACGGCUGUC | AGAAAUCCGUCUUU | CUGAGCCCCUACUCCUAUUCCA | CAAGGAAUGAGGG (SEQ |
| | GUCACCA (SEQ | CAUUGACGG (SEQ | CUAUGGCCGUUGUUACUAAUCC | ID NO: 100) |
| | ID NO: 63) | ID NO: 39) | (SEQ ID NO: 80) | |
| 64 | CACCACGGCUGUC | AGAAAUCCGUCUUU | CUGAGCCCCUACUCCUAUUCCA | CAAGGAAUGAGGGACUUC |
| | GUCACCA (SEQ | CAUUGACGG (SEQ | CUAUGGCCGUUGUUACUAAUCC | UC (SEQ ID NO: 91) |
| | ID NO: 63) | ID NO: 39) | (SEQ ID NO: 80) | |
| 65 | CACCACGGCUGUC | AGAAAUCCGUCUUU | ACUCCUAUUCUACUAUGGCCGU | CAAGGAAUGAGGG (SEQ |
| | GUCACCA (SEQ | CAUUGACGG (SEQ | UGUUACUAAUCC (SEQ ID | ID NO: 100) |
| | ID NO: 63) | ID NO: 39) | NO: 81) | |
| 66 | CACCACGGCUGUC | AGAAAUCCGUCUUU | ACUCCUAUUCUACUAUGGCCGU | CAAGGAAUGAGGGACUUC |
| | GUCACCA (SEQ | CAUUGACGG (SEQ | UGUUACUAAUCC (SEQ ID | UC (SEQ ID NO: 91) |
| | ID NO: 63) | ID NO: 39) | NO: 81) | |
| 67 | CACCACGGCUGUC | AGAAAUCCGUCUUU | CUGAGCCCCUACUCCUAUUCUA | CAAGGAAUGAGGG (SEQ |
| | GUCACCA (SEQ | CAUUGACGG (SEQ | CUAUGGCCGUUGUUACUAAUCC | ID NO: 100) |
| | ID NO: 63) | ID NO: 39) | (SEQ ID NO: 82) | |
| 68 | CACCACGGCUGUC | AGAAAUCCGUCUUU | CUGAGCCCCUACUCCUAUUCUA | CAAGGAAUGAGGGACUUC |
| | GUCACCA (SEQ | CAUUGACGG (SEQ | CUAUGGCCGUUGUUACUAAUCC | UC (SEQ ID NO: 91) |
| | ID NO: 63) | ID NO: 39) | (SEQ ID NO: 82) | |
| 69 | CACCACGGCUGUC | AGAAAUCCGUCUUU | ACUCCUAUUCUACUAUGGCCGU | CAAGGAAUGAGGG (SEQ |
| | GUCACCA (SEQ | CAUUGACGG (SEQ | UGUUACCAAUCC (SEQ ID | ID NO: 100) |
| | ID NO: 63) | ID NO: 39) | NO: 83) | |
| 70 | CACCACGGCUGUC | AGAAAUCCGUCUUU | ACUCCUAUUCUACUAUGGCCGU | CAAGGAAUGAGGGACUUC |
| | GUCACCA (SEQ | CAUUGACGG (SEQ | UGUUACCAAUCC (SEQ ID | UC (SEQ ID NO: 91) |
| | ID NO: 63) | ID NO: 39) | NO: 83) | |
| 71 | CACCACGGCUGUC | AGAAAUCCGUCUUU | CUGAGCCCCUACUCCUAUUCUA | CAAGGAAUGAGGG (SEQ |
| | GUCACCA (SEQ | CAUUGACGG (SEQ | CUAUGGCCGUUGUUACCAAUCC | ID NO: 100) |
| | ID NO: 63) | ID NO: 39) | (SEQ ID NO: 84) | |
| 72 | CACCACGGCUGUC | AGAAAUCCGUCUUU | CUGAGCCCCUACUCCUAUUCUA | CAAGGAAUGAGGGACUUC |
| | GUCACCA (SEQ | CAUUGACGG (SEQ | CUAUGGCCGUUGUUACCAAUCC | UC (SEQ ID NO: 91) |
| | ID NO: 63) | ID NO: 39) | (SEQ ID NO: 84) | |
| 73 | CACCACGGCUGUC | AGAAAUCCGUCUUU | ACUCCUAUUCUACUAUGGCCGU | CAAGGAAUGAGGG (SEQ |
| | GUCACCA (SEQ | CAUUGACGG (SEQ | UGUCACCAAUCC (SEQ ID | ID NO: 100) |
| | ID NO: 63) | ID NO: 39) | NO: 85) | |
| 74 | CACCACGGCUGUC | AGAAAUCCGUCUUU | ACUCCUAUUCUACUAUGGCCGU | CAAGGAAUGAGGGACUUC |
| | GUCACCA (SEQ | CAUUGACGG (SEQ | UGUCACCAAUCC (SEQ ID | UC (SEQ ID NO: 91) |
| | ID NO: 63) | ID NO: 39) | NO: 85) | |
| 75 | CACCACGGCUGUC | AGAAAUCCGUCUUU | CUGAGCCCCUACUCCUAUUCUA | CAAGGAAUGAGGG (SEQ |
| | GUCACCA (SEQ | CAUUGACGG (SEQ | CUAUGGCCGUUGUCACCAAUCC | ID NO: 100) |
| | ID NO: 63) | ID NO: 39) | (SEQ ID NO: 86) | |
| 76 | CACCACGGCUGUC | AGAAAUCCGUCUUU | CUGAGCCCCUACUCCUAUUCUA | CAAGGAAUGAGGGACUUC |
| | GUCACCA (SEQ | CAUUGACGG (SEQ | CUAUGGCCGUUGUCACCAAUCC | UC (SEQ ID NO: 91) |
| | ID NO: 63) | ID NO: 39) | (SEQ ID NO: 86) | |
| 77 | CACCACGGCUGUC | AGAAAUCCGUCUUU | ACUCCUAUUCUACUAUGGCCGU | CAAGGAAUGAGGG (SEQ |
| | GUCACCA (SEQ | CAUUGACGG (SEQ | CGUCACCAAUCC (SEQ ID | ID NO: 100) |
| | ID NO: 63) | ID NO: 39) | NO: 87) | |
| 78 | CACCACGGCUGUC | AGAAAUCCGUCUUU | ACUCCUAUUCUACUAUGGCCGU | CAAGGAAUGAGGGACUUC |
| | GUCACCA (SEQ | CAUUGACGG (SEQ | CGUCACCAAUCC (SEQ ID | UC (SEQ ID NO: 91) |
| | ID NO: 63) | ID NO: 39) | NO: 87) | |
| 79 | CACCACGGCUGUC | AGAAAUCCGUCUUU | CUGAGCCCCUACUCCUAUUCUA | CAAGGAAUGAGGG (SEQ |
| | GUCACCA (SEQ | CAUUGACGG (SEQ | CUAUGGCCGUCGUCACCAAUCC | ID NO: 100) |
| | ID NO: 63) | ID NO: 39) | (SEQ ID NO: 88) | |
| 80 | CACCACGGCUGUC | AGAAAUCCGUCUUU | CUGAGCCCCUACUCCUAUUCUA | CAAGGAAUGAGGGACUUC |
| | GUCACCA (SEQ | CAUUGACGG (SEQ | CUAUGGCCGUCGUCACCAAUCC | UC (SEQ ID NO: 91) |
| | ID NO: 63) | ID NO: 39) | (SEQ ID NO: 88) | |
| 81 | CACCACGGCUGUC | AGAAAUCCGUCUUU | ACUCCUACUCUACUAUGGCCGU | CAAGGAAUGAGGG (SEQ |
| | GUCACCA (SEQ | CAUUGACGG (SEQ | CGUCACCAAUCC (SEQ ID | ID NO: 100) |
| | ID NO: 63) | ID NO: 39) | NO: 89) | |
| 82 | CACCACGGCUGUC | AGAAAUCCGUCUUU | ACUCCUACUCUACUAUGGCCGU | CAAGGAAUGAGGGACUUC |
| | GUCACCA (SEQ | CAUUGACGG (SEQ | CGUCACCAAUCC (SEQ ID | UC (SEQ ID NO: 91) |
| | ID NO: 63) | ID NO: 39) | NO: 89) | |
| 83 | CACCACGGCUGUC | AGAAAUCCGUCUUU | CUGAGCCCCUACUCCUACUCUA | CAAGGAAUGAGGG (SEQ |
| | GUCACCA (SEQ | CAUUGACGG (SEQ | CUAUGGCCGUCGUCACCAAUCC | ID NO: 100) |
| | ID NO: 63) | ID NO: 39) | (SEQ ID NO: 90) | |
| 84 | CACCACGGCUGUC | AGAAAUCCGUCUUU | CUGAGCCCCUACUCCUACUCUA | CAAGGAAUGAGGGACUUC |
| | GUCACCA (SEQ | CAUUGACGG (SEQ | CUAUGGCCGUCGUCACCAAUCC | UC (SEQ ID NO: 91) |
| | ID NO: 63) | ID NO: 39) | (SEQ ID NO: 90) | |

TABLE 11
Sequences of Exemplary Editing Template RNAs

| Editing | | SEQ | | SEQ |
|---|---|---|---|---|
| Template | | ID | | ID |
| RNAS | Unmodified | NO | Modified | NO |
| 1 | ACUCCUAUUCCACCAUGGCUGUU | 101 | mA*mC*mU*rCrCrUrArUrUrCrCrArCrCrArUr | 166 |
| | GUUACUAAUCCCAAGGAAUGAGG | | GrGrCrUrGrUrUrGrUrUrArCrUrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCmA*mC*mC*rA | |
| 2 | ACUCCUAUUCCACCAUGGCUGUU | 102 | mA*mC*mU*rCrCrUrArUrUrCrCrArCrCrArUr | 167 |
| | GUUACUAAUCCCAAGGAAUGAGG | | GrGrCrUrGrUrUrGrUrUrArCrUrArArUrCrCr | |
| | GACUUCUCCUCCAGUGGACCUGA | | CrArArGrGrArArUrGrArGrGrGrArCrUrUrCr | |
| | AGAAAUCCGUCUUUCAUUGACGG | | UrCrCrUrCrCrArGrUrGrGrArCrCrUrGrArAr | |
| | CACCACGGCUGUCGUCACCA | | GrArArArUrCrCrGrUrCrUrUrUrCrArUrUrGr | |
| | | | ArCrGrGrCrArCrCrArCrGrGrCrUrGrUrCrGr | |
| | | | UrCmA*mC*mC*rA | |
| 3 | ACUCCUAUUCCACCAUGGCUGUU | 103 | mA*mC*mU*rCrCrUrArUrUrCrCrArCrCrArUr | 168 |
| | GUUACUAAUCCCAAGGAAUGAGG | | GrGrCrUrGrUrUrGrUrUrArCrUrArArUrCrCr | |
| | GACUUCUCCUCCAGUGGACCUGA | | CrArArGrGrArArUrGrArGrGrGrArCrUrUrCr | |
| | AGGACGAGGGAUGGGAGAAAUCC | | UrCrCrUrCrCrArGrUrGrGrArCrCrUrGrArAr | |
| | GUCUUUCAUUGACGGCACCACGG | | GrGrArCrGrArGrGrGrArUrGrGrGrArGrArAr | |
| | CUGUCGUCACCA | | ArUrCrCrGrUrCrUrUrUrCrArUrUrGrArCrGr | |
| | | | GrCrArCrCrArCrGrGrCrUrGrUrCrGrUrCmA* | |
| | | | mC*mC*rA | |
| 4 | CUGAGCCCCUACUCCUAUUCCAC | 104 | mC*mU*mG*rArGrCrCrCrCrUrArCrUrCrCrUr | 169 |
| | CAUGGCUGUUGUUACUAAUCCCA | | ArUrUrCrCrArCrCrArUrGrGrCrUrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCmA*mC*m | |
| | | | C*rA | |
| 5 | CUGAGCCCCUACUCCUAUUCCAC | 105 | mC*mU*mG*rArGrCrCrCrCrUrArCrUrCrCrUr | 170 |
| | CAUGGCUGUUGUUACUAAUCCCA | | ArUrUrCrCrArCrCrArUrGrGrCrUrGrUrUrGr | |
| | AGGAAUGAGGGACUUCUCCUCCA | | UrUrArCrUrArArUrCrCrCrArArGrGrArArUr | |
| | GUGGACCUGAAGAAAUCCGUCUU | | GrArGrGrGrArCrUrUrCrUrCrCrUrCrCrArGr | |
| | UCAUUGACGGCACCACGGCUGUC | | UrGrGrArCrCrUrGrArArGrArArArUrCrCrGr | |
| | GUCACCA | | UrCrUrUrUrCrArUrUrGrArCrGrGrCrArCrCr | |
| | | | ArCrGrGrCrUrGrUrCrGrUrCmA*mC*mC*rA | |
| 6 | CUGAGCCCCUACUCCUAUUCCAC | 106 | mC*mU*mG*rArGrCrCrCrCrUrArCrUrCrCrUr | 171 |
| | CAUGGCUGUUGUUACUAAUCCCA | | ArUrUrCrCrArCrCrArUrGrGrCrUrGrUrUrGr | |
| | AGGAAUGAGGGACUUCUCCUCCA | | UrUrArCrUrArArUrCrCrCrArArGrGrArArUr | |
| | GUGGACCUGAAGGACGAGGGAUG | | GrArGrGrGrArCrUrUrCrUrCrCrUrCrCrArGr | |
| | GGAGAAAUCCGUCUUUCAUUGAC | | UrGrGrArCrCrUrGrArArGrGrArCrGrArGrGr | |
| | GGCACCACGGCUGUCGUCACCA | | GrArUrGrGrGrArGrArArArUrCrCrGrUrCrUr | |
| | | | UrUrCrArUrUrGrArCrGrGrCrArCrCrArCrGr | |
| | | | GrCrUrGrUrCrGrUrCmA*mC*mC*rA | |
| 7 | UGCCGCCCUGCUGAGCCCCUACU | 107 | mU*mG*mC*rCrGrCrCrCrUrGrCrUrGrArGrCr | 172 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCAGAAAUCCGUCUUUCAUU | | CrCrCrArArGrGrArArUrGrArGrGrGrArCrUr | |
| | GACGGCACCACGGCUGUCGUCAC | | UrCrUrCrArGrArArArUrCrCrGrUrCrUrUrUr | |
| | CA | | CrArUrUrGrArCrGrGrCrArCrCrArCrGrGrCr | |
| | | | UrGrUrCrGrUrCmA*mC*mC*rA | |
| 8 | UGCCGCCCUGCUGAGCCCCUACU | 108 | mU*mG*mC*rCrGrCrCrCrUrGrCrUrGrArGrCr | 173 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCCUCCAGUGGACCUGAAGA | | CrCrCrArArGrGrArArUrGrArGrGrGrArCrUr | |
| | AAUCCGUCUUUCAUUGACGGCAC | | UrCrUrCrCrUrCrCrArGrUrGrGrArCrCrUrGr | |
| | CACGGCUGUCGUCACCA | | ArArGrArArArUrCrCrGrUrCrUrUrUrCrArUr | |
| | | | UrGrArCrGrGrCrArCrCrArCrGrGrCrUrGrUr | |
| | | | CrGrUrCmA*mC*mC*rA | |
| 9 | UGCCGCCCUGCUGAGCCCCUACU | 109 | mU*mG*mC*rCrGrCrCrCrUrGrCrUrGrArGrCr | 174 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCCUCCAGUGGACCUGAAGG | | CrCrCrArArGrGrArArUrGrArGrGrGrArCrUr | |
| | ACGAGGGAUGGGAGAAAUCCGUC | | UrCrUrCrCrUrCrCrArGrUrGrGrArCrCrUrGr | |
| | UUUCAUUGACGGCACCACGGCUG | | ArArGrGrArCrGrArGrGrGrArUrGrGrGrArGr | |
| | UCGUCACCA | | ArArArUrCrCrGrUrCrUrUrUrCrArUrUrGrAr | |
| | | | CrGrGrCrArCrCrArCrGrGrCrUrGrUrCrGrUr | |
| | | | CmA*mC*mC*rA | |
| 10 | UGCCGCCCUGCUGAGCCCCUACU | 110 | mU*mG*mC*rCrGrCrCrCrUrGrCrUrGrArGrCr | 175 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCAGAAAUCCGUCUUUCAUU | | CrCrCrArArGrGrArArUrGrArGrGrGrArCrUr | |
| | GACGGCACCACGGCUGUCGUCAC | | UrCrUrCrArGrArArArUrCrCrGrUrCrUrUrUr | |
| | CA | | CrArUrUrGrArCrGrGrCrArCrCrArCrGrGrCr | |
| | | | UrGrUrCrGrUrCmA*mC*mC*rA | |
| 11 | UGCCGCCCUGCUGAGCCCCUACU | 111 | mU*mG*mC*rCrGrCrCrCrUrGrCrUrGrArGrCr | 176 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCCUCCAGUGGACCUGAAGA | | CrCrCrArArGrGrArArUrGrArGrGrGrArCrUr | |
| | AAUCCGUCUUUCAUUGACGGCAC | | UrCrUrCrCrUrCrCrArGrUrGrGrArCrCrUrGr | |
| | CACGGCUGUCGUCACCA | | ArArGrArArArUrCrCrGrUrCrUrUrUrCrArUr | |
| | | | UrGrArCrGrGrCrArCrCrArCrGrGrCrUrGrUr | |
| | | | CrGrUrCmA*mC*mC*rA | |
| 12 | UGCCGCCCUGCUGAGCCCCUACU | 112 | mU*mG*mC*rCrGrCrCrCrUrGrCrUrGrArGrCr | 177 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCCUCCAGUGGACCUGAAGG | | CrCrCrArArGrGrArArUrGrArGrGrGrArCrUr | |
| | ACGAGGGAUGGGAGAAAUCCGUC | | UrCrUrCrCrUrCrCrArGrUrGrGrArCrCrUrGr | |
| | UUUCAUUGACGGCACCACGGCUG | | ArArGrGrArCrGrArGrGrGrArUrGrGrGrArGr | |
| | UCGUCACCA | | ArArArUrCrCrGrUrCrUrUrUrCrArUrUrGrAr | |
| | | | CrGrGrCrArCrCrArCrGrGrCrUrGrUrCrGrUr | |
| | | | CmA*mC*mC*rA | |
| 13 | CUGAGCCCCUACUCCUAUUCCAC | 113 | mC*mU*mG*rArGrCrCrCrCrUrArCrUrCrCrUr | 178 |
| | CAUGGCCGUUGUUACUAAUCCCA | | ArUrUrCrCrArCrCrArUrGrGrCrCrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCmA*mC*m | |
| | | | C*rA | |
| 14 | CUGAGCCCCUACUCCUAUUCCAC | 114 | mC*mU*mG*rArGrCrCrCrCrUrArCrUrCrCrUr | 179 |
| | CAUGGCCGUUGUUACUAAUCCCA | | ArUrUrCrCrArCrCrArUrGrGrCrCrGrUrUrGr | |
| | AGGAAUGAGGGACUUCUCCUCCA | | UrUrArCrUrArArUrCrCrCrArArGrGrArArUr | |
| | GUGGACCUGAAGAAAUCCGUCUU | | GrArGrGrGrArCrUrUrCrUrCrCrUrCrCrArGr | |
| | UCAUUGACGGCACCACGGCUGUC | | UrGrGrArCrCrUrGrArArGrArArArUrCrCrGr | |
| | GUCACCA | | UrCrUrUrUrCrArUrUrGrArCrGrGrCrArCrCr | |
| | | | ArCrGrGrCrUrGrUrCrGrUrCmA*mC*mC*rA | |
| 15 | CUGAGCCCCUACUCCUAUUCCAC | 115 | mC*mU*mG*rArGrCrCrCrCrUrArCrUrCrCrUr | 180 |
| | CAUGGCCGUUGUUACUAAUCCCA | | ArUrUrCrCrArCrCrArUrGrGrCrCrGrUrUrGr | |
| | AGGAAUGAGGGACUUCUCCUCCA | | UrUrArCrUrArArUrCrCrCrArArGrGrArArUr | |
| | GUGGACCUGAAGGACGAGGGAUG | | GrArGrGrGrArCrUrUrCrUrCrCrUrCrCrArGr | |
| | GGAGAAAUCCGUCUUUCAUUGAC | | UrGrGrArCrCrUrGrArArGrGrArCrGrArGrGr | |
| | GGCACCACGGCUGUCGUCACCA | | GrArUrGrGrGrArGrArArArUrCrCrGrUrCrUr | |
| | | | UrUrCrArUrUrGrArCrGrGrCrArCrCrArCrGr | |
| | | | GrCrUrGrUrCrGrUrCmA*mC*mC*rA | |
| 16 | UGCCGCCCUAUUAUCACCCUACU | 116 | mU*mG*mC*rCrGrCrCrCrUrArUrUrArUrCrAr | 181 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCAGAAAUCCGUCUUUCAUU | | CrCrCrArArGrGrArArUrGrArGrGrGrArCrUr | |
| | GACGGCACCACGGCUGUCGUCAC | | UrCrUrCrArGrArArArUrCrCrGrUrCrUrUrUr | |
| | CA | | CrArUrUrGrArCrGrGrCrArCrCrArCrGrGrCr | |
| | | | UrGrUrCrGrUrCmA*mC*mC*rA | |
| 17 | UGCCGCCCUAUUAUCACCCUACU | 117 | mU*mG*mC*rCrGrCrCrCrUrArUrUrArUrCrAr | 182 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCCUCCAGUGGACCUGAAGA | | CrCrCrArArGrGrArArUrGrArGrGrGrArCrUr | |
| | AAUCCGUCUUUCAUUGACGGCAC | | UrCrUrCrCrUrCrCrArGrUrGrGrArCrCrUrGr | |
| | CACGGCUGUCGUCACCA | | ArArGrArArArUrCrCrGrUrCrUrUrUrCrArUr | |
| | | | UrGrArCrGrGrCrArCrCrArCrGrGrCrUrGrUr | |
| | | | CrGrUrCmA*mC*mC*rA | |
| 18 | UGCCGCCCUAUUAUCACCCUACU | 118 | mU*mG*mC*rCrGrCrCrCrUrArUrUrArUrCrAr | 183 |
| | CCUAUUCCACCAUGGCCGUUGUU | | CrCrCrUrArCrUrCrCrUrArUrUrCrCrArCrCr | |
| | ACUAAUCCCAAGGAAUGAGGGAC | | ArUrGrGrCrCrGrUrUrGrUrUrArCrUrArArUr | |
| | UUCUCCUCCAGUGGACCUGAAGG | | CrCrCrArArGrGrArArUrGrArGrGrGrArCrUr | |
| | ACGAGGGAUGGGAGAAAUCCGUC | | UrCrUrCrCrUrCrCrArGrUrGrGrArCrCrUrGr | |
| | UUUCAUUGACGGCACCACGGCUG | | ArArGrGrArCrGrArGrGrGrArUrGrGrGrArGr | |
| | UCGUCACCA | | ArArArUrCrCrGrUrCrUrUrUrCrArUrUrGrAr | |
| | | | CrGrGrCrArCrCrArCrGrGrCrUrGrUrCrGrUr | |
| | | | CmA*mC*mC*rA | |
| 19 | CUGAGCCCCUACUCCUAUUCCAC | 104 | mC*mU*mG*rArGrCrCrCrCrUrArCrUrCrCrUr | 184 |
| | CAUGGCUGUUGUUACUAAUCCCA | | ArUrUrCrCrArCrCrArUrGrGrCrUrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 20 | CUGAGCCCCUACUCCUAUUCCAC | 105 | mC*mU*mG*rArGrCrCrCrCrUrArCrUrCrCrUr | 185 |
| | CAUGGCUGUUGUUACUAAUCCCA | | ArUrUrCrCrArCrCrArUrGrGrCrUrGrUrUrGr | |
| | AGGAAUGAGGGACUUCUCCUCCA | | UrUrArCrUrArArUrCrCrCrArArGrGrArArUr | |
| | GUGGACCUGAAGAAAUCCGUCUU | | GrArGrGrGrArCrUrUrCrUrCrCrUrCrCrArGr | |
| | UCAUUGACGGCACCACGGCUGUC | | UrGrGrArCrCrUrGrArArGrArArArUrCrCrGr | |
| | GUCACCA | | UrCrUrUrUrCrArUrUrGrArCrGrGrCrArCrCr | |
| | | | ArCrGrGrCrUrGrUrCrGrUrCrA*mC*mC*mA | |
| 21 | CUGAGCCCCUACUCCUAUUCCAC | 106 | mC*mU*mG*rArGrCrCrCrCrUrArCrUrCrCrUr | 186 |
| | CAUGGCUGUUGUUACUAAUCCCA | | ArUrUrCrCrArCrCrArUrGrGrCrUrGrUrUrGr | |
| | AGGAAUGAGGGACUUCUCCUCCA | | UrUrArCrUrArArUrCrCrCrArArGrGrArArUr | |
| | GUGGACCUGAAGGACGAGGGAUG | | GrArGrGrGrArCrUrUrCrUrCrCrUrCrCrArGr | |
| | GGAGAAAUCCGUCUUUCAUUGAC | | UrGrGrArCrCrUrGrArArGrGrArCrGrArGrGr | |
| | GGCACCACGGCUGUCGUCACCA | | GrArUrGrGrGrArGrArArArUrCrCrGrUrCrUr | |
| | | | UrUrCrArUrUrGrArCrGrGrCrArCrCrArCrGr | |
| | | | GrCrUrGrUrCrGrUrCrA*mC*mC*mA | |
| 22 | UGCCGCCCUGCUGAGCCCCUACU | 107 | mU*mG*mC*rCrGrCrCrCrUrGrCrUrGrArGrCr | 187 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCAGAAAUCCGUCUUUCAUU | | CrCrCrArArGrGrArArUrGrArGrGrGrArCrUr | |
| | GACGGCACCACGGCUGUCGUCAC | | UrCrUrCrArGrArArArUrCrCrGrUrCrUrUrUr | |
| | CA | | CrArUrUrGrArCrGrGrCrArCrCrArCrGrGrCr | |
| | | | UrGrUrCrGrUrCrA*mC*mC*mA | |
| 23 | UGCCGCCCUGCUGAGCCCCUACU | 108 | mU*mG*mC*rCrGrCrCrCrUrGrCrUrGrArGrCr | 188 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCCUCCAGUGGACCUGAAGA | | CrCrCrArArGrGrArArUrGrArGrGrGrArCrUr | |
| | AAUCCGUCUUUCAUUGACGGCAC | | UrCrUrCrCrUrCrCrArGrUrGrGrArCrCrUrGr | |
| | CACGGCUGUCGUCACCA | | ArArGrArArArUrCrCrGrUrCrUrUrUrCrArUr | |
| | | | UrGrArCrGrGrCrArCrCrArCrGrGrCrUrGrUr | |
| | | | CrGrUrCrA*mC*mC*mA | |
| 24 | UGCCGCCCUGCUGAGCCCCUACU | 109 | mU*mG*mC*rCrGrCrCrCrUrGrCrUrGrArGrCr | 189 |
| | CCUAUUCCACCAUGGCUGUUGUU | | CrCrCrUrArCrUrCrCrUrArUrUrCrCrArCrCr | |
| | ACUAAUCCCAAGGAAUGAGGGAC | | ArUrGrGrCrUrGrUrUrGrUrUrArCrUrArArUr | |
| | UUCUCCUCCAGUGGACCUGAAGG | | CrCrCrArArGrGrArArUrGrArGrGrGrArCrUr | |
| | ACGAGGGAUGGGAGAAAUCCGUC | | UrCrUrCrCrUrCrCrArGrUrGrGrArCrCrUrGr | |
| | UUUCAUUGACGGCACCACGGCUG | | ArArGrGrArCrGrArGrGrGrArUrGrGrGrArGr | |
| | UCGUCACCA | | ArArArUrCrCrGrUrCrUrUrUrCrArUrUrGrAr | |
| | | | CrGrGrCrArCrCrArCrGrGrCrUrGrUrCrGrUr | |
| | | | CrA*mC*mC*mA | |
| 25 | UCAUUCCUUGGGAUUGGUGACAA | 119 | mU*mC*mA*rUrUrCrCrUrUrGrGrGrArUrUrGr | 190 |
| | CGGCCAUGGUGGAAUAGGAGUAG | | GrUrGrArCrArArCrGrGrCrCrArUrGrGrUrGr | |
| | GGGCUCAGAGAAAUCCGUCUUUC | | GrArArUrArGrGrArGrUrArGrGrGrGrCrUrCr | |
| | AUUGACGGGGAUUGGUGACGACA | | ArGrArGrArArArUrCrCrGrUrCrUrUrUrCrAr | |
| | GCCGU | | UrUrGrArCrGrGrGrGrArUrUrGrGrUrGrArCr | |
| | | | GrArCrArGmC*mC*mG*rU | |
| 26 | UCAUUCCUUGGGAUUGGUGACAA | 120 | mU*mC*mA*rUrUrCrCrUrUrGrGrGrArUrUrGr | 191 |
| | CGGCCAUGGUGGAAUAGGAGUAG | | GrUrGrArCrArArCrGrGrCrCrArUrGrGrUrGr | |
| | GGGCUCAGCAGGGCGGCAAUGGU | | GrArArUrArGrGrArGrUrArGrGrGrGrCrUrCr | |
| | AGAAAUCCGUCUUUCAUUGACGG | | ArGrCrArGrGrGrCrGrGrCrArArUrGrGrUrAr | |
| | GGAUUGGUGACGACAGCCGU | | GrArArArUrCrCrGrUrCrUrUrUrCrArUrUrGr | |
| | | | ArCrGrGrGrGrArUrUrGrGrUrGrArCrGrArCr | |
| | | | ArGmC*mC*mG*rU | |
| 27 | UCAUUCCUUGGGAUUGGUGACAA | 121 | mU*mC*mA*rUrUrCrCrUrUrGrGrGrArUrUrGr | 192 |
| | CGGCCAUGGUGGAAUAGGAGUAG | | GrUrGrArCrArArCrGrGrCrCrArUrGrGrUrGr | |
| | GGGCUCAGCAGGGCGGCAAUGGU | | GrArArUrArGrGrArGrUrArGrGrGrGrCrUrCr | |
| | GUAGCGGCGGGGGCCAGAAAUCC | | ArGrCrArGrGrGrCrGrGrCrArArUrGrGrUrGr | |
| | GUCUUUCAUUGACGGGGAUUGGU | | UrArGrCrGrGrCrGrGrGrGrGrCrCrArGrArAr | |
| | GACGACAGCCGU | | ArUrCrCrGrUrCrUrUrUrCrArUrUrGrArCrGr | |
| | | | GrGrGrArUrUrGrGrUrGrArCrGrArCrArGmC* | |
| | | | mC*mG*rU | |
| 28 | GAGAAGUCCCUCAUUCCUUGGGA | 122 | mG*mA*mG*rArArGrUrCrCrCrUrCrArUrUrCr | 193 |
| | UUGGUGACAACGGCCAUGGUGGA | | CrUrUrGrGrGrArUrUrGrGrUrGrArCrArArCr | |
| | AUAGGAGUAGGGGCUCAGAGAAA | | GrGrCrCrArUrGrGrUrGrGrArArUrArGrGrAr | |
| | UCCGUCUUUCAUUGACGGGGAUU | | GrUrArGrGrGrGrCrUrCrArGrArGrArArArUr | |
| | GGUGACGACAGCCGU | | CrCrGrUrCrUrUrUrCrArUrUrGrArCrGrGrGr | |
| | | | GrArUrUrGrGrUrGrArCrGrArCrArGmC*mC*m | |
| | | | G*rU | |
| 29 | GAGAAGUCCCUCAUUCCUUGGGA | 123 | mG*mA*mG*rArArGrUrCrCrCrUrCrArUrUrCr | 194 |
| | UUGGUGACAACGGCCAUGGUGGA | | CrUrUrGrGrGrArUrUrGrGrUrGrArCrArArCr | |
| | AUAGGAGUAGGGGCUCAGCAGGG | | GrGrCrCrArUrGrGrUrGrGrArArUrArGrGrAr | |
| | CGGCAAUGGUAGAAAUCCGUCUU | | GrUrArGrGrGrGrCrUrCrArGrCrArGrGrGrCr | |
| | UCAUUGACGGGGAUUGGUGACGA | | GrGrCrArArUrGrGrUrArGrArArArUrCrCrGr | |
| | CAGCCGU | | UrCrUrUrUrCrArUrUrGrArCrGrGrGrGrArUr | |
| | | | UrGrGrUrGrArCrGrArCrArGmC*mC*mG*rU | |
| 30 | GAGAAGUCCCUCAUUCCUUGGGA | 124 | mG*mA*mG*rArArGrUrCrCrCrUrCrArUrUrCr | 195 |
| | UUGGUGACAACGGCCAUGGUGGA | | CrUrUrGrGrGrArUrUrGrGrUrGrArCrArArCr | |
| | AUAGGAGUAGGGGCUCAGCAGGG | | GrGrCrCrArUrGrGrUrGrGrArArUrArGrGrAr | |
| | CGGCAAUGGUGUAGCGGCGGGGG | | GrUrArGrGrGrGrCrUrCrArGrCrArGrGrGrCr | |
| | CCAGAAAUCCGUCUUUCAUUGAC | | GrGrCrArArUrGrGrUrGrUrArGrCrGrGrCrGr | |
| | GGGGAUUGGUGACGACAGCCGU | | GrGrGrGrCrCrArGrArArArUrCrCrGrUrCrUr | |
| | | | UrUrCrArUrUrGrArCrGrGrGrGrArUrUrGrGr | |
| | | | UrGrArCrGrArCrArGmC*mC*mG*rU | |
| 31 | UCCACUGGAGGAGAAGUCCCUCA | 125 | mU*mC*mC*rArCrUrGrGrArGrGrArGrArArGr | 196 |
| | UUCCUUGGGAUUGGUGACAACGG | | UrCrCrCrUrCrArUrUrCrCrUrUrGrGrGrArUr | |
| | CCAUGGUGGAAUAGGAGUAGGGG | | UrGrGrUrGrArCrArArCrGrGrCrCrArUrGrGr | |
| | CUCAGAGAAAUCCGUCUUUCAUU | | UrGrGrArArUrArGrGrArGrUrArGrGrGrGrCr | |
| | GACGGGGAUUGGUGACGACAGCC | | UrCrArGrArGrArArArUrCrCrGrUrCrUrUrUr | |
| | GU | | CrArUrUrGrArCrGrGrGrGrArUrUrGrGrUrGr | |
| | | | ArCrGrArCrArGmC*mC*mG*rU | |
| 32 | UCCACUGGAGGAGAAGUCCCUCA | 126 | mU*mC*mC*rArCrUrGrGrArGrGrArGrArArGr | 197 |
| | UUCCUUGGGAUUGGUGACAACGG | | UrCrCrCrUrCrArUrUrCrCrUrUrGrGrGrArUr | |
| | CCAUGGUGGAAUAGGAGUAGGGG | | UrGrGrUrGrArCrArArCrGrGrCrCrArUrGrGr | |
| | CUCAGCAGGGCGGCAAUGGUAGA | | UrGrGrArArUrArGrGrArGrUrArGrGrGrGrCr | |
| | AAUCCGUCUUUCAUUGACGGGGA | | UrCrArGrCrArGrGrGrCrGrGrCrArArUrGrGr | |
| | UUGGUGACGACAGCCGU | | UrArGrArArArUrCrCrGrUrCrUrUrUrCrArUr | |
| | | | UrGrArCrGrGrGrGrArUrUrGrGrUrGrArCrGr | |
| | | | ArCrArGmC*mC*mG*rU | |
| 33 | UCCACUGGAGGAGAAGUCCCUCA | 127 | mU*mC*mC*rArCrUrGrGrArGrGrArGrArArGr | 198 |
| | UUCCUUGGGAUUGGUGACAACGG | | UrCrCrCrUrCrArUrUrCrCrUrUrGrGrGrArUr | |
| | CCAUGGUGGAAUAGGAGUAGGGG | | UrGrGrUrGrArCrArArCrGrGrCrCrArUrGrGr | |
| | CUCAGCAGGGCGGCAAUGGUGUA | | UrGrGrArArUrArGrGrArGrUrArGrGrGrGrCr | |
| | GCGGCGGGGGCCAGAAAUCCGUC | | UrCrArGrCrArGrGrGrCrGrGrCrArArUrGrGr | |
| | UUUCAUUGACGGGGAUUGGUGAC | | UrGrUrArGrCrGrGrCrGrGrGrGrGrCrCrArGr | |
| | GACAGCCGU | | ArArArUrCrCrGrUrCrUrUrUrCrArUrUrGrAr | |
| | | | CrGrGrGrGrArUrUrGrGrUrGrArCrGrArCrAr | |
| | | | GmC*mC*mG*rU | |
| 34 | UCCCUCAUUCCUUGGGAUUGGUA | 128 | mU*mC*mC*rCrUrCrArUrUrCrCrUrUrGrGrGr | 199 |
| | ACAACGGCCAUGGUGGAAUAGGA | | ArUrUrGrGrUrArArCrArArCrGrGrCrCrArUr | |
| | GUAGGGGCAGAAAUCCGUCUUUC | | GrGrUrGrGrArArUrArGrGrArGrUrArGrGrGr | |
| | AUUGACGGCUUGGGAUUGGUGAC | | GrCrArGrArArArUrCrCrGrUrCrUrUrUrCrAr | |
| | GACAG | | UrUrGrArCrGrGrCrUrUrGrGrGrArUrUrGrGr | |
| | | | UrGrArCrGmA*mC*mA*rG | |
| 35 | UCCCUCAUUCCUUGGGAUUGGUA | 129 | mU*mC*mC*rCrUrCrArUrUrCrCrUrUrGrGrGr | 200 |
| | ACAACGGCCAUGGUGGAAUAGGA | | ArUrUrGrGrUrArArCrArArCrGrGrCrCrArUr | |
| | GUAGGGGCUCAGCAGGGCGGCAA | | GrGrUrGrGrArArUrArGrGrArGrUrArGrGrGr | |
| | AGAAAUCCGUCUUUCAUUGACGG | | GrCrUrCrArGrCrArGrGrGrCrGrGrCrArArAr | |
| | CUUGGGAUUGGUGACGACAG | | GrArArArUrCrCrGrUrCrUrUrUrCrArUrUrGr | |
| | | | ArCrGrGrCrUrUrGrGrGrArUrUrGrGrUrGrAr | |
| | | | CrGmA*mC*mA*rG | |
| 36 | UCCCUCAUUCCUUGGGAUUGGUA | 130 | mU*mC*mC*rCrUrCrArUrUrCrCrUrUrGrGrGr | 201 |
| | ACAACGGCCAUGGUGGAAUAGGA | | ArUrUrGrGrUrArArCrArArCrGrGrCrCrArUr | |
| | GUAGGGGCUCAGCAGGGCGGCAA | | GrGrUrGrGrArArUrArGrGrArGrUrArGrGrGr | |
| | UGGUGUAGCGGCGGGAGAAAUCC | | GrCrUrCrArGrCrArGrGrGrCrGrGrCrArArUr | |
| | GUCUUUCAUUGACGGCUUGGGAU | | GrGrUrGrUrArGrCrGrGrCrGrGrGrArGrArAr | |
| | UGGUGACGACAG | | ArUrCrCrGrUrCrUrUrUrCrArUrUrGrArCrGr | |
| | | | GrCrUrUrGrGrGrArUrUrGrGrUrGrArCrGmA* | |
| | | | mC*mA*rG | |
| 37 | GGAGGAGAAGUCCCUCAUUCCUU | 131 | mG*mG*mA*rGrGrArGrArArGrUrCrCrCrUrCr | 202 |
| | GGGAUUGGUAACAACGGCCAUGG | | ArUrUrCrCrUrUrGrGrGrArUrUrGrGrUrArAr | |
| | UGGAAUAGGAGUAGGGGCAGAAA | | CrArArCrGrGrCrCrArUrGrGrUrGrGrArArUr | |
| | UCCGUCUUUCAUUGACGGCUUGG | | ArGrGrArGrUrArGrGrGrGrCrArGrArArArUr | |
| | GAUUGGUGACGACAG | | CrCrGrUrCrUrUrUrCrArUrUrGrArCrGrGrCr | |
| | | | UrUrGrGrGrArUrUrGrGrUrGrArCrGmA*mC*m | |
| | | | A*rG | |
| 38 | GGAGGAGAAGUCCCUCAUUCCUU | 132 | mG*mG*mA*rGrGrArGrArArGrUrCrCrCrUrCr | 203 |
| | GGGAUUGGUAACAACGGCCAUGG | | ArUrUrCrCrUrUrGrGrGrArUrUrGrGrUrArAr | |
| | UGGAAUAGGAGUAGGGGCUCAGC | | CrArArCrGrGrCrCrArUrGrGrUrGrGrArArUr | |
| | AGGGCGGCAAAGAAAUCCGUCUU | | ArGrGrArGrUrArGrGrGrGrCrUrCrArGrCrAr | |
| | UCAUUGACGGCUUGGGAUUGGUG | | GrGrGrCrGrGrCrArArArGrArArArUrCrCrGr | |
| | ACGACAG | | UrCrUrUrUrCrArUrUrGrArCrGrGrCrUrUrGr | |
| | | | GrGrArUrUrGrGrUrGrArCrGmA*mC*mA*rG | |
| 39 | GGAGGAGAAGUCCCUCAUUCCUU | 133 | mG*mG*mA*rGrGrArGrArArGrUrCrCrCrUrCr | 20 |
| | GGGAUUGGUAACAACGGCCAUGG | | ArUrUrCrCrUrUrGrGrGrArUrUrGrGrUrArAr | |
| | UGGAAUAGGAGUAGGGGCUCAGC | | CrArArCrGrGrCrCrArUrGrGrUrGrGrArArUr | |
| | AGGGCGGCAAUGGUGUAGCGGCG | | ArGrGrArGrUrArGrGrGrGrCrUrCrArGrCrAr | |
| | GGAGAAAUCCGUCUUUCAUUGAC | | GrGrGrCrGrGrCrArArUrGrGrUrGrUrArGrCr | |
| | GGCUUGGGAUUGGUGACGACAG | | GrGrCrGrGrGrArGrArArArUrCrCrGrUrCrUr | |
| | | | UrUrCrArUrUrGrArCrGrGrCrUrUrGrGrGrAr | |
| | | | UrUrGrGrUrGrArCrGmA*mC*mA*rG | |
| 40 | CAGGUCCACUGGAGGAGAAGUCC | 134 | mC*mA*mG*rGrUrCrCrArCrUrGrGrArGrGrAr | 205 |
| | CUCAUUCCUUGGGAUUGGUAACA | | GrArArGrUrCrCrCrUrCrArUrUrCrCrUrUrGr | |
| | ACGGCCAUGGUGGAAUAGGAGUA | | GrGrArUrUrGrGrUrArArCrArArCrGrGrCrCr | |
| | GGGGCAGAAAUCCGUCUUUCAUU | | ArUrGrGrUrGrGrArArUrArGrGrArGrUrArGr | |
| | GACGGCUUGGGAUUGGUGACGAC | | GrGrGrCrArGrArArArUrCrCrGrUrCrUrUrUr | |
| | AG | | CrArUrUrGrArCrGrGrCrUrUrGrGrGrArUrUr | |
| | | | GrGrUrGrArCrGmA*mC*mA*rG | |
| 41 | CAGGUCCACUGGAGGAGAAGUCC | 135 | mC*mA*mG*rGrUrCrCrArCrUrGrGrArGrGrAr | 206 |
| | CUCAUUCCUUGGGAUUGGUAACA | | GrArArGrUrCrCrCrUrCrArUrUrCrCrUrUrGr | |
| | ACGGCCAUGGUGGAAUAGGAGUA | | GrGrArUrUrGrGrUrArArCrArArCrGrGrCrCr | |
| | GGGGCUCAGCAGGGCGGCAAAGA | | ArUrGrGrUrGrGrArArUrArGrGrArGrUrArGr | |
| | AAUCCGUCUUUCAUUGACGGCUU | | GrGrGrCrUrCrArGrCrArGrGrGrCrGrGrCrAr | |
| | GGGAUUGGUGACGACAG | | ArArGrArArArUrCrCrGrUrCrUrUrUrCrArUr | |
| | | | UrGrArCrGrGrCrUrUrGrGrGrArUrUrGrGrUr | |
| | | | GrArCrGmA*mC*mA*rG | |
| 42 | CAGGUCCACUGGAGGAGAAGUCC | 136 | mC*mA*mG*rGrUrCrCrArCrUrGrGrArGrGrAr | 207 |
| | CUCAUUCCUUGGGAUUGGUAACA | | GrArArGrUrCrCrCrUrCrArUrUrCrCrUrUrGr | |
| | ACGGCCAUGGUGGAAUAGGAGUA | | GrGrArUrUrGrGrUrArArCrArArCrGrGrCrCr | |
| | GGGGCUCAGCAGGGCGGCAAUGG | | ArUrGrGrUrGrGrArArUrArGrGrArGrUrArGr | |
| | UGUAGCGGCGGGAGAAAUCCGUC | | GrGrGrCrUrCrArGrCrArGrGrGrCrGrGrCrAr | |
| | UUUCAUUGACGGCUUGGGAUUGG | | ArUrGrGrUrGrUrArGrCrGrGrCrGrGrGrArGr | |
| | UGACGACAG | | ArArArUrCrCrGrUrCrUrUrUrCrArUrUrGrAr | |
| | | | CrGrGrCrUrUrGrGrGrArUrUrGrGrUrGrArCr | |
| | | | GmA*mC*mA*rG | |
| 54 | ACUCCUAUUCCACCAUGGCUGUU | 137 | mA*mC*mU*rCrCrUrArUrUrCrCrArCrCrArUr | 208 |
| | GUUACUAAUCCCAAGGAAUGAGG | | GrGrCrUrGrUrUrGrUrUrArCrUrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 55 | ACUCCUAUUCCACCAUGGCUGUU | 101 | mA*mC*mU*rCrCrUrArUrUrCrCrArCrCrArUr | 209 |
| | GUUACUAAUCCCAAGGAAUGAGG | | GrGrCrUrGrUrUrGrUrUrArCrUrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 56 | CUGAGCCCCUACUCCUAUUCCAC | 138 | mC*mU*mG*rArGrCrCrCrCrUrArCrUrCrCrUr | 210 |
| | CAUGGCUGUUGUUACUAAUCCCA | | ArUrUrCrCrArCrCrArUrGrGrCrUrGrUrUrGr | |
| | AGGAAUGAGGGAGAAAUCCGUCU | | UrUrArCrUrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 57 | ACUCCUAUUCCACCAUGGCCGUU | 139 | mA*mC*mU*rCrCrUrArUrUrCrCrArCrCrArUr | 211 |
| | GUUACUAAUCCCAAGGAAUGAGG | | GrGrCrCrGrUrUrGrUrUrArCrUrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 58 | ACUCCUAUUCCACCAUGGCCGUU | 140 | mA*mC*mU*rCrCrUrArUrUrCrCrArCrCrArUr | 212 |
| | GUUACUAAUCCCAAGGAAUGAGG | | GrGrCrCrGrUrUrGrUrUrArCrUrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 59 | CUGAGCCCCUACUCCUAUUCCAC | 141 | mC*mU*mG*rArGrCrCrCrCrUrArCrUrCrCrUr | 213 |
| | CAUGGCCGUUGUUACUAAUCCCA | | ArCrUrCrUrArCrUrArUrGrGrCrCrGrUrCrGr | |
| | AGGAAUGAGGGAGAAAUCCGUCU | | UrCrArCrCrArArUrCrCrCrArArGrGrArArUr | |
| | UUCAUUGACGGCACCACGGCUGU | | GrArGrGrGrArCrUrUrCrUrCrArGrArArArUr | |
| | CGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 60 | CUGAGCCCCUACUCCUAUUCCAC | 113 | mC*mU*mG*rArGrCrCrCrCrUrArCrUrCrCrUr | 214 |
| | CAUGGCCGUUGUUACUAAUCCCA | | ArUrUrCrCrArCrCrArUrGrGrCrCrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 61 | ACUCCUAUUCCACUAUGGCCGUU | 142 | mA*mC*mU*rCrCrUrArUrUrCrCrArCrUrArUr | 215 |
| | GUUACUAAUCCCAAGGAAUGAGG | | GrGrCrCrGrUrUrGrUrUrArCrUrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 62 | ACUCCUAUUCCACUAUGGCCGUU | 143 | mA*mC*mU*rCrCrUrArUrUrCrCrArCrUrArUr | 216 |
| | GUUACUAAUCCCAAGGAAUGAGG | | GrGrCrCrGrUrUrGrUrUrArCrUrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 63 | CUGAGCCCCUACUCCUAUUCCAC | 144 | mC*mU*mG*rArGrCrCrCrCrUrArCrUrCrCrUr | 217 |
| | UAUGGCCGUUGUUACUAAUCCCA | | ArUrUrCrCrArCrUrArUrGrGrCrCrGrUrUrGr | |
| | AGGAAUGAGGGAGAAAUCCGUCU | | UrUrArCrUrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 64 | CUGAGCCCCUACUCCUAUUCCAC | 145 | mC*mU*mG*rArGrCrCrCrCrUrArCrUrCrCrUr | 218 |
| | UAUGGCCGUUGUUACUAAUCCCA | | ArUrUrCrCrArCrUrArUrGrGrCrCrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 65 | ACUCCUAUUCUACUAUGGCCGUU | 146 | mA*mC*mU*rCrCrUrArUrUrCrUrArCrUrArUr | 219 |
| | GUUACUAAUCCCAAGGAAUGAGG | | GrGrCrCrGrUrUrGrUrUrArCrUrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 66 | ACUCCUAUUCUACUAUGGCCGUU | 147 | mA*mC*mU*rCrCrUrArUrUrCrUrArCrUrArUr | 220 |
| | GUUACUAAUCCCAAGGAAUGAGG | | GrGrCrCrGrUrUrGrUrUrArCrUrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 67 | CUGAGCCCCUACUCCUAUUCUAC | 148 | mC*mU*mG*rArGrCrCrCrCrUrArCrUrCrCrUr | 221 |
| | UAUGGCCGUUGUUACUAAUCCCA | | ArUrUrCrUrArCrUrArUrGrGrCrCrGrUrUrGr | |
| | AGGAAUGAGGGAGAAAUCCGUCU | | UrUrArCrUrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 68 | CUGAGCCCCUACUCCUAUUCUAC | 149 | mC*mU*mG*rArGrCrCrCrCrUrArCrUrCrCrUr | 222 |
| | UAUGGCCGUUGUUACUAAUCCCA | | ArUrUrCrUrArCrUrArUrGrGrCrCrGrUrUrGr | |
| | AGGAAUGAGGGACUUCUCAGAAA | | UrUrArCrUrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 69 | ACUCCUAUUCUACUAUGGCCGUU | 150 | mA*mC*mU*rCrCrUrArUrUrCrUrArCrUrArUr | 223 |
| | GUUACCAAUCCCAAGGAAUGAGG | | GrGrCrCrGrUrUrGrUrUrArCrCrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 70 | ACUCCUAUUCUACUAUGGCCGUU | 151 | mA*mC*mU*rCrCrUrArUrUrCrUrArCrUrArUr | 224 |
| | GUUACCAAUCCCAAGGAAUGAGG | | GrGrCrCrGrUrUrGrUrUrArCrCrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 71 | CUGAGCCCCUACUCCUAUUCUAC | 152 | mC*mU*mG*rArGrCrCrCrCrUrArCrUrCrCrUr | 225 |
| | UAUGGCCGUUGUUACCAAUCCCA | | ArUrUrCrUrArCrUrArUrGrGrCrCrGrUrUrGr | |
| | AGGAAUGAGGGAGAAAUCCGUCU | | UrUrArCrCrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 72 | CUGAGCCCCUACUCCUAUUCUAC | 153 | mC*mU*mG*rArGrCrCrCrCrUrArCrUrCrCrUr | 226 |
| | UAUGGCCGUUGUUACCAAUCCCA | | ArUrUrCrUrArCrUrArUrGrGrCrCrGrUrUrGr | |
| | AGGAAUGAGGGACUUCUCAGAAA | | UrUrArCrCrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 73 | ACUCCUAUUCUACUAUGGCCGUU | 154 | mA*mC*mU*rCrCrUrArUrUrCrUrArCrUrArUr | 227 |
| | GUCACCAAUCCCAAGGAAUGAGG | | GrGrCrCrGrUrUrGrUrCrArCrCrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 74 | ACUCCUAUUCUACUAUGGCCGUU | 155 | mA*mC*mU*rCrCrUrArUrUrCrUrArCrUrArUr | 228 |
| | GUCACCAAUCCCAAGGAAUGAGG | | GrGrCrCrGrUrUrGrUrCrArCrCrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 75 | CUGAGCCCCUACUCCUAUUCUAC | 156 | mC*mU*mG*rArGrCrCrCrCrUrArCrUrCrCrUr | 229 |
| | UAUGGCCGUUGUCACCAAUCCCA | | ArUrUrCrUrArCrUrArUrGrGrCrCrGrUrUrGr | |
| | AGGAAUGAGGGAGAAAUCCGUCU | | UrCrArCrCrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 76 | CUGAGCCCCUACUCCUAUUCUAC | 157 | mC*mU*mG*rArGrCrCrCrCrUrArCrUrCrCrUr | 230 |
| | UAUGGCCGUUGUCACCAAUCCCA | | ArUrUrCrUrArCrUrArUrGrGrCrCrGrUrUrGr | |
| | AGGAAUGAGGGACUUCUCAGAAA | | UrCrArCrCrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 77 | ACUCCUAUUCUACUAUGGCCGUC | 158 | mA*mC*mU*rCrCrUrArUrUrCrUrArCrUrArUr | 231 |
| | GUCACCAAUCCCAAGGAAUGAGG | | GrGrCrCrGrUrCrGrUrCrArCrCrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 78 | ACUCCUAUUCUACUAUGGCCGUC | 159 | mA*mC*mU*rCrCrUrArUrUrCrUrArCrUrArUr | 232 |
| | GUCACCAAUCCCAAGGAAUGAGG | | GrGrCrCrGrUrCrGrUrCrArCrCrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 79 | CUGAGCCCCUACUCCUAUUCUAC | 160 | mC*mU*mG*rArGrCrCrCrCrUrArCrUrCrCrUr | 233 |
| | UAUGGCCGUCGUCACCAAUCCCA | | ArUrUrCrUrArCrUrArUrGrGrCrCrGrUrCrGr | |
| | AGGAAUGAGGGAGAAAUCCGUCU | | UrCrArCrCrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 80 | CUGAGCCCCUACUCCUAUUCUAC | 161 | mC*mU*mG*rArGrCrCrCrCrUrArCrUrCrCrUr | 234 |
| | UAUGGCCGUCGUCACCAAUCCCA | | ArUrUrCrUrArCrUrArUrGrGrCrCrGrUrCrGr | |
| | AGGAAUGAGGGACUUCUCAGAAA | | UrCrArCrCrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |
| 81 | ACUCCUACUCUACUAUGGCCGUC | 162 | mA*mC*mU*rCrCrUrArCrUrCrUrArCrUrArUr | 235 |
| | GUCACCAAUCCCAAGGAAUGAGG | | GrGrCrCrGrUrCrGrUrCrArCrCrArArUrCrCr | |
| | GAGAAAUCCGUCUUUCAUUGACG | | CrArArGrGrArArUrGrArGrGrGrArGrArArAr | |
| | GCACCACGGCUGUCGUCACCA | | UrCrCrGrUrCrUrUrUrCrArUrUrGrArCrGrGr | |
| | | | CrArCrCrArCrGrGrCrUrGrUrCrGrUrCrA*mC | |
| | | | *mC*mA | |
| 82 | ACUCCUACUCUACUAUGGCCGUC | 163 | mA*mC*mU*rCrCrUrArCrUrCrUrArCrUrArUr | 236 |
| | GUCACCAAUCCCAAGGAAUGAGG | | GrGrCrCrGrUrCrGrUrCrArCrCrArArUrCrCr | |
| | GACUUCUCAGAAAUCCGUCUUUC | | CrArArGrGrArArUrGrArGrGrGrArCrUrUrCr | |
| | AUUGACGGCACCACGGCUGUCGU | | UrCrArGrArArArUrCrCrGrUrCrUrUrUrCrAr | |
| | CACCA | | UrUrGrArCrGrGrCrArCrCrArCrGrGrCrUrGr | |
| | | | UrCrGrUrCrA*mC*mC*mA | |
| 83 | CUGAGCCCCUACUCCUACUCUAC | 164 | mC*mU*mG*rArGrCrCrCrCrUrArCrUrCrCrUr | 237 |
| | UAUGGCCGUCGUCACCAAUCCCA | | ArCrUrCrUrArCrUrArUrGrGrCrCrGrUrCrGr | |
| | AGGAAUGAGGGAGAAAUCCGUCU | | UrCrArCrCrArArUrCrCrCrArArGrGrArArUr | |
| | UUCAUUGACGGCACCACGGCUGU | | GrArGrGrGrArGrArArArUrCrCrGrUrCrUrUr | |
| | CGUCACCA | | UrCrArUrUrGrArCrGrGrCrArCrCrArCrGrGr | |
| | | | CrUrGrUrCrGrUrCrA*mC*mC*mA | |
| 84 | CUGAGCCCCUACUCCUACUCUAC | 165 | mC*mU*mG*rArGrCrCrCrCrUrArCrUrCrCrUr | |
| | UAUGGCCGUCGUCACCAAUCCCA | | ArCrUrCrUrArCrUrArUrGrGrCrCrGrUrCrGr | |
| | AGGAAUGAGGGACUUCUCAGAAA | | UrCrArCrCrArArUrCrCrCrArArGrGrArArUr | |
| | UCCGUCUUUCAUUGACGGCACCA | | GrArGrGrGrArCrUrUrCrUrCrArGrArArArUr | |
| | CGGCUGUCGUCACCA | | CrCrGrUrCrUrUrUrCrArUrUrGrArCrGrGrCr | |
| | | | ArCrCrArCrGrGrCrUrGrUrCrGrUrCrA*mC*m | |
| | | | C*mA | |

M: 2′O-methyl nucleotide
*phosphorothioate bond between nucleotides

Approximately 16 hours prior to transfection, 25,000 HEK293T cells in DMEM/10% FBS+Pen/Strep were plated into each well of a 96-well plate. On the day of transfection, the cells were 70-90% confluent. For each well to be transfected, a mixture of TransIT-X2® (Mirus Bio) and Opti-MEM™ media (Thermo Fisher) was prepared and then incubated at room temperature for 5-20 minutes (Solution 1). After incubation, the TransIT-X2® (Mirus Bio): OptiMEM™ mixture was added to a separate mixture containing variant Cas12i2-RT fusion working solution, synthetic RNA working solution and OptiMEM™ media (Solution 2). The Solution 1 and Solution 2 mixtures were mixed by pipetting up and down and then incubated at room temperature for 25 minutes. Following incubation, Solution 1 and Solution 2 mixture were added dropwise to each well of a 96 well plate containing the cells. A mixture of 100 ng of the variant Cas12i2 or variant Cas12i2-RT plasmid DNA and 9 pmol of synthesized editing template RNA (IDT) was added to cells. 72 hours post transfection, cells were trypsinized by adding TrypLE™ (Thermo Fisher) to the center of each well and incubated for approximately 5 minutes. Growth media was then added to each well and mixed to resuspend cells. The cells were then spun down at 400 g for 10 minutes, and the supernatant was discarded. QuickExtract™ buffer (Lucigen) was added to ⅕ the amount of the original cell suspension volume. Cells were incubated at 65 °C for 15 minutes, 68 °C for 15 minutes, and 98 °C for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

As shown in FIG. 3, the Cas12i2-RT fusion in the presence of editing template RNA sequences were capable of introducing the encoded substitutions at the TTR locus. Percentage of NGS reads is shown on the y-axis, total edits are shown as in grey bars, and encoded edits are shown as black bars. The data shown is an average of two biological replicates, each of which had three technical replicates. Precise editing efficiency for editing template RNAs that contained PS-2′OMe modifications on the first three and penultimate three nucleotides ranged from 2-8%, with shorter PBS lengths and shorter RTT lengths demonstrating higher precise editing than longer lengths (FIG. 3). Precise editing increased up to 13% with editing template RNAs that contained PS-2′OMe modifications on the first three and last three nucleotides, with similar patterns observed for RTT and PBS lengths (FIG. 4). Precise editing was more efficient for editing template RNAs containing SNPs at positions (relative to the 5′ PAM) 6-10-13-16-19 and 4-6-10-13-16-19 (FIG. 4). See SEQ ID NO: 351 for the indicated SNPs.

The percentages of average indel and the percentages of average precise edits (RT) of exemplary editing template RNAs are provided in Table 18 below.

TABLE 18
Editing Efficiencies of Exemplary Editing Template
RNAs with Cas12i2-RT Fusion Polypeptide

| Editing Template | % Average | % Average |
|---|---|---|
| RNAS | Indel | RT |
| 1 | 41.11425102 | 6.494795332 |
| 2 | 34.80770577 | 4.76937634 |
| 3 | 17.15532862 | 0.276128579 |
| 4 | 40.5923614 | 3.898602006 |
| 5 | 0.624954217 | 0.000333333 |
| 6 | 22.81144586 | 0.333624226 |
| 7 | 40.7270568 | 3.193685248 |
| 8 | 49.29980007 | 2.03534 |
| 9 | 21.45778527 | 0.110743046 |
| 10 | 40.78148514 | 4.61822948 |
| 11 | 45.94384134 | 1.343786436 |
| 12 | 22.79376419 | 0.141407856 |
| 13 | 37.39486575 | 9.948985367 |
| 14 | 50.37264393 | 6.304987388 |
| 15 | 15.08066667 | 0.58 |
| 16 | 44.21160003 | 2.958379531 |
| 17 | 46.60173588 | 1.359570084 |
| 18 | 29.52390517 | 0.180732611 |
| 19 | 43.76912563 | 9.936445027 |
| 20 | 49.4707616 | 4.701003438 |
| 21 | 28.1526213 | 1.04652889 |
| 22 | 49.93085323 | 5.538858481 |
| 23 | 56.48624081 | 2.283111191 |
| 24 | 31.43672226 | 0.272416153 |
| 54 | 53.985699 | 3.023943642 |
| 55 | 45.92466788 | 9.28407334 |
| 56 | 53.1543493 | 2.617138948 |
| 57 | 55.78698163 | 5.058803477 |
| 58 | 46.66083495 | 10.70199875 |
| 59 | 51.18099057 | 6.95560716 |
| 60 | 43.38101463 | 13.1224373 |
| 61 | 46.50273088 | 1.042229408 |
| 62 | 53.0876189 | 4.565422337 |
| 63 | 52.9442106 | 4.566140337 |
| 64 | 49.704 | 12.96833333 |
| 65 | 60.95203823 | 0.462587823 |
| 66 | 56.61880018 | 0.987119597 |
| 67 | 55.94077167 | 2.11623653 |
| 68 | 49.87163475 | 7.614328538 |
| 69 | 52.7630732 | 0.28361387 |
| 70 | 57.06416198 | 1.09238128 |
| 71 | 0.442666667 | 0.034333333 |
| 72 | 50.39904928 | 7.68119229 |
| 73 | 60.93224207 | 0.365695132 |
| 74 | 58.71624758 | 1.15652336 |
| 75 | 56.94960553 | 2.165707305 |
| 76 | 54.02466247 | 7.876604473 |
| 77 | 51.9880839 | 0.401941945 |
| 78 | 58.103216 | 1.48897173 |
| 79 | 48.568949 | 1.74493719 |
| 80 | 48.69227598 | 7.966633592 |
| 81 | 58.78333507 | 0.230750637 |
| 82 | 58.18099127 | 1.416507507 |
| 83 | 55.97514395 | 1.380739295 |
| 84 | 50.4262951 | 5.67587556 |

Overall, this Example shows that the T119M edit was incorporated into the human TTR genomic locus using the exemplary editing template RNAs and an exemplary Cas12i2-RT fusion in HEK293T cells.

### Example 2: RNA-Templated Editing of the TTR 2Ene in HEK293T Cells Using Chemically Modified Editing Template RNAs and mRNA Delivery of Cas12i2 Nuclease-RT Fusion

This Example describes editing of the human TTR gene with an mRNA-encoded Cas12i2-RT fusion protein and editing template RNAs containing terminal phosphorothioate backbone linkages and 2′O-methyl nucleotides.

The variant Cas12i2 of SEQ ID NO: 352 and a variant Cas12i2 of SEQ ID NO: 14 fused with mutant MMLV reverse transcriptase (RT) were individually cloned into in vitro transcription (IVT) backbones and transcribed into mRNA using an in vitro reverse transcription kit. The following editing template RNA sequences were tested. The tested variant Cas1212-MMLV fusion polypeptides, as ell as editing template RNAs, are provided in Table 12 below.

TABLE 12
Exemplary Variant Cas1212-MMLV Fusion Polypeptides and Editing
Template RNAs and Control Guide Sequences

| | | | SEQ |
|---|---|---|---|
| | Shorthand | | ID |
| Description | Description | Editing Template RNA sequence | NO |
| Cas12i2 variant | C_MMLV | See Example 1 above | 59 |
| (H485A/ | Cas 12i2 | | |
| D581R/ | variant 1 | | |
| I926R/V1030G | | | |
| )-NLS- | | | |
| 2xSGGS- | | | |
| XTEN (16 aa)- | | | |
| 2xSGGS- | | | |
| mutMMLVRT | | | |
| -NLS | | | |
| Cas1212 variant | C_MMLV_C | MSSAIKSYKSVLRPNERKNQLLKSTIQCLEDGSAFFFKMLQ | 353 |
| (H485A/D581 | as 1212 | GLFGGITPEIVRESTEQEKQQQDIALWCAVNWFRPVSQDSL | |
| R/G624R/ | variant 2 | THTIASDNLVEKFEEYYGGTASDAIKQYFSASIGESYYWND | |
| F626R/P868T/ | | CRQQYYDLCRELGVEVSDLTHDLEILCREKCLAVATESNQN | |
| I926R/V1030G | | NSIISVLFGTGEKEDRSVKLRITKKILEAISNLKEIPKNVA | |
| /E1035R/S104 | | PIQEIILNVAKATKETFRQVYAGNLGAPSTLEKFIAKDGQK | |
| 6G)-NLS- | | EFDLKKLQTDLKKVIRGKSKERDWCCQEELRSYVEQNTIQY | |
| 2xSGGS- | | DLWAWGEMENKAHTALKIKSTRNYNFAKQRLEQFKEIQSLN | |
| XTEN (16 aa)- | | NLLVVKKLNDFFDSEFFSGEETYTICVHHLGGKDLSKLYKA | |
| 2xSGGS- | | WEDDPADPENAIVVLCDDLKNNFKKEPIRNILRYIFTIRQE | |
| mutMMLVRT | | CSAQDILAAAKYNQQLDRYKSQKANPSVLGNQGFTWTNAVI | |
| -NLS | | LPEKAQRNDRPNSLDLRIWLYLKLRHPDGRWKKAHIPFYDT | |
| | | RFFQEIYAAGNSPVDTCQFRTPRFGYHLPKLTDQTAIRVNK | |
| | | KHVKAAKTEARIRLAIQQGTLPVSNLKITEISATINSKGQV | |
| | | RIPVKFRVGRQKGTLQIGDRFCGYDQNQTASHAYSLWEVVK | |
| | | EGQYHKELRCRVRFISSGDIVSITENRGNQFDQLSYEGLAY | |
| | | PQYADWRKKASKFVSLWQITKKNKKKEIVTVEAKEKFDAIC | |
| | | KYQPRLYKENKEYAYLLRDIVRGKSLVELQQIRQEIFRFIE | |
| | | QDCGVTRLGSLSLSTLETVKAVKGIIYSYFSTALNASKNNP | |
| | | ISDEQRKEFDPELFALLEKLELIRTRKKKQKVERIANSLIQ | |
| | | TCLENNIKFIRGEGDLSTTNNATKKKANSRSMDWLARGVEN | |
| | | KIRQLATMHNITLFGCGSLYTSHQDPLVHRNPDKAMKCRWA | |
| | | AIPVKDIGDWVLRKLSQNLRAKNRGTGEYYHQGVKEFLSHY | |
| | | ELQDLEEELLKWRSDRKSNIPCWVLQNRLAEKLGNKEAVVY | |
| | | IPVRGGRIYFATHKVATGAVSIVFDQKQVWVCNADHVAAAN | |
| | | IALTGKGIGRQSSDEENPDGGRIKLQLTSKRPAATKKAGQA | |
| | | KKKKSGGSSGGSSGSETPGTSESATPESSGGSSGGSTLNIE | |
| | | DEYRLHETSKEPDVSLGSTWLSDFPQAWAETGGMGLAVRQA | |
| | | PLIIPLKATSTPVSIKQYPMSQEARLGIKPHIQRLLDQGIL | |
| | | VPCQSPWNTPLLPVKKPGTNDYRPVQDLREVNKRVEDIHPT | |
| | | VPNPYNLLSGLPPSHQWYTVLDLKDAFFCLRLHPTSQPLFA | |
| | | FEWRDPEMGISGQLTWTRLPQGFKNSPTLFNEALHRDLADF | |
| | | RIQHPDLILLQYVDDLLLAATSELDCQQGTRALLQTLGNLG | |
| | | YRASAKKAQICQKQVKYLGYLLKEGQRWLTEARKETVMGQP | |
| | | TPKTPRQLREFLGKAGFCRLFIPGFAEMAAPLYPLTKPGTL | |
| | | FNWGPDQQKAYQEIKQALLTAPALGLPDLTKPFELFVDEKQ | |
| | | GYAKGVLTQKLGPWRRPVAYLSKKLDPVAAGWPPCLRMVAA | |
| | | IAVLTKDAGKLTMGQPLVILAPHAVEALVKQPPDRWLSNAR | |
| | | MTHYQALLLDTDRVQFGPVVALNPATLLPLPEEGLQHNCLD | |
| | | ILAEAHGTRPDLTDQPLPDADHTWYTDGSSLLQEGQRKAGA | |
| | | AVTTETEVIWAKALPAGTSAQRAELIALTQALKMAEGKKLN | |
| | | VYTDSRYAFATAHIHGEIYRRRGWLTSEGKEIKNKDEILAL | |
| | | LKALFLPKRLSIIHCPGHQKGHSAEARGNRMADQAARKAAI | |
| | | TETPDTSTLLIENSSPMKRTADGSEFESPKKKRKV | |
| Editing | | mC*mU*mG*rArGrCrCrCrCrUrArCrUrCrCrUrArUrU | 214 |
| Template | | rCrCrArCrCrArUrGrGrCrCrGrUrUrGrUrUrArCrUr | |
| RNA 60 | | ArArUrCrCrCrArArGrGrArArUrGrArGrGrGrArCrU | |
| | | rUrCrUrCrArGrArArArUrCrCrGrUrCrUrUrUrCrAr | |
| | | UrUrGrArCrGrGrCrArCrCrArCrGrGrCrUrGrUrCrG | |
| | | rUrCrA*mC*mC*mA | |
| Editing | | mC*mU*mG*rArGrCrCrCrCrUrArCrUrCrCrUrArUrU | 218 |
| Template | | rCrCrArCrUrArUrGrGrCrCrGrUrUrGrUrUrArCrUr | |
| RNA 64 | | ArArUrCrCrCrArArGrGrArArUrGrArGrGrGrArCrU | |
| | | rUrCrUrCrArGrArArArUrCrCrGrUrCrUrUrUrCrAr | |
| | | UrUrGrArCrGrGrCrArCrCrArCrGrGrCrUrGrUrCrG | |
| | | rUrCrA*mC*mC*mA | |
| Guide control | | mA*mG*mA*mAmAmUmCmCmGmUmCmUmUmUmCmAmUmUmG | 354 |
| | | mAmCmGmGmCmAmCmCmAmCmGmGmCmUmGmUmCmGmUmCm | |
| | | A*mC*mC*mA | |

HEK293T cells were harvested using TrypLE and counted. Cells were washed once with PBS and resuspended in SF buffer+supplement (Lonza #V4XC-2032)+transfection enhancer oligo (final concentration 4 μM) at a concentration of 16,480 cells/μL. Resuspended cells were dispensed at 3e5 cells/reaction into Lonza 16-well nucleocuvette strips. 1 μL of Cas12i2-RT mRNA (1 mg/mL) was mixed with 1 μL of an editing template RNA (1 mM) and added to each reaction.

The strips were electroporated using an electroporation device (program CM-130, Lonza 4D-nucleofector). Immediately following electroporation, 80 μL of pre-warmed DMEM±10% FBS was added to each well and mixed gently by pipetting. For each technical replicate plate, plated 10 μL (30,000 cells) of diluted nucleofected cells into pre-warmed 96-well plate with wells containing 100 μL DMEM+10% FBS. Editing plates were incubated for 3 days at 37 °C with 5% CO₂.

After 3 days, wells were harvested using TrypLE and transferred to 96-well Twin tec PCR plates. Media was flicked off and cells were resuspended in 20 μL DNA extraction buffer (QuickExtract®). Samples were then cycled in a PCR instrument at 65 °C for 15 min, 68 °C for 15 min, 98 °C for 10 min. Samples were then frozen at −20 °C. Samples for NGS were prepared as described in Example 1.

As shown in FIG. 5, the Cas12i2-RT fusion in the presence of editing template RNA sequences were capable of introducing the encoded substitutions at the TTR locus. Percentage of NGS reads is shown on the y-axis, total edits are shown as in grey bars, and encoded edits are shown as black bars. The data shown is an average of three technical replicates across one bioreplicate. Precise editing efficiency for the editing template RNAs that contained PS-2′OMe modifications ranged from 3-13% (FIG. 5).

This Example shows that specific edits were incorporated into the human TTR genomic locus using editing template RNAs and a variant Cas12i2-RT fusion polypeptide in HEK293T cells.

### Example 3—RNA-Templated Editing of the TTR Gene in Induced Pluripotent Stem Cells (iPSCs) Using Chemically Modified Editing Template RNAs and mRNA Delivery of Cas12i2 Nuclease-RT Fusion Effector

This Example describes editing of the human TTR gene in iPSCs using the mRNA-encoded Cas12i2-RT fusion protein of SEQ ID NO: 59 and editing template RNAs of SEQ ID NOs: 214 and 218.

Prior to electroporation, undifferentiated iPSCs were grown on tissue culture-treated plates coated with Matrigel hESC qualified matrix (Corning 354277) in mTeSR Plus media (Stemcell Technologies 100-0276). Cells were passaged using ReLeSR (Stemcell Technologies 05872) or Gentle Cell Dissociation Reagent (Stemcell Technologies 100-0485) per the manufacturer's instructions.

The day of electroporation, iPSCs were harvested and counted. Cells were washed once with PBS and resuspended in P3 buffer+supplement (Lonza #V4XP-3032) at a concentration of 16,667 cells/μL. Resuspended cells were dispensed at 300,000 cells/reaction into Lonza 16-well nucleocuvette strips. Cas12i2-RT mRNA (1 mg/mL) was mixed with editing template RNA (1 mM) at a 1:1 ratio and added to each reaction.

The strips were electroporated using an electroporation device (program CA-137, Lonza 4D-nucleofector). Immediately following electroporation, 20 μL of cells/cargo mixture were transferred into one well each in 24-well Matrigel-coated plate with 500 μL Single-Cell Plating Medium (CloneR diluted in mTeSR Plus at 1:10 volume dilution). Editing plates were incubated for 3 days at 37 °C with 5% CO₂, with daily media changes of mTeSR Plus.

After 3 days, cells were harvested using GCDR, transferred to 96-well plates, spun down at 400× g for 10 min, then resuspended in DNA extraction buffer (QuickExtract). Samples were then cycled in a PCR instrument at 65 °C for 15 min, 68 °C for 15 min, 98 °C for 10 min. Samples were then frozen at −20 °C. Samples for NGS were prepared as described in Example 1.

FIG. 6 shows indels (light and medium grey bars) and precise edits (black bars) induced by the Cas12i2-RT fusion and editing template RNAs in iPSCs. As shown in FIG. 6, the Cas12i2-RT fusion of SEQ ID NO: 59 was capable of introducing precise edits encoded by the editing template RNA sequences of SEQ ID NO: 214 and SEQ ID NO: 218 into the TTR gene in iPSCs. Precise edits induced by the editing template RNA of SEQ ID NO: 214 exceeded those by the editing template RNA of SEQ ID NO: 218. Precise edits were not incorporated into the TTR gene by mRNA encoding the variant Cas12i2 of SEQ ID NO: 11, which was used as a control.

Therefore, this Example shows that the T119M edit was incorporated into the human TTR genomic locus using the editing template RNAs of SEQ ID NO: 214 and SEQ ID NO: 218 in iPSCs.

### Example 4—RNA-Templated Editing of the TTR Gene in Activated Primary T Cells Using Chemically Modified Editing Template RNAs and mRNA Delivery of Cas12i2 Nuclease-RT Fusion Effector

This Example describes editing of the human TTR gene in T cells using the mRNA-encoded Cas12i2-RT fusion protein of SEQ ID NO: 59 and editing template RNAs of SEQ ID NOs: 214 and 218.

Frozen human Peripheral Blood Mononuclear Cells (PBMCs) (StemCell Technologies; #70025) from a donor were revived and counted using an automated cell counter. T cells were isolated from PBMCs using the EasySep Human T Cell Isolation Kit (StemCell Technologies: #17951). Following isolation, a sample was collected and stained for CD3ε for flow cytometry analysis of surface expression to determine T cell purity of the isolated cells. Cell density was adjusted to 1,000,000 cells/mL, and cells were stimulated for 3 days with a cocktail of anti-CD3:CD28 antibodies. Cells were cultured in fresh complete ImmunoCult-XF Cell Expansion Medium (StemCell Technologies: #10981) with 10 ng/mL IL-2 and 2 mM L-Glutamine and supplemented with 25 μL/mL of ImmunoCult Human CD3/CD28 T Cell Activator (StemCell Technologies; #10971).

The day of electroporation, T cells were harvested and counted. A sample of cells was collected and stained for CD25 for flow cytometry analysis to determine activation efficiency. Cells were washed once with PBS and resuspended in P3 buffer+supplement (Lonza #V4XP-3032) at a concentration of 11,111 cells/μL. Resuspended cells were dispensed at 200,000 cells/reaction into Lonza 16-well nucleocuvette strips. Cas12i2-RT mRNA (1 mg/mL) was mixed with editing template RNA (1 mM) at a 1:1 ratio and added to each reaction.

The strips were electroporated using an electroporation device (program EO-115, Lonza 4D-nucleofector). Immediately following electroporation, pre-warmed ImmunoCult-XF+IL-2+L-Glutamine was added to cells and mixed gently by pipetting. For each technical replicate plate, ~50,000 diluted nucleofected cells were plated into the pre-warmed 96-well plate with wells containing ImmunoCult-XF+IL-2+L-Glutamine. Editing plates were incubated for 3 days at 37 °C with 5% CO₂.

After 3 days, wells were mixed by pipetting, transferred to 96-well plates, spun down at 400× g for 10 min, and resuspended in DNA extraction buffer (QuickExtract). Samples were then cycled in a PCR instrument at 65 °C for 15 min, 68 °C for 15 min, 98 °C for 10 min. Samples were then frozen at −20 °C. Samples for NGS were prepared as described in Example 1.

FIG. 7 shows indels (light and medium grey bars) and precise edits (black bars) induced by the Cas12i2-RT fusion and editing template RNAs in T cells. As shown in FIG. 7, the Cas12i2-RT fusion of SEQ ID NO: 59 was capable of introducing precise edits encoded by the editing template RNA sequences of SEQ ID NO: 214 and SEQ ID NO: 218 into the TTR gene in T cells. Precise edits were not incorporated into the TTR gene by mRNA encoding the variant Cas12i2 of SEQ ID NO: 11, which was used as a control.

Therefore, this Example shows that the T119M edit was incorporated into the human TTR genomic locus using the editing template RNAs of SEQ ID NO: 214 and SEQ ID NO: 218 in T cells.

### Example 5: RNA-Templated Editing of the TTR Gene in HEK293T Cells Using Chemically Modified Editing Template RNAs and DNA Delivery of Variant Cas12i4-RT Fusion Polypeptide

This Example describes editing of the human TTR gene with a plasmid-encoded variant Cas12i4-RT fusion polypeptide and editing template RNAs containing terminal phosphorothioate backbone linkages and 2′O-methyl nucleotides.

Variant Cas12i4 (SEQ ID NO: 16) and a fusion polypeptide of the variant Cas12i4 and mutant MMLV reverse transcriptase (RT) was cloned into the pcda3.1 backbone tO (Invitrogen). Configuration of the C-terminal RT fusion to the variant Cas12i4 is shown in Table 13. Working solutions of plasmids for expression of the variant Cas12i4 and the variant Cas12i4-RT fusion polypeptide were prepared in water.

TABLE 13
Cas12i4-RT Fusion Polypeptide Exemplary Design and Sequence

| Full | Shorthand | | SEQ |
|---|---|---|---|
| Description | Description | Sequence | ID NO |
| Cas1214 | C MMLV | MASISRPYGTKLRPDARKKEMLDKFFNTLTKGQRVFADLALC | 238 |
| variant-NLS | Cas 12i4 | IYGSLTLEMAKSLEPESDSELVCAIGWFRLVDKTIWSKDGIK | |
| -2xSGGS- | variant | QENLVKQYEAYSGKEASEVVKTYLNSPSSDKYVWIDCRQKFL | |
| XTEN (16 aa) | | RFQRELGTRNLSEDFECMLFEQYIRLTKGEIEGYAAISNMFG | |
| -2xSGGS- | | NGEKEDRSKKRMYATRMKDWLEANENITWEQYREALKNQLNA | |
| mutMMLVR | | KNLEQVVANYKGNAGGADPFFKYSFSKEGMVSKKEHAQQLDK | |
| T-NLS | | FKTVLKNKARDLNFPNKEKLKQYLEAEIGIPVDANVYSQMFS | |
| | | NGVSEVQPKTTRNMSFSNEKLDLLTELKDLNKGDGFEYAREV | |
| | | LNGFFDSELHTTEDKFNITSRYLGGDKSNRLSKLYKIWKKEG | |
| | | VDCEEGIQQFCEAVKDKMGQIPIRNVLKYLWQFRETVSAEDF | |
| | | EAAAKANHLEEKISRVKAHPIVISNRYWAFGTSALVGNIMPA | |
| | | DKRHQGEYAGQNFKMWLRAELHYDGKKAKHHLPFYNARFFEE | |
| | | VYCYHPSVAEITPFKTKQFGCEIGKDIPDYVSVALKDNPYKK | |
| | | ATKRILRAIYNPVANTTRVDKTTNCSFMIKRENDEYKLVINR | |
| | | KISRDRPKRIEVGRTIMGYDRNQTASDTYWIGRLVPPGTRGA | |
| | | YRIGEWSVQYIKSGPVLSSTQGVNNSTTDQLVYNGMPSSSER | |
| | | FKAWKKARMAFIRKLIRQLNDEGLESKGQDYIPENPSSFDVR | |
| | | GETLYVFNSNYLKALVSKHRKAKKPVEGILDEIEAWTSKDKD | |
| | | SCSLMRLSSLSDASMQGIASLKSLINSYFNKNGCKTIEDKEK | |
| | | FNPVLYAKLVEVEQRRTNKRSEKVGRIAGSLEQLALLNGVEV | |
| | | VIGEADLGEVEKGKSKKQNSRNMDWCAKQVAQRLEYKLAFHG | |
| | | IGYFGVNPMYTSHQDPFEHRRVADHIVMRARFEEVNVENIAE | |
| | | WHVRNFSNYLRADSGTGLYYKQATMDFLKHYGLEEHAEGLEN | |
| | | KKIKFYDFRKILEDKNLTSVIIPKRGGRIYMATNPVTSDSTP | |
| | | ITYAGKTYNRCNADEVAAANIVISVLAPRSKKNREQDDIPLI | |
| | | TKKAESKSPPKDRKRSKTSQLPQKKRPAATKKAGQAKKKKSG | |
| | | GSSGGSSGSETPGTSESATPESSGGSSG | |
| | | GSTLNIEDEYRLHETSKEPDVSLGSTWLSDFPQAWAETGGMG | |
| | | LAVRQAPLIIPLKATSTPVSIKQYPMSQEARLGIKPHIQRLL | |
| | | DQGILVPCQSPWNTPLLPVKKPGTNDYRPVQDLREVNKRVED | |
| | | IHPTVPNPYNLLSGLPPSHQWYTVLDLKDAFFCLRLHPTSQP | |
| | | LFAFEWRDPEMGISGQLTWTRLPQGFKNSPTLFNEALHRDLA | |
| | | DFRIQHPDLILLQYVDDLLLAATSELDCQQGTRALLQTLGNL | |
| | | GYRASAKKAQICQKQVKYLGYLLKEGQRWLTEARKETVMGQP | |
| | | TPKTPRQLREFLGKAGFCRLFIPGFAEMAAPLYPLTKPGTLE | |
| | | NWGPDQQKAYQEIKQALLTAPALGLPDLTKPFELFVDEKQGY | |
| | | AKGVLTQKLGPWRRPVAYLSKKLDPVAAGWPPCLRMVAAIAV | |
| | | LTKDAGKLTMGQPLVILAPHAVEALVKQPPDRWLSNARMTHY | |
| | | QALLLDTDRVQFGPVVALNPATLLPLPEEGLQHNCLDILAEA | |
| | | HGTRPDLTDQPLPDADHTWYTDGSSLLQEGQRKAGAAVTTET | |
| | | EVIWAKALPAGTSAQRAELIALTQALKMAEGKKLNVYTDSRY | |
| | | AFATAHIHGEIYRRRGWLTSEGKEIKNKDEILALLKALFLPK | |
| | | RLSIIHCPGHQKGHSAEARGNRMADQAARKAAITETPDTSTL | |
| | | LIENSSPMKRTADGSEFESPKKKRKV | |

Configurations of the tested editing template RNAs are shown in Table 7. The editing template RNA sequences were synthesized by IDT. A reverse transcription template (RTT) sequence and primer binding site (PBS) were fused to the 5′ end of an RNA guide (e.g., the 5′ end of a direct repeat sequence and a spacer sequence). The RTT sequence was designed to introduce a specific substitution to change the amino acid at position 119 from Threonine to Methionine (T119M) in the human TTR gene, as well as various silent mutations nearby to enhance the editing efficiency and prevent re-targeting of the edited genomic site. The sequences of the editing template RNAs are shown in Table 8. In Table 8, the PBS length, the RTT length of the donor RNA and the positions of substitutions relative to PAM sequence are indicated. A working solution of synthetic RNA composed of each editing template RNA was prepared in water.

TABLE 14
Editing Template RNA Exemplary Designs

| Configuration | Description |
|---|---|
| RTT sequence - PBS - DR - spacer | RTT sequence: 44 nucleotides |
| | PBS: 20 nucleotides |

Tables 15-17 provide information of exemplary editing template RNAs for use in editing TTR via the Cas12i4-RT fusion polypeptide disclosed herein.

TABLE 15
Targets of Exemplary Editing Template RNAs for Cas12i4/RT-
Mediated TTR Editing

| | | | Posi- | | Total | | |
|---|---|---|---|---|---|---|---|
| | | | tion | | number | | |
| Editing | | | of SNP | Position | of | | SEQ |
| Template | | Target | within | of silent | mis- | | ID |
| RNA | PAM | Strand* | target | mutations | matches | Target Sequence | NO |
| 85 | TA | S | 15 | 10, 13, 19 | 4 | CTCCTATTCCACCACGGCTG | 240 |
| 86 | TA | S | 15 | 8, 9, 10, | 6 | CTCCTATTCCACCACGGCTG | 240 |
| | | | | 13, 19 | | | |
| 87 | TA | S | 15 | 2, 3, 4, 8, 9, | 9 | CTCCTATTCCACCACGGCTG | 240 |
| | | | | 10, 13, 19 | | | |
| 88 | TC | S | 12 | 10.16, 19 | 4 | CTATTCCACCACGGCTGTCG | 241 |
| 89 | TC | S | 12 | 7, 10, 16, 19 | 5 | CTATTCCACCACGGCTGTCG | 241 |
| 90 | TC | S | 12 | 5, 6, 7, 10, | 7 | CTATTCCACCACGGCTGTCG | 241 |
| | | | | 16, 19 | | | |
| 91 | TA | S | 9 | 13, 16, 19 | 4 | TTCCACCACGGCTGTCGTCA | 242 |
| 92 | TA | S | 9 | 7, 13, 16, 19 | 5 | TTCCACCACGGCTGTCGTCA | 242 |
| 93 | TA | S | 9 | 4, 7, 13, 16, 19 | 6 | TTCCACCACGGCTGTCGTCA | 242 |
| 94 | TT | S | 7 | 14, 17, 20 | 4 | CCACCACGGCTGTCGTCACC | 243 |
| 95 | TT | S | 7 | 11.14, 17, 20 | 5 | CCACCACGGCTGTCGTCACC | 243 |
| 96 | TT | S | 7 | 5, 11, 14, | 6 | CCACCACGGCTGTCGTCACC | 243 |
| | | | | 17, 20 | | | |
| 97 | TC | S | 6 | 13, 16, 19 | 4 | CACCACGGCTGTCGTCACCA | 60 |
| 98 | TC | S | 6 | 10, 13, 16, 19 | 5 | CACCACGGCTGTCGTCACCA | 60 |
| 99 | TC | S | 6 | 4, 10, 13, | 6 | CACCACGGCTGTCGTCACCA | 60 |
| | | | | 16, 19 | | | |
| 100 | TT | AS | 20 | 10.13, 16 | 4 | GGGATTGGTGACGACAGCCG | 244 |
| 101 | TT | AS | 20 | 7, 10, 13, 16 | 5 | GGGATTGGTGACGACAGCCG | 244 |
| 102 | TT | AS | 20 | 4, 7, 10, | 6 | GGGATTGGTGACGACAGCCG | 244 |
| | | | | 13, 16 | | | |
| 103 | TG | AS | 19 | 9, 12, 15 | 4 | GGATTGGTGACGACAGCCGT | 61 |
| 104 | TG | AS | 19 | 6, 9, 12, 15 | 5 | GGATTGGTGACGACAGCCGT | 61 |
| 105 | TG | AS | 19 | 3, 6, 9, | 6 | GGATTGGTGACGACAGCCGT | 61 |
| | | | | 12, 15 | | | |
| 106 | TT | AS | 14 | 10, 16, 19 | 4 | GGTGACGACAGCCGTGGTGG | 245 |
| 107 | TT | AS | 14 | 7, 10, 16, 19 | 5 | GGTGACGACAGCCGTGGTGG | 245 |
| 108 | TT | AS | 14 | 4, 7, 10, | 6 | GGTGACGACAGCCGTGGTGG | 245 |
| | | | | 16, 19 | | | |
| 109 | TG | AS | 13 | 6, 9, 15, 18 | 5 | GTGACGACAGCCGTGGTGGA | 246 |
| 110 | TG | AS | 13 | 9, 15, 18, | 6 | GTGACGACAGCCGTGGTGGA | 246 |
| | | | | 19, 20 | | | |
| 111 | TG | AS | 13 | 3, 6, 9, 15, | 8 | GTGACGACAGCCGTGGTGGA | 246 |
| | | | | 18, 19, 20 | | | |
| 112 | TG | AS | 10 | 6, 12, 15 | 4 | ACGACAGCCGTGGTGGAATA | 247 |
| 113 | TG | AS | 10 | 6, 12, 15, | 6 | ACGACAGCCGTGGTGGAATA | 247 |
| | | | | 16, 17 | | | |
| 114 | TG | AS | 10 | 3, 6, 12, 15, | 7 | ACGACAGCCGTGGTGGAATA | 247 |
| | | | | 16, 17 | | | |

Target strand refers to the strand where the target sequence locates.
S: sense strand;
AS: antisense strand.

TABLE 16
Sequences of Functional Elements in Exemplary Cas12i4 Editing
Template RNAs

| Editing | | | | |
|---|---|---|---|---|
| Template | | | | |
| RNAS | Spacer | DR | RT Template | PBS |
| 85 | CUCCUAUUCCACCA | AGACAUGUGUCCUCA | GCCGCCCUGCUGAGCCCCUACUC | CACCAAUCCCAAGGA |
| | CGGCUG (SEQ ID | GUGACAC (SEQ ID | CUAUUCUACUAUGGCCGUCGU | AUGAG (SEQ ID |
| | NO: 248) | NO: 58) | (SEQ ID NO: 256) | NO: 283) |
| 86 | CUCCUAUUCCACCA | AGACAUGUGUCCUCA | GCCGCCCUGCUGAGCCCCUACUC | CACCAAUCCCAAGGA |
| | CGGCUG (SEQ ID | GUGACAC (SEQ ID | CUAUAGUACUAUGGCCGUCGU | AUGAG (SEQ ID |
| | NO: 248) | NO: 58) | (SEQ ID NO: 257) | NO: 283) |
| 87 | CUCCUAUUCCACCA | AGACAUGUGUCCUCA | GCCGCCCUGCUGAGCCCCUACAG | CACCAAUCCCAAGGA |
| | CGGCUG (SEQ ID | GUGACAC (SEQ ID | UUAUAGUACUAUGGCCGUCGU | AUGAG (SEQ ID |
| | NO: 248) | NO: 58) | (SEQ ID NO: 258) | NO: 283) |
| 88 | CUAUUCCACCACGG | AGACAUGUGUCCUCA | GCCCUGCUGAGCCCCUACUCCUA | CAAUCCCAAGGAAUG |
| | CUGUCG (SEQ ID | GUGACAC (SEQ ID | UUCCACUAUGGCCGUUGUCAC | AGGGA (SEQ ID |
| | NO: 249) | NO: 58) | (SEQ ID NO: 259) | NO: 284) |
| 89 | CUAUUCCACCACGG | AGACAUGUGUCCUCA | GCCCUGCUGAGCCCCUACUCCUA | CAAUCCCAAGGAAUG |
| | CUGUCG (SEQ ID | GUGACAC (SEQ ID | UUCUACUAUGGCCGUUGUCAC | AGGGA (SEQ ID |
| | NO: 249) | NO: 58) | (SEQ ID NO: 260) | NO: 284) |
| 90 | CUAUUCCACCACGG | AGACAUGUGUCCUCA | GCCCUGCUGAGCCCCUACUCCUA | CAAUCCCAAGGAAUG |
| | CUGUCG (SEQ ID | GUGACAC (SEQ ID | UAGUACUAUGGCCGUUGUCAC | AGGGA (SEQ ID |
| | NO: 249) | NO: 58) | (SEQ ID NO: 261) | NO: 284) |
| 91 | UUCCACCACGGCUG | AGACAUGUGUCCUCA | CUGCUGAGCCCCUACUCCUAUUC | UCCCAAGGAAUGAGG |
| | UCGUCA (SEQ ID | GUGACAC (SEQ ID | CACCAUGGCCGUUGUUACCAA | GACUU (SEQ ID |
| | NO: 250) | NO: 58) | (SEQ ID NO: 262) | NO: 285) |
| 92 | UUCCACCACGGCUG | AGACAUGUGUCCUCA | CUGCUGAGCCCCUACUCCUAUUC | UCCCAAGGAAUGAGG |
| | UCGUCA (SEQ ID | GUGACAC (SEQ ID | CACUAUGGCCGUUGUUACCAA | GACUU (SEQ ID |
| | NO: 250) | NO: 58) | (SEQ ID NO: 263) | NO: 285) |
| 93 | UUCCACCACGGCUG | AGACAUGUGUCCUCA | UACUAUGGCCGUUGUUACCAA | UCCCAAGGAAUGAGG |
| | UCGUCA (SEQ ID | GUGACAC (SEQ ID | CUGCUGAGCCCCUACUCCUAUUC | GACUU (SEQ ID |
| | NO: 250) | NO: 58) | (SEQ ID NO: 264) | NO: 285) |
| 94 | CCACCACGGCUGUC | AGACAUGUGUCCUCA | GCUGAGCCCCUACUCCUAUUCCA | CCAAGGAAUGAGGGA |
| | GUCACC (SEQ ID | GUGACAC (SEQ ID | CCAUGGCUGUUGUUACUAAUC | CUUCU (SEQ ID |
| | NO: 251) | NO: 58) | (SEQ ID NO: 265) | NO: 286) |
| 95 | CCACCACGGCUGUC | AGACAUGUGUCCUCA | CCAUGGCCGUUGUUACUAAUC | CCAAGGAAUGAGGGA |
| | GUCACC (SEQ ID | GUGACAC (SEQ ID | GCUGAGCCCCUACUCCUAUUCCA | CUUCU (SEQ ID |
| | NO: 251) | NO: 58) | (SEQ ID NO: 266) | NO: 286) |
| 96 | CCACCACGGCUGUC | AGACAUGUGUCCUCA | GCUGAGCCCCUACUCCUAUUCCA | CCAAGGAAUGAGGGA |
| | GUCACC (SEQ ID | GUGACAC (SEQ ID | CUAUGGCCGUUGUUACUAAUC | CUUCU (SEQ ID |
| | NO: 251) | NO: 58) | (SEQ ID NO: 267) | NO: 286) |
| 97 | CACCACGGCUGUCG | AGACAUGUGUCCUCA | CUGAGCCCCUACUCCUAUUCCAC | CAAGGAAUGAGGGAC |
| | UCACCA (SEQ ID | GUGACAC (SEQ ID | CAUGGCUGUUGUUACUAAUCC | UUCUC (SEQ ID |
| | NO : 63) | NO: 58) | (SEQ ID NO: 67) | NO: 91) |
| 98 | CACCACGGCUGUCG | AGACAUGUGUCCUCA | CUGAGCCCCUACUCCUAUUCCAC | CAAGGAAUGAGGGAC |
| | UCACCA (SEQ ID | GUGACAC (SEQ ID | CAUGGCCGUUGUUACUAAUCC | UUCUC (SEQ ID |
| | NO: 63) | NO: 58) | (SEQ ID NO: 70) | NO: 91) |
| 99 | CACCACGGCUGUCG | AGACAUGUGUCCUCA | CUGAGCCCCUACUCCUAUUCCAC | CAAGGAAUGAGGGAC |
| | UCACCA (SEQ ID | GUGACAC (SEQ ID | UAUGGCCGUUGUUACUAAUCC | UUCUC (SEQ ID |
| | NO: 63) | NO: 58) | (SEQ ID NO: 80) | NO: 91) |
| 100 | GGGAUUGGUGACGA | AGACAUGUGUCCUCA | GGAGAAGUCCCUCAUUCCUUGGG | GGAAUAGGAGUAGGG |
| | CAGCCG (SEQ ID | GUGACAC (SEQ ID | AUUGGUAACAACGGCCAUGGU | GCUCA (SEQ ID |
| | NO: 252) | NO: 58) | (SEQ ID NO: 268) | NO: 287) |
| 101 | GGGAUUGGUGACGA | AGACAUGUGUCCUCA | GGAGAAGUCCCUCAUUCCUUGGG | GGAAUAGGAGUAGGG |
| | CAGCCG (SEQ ID | GUGACAC (SEQ ID | AUUAGUAACAACGGCCAUGGU | GCUCA (SEQ ID |
| | NO: 252) | NO: 58) | (SEQ ID NO: 269) | NO: 287) |
| 102 | GGGAUUGGUGACGA | AGACAUGUGUCCUCA | GGAGAAGUCCCUCAUUCCUUGGG | GGAAUAGGAGUAGGG |
| | CAGCCG (SEQ ID | GUGACAC (SEQ ID | GUUAGUAACAACGGCCAUGGU | GCUCA (SEQ ID |
| | NO: 252) | NO: 58) | (SEQ ID NO: 270) | NO: 287) |
| 103 | GGAUUGGUGACGAC | AGACAUGUGUCCUCA | UUGGUAACAACGGCCAUGGUG | GAAUAGGAGUAGGGG |
| | AGCCGU (SEQ ID | GUGACAC (SEQ ID | GAGAAGUCCCUCAUUCCUUGGGA | CUCAG (SEQ ID |
| | NO: 64) | NO: 58) | (SEQ ID NO: 271) | NO: 94) |
| 104 | GGAUUGGUGACGAC | AGACAUGUGUCCUCA | GAGAAGUCCCUCAUUCCUUGGGA | GAAUAGGAGUAGGGG |
| | AGCCGU (SEQ ID | GUGACAC (SEQ ID | UUAGUAACAACGGCCAUGGUG | CUCAG (SEQ ID |
| | NO: 64) | NO: 58) | (SEQ ID NO: 272) | NO: 94) |
| 105 | GGAUUGGUGACGAC | AGACAUGUGUCCUCA | GAGAAGUCCCUCAUUCCUUGGGG | GAAUAGGAGUAGGGG |
| | AGCCGU (SEQ ID | GUGACAC (SEQ ID | UUAGUAACAACGGCCAUGGUG | CUCAG (SEQ ID |
| | NO: 64) | NO: 58) | (SEQ ID NO: 273) | NO: 94) |
| 106 | GGUGACGACAGCCG | AGACAUGUGUCCUCA | GUCCCUCAUUCCUUGGGAUUGGU | GGAGUAGGGGCUCAG |
| | UGGUGG (SEQ ID | GUGACAC (SEQ ID | GACGACGGCCAUAGUAGAAUA | CAGGG (SEQ ID |
| | NO: 253) | NO: 58) | (SEQ ID NO: 274) | NO: 288) |
| 107 | GGUGACGACAGCCG | AGACAUGUGUCCUCA | GUCCCUCAUUCCUUGGGAUUGGU | GGAGUAGGGGCUCAG |
| | UGGUGG (SEQ ID | GUGACAC (SEQ ID | GACAACGGCCAUAGUAGAAUA | CAGGG (SEQ ID |
| | NO: 253) | NO: 58) | (SEQ ID NO: 275) | NO: 288) |
| 108 | GGUGACGACAGCCG | AGACAUGUGUCCUCA | GUCCCUCAUUCCUUGGGAUUGGU | GGAGUAGGGGCUCAG |
| | UGGUGG (SEQ ID | GUGACAC (SEQ ID | AACAACGGCCAUAGUAGAAUA | CAGGG (SEQ ID |
| | NO: 253) | NO: 58) | (SEQ ID NO: 276) | NO: 288) |
| 109 | GUGACGACAGCCGU | AGACAUGUGUCCUCA | UCCCUCAUUCCUUGGGAUUGGUG | GAGUAGGGGCUCAGC |
| | GGUGGA (SEQ ID | GUGACAC (SEQ ID | ACAACGGCCAUAGUAGAAUAG | AGGGC (SEQ ID |
| | NO: 254) | NO: 58) | (SEQ ID NO: 277) | NO: 289) |
| 110 | GUGACGACAGCCGU | AGACAUGUGUCCUCA | UCCCUCAUUCCUUGGGAUUGGUG | GAGUAGGGGCUCAGC |
| | GGUGGA (SEQ ID | GUGACAC (SEQ ID | ACGACGGCCAUAGUACUAUAG | AGGGC (SEQ ID |
| | NO: 254) | NO: 58) | (SEQ ID NO: 278) | NO: 289) |
| 111 | GUGACGACAGCCGU | AGACAUGUGUCCUCA | UCCCUCAUUCCUUGGGAUUGGUA | GAGUAGGGGCUCAGC |
| | GGUGGA (SEQ ID | GUGACAC (SEQ ID | ACAACGGCCAUAGUACUAUAG | AGGGC (SEQ ID |
| | NO: 254) | NO: 58) | (SEQ ID NO: 279) | NO: 289) |
| 112 | ACGACAGCCGUGGU | AGACAUGUGUCCUCA | CUCAUUCCUUGGGAUUGGUGACG | UAGGGGCUCAGCAGG |
| | GGAAUA (SEQ ID | GUGACAC (SEQ ID | ACGGCCAUAGUAGAAUAGGAG | GCGGC (SEQ ID |
| | NO: 255) | NO: 58) | (SEQ ID NO: 280) | NO: 290) |
| 113 | ACGACAGCCGUGGU | AGACAUGUGUCCUCA | CUCAUUCCUUGGGAUUGGUGACG | UAGGGGCUCAGCAGG |
| | GGAAUA (SEQ ID | GUGACAC (SEQ ID | ACGGCCAUAGUACUAUAGGAG | GCGGC (SEQ ID |
| | NO: 255) | NO: 58) | (SEQ ID NO: 281) | NO: 290) |
| 114 | ACGACAGCCGUGGU | AGACAUGUGUCCUCA | CUCAUUCCUUGGGAUUGGUGACA | UAGGGGCUCAGCAGG |
| | GGAAUA | GUGACAC (SEQ ID | ACGGCCAUAGUACUAUAGGAG | GCGGC (SEQ ID |
| | (SEQ ID | NO: 58) | (SEQ ID NO: 282) | NO: 290) |
| | NO: 255) | | | |

TABLE 17
Sequence of Exemplary Cas12i4 Editing Template RNAs

| Editing | | SEQ | | SEQ |
|---|---|---|---|---|
| Template | | ID | | ID |
| RNAs | Unmodified guide | NO | Modified | NO |
| 85 | GCCGCCCUGCUGAGCCCCUACUCCU | 291 | mG*mC*mC*rGrCrCrCrUrGrCrUrGrArGrCr | 321 |
| | AUUCUACUAUGGCCGUCGUCACCAA | | CrCrCrUrArCrUrCrCrUrArUrUrCrUrArCr | |
| | UCCCAAGGAAUGAGAGACAUGUGUC | | UrArUrGrGrCrCrGrUrCrGrUrCrArCrCrAr | |
| | CUCAGUGACACCUCCUAUUCCACCA | | ArUrCrCrCrArArGrGrArArUrGrArGrArGr | |
| | CGGCUG | | ArCrArUrGrUrGrUrCrCrUrCrArGrUrGrAr | |
| | | | CrArCrCrUrCrCrUrArUrUrCrCrArCrCrAr | |
| | | | CrGrG*mC*mU*mG | |
| 86 | GCCGCCCUGCUGAGCCCCUACUCCU | 292 | mG*mC*mC*rGrCrCrCrUrGrCrUrGrArGrCr | 322 |
| | AUAGUACUAUGGCCGUCGUCACCAA | | CrCrCrUrArCrUrCrCrUrArUrArGrUrArCr | |
| | UCCCAAGGAAUGAGAGACAUGUGUC | | UrArUrGrGrCrCrGrUrCrGrUrCrArCrCrAr | |
| | CUCAGUGACACCUCCUAUUCCACCA | | ArUrCrCrCrArArGrGrArArUrGrArGrArGr | |
| | CGGCUG | | ArCrArUrGrUrGrUrCrCrUrCrArGrUrGrAr | |
| | | | CrArCrCrUrCrCrUrArUrUrCrCrArCrCrAr | |
| | | | CrGrG*mC*mU*mG | |
| 87 | GCCGCCCUGCUGAGCCCCUACAGUU | 293 | mG*mC*mC*rGrCrCrCrUrGrCrUrGrArGrCr | 323 |
| | AUAGUACUAUGGCCGUCGUCACCAA | | CrCrCrUrArCrArGrUrUrArUrArGrUrArCr | |
| | UCCCAAGGAAUGAGAGACAUGUGUC | | UrArUrGrGrCrCrGrUrCrGrUrCrArCrCrAr | |
| | CUCAGUGACACCUCCUAUUCCACCA | | ArUrCrCrCrArArGrGrArArUrGrArGrArGr | |
| | CGGCUG | | ArCrArUrGrUrGrUrCrCrUrCrArGrUrGrAr | |
| | | | CrArCrCrUrCrCrUrArUrUrCrCrArCrCrAr | |
| | | | CrGrG*mC*mU*mG | |
| 88 | GCCCUGCUGAGCCCCUACUCCUAUU | 294 | mG*mC*mC*rCrUrGrCrUrGrArGrCrCrCrCr | 324 |
| | CCACUAUGGCCGUUGUCACCAAUCC | | UrArCrUrCrCrUrArUrUrCrCrArCrUrArUr | |
| | CAAGGAAUGAGGGAAGACAUGUGUC | | GrGrCrCrGrUrUrGrUrCrArCrCrArArUrCr | |
| | CUCAGUGACACCUAUUCCACCACGG | | CrCrArArGrGrArArUrGrArGrGrGrArArGr | |
| | CUGUCG | | ArCrArUrGrUrGrUrCrCrUrCrArGrUrGrAr | |
| | | | CrArCrCrUrArUrUrCrCrArCrCrArCrGrGr | |
| | | | CrUrG*mU*mC*mG | |
| 89 | GCCCUGCUGAGCCCCUACUCCUAUU | 295 | mG*mC*mC*rCrUrGrCrUrGrArGrCrCrCrCr | 325 |
| | CUACUAUGGCCGUUGUCACCAAUCC | | UrArCrUrCrCrUrArUrUrCrUrArCrUrArUr | |
| | CAAGGAAUGAGGGAAGACAUGUGUC | | GrGrCrCrGrUrUrGrUrCrArCrCrArArUrCr | |
| | CUCAGUGACACCUAUUCCACCACGG | | CrCrArArGrGrArArUrGrArGrGrGrArArGr | |
| | CUGUCG | | ArCrArUrGrUrGrUrCrCrUrCrArGrUrGrAr | |
| | | | CrArCrCrUrArUrUrCrCrArCrCrArCrGrGr | |
| | | | CrUrG*mU*mC*mG | |
| 90 | GCCCUGCUGAGCCCCUACUCCUAUA | 296 | mG*mC*mC*rCrUrGrCrUrGrArGrCrCrCrCr | 326 |
| | GUACUAUGGCCGUUGUCACCAAUCC | | UrArCrUrCrCrUrArUrArGrUrArCrUrArUr | |
| | CAAGGAAUGAGGGAAGACAUGUGUC | | GrGrCrCrGrUrUrGrUrCrArCrCrArArUrCr | |
| | CUCAGUGACACCUAUUCCACCACGG | | CrCrArArGrGrArArUrGrArGrGrGrArArGr | |
| | CUGUCG | | ArCrArUrGrUrGrUrCrCrUrCrArGrUrGrAr | |
| | | | CrArCrCrUrArUrUrCrCrArCrCrArCrGrGr | |
| | | | CrUrG*mU*mC*mG | |
| 91 | CUGCUGAGCCCCUACUCCUAUUCCA | 297 | mC*mU*mG*rCrUrGrArGrCrCrCrCrUrArCr | 327 |
| | CCAUGGCCGUUGUUACCAAUCCCAA | | UrCrCrUrArUrUrCrCrArCrCrArUrGrGrCr | |
| | GGAAUGAGGGACUUAGACAUGUGUC | | CrGrUrUrGrUrUrArCrCrArArUrCrCrCrAr | |
| | CUCAGUGACACUUCCACCACGGCUG | | ArGrGrArArUrGrArGrGrGrArCrUrUrArGr | |
| | UCGUCA | | ArCrArUrGrUrGrUrCrCrUrCrArGrUrGrAr | |
| | | | CrArCrUrUrCrCrArCrCrArCrGrGrCrUrGr | |
| | | | UrCrG*mU*mC*mA | |
| 92 | CUGCUGAGCCCCUACUCCUAUUCCA | 298 | mC*mU*mG*rCrUrGrArGrCrCrCrCrUrArCr | 328 |
| | CUAUGGCCGUUGUUACCAAUCCCAA | | UrCrCrUrArUrUrCrCrArCrUrArUrGrGrCr | |
| | GGAAUGAGGGACUUAGACAUGUGUC | | CrGrUrUrGrUrUrArCrCrArArUrCrCrCrAr | |
| | CUCAGUGACACUUCCACCACGGCUG | | ArGrGrArArUrGrArGrGrGrArCrUrUrArGr | |
| | UCGUCA | | ArCrArUrGrUrGrUrCrCrUrCrArGrUrGrAr | |
| | | | CrArCrUrUrCrCrArCrCrArCrGrGrCrUrGr | |
| | | | UrCrG*mU*mC*mA | |
| 93 | CUGCUGAGCCCCUACUCCUAUUCUA | 299 | mC*mU*mG*rCrUrGrArGrCrCrCrCrUrArCr | 329 |
| | CUAUGGCCGUUGUUACCAAUCCCAA | | UrCrCrUrArUrUrCrUrArCrUrArUrGrGrCr | |
| | GGAAUGAGGGACUUAGACAUGUGUC | | CrGrUrUrGrUrUrArCrCrArArUrCrCrCrAr | |
| | CUCAGUGACACUUCCACCACGGCUG | | ArGrGrArArUrGrArGrGrGrArCrUrUrArGr | |
| | UCGUCA | | ArCrArUrGrUrGrUrCrCrUrCrArGrUrGrAr | |
| | | | CrArCrUrUrCrCrArCrCrArCrGrGrCrUrGr | |
| | | | UrCrG*mU*mC*mA | |
| 94 | GCUGAGCCCCUACUCCUAUUCCACC | 300 | mG*mC*mU*rGrArGrCrCrCrCrUrArCrUrCr | 330 |
| | AUGGCUGUUGUUACUAAUCCCAAGG | | CrUrArUrUrCrCrArCrCrArUrGrGrCrUrGr | |
| | AAUGAGGGACUUCUAGACAUGUGUC | | UrUrGrUrUrArCrUrArArUrCrCrCrArArGr | |
| | CUCAGUGACACCCACCACGGCUGUC | | GrArArUrGrArGrGrGrArCrUrUrCrUrArGr | |
| | GUCACC | | ArCrArUrGrUrGrUrCrCrUrCrArGrUrGrAr | |
| | | | CrArCrCrCrArCrCrArCrGrGrCrUrGrUrCr | |
| | | | GrUrC*mA*mC*mC | |
| 95 | GCUGAGCCCCUACUCCUAUUCCACC | 301 | mG*mC*mU*rGrArGrCrCrCrCrUrArCrUrCr | 331 |
| | AUGGCCGUUGUUACUAAUCCCAAGG | | CrUrArUrUrCrCrArCrCrArUrGrGrCrCrGr | |
| | AAUGAGGGACUUCUAGACAUGUGUC | | UrUrGrUrUrArCrUrArArUrCrCrCrArArGr | |
| | CUCAGUGACACCCACCACGGCUGUC | | GrArArUrGrArGrGrGrArCrUrUrCrUrArGr | |
| | GUCACC | | ArCrArUrGrUrGrUrCrCrUrCrArGrUrGrAr | |
| | | | CrArCrCrCrArCrCrArCrGrGrCrUrGrUrCr | |
| | | | GrUrC*mA*mC*mC | |
| 96 | GCUGAGCCCCUACUCCUAUUCCACU | 302 | mG*mC*mU*rGrArGrCrCrCrCrUrArCrUrCr | 332 |
| | AUGGCCGUUGUUACUAAUCCCAAGG | | CrUrArUrUrCrCrArCrUrArUrGrGrCrCrGr | |
| | AAUGAGGGACUUCUAGACAUGUGUC | | UrUrGrUrUrArCrUrArArUrCrCrCrArArGr | |
| | CUCAGUGACACCCACCACGGCUGUC | | GrArArUrGrArGrGrGrArCrUrUrCrUrArGr | |
| | GUCACC | | ArCrArUrGrUrGrUrCrCrUrCrArGrUrGrAr | |
| | | | CrArCrCrCrArCrCrArCrGrGrCrUrGrUrCr | |
| | | | GrUrC*mA*mC*mC | |
| 97 | CUGAGCCCCUACUCCUAUUCCACCA | 303 | mC*mU*mG*rArGrCrCrCrCrUrArCrUrCrCr | 333 |
| | UGGCUGUUGUUACUAAUCCCAAGGA | | UrArUrUrCrCrArCrCrArUrGrGrCrUrGrUr | |
| | AUGAGGGACUUCUCAGACAUGUGUC | | UrGrUrUrArCrUrArArUrCrCrCrArArGrGr | |
| | CUCAGUGACACCACCACGGCUGUCG | | ArArUrGrArGrGrGrArCrUrUrCrUrCrArGr | |
| | UCACCA | | ArCrArUrGrUrGrUrCrCrUrCrArGrUrGrAr | |
| | | | CrArCrCrArCrCrArCrGrGrCrUrGrUrCrGr | |
| | | | UrCrA*mC*mC*mA | |
| 98 | CUGAGCCCCUACUCCUAUUCCACCA | 304 | mC*mU*mG*rArGrCrCrCrCrUrArCrUrCrCr | 334 |
| | UGGCCGUUGUUACUAAUCCCAAGGA | | UrArUrUrCrCrArCrCrArUrGrGrCrCrGrUr | |
| | AUGAGGGACUUCUCAGACAUGUGUC | | UrGrUrUrArCrUrArArUrCrCrCrArArGrGr | |
| | CUCAGUGACACCACCACGGCUGUCG | | ArArUrGrArGrGrGrArCrUrUrCrUrCrArGr | |
| | UCACCA | | ArCrArUrGrUrGrUrCrCrUrCrArGrUrGrAr | |
| | | | CrArCrCrArCrCrArCrGrGrCrUrGrUrCrGr | |
| | | | UrCrA*mC*mC*mA | |
| 99 | CUGAGCCCCUACUCCUAUUCCACUA | 305 | mC*mU*mG*rArGrCrCrCrCrUrArCrUrCrCr | 335 |
| | UGGCCGUUGUUACUAAUCCCAAGGA | | UrArUrUrCrCrArCrUrArUrGrGrCrCrGrUr | |
| | AUGAGGGACUUCUCAGACAUGUGUC | | UrGrUrUrArCrUrArArUrCrCrCrArArGrGr | |
| | CUCAGUGACACCACCACGGCUGUCG | | ArArUrGrArGrGrGrArCrUrUrCrUrCrArGr | |
| | UCACCA | | ArCrArUrGrUrGrUrCrCrUrCrArGrUrGrAr | |
| | | | CrArCrCrArCrCrArCrGrGrCrUrGrUrCrGr | |
| | | | UrCrA*mC*mC*mA | |
| 100 | GGAGAAGUCCCUCAUUCCUUGGGAU | 306 | mG*mG*mA*rGrArArGrUrCrCrCrUrCrArUr | 336 |
| | UGGUAACAACGGCCAUGGUGGAAUA | | UrCrCrUrUrGrGrGrArUrUrGrGrUrArArCr | |
| | GGAGUAGGGGCUCAAGACAUGUGUC | | ArArCrGrGrCrCrArUrGrGrUrGrGrArArUr | |
| | CUCAGUGACACGGGAUUGGUGACGA | | ArGrGrArGrUrArGrGrGrGrCrUrCrArArGr | |
| | CAGCCG | | ArCrArUrGrUrGrUrCrCrUrCrArGrUrGrAr | |
| | | | CrArCrGrGrGrArUrUrGrGrUrGrArCrGrAr | |
| | | | CrArG*mC*mC*mG | |
| 101 | GGAGAAGUCCCUCAUUCCUUGGGAU | 307 | mG*mG*mA*rGrArArGrUrCrCrCrUrCrArUr | 337 |
| | UAGUAACAACGGCCAUGGUGGAAUA | | UrCrCrUrUrGrGrGrArUrUrArGrUrArArCr | |
| | GGAGUAGGGGCUCAAGACAUGUGUC | | ArArCrGrGrCrCrArUrGrGrUrGrGrArArUr | |
| | CUCAGUGACACGGGAUUGGUGACGA | | ArGrGrArGrUrArGrGrGrGrCrUrCrArArGr | |
| | CAGCCG | | ArCrArUrGrUrGrUrCrCrUrCrArGrUrGrAr | |
| | | | CrArCrGrGrGrArUrUrGrGrUrGrArCrGrAr | |
| | | | CrArG*mC*mC*mG | |
| 102 | GGAGAAGUCCCUCAUUCCUUGGGGU | 308 | mG*mG*mA*rGrArArGrUrCrCrCrUrCrArUr | 338 |
| | UAGUAACAACGGCCAUGGUGGAAUA | | UrCrCrUrUrGrGrGrGrUrUrArGrUrArArCr | |
| | GGAGUAGGGGCUCAAGACAUGUGUC | | ArArCrGrGrCrCrArUrGrGrUrGrGrArArUr | |
| | CUCAGUGACACGGGAUUGGUGACGA | | ArGrGrArGrUrArGrGrGrGrCrUrCrArArGr | |
| | CAGCCG | | ArCrArUrGrUrGrUrCrCrUrCrArGrUrGrAr | |
| | | | CrArCrGrGrGrArUrUrGrGrUrGrArCrGrAr | |
| | | | CrArG*mC*mC*mG | |
| 103 | GAGAAGUCCCUCAUUCCUUGGGAUU | 309 | mG*mA*mG*rArArGrUrCrCrCrUrCrArUrUr | 339 |
| | GGUAACAACGGCCAUGGUGGAAUAG | | CrCrUrUrGrGrGrArUrUrGrGrUrArArCrAr | |
| | GAGUAGGGGCUCAGAGACAUGUGUC | | ArCrGrGrCrCrArUrGrGrUrGrGrArArUrAr | |
| | CUCAGUGACACGGAUUGGUGACGAC | | GrGrArGrUrArGrGrGrGrCrUrCrArGrArGr | |
| | AGCCGU | | ArCrArUrGrUrGrUrCrCrUrCrArGrUrGrAr | |
| | | | CrArCrGrGrArUrUrGrGrUrGrArCrGrArCr | |
| | | | ArGrC*mC*mG*mU | |
| 104 | GAGAAGUCCCUCAUUCCUUGGGAUU | 310 | mG*mA*mG*rArArGrUrCrCrCrUrCrArUrUr | 340 |
| | AGUAACAACGGCCAUGGUGGAAUAG | | CrCrUrUrGrGrGrArUrUrArGrUrArArCrAr | |
| | GAGUAGGGGCUCAGAGACAUGUGUC | | ArCrGrGrCrCrArUrGrGrUrGrGrArArUrAr | |
| | CUCAGUGACACGGAUUGGUGACGAC | | GrGrArGrUrArGrGrGrGrCrUrCrArGrArGr | |
| | AGCCGU | | ArCrArUrGrUrGrUrCrCrUrCrArGrUrGrAr | |
| | | | CrArCrGrGrArUrUrGrGrUrGrArCrGrArCr | |
| | | | ArGrC*mC*mG*mU | |
| 105 | GAGAAGUCCCUCAUUCCUUGGGGUU | 311 | mG*mA*mG*rArArGrUrCrCrCrUrCrArUrUr | 341 |
| | AGUAACAACGGCCAUGGUGGAAUAG | | CrCrUrUrGrGrGrGrUrUrArGrUrArArCrAr | |
| | GAGUAGGGGCUCAGAGACAUGUGUC | | ArCrGrGrCrCrArUrGrGrUrGrGrArArUrAr | |
| | CUCAGUGACACGGAUUGGUGACGAC | | GrGrArGrUrArGrGrGrGrCrUrCrArGrArGr | |
| | AGCCGU | | ArCrArUrGrUrGrUrCrCrUrCrArGrUrGrAr | |
| | | | CrArCrGrGrArUrUrGrGrUrGrArCrGrArCr | |
| | | | ArGrC*mC*mG*mU | |
| 106 | GUCCCUCAUUCCUUGGGAUUGGUGA | 312 | mG*mU*mC*rCrCrUrCrArUrUrCrCrUrUrGr | 342 |
| | CGACGGCCAUAGUAGAAUAGGAGUA | | GrGrArUrUrGrGrUrGrArCrGrArCrGrGrCr | |
| | GGGGCUCAGCAGGGAGACAUGUGUC | | CrArUrArGrUrArGrArArUrArGrGrArGrUr | |
| | CUCAGUGACACGGUGACGACAGCCG | | ArGrGrGrGrCrUrCrArGrCrArGrGrGrArGr | |
| | UGGUGG | | ArCrArUrGrUrGrUrCrCrUrCrArGrUrGrAr | |
| | | | CrArCrGrGrUrGrArCrGrArCrArGrCrCrGr | |
| | | | UrGrG*mU*mG*mG | |
| 107 | GUCCCUCAUUCCUUGGGAUUGGUGA | 313 | mG*mU*mC*rCrCrUrCrArUrUrCrCrUrUrGr | 343 |
| | CAACGGCCAUAGUAGAAUAGGAGUA | | GrGrArUrUrGrGrUrGrArCrArArCrGrGrCr | |
| | GGGGCUCAGCAGGGAGACAUGUGUC | | CrArUrArGrUrArGrArArUrArGrGrArGrUr | |
| | CUCAGUGACACGGUGACGACAGCCG | | ArGrGrGrGrCrUrCrArGrCrArGrGrGrArGr | |
| | UGGUGG | | ArCrArUrGrUrGrUrCrCrUrCrArGrUrGrAr | |
| | | | CrArCrGrGrUrGrArCrGrArCrArGrCrCrGr | |
| | | | UrGrG*mU*mG*mG | |
| 108 | GUCCCUCAUUCCUUGGGAUUGGUAA | 314 | mG*mU*mC*rCrCrUrCrArUrUrCrCrUrUrGr | 344 |
| | CAACGGCCAUAGUAGAAUAGGAGUA | | GrGrArUrUrGrGrUrArArCrArArCrGrGrCr | |
| | GGGGCUCAGCAGGGAGACAUGUGUC | | CrArUrArGrUrArGrArArUrArGrGrArGrUr | |
| | CUCAGUGACACGGUGACGACAGCCG | | ArGrGrGrGrCrUrCrArGrCrArGrGrGrArGr | |
| | UGGUGG | | ArCrArUrGrUrGrUrCrCrUrCrArGrUrGrAr | |
| | | | CrArCrGrGrUrGrArCrGrArCrArGrCrCrGr | |
| | | | UrGrG*mU*mG*mG | |
| 109 | UCCCUCAUUCCUUGGGAUUGGUGAC | 315 | mU*mC*mC*rCrUrCrArUrUrCrCrUrUrGrGr | 345 |
| | AACGGCCAUAGUAGAAUAGGAGUAG | | GrArUrUrGrGrUrGrArCrArArCrGrGrCrCr | |
| | GGGCUCAGCAGGGCAGACAUGUGUC | | ArUrArGrUrArGrArArUrArGrGrArGrUrAr | |
| | CUCAGUGACACGUGACGACAGCCGU | | GrGrGrGrCrUrCrArGrCrArGrGrGrCrArGr | |
| | GGUGGA | | ArCrArUrGrUrGrUrCrCrUrCrArGrUrGrAr | |
| | | | CrArCrGrUrGrArCrGrArCrArGrCrCrGrUr | |
| | | | GrGrU*mG*mG*mA | |
| 110 | UCCCUCAUUCCUUGGGAUUGGUGAC | 316 | mU*mC*mC*rCrUrCrArUrUrCrCrUrUrGrGr | 346 |
| | GACGGCCAUAGUACUAUAGGAGUAG | | GrArUrUrGrGrUrGrArCrGrArCrGrGrCrCr | |
| | GGGCUCAGCAGGGCAGACAUGUGUC | | ArUrArGrUrArCrUrArUrArGrGrArGrUrAr | |
| | CUCAGUGACACGUGACGACAGCCGU | | GrGrGrGrCrUrCrArGrCrArGrGrGrCrArGr | |
| | GGUGGA | | ArCrArUrGrUrGrUrCrCrUrCrArGrUrGrAr | |
| | | | CrArCrGrUrGrArCrGrArCrArGrCrCrGrUr | |
| | | | GrGrU*mG*mG*mA | |
| 111 | UCCCUCAUUCCUUGGGAUUGGUAAC | 317 | mU*mC*mC*rCrUrCrArUrUrCrCrUrUrGrGr | 347 |
| | AACGGCCAUAGUACUAUAGGAGUAG | | GrArUrUrGrGrUrArArCrArArCrGrGrCrCr | |
| | GGGCUCAGCAGGGCAGACAUGUGUC | | ArUrArGrUrArCrUrArUrArGrGrArGrUrAr | |
| | CUCAGUGACACGUGACGACAGCCGU | | GrGrGrGrCrUrCrArGrCrArGrGrGrCrArGr | |
| | GGUGGA | | ArCrArUrGrUrGrUrCrCrUrCrArGrUrGrAr | |
| | | | CrArCrGrUrGrArCrGrArCrArGrCrCrGrUr | |
| | | | GrGrU*mG*mG*mA | |
| 112 | CUCAUUCCUUGGGAUUGGUGACGAC | 318 | mC*mU*mC*rArUrUrCrCrUrUrGrGrGrArUr | 348 |
| | GGCCAUAGUAGAAUAGGAGUAGGGG | | UrGrGrUrGrArCrGrArCrGrGrCrCrArUrAr | |
| | CUCAGCAGGGCGGCAGACAUGUGUC | | GrUrArGrArArUrArGrGrArGrUrArGrGrGr | |
| | CUCAGUGACACACGACAGCCGUGGU | | GrCrUrCrArGrCrArGrGrGrCrGrGrCrArGr | |
| | GGAAUA | | ArCrArUrGrUrGrUrCrCrUrCrArGrUrGrAr | |
| | | | CrArCrArCrGrArCrArGrCrCrGrUrGrGrUr | |
| | | | GrGrA*mA*mU*mA | |
| 113 | CUCAUUCCUUGGGAUUGGUGACGAC | 319 | mC*mU*mC*rArUrUrCrCrUrUrGrGrGrArUr | 349 |
| | GGCCAUAGUACUAUAGGAGUAGGGG | | UrGrGrUrGrArCrGrArCrGrGrCrCrArUrAr | |
| | CUCAGCAGGGCGGCAGACAUGUGUC | | GrUrArCrUrArUrArGrGrArGrUrArGrGrGr | |
| | CUCAGUGACACACGACAGCCGUGGU | | GrCrUrCrArGrCrArGrGrGrCrGrGrCrArGr | |
| | GGAAUA | | ArCrArUrGrUrGrUrCrCrUrCrArGrUrGrAr | |
| | | | CrArCrArCrGrArCrArGrCrCrGrUrGrGrUr | |
| | | | GrGrA*mA*mU*mA | |
| 114 | CUCAUUCCUUGGGAUUGGUGACAAC | 320 | mC*mU*mC*rArUrUrCrCrUrUrGrGrGrArUr | 350 |
| | GGCCAUAGUACUAUAGGAGUAGGGG | | UrGrGrUrGrArCrArArCrGrGrCrCrArUrAr | |
| | CUCAGCAGGGCGGCAGACAUGUGUC | | GrUrArCrUrArUrArGrGrArGrUrArGrGrGr | |
| | CUCAGUGACACACGACAGCCGUGGU | | GrCrUrCrArGrCrArGrGrGrCrGrGrCrArGr | |
| | GGAAUA | | ArCrArUrGrUrGrUrCrCrUrCrArGrUrGrAr | |
| | | | CrArCrArCrGrArCrArGrCrCrGrUrGrGrUr | |
| | | | GrGrA*mA*mU*mA | |

m: 2′O-methyl nucleotide
*phosphorothioate bond between nucleotides

HEK293T cells were transfected and analyzed as described in Example 1. As shown in FIG. 8, variant Cas12i4-RT fusion polypeptide in the presence of editing template RNA sequences was capable of introducing the encoded substitutions at the TTR locus. Percentage of NGS reads is shown on the y-axis, total edits are shown as in grey bars, and encoded edits are shown as black bars. The data shown is an average of two biological replicates, each of which had three technical replicates. Precise editing efficiency for editing template RNAs that contained PS-2′OMe modifications on the first three and penultimate three nucleotides ranged from 2-10%, with different editing template RNAs displaying different levels of indels and precise edits.

The percentages of average indels and the percentages of average precise edits (RT) of exemplary editing template RNAs are provided in Table 19 below.

TABLE 19
Editing Efficiencies of Exemplary Editing Template
RNAs with Cas12i4-RT Fusion Polypeptide

| Editing Template | % Average | % Average |
|---|---|---|
| RNAS | Indel | RT |
| 85 | 23.19315706 | 9.370030185 |
| 86 | 22.87299289 | 7.6768571 |
| 87 | 26.02383294 | 3.206344529 |
| 88 | 5.496842494 | 0.187086192 |
| 89 | 5.735869841 | 0.182283761 |
| 90 | 4.470748084 | 0.127679882 |
| 91 | 8.124759743 | 3.083261612 |
| 92 | 7.863309249 | 2.924513952 |
| 93 | 8.426114411 | 2.37311705 |
| 94 | 5.606878574 | 0.153267199 |
| 95 | 7.378622415 | 0.204823005 |
| 96 | 6.514431252 | 0.22724429 |
| 97 | 41.79584821 | 7.655688657 |
| 98 | 43.51784573 | 9.799735872 |
| 99 | 37.8720169 | 8.597082505 |
| 100 | 14.29501663 | 0.258323688 |
| 101 | 13.44640947 | 0.223651918 |
| 102 | 10.65983381 | 0.051776527 |
| 103 | 24.36364863 | 0.568498514 |
| 104 | 27.02471583 | 0.779216287 |
| 105 | 22.67114731 | 0.304180219 |
| 106 | 2.204742333 | 0.035186606 |
| 107 | 2.720154332 | 0.067593305 |
| 108 | 2.622549706 | 0.051042382 |
| 109 | 2.730454743 | 0.096192855 |
| 110 | 2.505546745 | 0.059970098 |
| 111 | 2.256738032 | 0.076731596 |
| 112 | 1.288499727 | 0.027182442 |
| 113 | 1.558777496 | 0.04183775 |
| 114 | 1.538932611 | 0.029314399 |

This Example shows that specific edits were incorporated into the human TTR genomic locus using editing template RNAs and a variant Cas12i4-RT fusion polypeptide in HEK293T cells.

### Example 6: RNA-Templated Editing of the TTR Gene in Primary Human Hepatocytes Using Chemically Modified Editing Template RNAs and mRNA Delivery of a Cas12i2 Nuclease-RT Fusion Polypeptide

This Example describes editing of the human TTR gene in liver cells with an mRNA-encoded variant Cas12i2-RT or variant Cas12i4-RT fusion polypeptide and editing template RNAs.

A fusion of a variant Cas12i2 with mutant MMLV reverse transcriptase (RT) (SEQ ID NO: 353) or is cloned and transcribed into mRNA using the method of Example 2. The editing template RNAs are prepared as described in Example 1.

Primary human hepatocytes (PHH) are thawed and counted. Cells are washed once with PBS and resuspended in P3 buffer+supplement (Lonza #V4XP-3032)+transfection enhancer oligo (final concentration 4 μM) at a concentration of 6944 cells/μL. Resuspended cells are dispensed at 1.25e5 cells/reaction into Lonza 16-well nucleocuvette strips. 1 μL of Cas12i2-RT mRNA (1 mg/mL) are mixed with 1 μL of editing template RNA (1 mM) and added to each reaction.

The strips are electroporated using an electroporation device (program DS-150, Lonza 4D-nucleofector). Five minutes following electroporation. 45 μL of pre-warmed hepatocyte plating media are added to each well, mixed, and incubated for five minutes at room temperature. Following incubation, cells are plated into one well/condition into pre-warmed collagen-coated 96-well plate with wells containing 60 μL hepatocyte coating media. Editing plates are incubated for 4 h at 37 °C with 5% CO₂. Following incubation, media was exchanged for hepatocyte maintenance media, and cells are incubated for 72 h at 37 °C with 5% CO₂.

After 3 days, media is flicked off and cells are resuspended in 30 μL DNA extraction buffer (QuickExtract) and removed by scraping. Samples are then cycled in a PCR instrument at 65 °C for 15 min, 68 °C for 15 min, 98 °C for 10 min. Samples are then frozen at −20 °C.

Samples for NGS are prepared as described in Example 1. Presence of precise edits in NGS reads indicates that the editing template RNAs disclosed herein are capable of introducing the T119M SNP into primary human hepatocytes.

### Example 7—RNA-Templated Editing of the TTR Gene in Mice

In this Example, activity of i) the Cas12i2-RT fusion of SEQ ID NO: 59 or SEQ ID NO: 353 or the Cas12i-4-RT fusion of SEQ ID NO: 238 and ii) editing template RNAs is measured in two mouse models. For example, activity of the Cas12i2-RT fusion of SEQ ID NO: 353 and either editing template RNA 60 or editing template RNA 64 is measured.

In the first study, the editing template RNAs target human TTR in adult transgenic mice in which the mouse TTR coding sequence has been replaced with the human sequence encoding TTR with the V30M mutation. This model is not a disease model due to low levels of TTR expression, which cannot mimic the disease phenotype. However, the model provides a means of evaluating human-targeting therapeutic candidate editing template RNAs in a mouse model. The first study is summarized in Table 20. Delivery is via LNP.

TABLE 20
Study 1 Outline

| | Editing | | | | | |
|---|---|---|---|---|---|---|
| Group | Template | # | | | | Dose |
| # | RNA | Mice | Dose | Admin Route | Freq. | Day |
| 1 | 60 | 4 | 3 mg/kg | Tail vein, 0.2 mL | 1x | D 0 |
| 2 | 60 | 4 | 3 mg/kg | Tail vein, 0.2 mL | 2x | D 0, |
| | | | | | | D 7 |
| 3 | 64 | 4 | 3 mg/kg | Tail vein, 0.2 mL | 1x | D 0 |
| 4 | 64 | 4 | 3 mg/kg | Tail vein, 0.2 mL | 2x | D 0, |
| | | | | | | D 7 |
| 5 | Buffer | 4 | N/A | Tail vein, 0.2 mL | 1x | D 0 |
| | Control | | | | | |

### Endpoint

7 days post final dose

### Sample Collection:

Serum: Day −3, 24 h post, terminal
Tissue: Flash frozen liver, lung; fixed liver tissue

### Analysis

NGS—indel & RT editing analysis
Histology—H&E
ALT, AST, TTR protein assays

In the second study, the same transgenic TTR mouse model as Study 1 is used to evaluate the editing potential of the Cas12i2-RT fusion polypeptide in neonatal animals when the liver is in a highly replicative state. In this study, the endpoint is extended out to 4 weeks to allow time for the animals to develop and provide sufficient liver tissue for analysis. Delivery is via LNP.

TABLE 21
Study 2 Outline

| | Editing | | | |
|---|---|---|---|---|
| | Template | | | |
| Group # | RNA | # Mice | Dose | Admin Route |
| 1 | 60 | >10 pups, potentially | 3 mg/kg | Tail vein, 0.2 mL |
| | | multiple liters | | |
| 2 | 64 | >10 pups, potentially | 3 mg/kg | Tail vein, 0.2 mL |
| | | multiple liters | | |
| 3 | Buffer | >10 pups, potentially | N/A | Tail vein, 0.2 mL |
| | Control | multiple liters | | |

### Endpoint

4 weeks post dose

### Sample Collection:

Serum: Terminal
Tissue: Flash frozen liver, lung; fixed liver tissue

### Analysis

NGS—indel & RT editing
Histology—H&E
ALT, AST, TTR protein assays

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an.” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one. A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system for genetic editing of a transthyretin (TTR) gene, comprising
(a) a Cas12i polypeptide or a first nucleic acid encoding the Cas12i polypeptide;
(b) a reverse transcriptase (RT) polypeptide or a second nucleic acid encoding the RT polypeptide;
(c) an RNA guide or a third nucleic acid encoding the RNA guide, wherein the RNA guide comprises a spacer sequence specific to a target sequence within a TTR gene, the target sequence being adjacent to a protospacer adjacent motif (PAM) comprising the motif of 5′-TTN-3′, which is located 5′ to the target sequence; and
(d) a reverse transcription donor RNA (RT donor RNA) or a fourth nucleic acid encoding the RT donor RNA, wherein the RT donor RNA comprises a primer binding site (PBS) and a template sequence.

2. The gene editing system of claim 1, wherein the Cas12i polypeptide is a Cas12i2 or Cas12i4 polypeptide, which comprises an amino acid sequence at least 95% identical to SEQ ID NO: 9 or 15, respectively, and comprises one or more mutations relative to SEQ ID NO: 9 or 15, respectively; and/or
wherein the RT polypeptide is Moloney Murine Leukemia Virus (MMLV)-RT, mouse mammary tumor virus (MMTV)-RT, Marathon-RT, or RTx-RT.

3. The gene editing system of claim 2, wherein the Cas12i polypeptide is a Cas12i2 polypeptide and wherein the one or more mutations in the Cas12i2 polypeptide are at positions H485, D581, G624, F626, P868, I926, V1030, E1035, and/or S1046 of SEQ ID NO: 9.

4. The gene editing system of claim 3, wherein the one or more mutations are amino acid substitutions, which optionally are H485A, D581R, G624R, F626R, P868T, I926R, V1030G, E1035R, S1046G, or a combination thereof.

5. The gene editing system of claim 4, wherein the Cas12i2 polypeptide comprises:
(i) a mutation at position H485, which optionally is an amino acid substitution of H485A;
(ii) mutations at positions D581, D911, I926, and V1030, which optionally are amino acid substitutions of D581R, D911R, I926R, and V1030G;
(iii) mutations at positions D581, I926, and V1030, which optionally are amino acid substitutions of D581R, I926R, and V1030G;
(iv) mutations at positions D581, I926, V1030, and S1046, which optionally are amino acid substitutions of D581R, I926R, V1030G, and S1046G;
(v) mutations at positions D581, G624, F626, I926, V1030, E1035, and S1046, which optionally are amino acid substitutions of D581R, G624R, F626R, I926R, V1030G, E1035R, and S1046G; or
(vi) mutations at positions D581, G624, F626, P868, I926, V1030, E1035, and S1046, which optionally are amino acid substitutions of D581R, G624R, F626R, P868T, I926R, V1030G, E1035R, and S1046G.

6. The gene editing system of claim 5, wherein the Cas12i2 polypeptide comprises the amino acid substitutions of D581R, G624R, F626R, P868T, I926R, V1030G, E1035R, and S1046G, optionally wherein the Cas12i2 polypeptide comprises the amino acid sequence of SEQ ID NO: 14, or
wherein the Cas12i2 polypeptide comprises the amino acid substitutions of D581R, I926R, and V1030G, optionally wherein the Cas12i2 polypeptide comprises the amino acid sequence of SEQ ID NO: 11.

7.-13. (canceled)

14. The gene editing system of claim 1, wherein the gene editing system comprises a fusion polypeptide or a nucleic acid encoding the fusion polypeptide, and wherein the fusion polypeptide comprises the Cas12i polypeptide and the RT polypeptide.

15. The gene editing system of claim 14, wherein the Cas12i polypeptide is the Cas12i2 polypeptide; optionally wherein the fusion polypeptide comprises the amino acid sequence set forth in SEQ ID NO: 59 or 353.

16. (canceled)

17. The gene editing system of claim 14, which comprises the nucleic acid encoding the fusion polypeptide.

18. The gene editing system of claim 17, wherein the nucleic acid is a mRNA molecule or a DNA molecule.

19. (canceled)

20. The gene editing system of claim 1, wherein the target sequence comprises:
(i)
(SEQ ID NO: 60)
CACCACGGCTGTCGTCACCA,
(ii)
(SEQ ID NO: 61)
GGATTGGTGACGACAGCCGT,
(iii)
(SEQ ID NO: 62)
CTTGGGATTGGTGACGACAG,
(iv)
(SEQ ID NO: 240)
CTCCTATTCCACCACGGCTG,
(v)
(SEQ ID NO: 241)
CTATTCCACCACGGCTGTCG,
(vi)
(SEQ ID NO: 242)
TTCCACCACGGCTGTCGTCA,
(vii)
(SEQ ID NO: 243)
CCACCACGGCTGTCGTCACC,
(viii)
(SEQ ID NO: 244)
GGGATTGGTGACGACAGCCG,
(ix)
(SEQ ID NO: 245)
GGTGACGACAGCCGTGGTGG,
(x)
(SEQ ID NO: 246)
GTGACGACAGCCGTGGTGGAU,
or
(xi)
(SEQ ID NO: 247)
ACGACAGCCGTGGTGGAATA.

21. The gene editing system of claim 20, wherein the spacer sequence is set forth as:
(i)
(SEQ ID NO: 63)
CACCACGGCUGUCGUCACCA,
(ii)
(SEQ ID NO: 64)
GGAUUGGUGACGACAGCCGU,
iii)
(SEQ ID NO: 65)
CUUGGGAUUGGUGACGACAG,
(iv)
(SEQ ID NO: 248)
CUCCUAUUCCACCACGGCUG,
(v)
(SEQ ID NO: 249)
CUAUUCCACCACGGCUGUCG,
(vi)
(SEQ ID NO: 250)
UUCCACCACGGCUGUCGUCA,
(vii)
(SEQ ID NO: 251)
CCACCACGGCUGUCGUCACC,
(viii)
(SEQ ID NO: 252)
GGGAUUGGUGACGACAGCCG,
(ix)
(SEQ ID NO: 253)
GGUGACGACAGCCGUGGUGG,
(x)
(SEQ ID NO: 254)
GUGACGACAGCCGUGGUGGA,
or
(xi)
(SEQ ID NO: 255)
ACGACAGCCGUGGUGGAAUA

22. The gene editing system of claim 1, wherein the RNA guide comprises the spacer sequence and a direct repeat (DR) sequence, and wherein the direct repeat sequence is set forth as AGAAAUCCGUCUUUCAUUGACGG (SEQ ID NO: 39) or AGACAUGUGUCCUCAGUGACAC (SEQ ID NO: 58).

23.-25. (canceled)

26. The gene editing system of claim 1, wherein the PBS is 25-75-nucleotide in length; and/or
wherein the template sequence is 25-75-nucleotide in length.

27. (canceled)

28. The gene editing system of claim 26, wherein the PBS is set forth as any one of those listed in Table 10 or Table 16.

29.-30. (canceled)

31. The gene editing system of claim 1, wherein the template sequence is homologous to the genomic site of interest and comprises at least two nucleotide variations relative to the genomic site of interest, optionally wherein the template sequence comprises a nucleotide variation encoding the T119M substitution and one or more silent mutations.

32. The gene editing system of claim 31, wherein at least one nucleotide variation is located within the region of the template sequence that is complementary to the target sequence, optionally wherein the codon including the at least one nucleotide variation encodes the T119M substitution; and at least one nucleotide variation is located in the PAM.

33. (canceled)

34. The gene editing system of claim 1, wherein the template sequence comprises any one of those listed in Table 10 and Table 16: or wherein the template sequence comprises one or more mutations at position C₆ of SEQ ID NO: 351.

35. (canceled)

36. The gene editing system of claim 34, wherein the template sequence comprises one or more mutations at position C₆ of SEQ ID NO: 351; and wherein the one or more mutations further occur at positions C₄, C₁₀, C₁₃, C₁₆, or C₁₉ of SEQ ID NO: 351, or a combination thereof.

37.-38. (canceled)

39. The gene editing system of claim 1, which comprises a polyribonucleotide comprising the RNA guide and the RT donor RNA; wherein the RNA guide comprises the spacer sequence and the DR sequence; and wherein the RT donor RNA comprises the PBS and the template sequence.

40. (canceled)

41. The gene editing system of claim 39, wherein the polyribonucleotide further comprises a 5′ end protection means, a 3′ end protection means, or both, and/or wherein the polyribonucleotide comprises one or more modifications comprising 2′-O-methylation, PS bond, or a combination thereof.

42. The gene editing system of claim 39, wherein the polyribonucleotide comprises the nucleotide sequence of any one of those listed in Table 11 and Table 17.

43.-44. (canceled)

45. The gene editing system of claim 18, which comprise (i) the mRNA molecule encoding the fusion polypeptide, and (ii) a polyribonucleotide comprising the RNA guide and the RT donor RNA, and wherein the gene editing system comprises one or more lipid nanoparticles (LNPs), which are associated with the mRNAs and the polyribonucleotide.

46. (canceled)

47. The gene editing system of claim 18, which comprises the DNA molecule encoding the fusion polypeptide; wherein the DNA molecule further encodes a polyribonucleotide comprising the RNA guide and the RT donor RNA.

48. A pharmaceutical composition or a combination of pharmaceutical compositions, which collectively comprise the gene editing system set forth in claim 1.

49. (canceled)

50. A method for editing a transthyretin (TTR) gene in a cell, the method comprising contacting a host cell with the gene editing system for editing the TTR gene set forth in claim 1 or a pharmaceutical composition comprising the gene editing system to genetically edit the TTR gene in the host cell.

51.-55. (canceled)

56. A method for treating amyloidogenic transthyretin (ATTR) in a subject in need thereof, comprising administering to the subject a gene editing system for editing a transthyretin (TTR) gene set forth in claim 1.

57. (canceled)


SEQUENCE LISTING (357 sequences)

SEQ ID NO: 1 - DNA, 6945 bp, Homo sapiens
  source 1..6945: mol_type = genomic DNA
acagaagtccactcattcttggcaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 2 - DNA, 145 bp, Homo sapiens
  source 1..145: mol_type = genomic DNA
acagaagtccactcattcttggcaggatggcttctcatcgtctgctcctcctctgccttgctggactggtatttgtgtctgaggctggccctacggtgagtgtttctgtgacatcccattcctacatttaagattcacgctaaat

SEQ ID NO: 3 - DNA, 231 bp, Homo sapiens
  source 1..231: mol_type = genomic DNA
ttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgttt

SEQ ID NO: 4 - DNA, 236 bp, Homo sapiens
  source 1..236: mol_type = genomic DNA
ccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttg

SEQ ID NO: 5 - DNA, 304 bp, Homo sapiens
  source 1..304: mol_type = genomic DNA
cacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 6 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 7 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 8 - DNA, 444 bp, Homo sapiens
  source 1..444: mol_type = genomic DNA
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 9 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 10 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 11 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 12 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 13 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 14 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 15 - protein, 1074 aa, synthetic construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 16 - protein, 1074 aa, synthetic construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 17 - protein, 678 aa, Murine leukemia virus
MTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 18 - protein, 487 aa, Murine leukemia virus
MTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEILQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDL

SEQ ID NO: 19 - protein, 603 aa, Mouse mammary tumor virus
MFTLWGRDIMKDIKVRLMTDSPDDSQDLMIGAIESNLFADQISWKSDQPVWLNQWPLKQEKLQALQQLVTEQLQLGHLEESNSPWNTPVFVIKKKSGKWRLLQDLRAVNATMHDMGALQPGLPSPVAVPKGWEIIIIDLQDCFFNIKLHPEDCKRFAFSVPSPNFKRPYQRFQWKVLPQGMKNSPTLCQKFVDKAILTVRDKYQDSYIVHYMDDILLAHPSRSIVDEILTSMIQALNKHGLVVSTEKIQKYDNLKYLGTHIQGDSVSYQKLQIRTDKLRTLNDFQKLLGNINWIRPFLKLTTGELKPLFEILNGDSNPISTRKLTPEACKALQLMNERLSTARVKRLDLSQPWSLCILKTEYTPTACLWQDGVVEWIHLPHISPKVITPYDIFCTQLIIKGRHRSKELFSKDPDYIVVPYTKVQFDLLLQEKEDWPISLLGFLGEVHFHLPKDPLLTFTLQTAIIFPHMTSTTPLEKGIVIFTDGSANGRSVTYIQGREPIIKENTQNTAQQAEIVAVITAFEEVSQPFNLYTDSKYVTGLFPEIETATLSPRTKIYTELKHLQRLIHKRQEKFYIGHIRGHTGLPGPLAQGNAYADSLTRIL

SEQ ID NO: 20 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 21 - protein, 774 aa, synthetic construct
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 22 - protein, 774 aa, synthetic construct
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYDGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 23 - protein, 774 aa, synthetic construct
MILDTDYITEDGKPVIRIFKKENGEFKIEYDRTFEPYLYALLKDDSAIEEVKKITAERHSTVVTVKRVEKVQKKFLGRSVEVWKLYFTHPQDVPAIMDKIREHPAVIDIYEYDIPFAIRYLIDKGLVPMEGDEELKLLALDIGTPCHEGEVFAEGPILMISYADEEGTRVITWRNVDLPYVDVLSTEREMIQRFLRVVKEKDPDVLITYNGDNFDFAYLKKRCEKLGINFTLGREGSEPKIQRMGDRFAVEVKGRIHFDLYPVIRRTVNLPIYTLEAVYEAVFGQPKEKVYAEEITTAWETGENLERVARYSMEDAKVTYELGKEFMPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYITMTIKEIEEKYGFKLIYSDTDGFFATIPGAEAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIENQVLPAVERILRAYGYRKEDLWYQKTRQVGLSARLKPKGT

SEQ ID NO: 24 - protein, 683 aa, unidentified
MDHLLLKTQTQTEQVMNVTNPNSIYIKGRLYFKGYKKIELHCFVDTGASLCIASKFVIPEEHWVNAERPIMVKIADGSSITISKVCKDIDLIIAGEIFRIPTVYQQESGIDFIIGNNFCQLYEPFIQFTDRVIFTKNKSYPVHIAKLTRAVRVGTEGFLESMKKRSKTQQPEPVNISTNKIENPLEEIAILSEGRRLSEEKLFITQQRMQKIEELLEKVCSENPLDPNKTKQWMKASIKLSDPSKAIKVKPMKYSPMDREEFDKQIKELLDLKVIKPSKSPHMAPAFLVNNEAEKRRGKKRMVVNYKAMNKATVGDAYNLPNKDELLTLIRGKKIFSSFDCKSGFWQVLLDQESRPLTAFTCPQGHYEWNVVPFGLKQAPSIFQRHMDEAFRVFRKFCCVYVDDILVFSNNEEDHLLHVAMILQKCNQHGIILSKKKAQLFKKKINFLGLEIDEGTHKPQGHILEHINKFPDTLEDKKQLQRFLGILTYASDYIPKLAQIRKPLQAKLKENVPWRWTKEDTLYMQKVKKNLQGFPPLHHPLPEEKLIIETDASDDYWGGMLKAIKINEGTNTELICRYASGSFKAAEKNYHSNDKETLAVINTIKKFSIYLTPVHFLIRTDNTHFKSFVNLNYKGDSKLGRNIRWQAWLSHYSFDVEHIKGTDNHFADFLSREFNKVNSSGGS

SEQ ID NO: 25 - protein, 329 aa, synthetic construct
MGIHGVPAAMKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 26 - protein, 235 aa, Human immunodeficiency virus 2
MEKEGQLEEAPPTNPYNTPTFAIKKKDKNKWRMLIDFRELNKVTQDFTEIQLGIPHPAGLAKKRRITVLDVGDAYFSIPLHEDFRPYTAFTLPSVNNAEPGKRYIYKVLPQGWKGSPAIFQHTMRQVLEPFRKANKDVIIIQYMDDILIASDRTDLEHDRVVLQLKELLNGLGFSTPDEKFQKDPPIHWMGYELWPTKWKLQKIQLPQKEIWTVNDIQKLVGVLNWAAQLYPGIK

SEQ ID NO: 27 - protein, 235 aa, Human immunodeficiency virus 1
MEEEGKISKIGPENPYNTPVFAIKKKDSTKWRKLVDFRELNKRTQDFWEVQLGIPHPAGLKKKKSVTVLDVGDAYFSVPLDESFRKYTAFTIPSINNETPGIRYQYNVLPQGWKGSPAIFQSSMTKILEPFRIKNPEIVIYQYMDDLYVGSDLEIGQHRTKIEELRAHLLSWGFTTPDKKHQKEPPFLWMGYELHPDRWTVQPIDLPEKDSWTVNDIQKLVGKLNWASQIYAGIK

SEQ ID NO: 28 - protein, 441 aa, Human immunodeficiency virus 1
MPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 29 - protein, 486 aa, synthetic construct
STLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLP

SEQ ID NO: 30 - RNA, 36 nt, synthetic construct
  source 1..36: mol_type = other RNA
gttgcaaaacccaagaaatccgtctttcattgacgg

SEQ ID NO: 31 - RNA, 36 nt, synthetic construct
  source 1..36: mol_type = other RNA
aatagcggccctaagaaatccgtctttcattgacgg

SEQ ID NO: 32 - RNA, 36 nt, synthetic construct
  source 1..36: mol_type = other RNA
attggaactggcgagaaatccgtctttcattgacgg

SEQ ID NO: 33 - RNA, 36 nt, synthetic construct
  source 1..36: mol_type = other RNA
ccagcaacacctaagaaatccgtctttcattgacgg

SEQ ID NO: 34 - RNA, 36 nt, synthetic construct
  source 1..36: mol_type = other RNA
cggcgctcgaataggaaatccgtctttcattgacgg

SEQ ID NO: 35 - RNA, 36 nt, synthetic construct
  source 1..36: mol_type = other RNA
gtggcaacacctaagaaatccgtctttcattgacgg

SEQ ID NO: 36 - RNA, 36 nt, synthetic construct
  source 1..36: mol_type = other RNA
gttgcaacacctaagaaatccgtctttcattgacgg

SEQ ID NO: 37 - RNA, 36 nt, synthetic construct
  source 1..36: mol_type = other RNA
gttgcaatgcctaagaaatccgtctttcattgacgg

SEQ ID NO: 38 - RNA, 34 nt, synthetic construct
  source 1..34: mol_type = other RNA
gcaacacctaagaaatccgtctttcattgacggg

SEQ ID NO: 39 - RNA, 23 nt, synthetic construct
  source 1..23: mol_type = other RNA
agaaatccgtctttcattgacgg

SEQ ID NO: 40 - RNA, 36 nt, synthetic construct
  source 1..36: mol_type = other RNA
tctcaacgatagtcagacatgtgtcctcagtgacac

SEQ ID NO: 41 - RNA, 36 nt, synthetic construct
  source 1..36: mol_type = other RNA
ttttaacaacactcaggcatgtgtccacagtgacac

SEQ ID NO: 42 - RNA, 36 nt, synthetic construct
  source 1..36: mol_type = other RNA
ttgaacggatactcagacatgtgtttccagtgacac

SEQ ID NO: 43 - RNA, 36 nt, synthetic construct
  source 1..36: mol_type = other RNA
tgccctcaatagtcagatgtgtgtccacagtgacac

SEQ ID NO: 44 - RNA, 36 nt, synthetic construct
  source 1..36: mol_type = other RNA
tctcaatgatacttagatacgtgtcctcagtgacac

SEQ ID NO: 45 - RNA, 36 nt, synthetic construct
  source 1..36: mol_type = other RNA
tctcaatgatactcagacatgtgtccccagtgacac

SEQ ID NO: 46 - RNA, 36 nt, synthetic construct
  source 1..36: mol_type = other RNA
tctcaatgatactaagacatgtgtcctcagtgacac

SEQ ID NO: 47 - RNA, 36 nt, synthetic construct
  source 1..36: mol_type = other RNA
tctcaactatactcagacatgtgtcctcagtgacac

SEQ ID NO: 48 - RNA, 36 nt, synthetic construct
  source 1..36: mol_type = other RNA
tctcaacgatactcagacatgtgtcctcagtgacac

SEQ ID NO: 49 - RNA, 36 nt, synthetic construct
  source 1..36: mol_type = other RNA
tctcaacgatactaagatatgtgtcctcagcgacac

SEQ ID NO: 50 - RNA, 36 nt, synthetic construct
  source 1..36: mol_type = other RNA
tctcaacgatactaagatatgtgtccccagtgacac

SEQ ID NO: 51 - RNA, 36 nt, synthetic construct
  source 1..36: mol_type = other RNA
tctcaacgatactaagatatgtgtccacagtgacac

SEQ ID NO: 52 - RNA, 36 nt, synthetic construct
  source 1..36: mol_type = other RNA
tctcaacaatactcagacatgtgtccccagtgacac

SEQ ID NO: 53 - RNA, 36 nt, synthetic construct
  source 1..36: mol_type = other RNA
tctcaacaatactaaggcatgtgtccccagtgaccc

SEQ ID NO: 54 - RNA, 36 nt, synthetic construct
  source 1..36: mol_type = other RNA
tctcaaagatactcagacacgtgtccccagtgacac

SEQ ID NO: 55 - RNA, 36 nt, synthetic construct
  source 1..36: mol_type = other RNA
tctcaaaaatactcagacatgtgtcctcagtgacac

SEQ ID NO: 56 - RNA, 36 nt, synthetic construct
  source 1..36: mol_type = other RNA
gcgaaacaacagtcagacatgtgtccccagtgacac

SEQ ID NO: 57 - RNA, 36 nt, synthetic construct
  source 1..36: mol_type = other RNA
cctcaacgatattaagacatgtgtccgcagtgacac

SEQ ID NO: 58 - RNA, 22 nt, synthetic construct
  source 1..22: mol_type = other RNA
agacatgtgtcctcagtgacac

SEQ ID NO: 59 - protein, 1798 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTSKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 60 - DNA, 20 bp, Homo sapiens
  source 1..20: mol_type = genomic DNA
caccacggctgtcgtcacca

SEQ ID NO: 61 - DNA, 20 bp, Homo sapiens
  source 1..20: mol_type = genomic DNA
ggattggtgacgacagccgt

SEQ ID NO: 62 - DNA, 20 bp, Homo sapiens
  source 1..20: mol_type = genomic DNA
cttgggattggtgacgacag

SEQ ID NO: 63 - RNA, 20 nt, synthetic construct
  source 1..20: mol_type = other RNA
caccacggctgtcgtcacca

SEQ ID NO: 64 - RNA, 20 nt, synthetic construct
  source 1..20: mol_type = other RNA
ggattggtgacgacagccgt

SEQ ID NO: 65 - RNA, 20 nt, synthetic construct
  source 1..20: mol_type = other RNA
cttgggattggtgacgacag

SEQ ID NO: 66 - RNA, 34 nt, synthetic construct
  source 1..34: mol_type = other RNA
actcctattccaccatggctgttgttactaatcc

SEQ ID NO: 67 - RNA, 44 nt, synthetic construct
  source 1..44: mol_type = other RNA
ctgagcccctactcctattccaccatggctgttgttactaatcc

SEQ ID NO: 68 - RNA, 54 nt, synthetic construct
  source 1..54: mol_type = other RNA
tgccgccctgctgagcccctactcctattccaccatggctgttgttactaatcc

SEQ ID NO: 69 - RNA, 54 nt, synthetic construct
  source 1..54: mol_type = other RNA
tgccgccctgctgagcccctactcctattccaccatggccgttgttactaatcc

SEQ ID NO: 70 - RNA, 44 nt, synthetic construct
  source 1..44: mol_type = other RNA
ctgagcccctactcctattccaccatggccgttgttactaatcc

SEQ ID NO: 71 - RNA, 54 nt, synthetic construct
  source 1..54: mol_type = other RNA
tgccgccctattatcaccctactcctattccaccatggccgttgttactaatcc

SEQ ID NO: 72 - RNA, 34 nt, synthetic construct
  source 1..34: mol_type = other RNA
tcattccttgggattggtgacaacggccatggtg

SEQ ID NO: 73 - RNA, 44 nt, synthetic construct
  source 1..44: mol_type = other RNA
gagaagtccctcattccttgggattggtgacaacggccatggtg

SEQ ID NO: 74 - RNA, 54 nt, synthetic construct
  source 1..54: mol_type = other RNA
tccactggaggagaagtccctcattccttgggattggtgacaacggccatggtg

SEQ ID NO: 75 - RNA, 34 nt, synthetic construct
  source 1..34: mol_type = other RNA
tccctcattccttgggattggtaacaacggccat

SEQ ID NO: 76 - RNA, 44 nt, synthetic construct
  source 1..44: mol_type = other RNA
ggaggagaagtccctcattccttgggattggtaacaacggccat

SEQ ID NO: 77 - RNA, 54 nt, synthetic construct
  source 1..54: mol_type = other RNA
caggtccactggaggagaagtccctcattccttgggattggtaacaacggccat

SEQ ID NO: 78 - RNA, 34 nt, synthetic construct
  source 1..34: mol_type = other RNA
actcctattccaccatggccgttgttactaatcc

SEQ ID NO: 79 - RNA, 34 nt, synthetic construct
  source 1..34: mol_type = other RNA
actcctattccactatggccgttgttactaatcc

SEQ ID NO: 80 - RNA, 44 nt, synthetic construct
  source 1..44: mol_type = other RNA
ctgagcccctactcctattccactatggccgttgttactaatcc

SEQ ID NO: 81 - RNA, 34 nt, synthetic construct
  source 1..34: mol_type = other RNA
actcctattctactatggccgttgttactaatcc

SEQ ID NO: 82 - RNA, 44 nt, synthetic construct
  source 1..44: mol_type = other RNA
ctgagcccctactcctattctactatggccgttgttactaatcc

SEQ ID NO: 83 - RNA, 34 nt, synthetic construct
  source 1..34: mol_type = other RNA
actcctattctactatggccgttgttaccaatcc

SEQ ID NO: 84 - RNA, 44 nt, synthetic construct
  source 1..44: mol_type = other RNA
ctgagcccctactcctattctactatggccgttgttaccaatcc

SEQ ID NO: 85 - RNA, 34 nt, synthetic construct
  source 1..34: mol_type = other RNA
actcctattctactatggccgttgtcaccaatcc

SEQ ID NO: 86 - RNA, 44 nt, synthetic construct
  source 1..44: mol_type = other RNA
ctgagcccctactcctattctactatggccgttgtcaccaatcc

SEQ ID NO: 87 - RNA, 34 nt, synthetic construct
  source 1..34: mol_type = other RNA
actcctattctactatggccgtcgtcaccaatcc

SEQ ID NO: 88 - RNA, 44 nt, synthetic construct
  source 1..44: mol_type = other RNA
ctgagcccctactcctattctactatggccgtcgtcaccaatcc

SEQ ID NO: 89 - RNA, 34 nt, synthetic construct
  source 1..34: mol_type = other RNA
actcctactctactatggccgtcgtcaccaatcc

SEQ ID NO: 90 - RNA, 44 nt, synthetic construct
  source 1..44: mol_type = other RNA
ctgagcccctactcctactctactatggccgtcgtcaccaatcc

SEQ ID NO: 91 - RNA, 20 nt, synthetic construct
  source 1..20: mol_type = other RNA
caaggaatgagggacttctc

SEQ ID NO: 92 - RNA, 35 nt, synthetic construct
  source 1..35: mol_type = other RNA
caaggaatgagggacttctcctccagtggacctga

SEQ ID NO: 93 - RNA, 50 nt, synthetic construct
  source 1..50: mol_type = other RNA
caaggaatgagggacttctcctccagtggacctgaaggacgagggatggg

SEQ ID NO: 94 - RNA, 20 nt, synthetic construct
  source 1..20: mol_type = other RNA
gaataggagtaggggctcag

SEQ ID NO: 95 - RNA, 35 nt, synthetic construct
  source 1..35: mol_type = other RNA
gaataggagtaggggctcagcagggcggcaatggt

SEQ ID NO: 96 - RNA, 50 nt, synthetic construct
  source 1..50: mol_type = other RNA
gaataggagtaggggctcagcagggcggcaatggtgtagcggcgggggcc

SEQ ID NO: 97 - RNA, 20 nt, synthetic construct
  source 1..20: mol_type = other RNA
ggtggaataggagtaggggc

SEQ ID NO: 98 - RNA, 35 nt, synthetic construct
  source 1..35: mol_type = other RNA
ggtggaataggagtaggggctcagcagggcggcaa

SEQ ID NO: 99 - RNA, 50 nt, synthetic construct
  source 1..50: mol_type = other RNA
ggtggaataggagtaggggctcagcagggcggcaatggtgtagcggcggg

SEQ ID NO: 100 - RNA, 13 nt, synthetic construct
  source 1..13: mol_type = other RNA
caaggaatgaggg

SEQ ID NO: 101 - RNA, 97 nt, synthetic construct
  source 1..97: mol_type = other RNA
actcctattccaccatggctgttgttactaatcccaaggaatgagggacttctcagaaatccgtctttcattgacggcaccacggctgtcgtcacca

SEQ ID NO: 102 - RNA, 112 nt, synthetic construct
  source 1..112: mol_type = other RNA
actcctattccaccatggctgttgttactaatcccaaggaatgagggacttctcctccagtggacctgaagaaatccgtctttcattgacggcaccacggctgtcgtcacca

SEQ ID NO: 103 - RNA, 127 nt, synthetic construct
  source 1..127: mol_type = other RNA
actcctattccaccatggctgttgttactaatcccaaggaatgagggacttctcctccagtggacctgaaggacgagggatgggagaaatccgtctttcattgacggcaccacggctgtcgtcacca

SEQ ID NO: 104 - RNA, 107 nt, synthetic construct
  source 1..107: mol_type = other RNA
ctgagcccctactcctattccaccatggctgttgttactaatcccaaggaatgagggacttctcagaaatccgtctttcattgacggcaccacggctgtcgtcacca

SEQ ID NO: 105 - RNA, 122 nt, synthetic construct
  source 1..122: mol_type = other RNA
ctgagcccctactcctattccaccatggctgttgttactaatcccaaggaatgagggacttctcctccagtggacctgaagaaatccgtctttcattgacggcaccacggctgtcgtcacca

SEQ ID NO: 106 - RNA, 137 nt, synthetic construct
  source 1..137: mol_type = other RNA
ctgagcccctactcctattccaccatggctgttgttactaatcccaaggaatgagggacttctcctccagtggacctgaaggacgagggatgggagaaatccgtctttcattgacggcaccacggctgtcgtcacca

SEQ ID NO: 107 - RNA, 117 nt, synthetic construct
  source 1..117: mol_type = other RNA
tgccgccctgctgagcccctactcctattccaccatggctgttgttactaatcccaaggaatgagggacttctcagaaatccgtctttcattgacggcaccacggctgtcgtcacca

SEQ ID NO: 108 - RNA, 132 nt, synthetic construct
  source 1..132: mol_type = other RNA
tgccgccctgctgagcccctactcctattccaccatggctgttgttactaatcccaaggaatgagggacttctcctccagtggacctgaagaaatccgtctttcattgacggcaccacggctgtcgtcacca

SEQ ID NO: 109 - RNA, 147 nt, synthetic construct
  source 1..147: mol_type = other RNA
tgccgccctgctgagcccctactcctattccaccatggctgttgttactaatcccaaggaatgagggacttctcctccagtggacctgaaggacgagggatgggagaaatccgtctttcattgacggcaccacggctgtcgtcacca

SEQ ID NO: 110 - RNA, 117 nt, synthetic construct
  source 1..117: mol_type = other RNA
tgccgccctgctgagcccctactcctattccaccatggccgttgttactaatcccaaggaatgagggacttctcagaaatccgtctttcattgacggcaccacggctgtcgtcacca

SEQ ID NO: 111 - RNA, 132 nt, synthetic construct
  source 1..132: mol_type = other RNA
tgccgccctgctgagcccctactcctattccaccatggccgttgttactaatcccaaggaatgagggacttctcctccagtggacctgaagaaatccgtctttcattgacggcaccacggctgtcgtcacca

SEQ ID NO: 112 - RNA, 147 nt, synthetic construct
  source 1..147: mol_type = other RNA
tgccgccctgctgagcccctactcctattccaccatggccgttgttactaatcccaaggaatgagggacttctcctccagtggacctgaaggacgagggatgggagaaatccgtctttcattgacggcaccacggctgtcgtcacca

SEQ ID NO: 113 - RNA, 107 nt, synthetic construct
  source 1..107: mol_type = other RNA
ctgagcccctactcctattccaccatggccgttgttactaatcccaaggaatgagggacttctcagaaatccgtctttcattgacggcaccacggctgtcgtcacca

SEQ ID NO: 114 - RNA, 122 nt, synthetic construct
  source 1..122: mol_type = other RNA
ctgagcccctactcctattccaccatggccgttgttactaatcccaaggaatgagggacttctcctccagtggacctgaagaaatccgtctttcattgacggcaccacggctgtcgtcacca

SEQ ID NO: 115 - RNA, 137 nt, synthetic construct
  source 1..137: mol_type = other RNA
ctgagcccctactcctattccaccatggccgttgttactaatcccaaggaatgagggacttctcctccagtggacctgaaggacgagggatgggagaaatccgtctttcattgacggcaccacggctgtcgtcacca

SEQ ID NO: 116 - RNA, 117 nt, synthetic construct
  source 1..117: mol_type = other RNA
tgccgccctattatcaccctactcctattccaccatggccgttgttactaatcccaaggaatgagggacttctcagaaatccgtctttcattgacggcaccacggctgtcgtcacca

SEQ ID NO: 117 - RNA, 132 nt, synthetic construct
  source 1..132: mol_type = other RNA
tgccgccctattatcaccctactcctattccaccatggccgttgttactaatcccaaggaatgagggacttctcctccagtggacctgaagaaatccgtctttcattgacggcaccacggctgtcgtcacca

SEQ ID NO: 118 - RNA, 147 nt, synthetic construct
  source 1..147: mol_type = other RNA
tgccgccctattatcaccctactcctattccaccatggccgttgttactaatcccaaggaatgagggacttctcctccagtggacctgaaggacgagggatgggagaaatccgtctttcattgacggcaccacggctgtcgtcacca

SEQ ID NO: 119 - RNA, 97 nt, synthetic construct
  source 1..97: mol_type = other RNA
tcattccttgggattggtgacaacggccatggtggaataggagtaggggctcagagaaatccgtctttcattgacggggattggtgacgacagccgt

SEQ ID NO: 120 - RNA, 112 nt, synthetic construct
  source 1..112: mol_type = other RNA
tcattccttgggattggtgacaacggccatggtggaataggagtaggggctcagcagggcggcaatggtagaaatccgtctttcattgacggggattggtgacgacagccgt

SEQ ID NO: 121 - RNA, 127 nt, synthetic construct
  source 1..127: mol_type = other RNA
tcattccttgggattggtgacaacggccatggtggaataggagtaggggctcagcagggcggcaatggtgtagcggcgggggccagaaatccgtctttcattgacggggattggtgacgacagccgt

SEQ ID NO: 122 - RNA, 107 nt, synthetic construct
  source 1..107: mol_type = other RNA
gagaagtccctcattccttgggattggtgacaacggccatggtggaataggagtaggggctcagagaaatccgtctttcattgacggggattggtgacgacagccgt

SEQ ID NO: 123 - RNA, 122 nt, synthetic construct
  source 1..122: mol_type = other RNA
gagaagtccctcattccttgggattggtgacaacggccatggtggaataggagtaggggctcagcagggcggcaatggtagaaatccgtctttcattgacggggattggtgacgacagccgt

SEQ ID NO: 124 - RNA, 137 nt, synthetic construct
  source 1..137: mol_type = other RNA
gagaagtccctcattccttgggattggtgacaacggccatggtggaataggagtaggggctcagcagggcggcaatggtgtagcggcgggggccagaaatccgtctttcattgacggggattggtgacgacagccgt

SEQ ID NO: 125 - RNA, 117 nt, synthetic construct
  source 1..117: mol_type = other RNA
tccactggaggagaagtccctcattccttgggattggtgacaacggccatggtggaataggagtaggggctcagagaaatccgtctttcattgacggggattggtgacgacagccgt

SEQ ID NO: 126 - RNA, 132 nt, synthetic construct
  source 1..132: mol_type = other RNA
tccactggaggagaagtccctcattccttgggattggtgacaacggccatggtggaataggagtaggggctcagcagggcggcaatggtagaaatccgtctttcattgacggggattggtgacgacagccgt

SEQ ID NO: 127 - RNA, 147 nt, synthetic construct
  source 1..147: mol_type = other RNA
tccactggaggagaagtccctcattccttgggattggtgacaacggccatggtggaataggagtaggggctcagcagggcggcaatggtgtagcggcgggggccagaaatccgtctttcattgacggggattggtgacgacagccgt

SEQ ID NO: 128 - RNA, 97 nt, synthetic construct
  source 1..97: mol_type = other RNA
tccctcattccttgggattggtaacaacggccatggtggaataggagtaggggcagaaatccgtctttcattgacggcttgggattggtgacgacag

SEQ ID NO: 129 - RNA, 112 nt, synthetic construct
  source 1..112: mol_type = other RNA
tccctcattccttgggattggtaacaacggccatggtggaataggagtaggggctcagcagggcggcaaagaaatccgtctttcattgacggcttgggattggtgacgacag

SEQ ID NO: 130 - RNA, 127 nt, synthetic construct
  source 1..127: mol_type = other RNA
tccctcattccttgggattggtaacaacggccatggtggaataggagtaggggctcagcagggcggcaatggtgtagcggcgggagaaatccgtctttcattgacggcttgggattggtgacgacag

SEQ ID NO: 131 - RNA, 107 nt, synthetic construct
  source 1..107: mol_type = other RNA
ggaggagaagtccctcattccttgggattggtaacaacggccatggtggaataggagtaggggcagaaatccgtctttcattgacggcttgggattggtgacgacag

SEQ ID NO: 132 - RNA, 122 nt, synthetic construct
  source 1..122: mol_type = other RNA
ggaggagaagtccctcattccttgggattggtaacaacggccatggtggaataggagtaggggctcagcagggcggcaaagaaatccgtctttcattgacggcttgggattggtgacgacag

SEQ ID NO: 133 - RNA, 137 nt, synthetic construct
  source 1..137: mol_type = other RNA
ggaggagaagtccctcattccttgggattggtaacaacggccatggtggaataggagtaggggctcagcagggcggcaatggtgtagcggcgggagaaatccgtctttcattgacggcttgggattggtgacgacag

SEQ ID NO: 134 - RNA, 117 nt, synthetic construct
  source 1..117: mol_type = other RNA
caggtccactggaggagaagtccctcattccttgggattggtaacaacggccatggtggaataggagtaggggcagaaatccgtctttcattgacggcttgggattggtgacgacag

SEQ ID NO: 135 - RNA, 132 nt, synthetic construct
  source 1..132: mol_type = other RNA
caggtccactggaggagaagtccctcattccttgggattggtaacaacggccatggtggaataggagtaggggctcagcagggcggcaaagaaatccgtctttcattgacggcttgggattggtgacgacag

SEQ ID NO: 136 - RNA, 147 nt, synthetic construct
  source 1..147: mol_type = other RNA
caggtccactggaggagaagtccctcattccttgggattggtaacaacggccatggtggaataggagtaggggctcagcagggcggcaatggtgtagcggcgggagaaatccgtctttcattgacggcttgggattggtgacgacag

SEQ ID NO: 137 - RNA, 90 nt, synthetic construct
  source 1..90: mol_type = other RNA
actcctattccaccatggctgttgttactaatcccaaggaatgagggagaaatccgtctttcattgacggcaccacggctgtcgtcacca

SEQ ID NO: 138 - RNA, 100 nt, synthetic construct
  source 1..100: mol_type = other RNA
ctgagcccctactcctattccaccatggctgttgttactaatcccaaggaatgagggagaaatccgtctttcattgacggcaccacggctgtcgtcacca

SEQ ID NO: 139 - RNA, 90 nt, synthetic construct
  source 1..90: mol_type = other RNA
actcctattccaccatggccgttgttactaatcccaaggaatgagggagaaatccgtctttcattgacggcaccacggctgtcgtcacca

SEQ ID NO: 140 - RNA, 97 nt, synthetic construct
  source 1..97: mol_type = other RNA
actcctattccaccatggccgttgttactaatcccaaggaatgagggacttctcagaaatccgtctttcattgacggcaccacggctgtcgtcacca

SEQ ID NO: 141 - RNA, 100 nt, synthetic construct
  source 1..100: mol_type = other RNA
ctgagcccctactcctattccaccatggccgttgttactaatcccaaggaatgagggagaaatccgtctttcattgacggcaccacggctgtcgtcacca

SEQ ID NO: 142 - RNA, 90 nt, synthetic construct
  source 1..90: mol_type = other RNA
actcctattccactatggccgttgttactaatcccaaggaatgagggagaaatccgtctttcattgacggcaccacggctgtcgtcacca

SEQ ID NO: 143 - RNA, 97 nt, synthetic construct
  source 1..97: mol_type = other RNA
actcctattccactatggccgttgttactaatcccaaggaatgagggacttctcagaaatccgtctttcattgacggcaccacggctgtcgtcacca

SEQ ID NO: 144 - RNA, 100 nt, synthetic construct
  source 1..100: mol_type = other RNA
ctgagcccctactcctattccactatggccgttgttactaatcccaaggaatgagggagaaatccgtctttcattgacggcaccacggctgtcgtcacca

SEQ ID NO: 145 - RNA, 107 nt, synthetic construct
  source 1..107: mol_type = other RNA
ctgagcccctactcctattccactatggccgttgttactaatcccaaggaatgagggacttctcagaaatccgtctttcattgacggcaccacggctgtcgtcacca

SEQ ID NO: 146 - RNA, 90 nt, synthetic construct
  source 1..90: mol_type = other RNA
actcctattctactatggccgttgttactaatcccaaggaatgagggagaaatccgtctttcattgacggcaccacggctgtcgtcacca

SEQ ID NO: 147 - RNA, 97 nt, synthetic construct
  source 1..97: mol_type = other RNA
actcctattctactatggccgttgttactaatcccaaggaatgagggacttctcagaaatccgtctttcattgacggcaccacggctgtcgtcacca

SEQ ID NO: 148 - RNA, 100 nt, synthetic construct
  source 1..100: mol_type = other RNA
ctgagcccctactcctattctactatggccgttgttactaatcccaaggaatgagggagaaatccgtctttcattgacggcaccacggctgtcgtcacca

SEQ ID NO: 149 - RNA, 107 nt, synthetic construct
  source 1..107: mol_type = other RNA
ctgagcccctactcctattctactatggccgttgttactaatcccaaggaatgagggacttctcagaaatccgtctttcattgacggcaccacggctgtcgtcacca

SEQ ID NO: 150 - RNA, 90 nt, synthetic construct
  source 1..90: mol_type = other RNA
actcctattctactatggccgttgttaccaatcccaaggaatgagggagaaatccgtctttcattgacggcaccacggctgtcgtcacca

SEQ ID NO: 151 - RNA, 97 nt, synthetic construct
  source 1..97: mol_type = other RNA
actcctattctactatggccgttgttaccaatcccaaggaatgagggacttctcagaaatccgtctttcattgacggcaccacggctgtcgtcacca

SEQ ID NO: 152 - RNA, 100 nt, synthetic construct
  source 1..100: mol_type = other RNA
ctgagcccctactcctattctactatggccgttgttaccaatcccaaggaatgagggagaaatccgtctttcattgacggcaccacggctgtcgtcacca

SEQ ID NO: 153 - RNA, 107 nt, synthetic construct
  source 1..107: mol_type = other RNA
ctgagcccctactcctattctactatggccgttgttaccaatcccaaggaatgagggacttctcagaaatccgtctttcattgacggcaccacggctgtcgtcacca

SEQ ID NO: 154 - RNA, 90 nt, synthetic construct
  source 1..90: mol_type = other RNA
actcctattctactatggccgttgtcaccaatcccaaggaatgagggagaaatccgtctttcattgacggcaccacggctgtcgtcacca

SEQ ID NO: 155 - RNA, 97 nt, synthetic construct
  source 1..97: mol_type = other RNA
actcctattctactatggccgttgtcaccaatcccaaggaatgagggacttctcagaaatccgtctttcattgacggcaccacggctgtcgtcacca

SEQ ID NO: 156 - RNA, 100 nt, synthetic construct
  source 1..100: mol_type = other RNA
ctgagcccctactcctattctactatggccgttgtcaccaatcccaaggaatgagggagaaatccgtctttcattgacggcaccacggctgtcgtcacca

SEQ ID NO: 157 - RNA, 107 nt, synthetic construct
  source 1..107: mol_type = other RNA
ctgagcccctactcctattctactatggccgttgtcaccaatcccaaggaatgagggacttctcagaaatccgtctttcattgacggcaccacggctgtcgtcacca

SEQ ID NO: 158 - RNA, 90 nt, synthetic construct
  source 1..90: mol_type = other RNA
actcctattctactatggccgtcgtcaccaatcccaaggaatgagggagaaatccgtctttcattgacggcaccacggctgtcgtcacca

SEQ ID NO: 159 - RNA, 97 nt, synthetic construct
  source 1..97: mol_type = other RNA
actcctattctactatggccgtcgtcaccaatcccaaggaatgagggacttctcagaaatccgtctttcattgacggcaccacggctgtcgtcacca

SEQ ID NO: 160 - RNA, 100 nt, synthetic construct
  source 1..100: mol_type = other RNA
ctgagcccctactcctattctactatggccgtcgtcaccaatcccaaggaatgagggagaaatccgtctttcattgacggcaccacggctgtcgtcacca

SEQ ID NO: 161 - RNA, 107 nt, synthetic construct
  source 1..107: mol_type = other RNA
ctgagcccctactcctattctactatggccgtcgtcaccaatcccaaggaatgagggacttctcagaaatccgtctttcattgacggcaccacggctgtcgtcacca

SEQ ID NO: 162 - RNA, 90 nt, synthetic construct
  source 1..90: mol_type = other RNA
actcctactctactatggccgtcgtcaccaatcccaaggaatgagggagaaatccgtctttcattgacggcaccacggctgtcgtcacca

SEQ ID NO: 163 - RNA, 97 nt, synthetic construct
  source 1..97: mol_type = other RNA
actcctactctactatggccgtcgtcaccaatcccaaggaatgagggacttctcagaaatccgtctttcattgacggcaccacggctgtcgtcacca

SEQ ID NO: 164 - RNA, 100 nt, synthetic construct
  source 1..100: mol_type = other RNA
ctgagcccctactcctactctactatggccgtcgtcaccaatcccaaggaatgagggagaaatccgtctttcattgacggcaccacggctgtcgtcacca

SEQ ID NO: 165 - RNA, 107 nt, synthetic construct
  source 1..107: mol_type = other RNA
ctgagcccctactcctactctactatggccgtcgtcaccaatcccaaggaatgagggacttctcagaaatccgtctttcattgacggcaccacggctgtcgtcacca

SEQ ID NO: 166 - RNA, 97 nt, synthetic construct; identical to SEQ ID NO: 101
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = 2'-O-methyl modified nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage

SEQ ID NO: 167 - RNA, 112 nt, synthetic construct; identical to SEQ ID NO: 102
  source 1..112: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 109: mod_base = OTHER; note = 2'-O-methyl modified nucleotide with phosphorothioate linkage
  modified_base 110: mod_base = OTHER; note = 2'-O-methyl modified nucleotide with phosphorothioate linkage
  modified_base 111: mod_base = OTHER; note = 2'-O-methyl modified nucleotide with phosphorothioate linkage

SEQ ID NO: 168 - RNA, 127 nt, synthetic construct; identical to SEQ ID NO: 103
  source 1..127: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage

SEQ ID NO: 169 - RNA, 107 nt, synthetic construct; identical to SEQ ID NO: 104
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage

SEQ ID NO: 170 - RNA, 122 nt, synthetic construct; identical to SEQ ID NO: 105
  source 1..122: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage

SEQ ID NO: 171 - RNA, 137 nt, synthetic construct; identical to SEQ ID NO: 106
  source 1..137: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide with phosphorothioate linkage
  modified_base 136: mod_base = OTHER; note = 2'-O-methyl modified nucleotide with phosphorothioate linkage

SEQ ID NO: 172 - RNA, 117 nt, synthetic construct; identical to SEQ ID NO: 107
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage

SEQ ID NO: 173 - RNA, 132 nt, synthetic construct; identical to SEQ ID NO: 108
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage

SEQ ID NO: 174 - RNA, 147 nt, synthetic construct; identical to SEQ ID NO: 109
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = 2'-O-methyl modified nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage

SEQ ID NO: 175 - RNA, 117 nt, synthetic construct; identical to SEQ ID NO: 110
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage

SEQ ID NO: 176 - RNA, 132 nt, synthetic construct; identical to SEQ ID NO: 111
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage

SEQ ID NO: 177 - RNA, 147 nt, synthetic construct; identical to SEQ ID NO: 112
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = 2'-O-methyl modified nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage

SEQ ID NO: 178 - RNA, 107 nt, synthetic construct; identical to SEQ ID NO: 113
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage

SEQ ID NO: 179 - RNA, 122 nt, synthetic construct; identical to SEQ ID NO: 114
  source 1..122: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage

SEQ ID NO: 180 - RNA, 137 nt, synthetic construct; identical to SEQ ID NO: 115
  source 1..137: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide with phosphorothioate linkage
  modified_base 136: mod_base = OTHER; note = 2'-O-methyl modified nucleotide with phosphorothioate linkage

SEQ ID NO: 181 - RNA, 117 nt, synthetic construct; identical to SEQ ID NO: 116
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage

SEQ ID NO: 182 - RNA, 132 nt, synthetic construct; identical to SEQ ID NO: 117
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage

SEQ ID NO: 183 - RNA, 147 nt, synthetic construct; identical to SEQ ID NO: 118
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = 2'-O-methyl modified nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage

SEQ ID NO: 184 - RNA, 107 nt, synthetic construct; identical to SEQ ID NO: 104
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 185 - RNA, 122 nt, synthetic construct; identical to SEQ ID NO: 105
  source 1..122: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = Nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage
  modified_base 122: mod_base = OTHER; note = 2'-O-methyl modified nucleotide

SEQ ID NO: 186 - RNA, 137 nt, synthetic construct; identical to SEQ ID NO: 106
  source 1..137: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 134: mod_base = OTHER; note = Nucleotide with phosphorothioate linkage
  modified_base 135: mod_base = OTHER; note = 2'-O-methyl modified nucleotide with phosphorothioate linkage
  modified_base 136: mod_base = OTHER; note = 2'-O-methyl modified nucleotide with phosphorothioate linkage
  modified_base 137: mod_base = OTHER; note = 2'-O-methyl modified nucleotide

SEQ ID NO: 187 - RNA, 117 nt, synthetic construct; identical to SEQ ID NO: 107
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = Nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage
  modified_base 117: mod_base = OTHER; note = 2'-O-methyl modified nucleotide

SEQ ID NO: 188 - RNA, 132 nt, synthetic construct; identical to SEQ ID NO: 108
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = Nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage
  modified_base 132: mod_base = OTHER; note = 2'-O-methyl modified nucleotide

SEQ ID NO: 189 - RNA, 147 nt, synthetic construct; identical to SEQ ID NO: 109
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = Nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage
  modified_base 147: mod_base = OTHER; note = 2'-O-methyl modified nucleotide

SEQ ID NO: 190 - RNA, 97 nt, synthetic construct; identical to SEQ ID NO: 119
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = 2'-O-methyl modified nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage

SEQ ID NO: 191 - RNA, 112 nt, synthetic construct; identical to SEQ ID NO: 120
  source 1..112: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 109: mod_base = OTHER; note = 2'-O-methyl modified nucleotide with phosphorothioate linkage
  modified_base 110: mod_base = OTHER; note = 2'-O-methyl modified nucleotide with phosphorothioate linkage
  modified_base 111: mod_base = OTHER; note = 2'-O-methyl modified nucleotide with phosphorothioate linkage

SEQ ID NO: 192 - RNA, 127 nt, synthetic construct; identical to SEQ ID NO: 121
  source 1..127: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage

SEQ ID NO: 193 - RNA, 107 nt, synthetic construct; identical to SEQ ID NO: 122
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage

SEQ ID NO: 194 - RNA, 122 nt, synthetic construct; identical to SEQ ID NO: 123
  source 1..122: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage

SEQ ID NO: 195 - RNA, 137 nt, synthetic construct; identical to SEQ ID NO: 124
  source 1..137: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide with phosphorothioate linkage
  modified_base 136: mod_base = OTHER; note = 2'-O-methyl modified nucleotide with phosphorothioate linkage

SEQ ID NO: 196 - RNA, 117 nt, synthetic construct; identical to SEQ ID NO: 125
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage

SEQ ID NO: 197 - RNA, 132 nt, synthetic construct; identical to SEQ ID NO: 126
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage

SEQ ID NO: 198 - RNA, 147 nt, synthetic construct; identical to SEQ ID NO: 127
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = 2'-O-methyl modified nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage

SEQ ID NO: 199 - RNA, 97 nt, synthetic construct; identical to SEQ ID NO: 128
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = 2'-O-methyl modified nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage

SEQ ID NO: 200 - RNA, 112 nt, synthetic construct; identical to SEQ ID NO: 129
  source 1..112: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 109: mod_base = OTHER; note = 2'-O-methyl modified nucleotide with phosphorothioate linkage
  modified_base 110: mod_base = OTHER; note = 2'-O-methyl modified nucleotide with phosphorothioate linkage
  modified_base 111: mod_base = OTHER; note = 2'-O-methyl modified nucleotide with phosphorothioate linkage

SEQ ID NO: 201 - RNA, 127 nt, synthetic construct; identical to SEQ ID NO: 130
  source 1..127: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 124: mod_base = OTHER; note = 2'-O-methyl modified nucleotide with phosphorothioate linkage
  modified_base 125: mod_base = OTHER; note = 2'-O-methyl modified nucleotide with phosphorothioate linkage
  modified_base 126: mod_base = OTHER; note = 2'-O-methyl modified nucleotide with phosphorothioate linkage

SEQ ID NO: 202 - RNA, 107 nt, synthetic construct; identical to SEQ ID NO: 131
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage

SEQ ID NO: 203 - RNA, 122 nt, synthetic construct; identical to SEQ ID NO: 132
  source 1..122: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 119: mod_base = OTHER; note = 2'-O-methyl modified nucleotide with phosphorothioate linkage
  modified_base 120: mod_base = OTHER; note = 2'-O-methyl modified nucleotide with phosphorothioate linkage
  modified_base 121: mod_base = OTHER; note = 2'-O-methyl modified nucleotide with phosphorothioate linkage

SEQ ID NO: 204 - RNA, 137 nt, synthetic construct; identical to SEQ ID NO: 133
  source 1..137: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 134: mod_base = OTHER; note = 2'-O-methyl modified nucleotide with phosphorothioate linkage
  modified_base 135: mod_base = OTHER; note = 2'-O-methyl modified nucleotide with phosphorothioate linkage
  modified_base 136: mod_base = OTHER; note = 2'-O-methyl modified nucleotide with phosphorothioate linkage

SEQ ID NO: 205 - RNA, 117 nt, synthetic construct; identical to SEQ ID NO: 134
  source 1..117: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 114: mod_base = OTHER; note = 2'-O-methyl modified nucleotide with phosphorothioate linkage
  modified_base 115: mod_base = OTHER; note = 2'-O-methyl modified nucleotide with phosphorothioate linkage
  modified_base 116: mod_base = OTHER; note = 2'-O-methyl modified nucleotide with phosphorothioate linkage

SEQ ID NO: 206 - RNA, 132 nt, synthetic construct; identical to SEQ ID NO: 135
  source 1..132: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 129: mod_base = OTHER; note = 2'-O-methyl modified nucleotide with phosphorothioate linkage
  modified_base 130: mod_base = OTHER; note = 2'-O-methyl modified nucleotide with phosphorothioate linkage
  modified_base 131: mod_base = OTHER; note = 2'-O-methyl modified nucleotide with phosphorothioate linkage

SEQ ID NO: 207 - RNA, 147 nt, synthetic construct; identical to SEQ ID NO: 136
  source 1..147: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 144: mod_base = OTHER; note = 2'-O-methyl modified nucleotide with phosphorothioate linkage
  modified_base 145: mod_base = OTHER; note = 2'-O-methyl modified nucleotide with phosphorothioate linkage
  modified_base 146: mod_base = OTHER; note = 2'-O-methyl modified nucleotide with phosphorothioate linkage

SEQ ID NO: 208 - RNA, 90 nt, synthetic construct; identical to SEQ ID NO: 137
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 209 - RNA, 97 nt, synthetic construct; identical to SEQ ID NO: 101
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 210 - RNA, 100 nt, synthetic construct; identical to SEQ ID NO: 138
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 211 - RNA, 90 nt, synthetic construct; identical to SEQ ID NO: 139
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 212 - RNA, 97 nt, synthetic construct; identical to SEQ ID NO: 140
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 213 - RNA, 107 nt, synthetic construct; identical to SEQ ID NO: 165
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 214 - RNA, 107 nt, synthetic construct; identical to SEQ ID NO: 113
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 215 - RNA, 90 nt, synthetic construct; identical to SEQ ID NO: 142
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 216 - RNA, 97 nt, synthetic construct; identical to SEQ ID NO: 143
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 217 - RNA, 100 nt, synthetic construct; identical to SEQ ID NO: 144
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 218 - RNA, 107 nt, synthetic construct; identical to SEQ ID NO: 145
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 219 - RNA, 90 nt, synthetic construct; identical to SEQ ID NO: 146
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 220 - RNA, 97 nt, synthetic construct; identical to SEQ ID NO: 147
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 221 - RNA, 100 nt, synthetic construct; identical to SEQ ID NO: 148
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 222 - RNA, 107 nt, synthetic construct; identical to SEQ ID NO: 149
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 223 - RNA, 90 nt, synthetic construct; identical to SEQ ID NO: 150
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 224 - RNA, 97 nt, synthetic construct; identical to SEQ ID NO: 151
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 225 - RNA, 100 nt, synthetic construct; identical to SEQ ID NO: 152
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 226 - RNA, 107 nt, synthetic construct; identical to SEQ ID NO: 153
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 227 - RNA, 90 nt, synthetic construct; identical to SEQ ID NO: 154
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 228 - RNA, 97 nt, synthetic construct; identical to SEQ ID NO: 155
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 229 - RNA, 100 nt, synthetic construct; identical to SEQ ID NO: 156
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 230 - RNA, 107 nt, synthetic construct; identical to SEQ ID NO: 157
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 231 - RNA, 90 nt, synthetic construct; identical to SEQ ID NO: 158
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 232 - RNA, 97 nt, synthetic construct; identical to SEQ ID NO: 159
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 233 - RNA, 100 nt, synthetic construct; identical to SEQ ID NO: 160
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 234 - RNA, 107 nt, synthetic construct; identical to SEQ ID NO: 161
  source 1..107: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 104: mod_base = OTHER; note = Nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide with phosphorothioate linkage
  modified_base 107: mod_base = OTHER; note = 2'-O-methyl modified nucleotide

SEQ ID NO: 235 - RNA, 90 nt, synthetic construct; identical to SEQ ID NO: 162
  source 1..90: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 87: mod_base = OTHER; note = Nucleotide with phosphorothioate linkage
  modified_base 88: mod_base = OTHER; note = 2'-O-methyl modified nucleotide with phosphorothioate linkage
  modified_base 89: mod_base = OTHER; note = 2'-O-methyl modified nucleotide with phosphorothioate linkage
  modified_base 90: mod_base = OTHER; note = 2'-O-methyl modified nucleotide

SEQ ID NO: 236 - RNA, 97 nt, synthetic construct; identical to SEQ ID NO: 163
  source 1..97: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 94: mod_base = OTHER; note = Nucleotide with phosphorothioate linkage
  modified_base 95: mod_base = OTHER; note = 2'-O-methyl modified nucleotide with phosphorothioate linkage
  modified_base 96: mod_base = OTHER; note = 2'-O-methyl modified nucleotide with phosphorothioate linkage
  modified_base 97: mod_base = OTHER; note = 2'-O-methyl modified nucleotide

SEQ ID NO: 237 - RNA, 100 nt, synthetic construct; identical to SEQ ID NO: 164
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 97: mod_base = OTHER; note = Nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 238 - protein, 1818 aa, synthetic construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQKKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 239 - skipped

SEQ ID NO: 240 - DNA, 20 bp, Homo sapiens
  source 1..20: mol_type = genomic DNA
ctcctattccaccacggctg

SEQ ID NO: 241 - DNA, 20 bp, Homo sapiens
  source 1..20: mol_type = genomic DNA
ctattccaccacggctgtcg

SEQ ID NO: 242 - DNA, 20 bp, Homo sapiens
  source 1..20: mol_type = genomic DNA
ttccaccacggctgtcgtca

SEQ ID NO: 243 - DNA, 20 bp, Homo sapiens
  source 1..20: mol_type = genomic DNA
ccaccacggctgtcgtcacc

SEQ ID NO: 244 - DNA, 20 bp, Homo sapiens
  source 1..20: mol_type = genomic DNA
gggattggtgacgacagccg

SEQ ID NO: 245 - DNA, 20 bp, Homo sapiens
  source 1..20: mol_type = genomic DNA
ggtgacgacagccgtggtgg

SEQ ID NO: 246 - DNA, 20 bp, Homo sapiens
  source 1..20: mol_type = genomic DNA
gtgacgacagccgtggtgga

SEQ ID NO: 247 - DNA, 20 bp, Homo sapiens
  source 1..20: mol_type = genomic DNA
acgacagccgtggtggaata

SEQ ID NO: 248 - RNA, 20 nt, synthetic construct
  source 1..20: mol_type = other RNA
ctcctattccaccacggctg

SEQ ID NO: 249 - RNA, 20 nt, synthetic construct
  source 1..20: mol_type = other RNA
ctattccaccacggctgtcg

SEQ ID NO: 250 - RNA, 20 nt, synthetic construct
  source 1..20: mol_type = other RNA
ttccaccacggctgtcgtca

SEQ ID NO: 251 - RNA, 20 nt, synthetic construct
  source 1..20: mol_type = other RNA
ccaccacggctgtcgtcacc

SEQ ID NO: 252 - RNA, 20 nt, synthetic construct
  source 1..20: mol_type = other RNA
gggattggtgacgacagccg

SEQ ID NO: 253 - RNA, 20 nt, synthetic construct
  source 1..20: mol_type = other RNA
ggtgacgacagccgtggtgg

SEQ ID NO: 254 - RNA, 20 nt, synthetic construct
  source 1..20: mol_type = other RNA
gtgacgacagccgtggtgga

SEQ ID NO: 255 - RNA, 20 nt, synthetic construct
  source 1..20: mol_type = other RNA
acgacagccgtggtggaata

SEQ ID NO: 256 - RNA, 44 nt, synthetic construct
  source 1..44: mol_type = other RNA
gccgccctgctgagcccctactcctattctactatggccgtcgt

SEQ ID NO: 257 - RNA, 44 nt, synthetic construct
  source 1..44: mol_type = other RNA
gccgccctgctgagcccctactcctatagtactatggccgtcgt

SEQ ID NO: 258 - RNA, 44 nt, synthetic construct
  source 1..44: mol_type = other RNA
gccgccctgctgagcccctacagttatagtactatggccgtcgt

SEQ ID NO: 259 - RNA, 44 nt, synthetic construct
  source 1..44: mol_type = other RNA
gccctgctgagcccctactcctattccactatggccgttgtcac

SEQ ID NO: 260 - RNA, 44 nt, synthetic construct
  source 1..44: mol_type = other RNA
gccctgctgagcccctactcctattctactatggccgttgtcac

SEQ ID NO: 261 - RNA, 44 nt, synthetic construct
  source 1..44: mol_type = other RNA
gccctgctgagcccctactcctatagtactatggccgttgtcac

SEQ ID NO: 262 - RNA, 44 nt, synthetic construct
  source 1..44: mol_type = other RNA
ctgctgagcccctactcctattccaccatggccgttgttaccaa

SEQ ID NO: 263 - RNA, 44 nt, synthetic construct
  source 1..44: mol_type = other RNA
ctgctgagcccctactcctattccactatggccgttgttaccaa

SEQ ID NO: 264 - RNA, 44 nt, synthetic construct
  source 1..44: mol_type = other RNA
ctgctgagcccctactcctattctactatggccgttgttaccaa

SEQ ID NO: 265 - RNA, 44 nt, synthetic construct
  source 1..44: mol_type = other RNA
gctgagcccctactcctattccaccatggctgttgttactaatc

SEQ ID NO: 266 - RNA, 44 nt, synthetic construct
  source 1..44: mol_type = other RNA
gctgagcccctactcctattccaccatggccgttgttactaatc

SEQ ID NO: 267 - RNA, 44 nt, synthetic construct
  source 1..44: mol_type = other RNA
gctgagcccctactcctattccactatggccgttgttactaatc

SEQ ID NO: 268 - RNA, 44 nt, synthetic construct
  source 1..44: mol_type = other RNA
ggagaagtccctcattccttgggattggtaacaacggccatggt

SEQ ID NO: 269 - RNA, 44 nt, synthetic construct
  source 1..44: mol_type = other RNA
ggagaagtccctcattccttgggattagtaacaacggccatggt

SEQ ID NO: 270 - RNA, 44 nt, synthetic construct
  source 1..44: mol_type = other RNA
ggagaagtccctcattccttggggttagtaacaacggccatggt

SEQ ID NO: 271 - RNA, 44 nt, synthetic construct
  source 1..44: mol_type = other RNA
gagaagtccctcattccttgggattggtaacaacggccatggtg

SEQ ID NO: 272 - RNA, 44 nt, synthetic construct
  source 1..44: mol_type = other RNA
gagaagtccctcattccttgggattagtaacaacggccatggtg

SEQ ID NO: 273 - RNA, 44 nt, synthetic construct
  source 1..44: mol_type = other RNA
gagaagtccctcattccttggggttagtaacaacggccatggtg

SEQ ID NO: 274 - RNA, 44 nt, synthetic construct
  source 1..44: mol_type = other RNA
gtccctcattccttgggattggtgacgacggccatagtagaata

SEQ ID NO: 275 - RNA, 44 nt, synthetic construct
  source 1..44: mol_type = other RNA
gtccctcattccttgggattggtgacaacggccatagtagaata

SEQ ID NO: 276 - RNA, 44 nt, synthetic construct
  source 1..44: mol_type = other RNA
gtccctcattccttgggattggtaacaacggccatagtagaata

SEQ ID NO: 277 - RNA, 44 nt, synthetic construct
  source 1..44: mol_type = other RNA
tccctcattccttgggattggtgacaacggccatagtagaatag

SEQ ID NO: 278 - RNA, 44 nt, synthetic construct
  source 1..44: mol_type = other RNA
tccctcattccttgggattggtgacgacggccatagtactatag

SEQ ID NO: 279 - RNA, 44 nt, synthetic construct
  source 1..44: mol_type = other RNA
tccctcattccttgggattggtaacaacggccatagtactatag

SEQ ID NO: 280 - RNA, 44 nt, synthetic construct
  source 1..44: mol_type = other RNA
ctcattccttgggattggtgacgacggccatagtagaataggag

SEQ ID NO: 281 - RNA, 44 nt, synthetic construct
  source 1..44: mol_type = other RNA
ctcattccttgggattggtgacgacggccatagtactataggag

SEQ ID NO: 282 - RNA, 44 nt, synthetic construct
  source 1..44: mol_type = other RNA
ctcattccttgggattggtgacaacggccatagtactataggag

SEQ ID NO: 283 - RNA, 20 nt, synthetic construct
  source 1..20: mol_type = other RNA
caccaatcccaaggaatgag

SEQ ID NO: 284 - RNA, 20 nt, synthetic construct
  source 1..20: mol_type = other RNA
caatcccaaggaatgaggga

SEQ ID NO: 285 - RNA, 20 nt, synthetic construct
  source 1..20: mol_type = other RNA
tcccaaggaatgagggactt

SEQ ID NO: 286 - RNA, 20 nt, synthetic construct
  source 1..20: mol_type = other RNA
ccaaggaatgagggacttct

SEQ ID NO: 287 - RNA, 20 nt, synthetic construct
  source 1..20: mol_type = other RNA
ggaataggagtaggggctca

SEQ ID NO: 288 - RNA, 20 nt, synthetic construct
  source 1..20: mol_type = other RNA
ggagtaggggctcagcaggg

SEQ ID NO: 289 - RNA, 20 nt, synthetic construct
  source 1..20: mol_type = other RNA
gagtaggggctcagcagggc

SEQ ID NO: 290 - RNA, 20 nt, synthetic construct
  source 1..20: mol_type = other RNA
taggggctcagcagggcggc

SEQ ID NO: 291 - RNA, 106 nt, synthetic construct
  source 1..106: mol_type = other RNA
gccgccctgctgagcccctactcctattctactatggccgtcgtcaccaatcccaaggaatgagagacatgtgtcctcagtgacacctcctattccaccacggctg

SEQ ID NO: 292 - RNA, 106 nt, synthetic construct
  source 1..106: mol_type = other RNA
gccgccctgctgagcccctactcctatagtactatggccgtcgtcaccaatcccaaggaatgagagacatgtgtcctcagtgacacctcctattccaccacggctg

SEQ ID NO: 293 - RNA, 106 nt, synthetic construct
  source 1..106: mol_type = other RNA
gccgccctgctgagcccctacagttatagtactatggccgtcgtcaccaatcccaaggaatgagagacatgtgtcctcagtgacacctcctattccaccacggctg

SEQ ID NO: 294 - RNA, 106 nt, synthetic construct
  source 1..106: mol_type = other RNA
gccctgctgagcccctactcctattccactatggccgttgtcaccaatcccaaggaatgagggaagacatgtgtcctcagtgacacctattccaccacggctgtcg

SEQ ID NO: 295 - RNA, 106 nt, synthetic construct
  source 1..106: mol_type = other RNA
gccctgctgagcccctactcctattctactatggccgttgtcaccaatcccaaggaatgagggaagacatgtgtcctcagtgacacctattccaccacggctgtcg

SEQ ID NO: 296 - RNA, 106 nt, synthetic construct
  source 1..106: mol_type = other RNA
gccctgctgagcccctactcctatagtactatggccgttgtcaccaatcccaaggaatgagggaagacatgtgtcctcagtgacacctattccaccacggctgtcg

SEQ ID NO: 297 - RNA, 106 nt, synthetic construct
  source 1..106: mol_type = other RNA
ctgctgagcccctactcctattccaccatggccgttgttaccaatcccaaggaatgagggacttagacatgtgtcctcagtgacacttccaccacggctgtcgtca

SEQ ID NO: 298 - RNA, 106 nt, synthetic construct
  source 1..106: mol_type = other RNA
ctgctgagcccctactcctattccactatggccgttgttaccaatcccaaggaatgagggacttagacatgtgtcctcagtgacacttccaccacggctgtcgtca

SEQ ID NO: 299 - RNA, 106 nt, synthetic construct
  source 1..106: mol_type = other RNA
ctgctgagcccctactcctattctactatggccgttgttaccaatcccaaggaatgagggacttagacatgtgtcctcagtgacacttccaccacggctgtcgtca

SEQ ID NO: 300 - RNA, 106 nt, synthetic construct
  source 1..106: mol_type = other RNA
gctgagcccctactcctattccaccatggctgttgttactaatcccaaggaatgagggacttctagacatgtgtcctcagtgacacccaccacggctgtcgtcacc

SEQ ID NO: 301 - RNA, 106 nt, synthetic construct
  source 1..106: mol_type = other RNA
gctgagcccctactcctattccaccatggccgttgttactaatcccaaggaatgagggacttctagacatgtgtcctcagtgacacccaccacggctgtcgtcacc

SEQ ID NO: 302 - RNA, 106 nt, synthetic construct
  source 1..106: mol_type = other RNA
gctgagcccctactcctattccactatggccgttgttactaatcccaaggaatgagggacttctagacatgtgtcctcagtgacacccaccacggctgtcgtcacc

SEQ ID NO: 303 - RNA, 106 nt, synthetic construct
  source 1..106: mol_type = other RNA
ctgagcccctactcctattccaccatggctgttgttactaatcccaaggaatgagggacttctcagacatgtgtcctcagtgacaccaccacggctgtcgtcacca

SEQ ID NO: 304 - RNA, 106 nt, synthetic construct
  source 1..106: mol_type = other RNA
ctgagcccctactcctattccaccatggccgttgttactaatcccaaggaatgagggacttctcagacatgtgtcctcagtgacaccaccacggctgtcgtcacca

SEQ ID NO: 305 - RNA, 106 nt, synthetic construct
  source 1..106: mol_type = other RNA
ctgagcccctactcctattccactatggccgttgttactaatcccaaggaatgagggacttctcagacatgtgtcctcagtgacaccaccacggctgtcgtcacca

SEQ ID NO: 306 - RNA, 106 nt, synthetic construct
  source 1..106: mol_type = other RNA
ggagaagtccctcattccttgggattggtaacaacggccatggtggaataggagtaggggctcaagacatgtgtcctcagtgacacgggattggtgacgacagccg

SEQ ID NO: 307 - RNA, 106 nt, synthetic construct
  source 1..106: mol_type = other RNA
ggagaagtccctcattccttgggattagtaacaacggccatggtggaataggagtaggggctcaagacatgtgtcctcagtgacacgggattggtgacgacagccg

SEQ ID NO: 308 - RNA, 106 nt, synthetic construct
  source 1..106: mol_type = other RNA
ggagaagtccctcattccttggggttagtaacaacggccatggtggaataggagtaggggctcaagacatgtgtcctcagtgacacgggattggtgacgacagccg

SEQ ID NO: 309 - RNA, 106 nt, synthetic construct
  source 1..106: mol_type = other RNA
gagaagtccctcattccttgggattggtaacaacggccatggtggaataggagtaggggctcagagacatgtgtcctcagtgacacggattggtgacgacagccgt

SEQ ID NO: 310 - RNA, 106 nt, synthetic construct
  source 1..106: mol_type = other RNA
gagaagtccctcattccttgggattagtaacaacggccatggtggaataggagtaggggctcagagacatgtgtcctcagtgacacggattggtgacgacagccgt

SEQ ID NO: 311 - RNA, 106 nt, synthetic construct
  source 1..106: mol_type = other RNA
gagaagtccctcattccttggggttagtaacaacggccatggtggaataggagtaggggctcagagacatgtgtcctcagtgacacggattggtgacgacagccgt

SEQ ID NO: 312 - RNA, 106 nt, synthetic construct
  source 1..106: mol_type = other RNA
gtccctcattccttgggattggtgacgacggccatagtagaataggagtaggggctcagcagggagacatgtgtcctcagtgacacggtgacgacagccgtggtgg

SEQ ID NO: 313 - RNA, 106 nt, synthetic construct
  source 1..106: mol_type = other RNA
gtccctcattccttgggattggtgacaacggccatagtagaataggagtaggggctcagcagggagacatgtgtcctcagtgacacggtgacgacagccgtggtgg

SEQ ID NO: 314 - RNA, 106 nt, synthetic construct
  source 1..106: mol_type = other RNA
gtccctcattccttgggattggtaacaacggccatagtagaataggagtaggggctcagcagggagacatgtgtcctcagtgacacggtgacgacagccgtggtgg

SEQ ID NO: 315 - RNA, 106 nt, synthetic construct
  source 1..106: mol_type = other RNA
tccctcattccttgggattggtgacaacggccatagtagaataggagtaggggctcagcagggcagacatgtgtcctcagtgacacgtgacgacagccgtggtgga

SEQ ID NO: 316 - RNA, 106 nt, synthetic construct
  source 1..106: mol_type = other RNA
tccctcattccttgggattggtgacgacggccatagtactataggagtaggggctcagcagggcagacatgtgtcctcagtgacacgtgacgacagccgtggtgga

SEQ ID NO: 317 - RNA, 106 nt, synthetic construct
  source 1..106: mol_type = other RNA
tccctcattccttgggattggtaacaacggccatagtactataggagtaggggctcagcagggcagacatgtgtcctcagtgacacgtgacgacagccgtggtgga

SEQ ID NO: 318 - RNA, 106 nt, synthetic construct
  source 1..106: mol_type = other RNA
ctcattccttgggattggtgacgacggccatagtagaataggagtaggggctcagcagggcggcagacatgtgtcctcagtgacacacgacagccgtggtggaata

SEQ ID NO: 319 - RNA, 106 nt, synthetic construct
  source 1..106: mol_type = other RNA
ctcattccttgggattggtgacgacggccatagtactataggagtaggggctcagcagggcggcagacatgtgtcctcagtgacacacgacagccgtggtggaata

SEQ ID NO: 320 - RNA, 106 nt, synthetic construct
  source 1..106: mol_type = other RNA
ctcattccttgggattggtgacaacggccatagtactataggagtaggggctcagcagggcggcagacatgtgtcctcagtgacacacgacagccgtggtggaata

SEQ ID NO: 321 - RNA, 106 nt, synthetic construct; identical to SEQ ID NO: 291
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 322 - RNA, 106 nt, synthetic construct; identical to SEQ ID NO: 292
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 323 - RNA, 106 nt, synthetic construct; identical to SEQ ID NO: 293
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 324 - RNA, 106 nt, synthetic construct; identical to SEQ ID NO: 294
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 325 - RNA, 106 nt, synthetic construct; identical to SEQ ID NO: 295
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 326 - RNA, 106 nt, synthetic construct; identical to SEQ ID NO: 296
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 327 - RNA, 106 nt, synthetic construct; identical to SEQ ID NO: 297
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 328 - RNA, 106 nt, synthetic construct; identical to SEQ ID NO: 298
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 329 - RNA, 106 nt, synthetic construct; identical to SEQ ID NO: 299
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 330 - RNA, 106 nt, synthetic construct; identical to SEQ ID NO: 300
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 331 - RNA, 106 nt, synthetic construct; identical to SEQ ID NO: 301
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 332 - RNA, 106 nt, synthetic construct; identical to SEQ ID NO: 302
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 333 - RNA, 106 nt, synthetic construct; identical to SEQ ID NO: 303
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 334 - RNA, 106 nt, synthetic construct; identical to SEQ ID NO: 304
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 335 - RNA, 106 nt, synthetic construct; identical to SEQ ID NO: 305
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 336 - RNA, 106 nt, synthetic construct; identical to SEQ ID NO: 306
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 337 - RNA, 106 nt, synthetic construct; identical to SEQ ID NO: 307
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 338 - RNA, 106 nt, synthetic construct; identical to SEQ ID NO: 308
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 339 - RNA, 106 nt, synthetic construct; identical to SEQ ID NO: 309
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 340 - RNA, 106 nt, synthetic construct; identical to SEQ ID NO: 310
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 341 - RNA, 106 nt, synthetic construct; identical to SEQ ID NO: 311
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 342 - RNA, 106 nt, synthetic construct; identical to SEQ ID NO: 312
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 343 - RNA, 106 nt, synthetic construct; identical to SEQ ID NO: 313
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 344 - RNA, 106 nt, synthetic construct; identical to SEQ ID NO: 314
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 345 - RNA, 106 nt, synthetic construct; identical to SEQ ID NO: 315
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 346 - RNA, 106 nt, synthetic construct; identical to SEQ ID NO: 316
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 347 - RNA, 106 nt, synthetic construct; identical to SEQ ID NO: 317
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 348 - RNA, 106 nt, synthetic construct; identical to SEQ ID NO: 318
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 349 - RNA, 106 nt, synthetic construct; identical to SEQ ID NO: 319
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 350 - RNA, 106 nt, synthetic construct; identical to SEQ ID NO: 320
  source 1..106: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 103: mod_base = OTHER; note = Nucleotide with phosphorothioate linkage
  modified_base 104: mod_base = OTHER; note = 2'-O-methyl modified nucleotide with phosphorothioate linkage
  modified_base 105: mod_base = OTHER; note = 2'-O-methyl modified nucleotide with phosphorothioate linkage
  modified_base 106: mod_base = OTHER; note = 2'-O-methyl modified nucleotide

SEQ ID NO: 351 - DNA, 34 bp, synthetic construct
  source 1..34: mol_type = other DNA
actcctattccaccacggctgtcgtcaccaatcc

SEQ ID NO: 352 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 353 - protein, 1798 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTSKRPAATKKAGQAKKKKSGGSSGGSSGSETPGTSESATPESSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPMKRTADGSEFESPKKKRKV

SEQ ID NO: 354 - RNA, 43 nt, synthetic construct
  source 1..43: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
  modified_base 26: mod_base = OTHER; note = 2'-O-methyl modified nucleotide
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide with phosphorothioate linkage
  modified_base 41: mod_base = OTHER; note = 2'-O-methyl modified nucleotide with phosphorothioate linkage
  modified_base 42: mod_base = OTHER; note = 2'-O-methyl modified nucleotide with phosphorothioate linkage
  modified_base 43: mod_base = OTHER; note = 2'-O-methyl modified nucleotide
agaaatccgtctttcattgacggcaccacggctgtcgtcacca

SEQ ID NO: 355 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKAHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 356 - DNA, 23 bp, synthetic construct
  source 1..23: mol_type = other DNA
agaaatccgtctttcattgacgg

SEQ ID NO: 357 - DNA, 22 bp, synthetic construct
  source 1..22: mol_type = other DNA
agacatgtgtcctcagtgacac